(19) United States
(12) Patent Application Publication
(10) Pub. No.: US 2024/0155940 A1
(43) Pub. Date: May 9, 2024

(54) COMPOUND FOR ORGANIC OPTOELECTRONIC DEVICE, COMPOSITION FOR ORGANIC OPTOELECTRONIC DEVICE, ORGANIC OPTOELECTRONIC DEVICE AND DISPLAY DEVICE

(71) Applicant: SAMSUNG SDI CO., LTD., Yongin-si (KR)

(72) Inventors: Seonyeong GWAK, Suwon-si (KR); Chang Ju SHIN, Suwon-si (KR); Sunghyun JUNG, Suwon-si (KR); Hyung Sun KIM, Suwon-si (KR); Hanill LEE, Suwon-si (KR); Yoonman LEE, Suwon-si (KR); Byungku KIM, Suwon-si (KR); Wook KIM, Suwon-si (KR); Seungjae LEE, Suwon-si (KR); Suheon IM, Suwon-si (KR); Ho Kuk JUNG, Suwon-si (KR); Mijin LEE, Suwon-si (KR); Youngkyoung JO, Suwon-si (KR)

(21) Appl. No.: 18/377,352

(22) Filed: Oct. 6, 2023

(30) Foreign Application Priority Data
Oct. 7, 2022 (KR) 10-2022-0128379

(51) Int. Cl. H10K 85/60; C09K 11/06

(52) U.S. Cl. CPC H10K 85/6572; C09K 11/06; H10K 85/654; H10K 85/6574; H10K 85/6576; C07B 2200/05

(57) ABSTRACT

A compound for an organic optoelectronic device, a composition for an organic optoelectronic device including the same, an organic optoelectronic device, and a display device, the compound being represented by Chemical Formula 1:

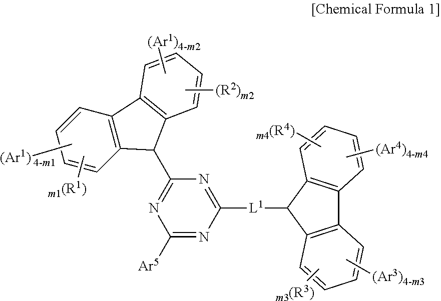

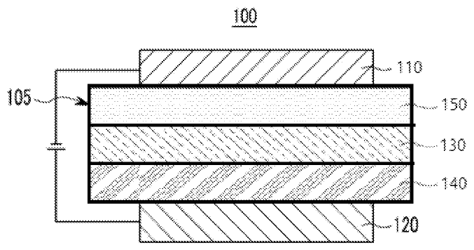

### CROSS-REFERENCE TO RELATED APPLICATION

This application claims priority to and the benefit of Korean Patent Application No. 10-2022-0128379 filed in the Korean Intellectual Property Office on Oct. 7, 2022, the entire contents of which are incorporated herein by reference.

### BACKGROUND

### 1. Field

Embodiments relate to a compound for an organic optoelectronic device, a composition for an organic optoelectronic device, an organic optoelectronic device, and a display device.

### 2. Description of the Related Art

An organic optoelectronic device (e.g., organic optoelectronic diode) is a device capable of converting electrical energy and optical energy to each other.

Organic optoelectronic devices may be divided into two types according to a principle of operation. One is a photoelectric device that generates electrical energy by separating excitons formed by light energy into electrons and holes, and transferring the electrons and holes to different electrodes, respectively and the other is a light emitting device that generates light energy from electrical energy by supplying voltage or current to the electrodes.

Examples of the organic optoelectronic device may include an organic photoelectric device, an organic light emitting diode, an organic solar cell, and an organic photoconductor drum.

Among them, organic light emitting diodes (OLEDs) are attracting much attention in recent years due to increasing demands for flat panel display devices. The organic light emitting diode is a device that converts electrical energy into light, and the performance of the organic light emitting diode is greatly influenced by an organic material between electrodes.

### SUMMARY

The embodiments may be realized by providing a compound for an organic optoelectronic device, the compound being represented by Chemical Formula 1:

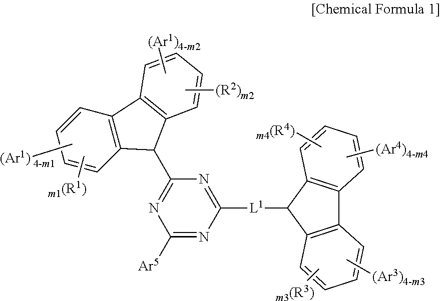

wherein, in Chemical Formula 1, R¹ to R⁴ and Ar¹ to Ar⁴ are each independently hydrogen, deuterium, a cyano group, a halogen, a substituted or unsubstituted amine group, a substituted or unsubstituted C1 to C30 alkyl group, a substituted or unsubstituted C6 to C30 aryl group, a substituted or unsubstituted C2 to C30 heterocyclic group, or a combination thereof, provided that at least one of R¹ to R⁴ is deuterium, a C1 to C30 alkyl group substituted with one or more deuterium, a C6 to C30 aryl group substituted with one or more deuterium, or a C2 to C30 heterocyclic group substituted with one or more deuterium, Ar⁵ is a substituted or unsubstituted C6 to C30 aryl group, a substituted or unsubstituted dibenzofuranyl group, or a substituted or unsubstituted dibenzothiophenyl group, L¹ is a phenylene group substituted with one or more deuterium or a biphenylene group substituted with one or more deuterium, and m1 to m4 are each independently an integer of 1 to 4.

The embodiments may be realized by providing a compound for an organic optoelectronic device, the compound being a compound of Group 1:

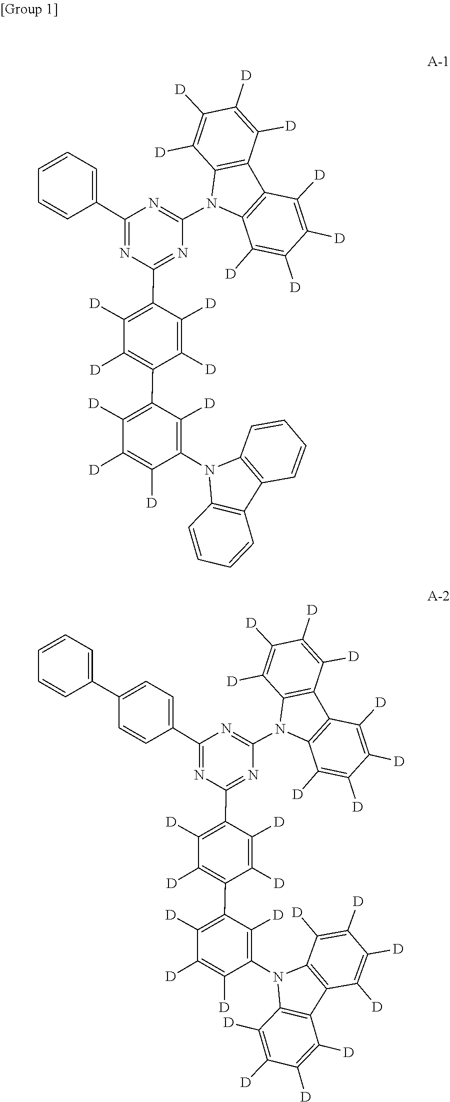 [SMILES: [2H]c1c([2H])c(-c2c([2H])c([2H])c(-c3nc(-c4ccccc4)nc(-n4c5c([2H])c([2H])c([2H])c([2H])c5c5c([2H])c([2H])c([2H])c([2H])c54)n3)c([2H])c2[2H])c([2H])c(-n2c3ccccc3c3ccccc32)c1[2H] ; [2H]c1c([2H])c(-c2c([2H])c([2H])c(-c3nc(-c4ccc(-c5ccccc5)cc4)nc(-n4c5c([2H])c([2H])c([2H])c([2H])c5c5c([2H])c([2H])c([2H])c([2H])c54)n3)c([2H])c2[2H])c([2H])c(-n2c3c([2H])c([2H])c([2H])c([2H])c3c3c([2H])c([2H])c([2H])c([2H])c32)c1[2H]]

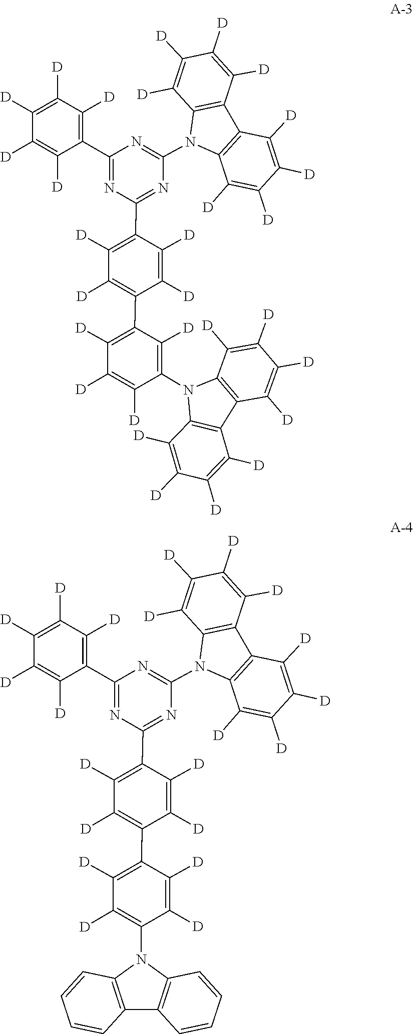

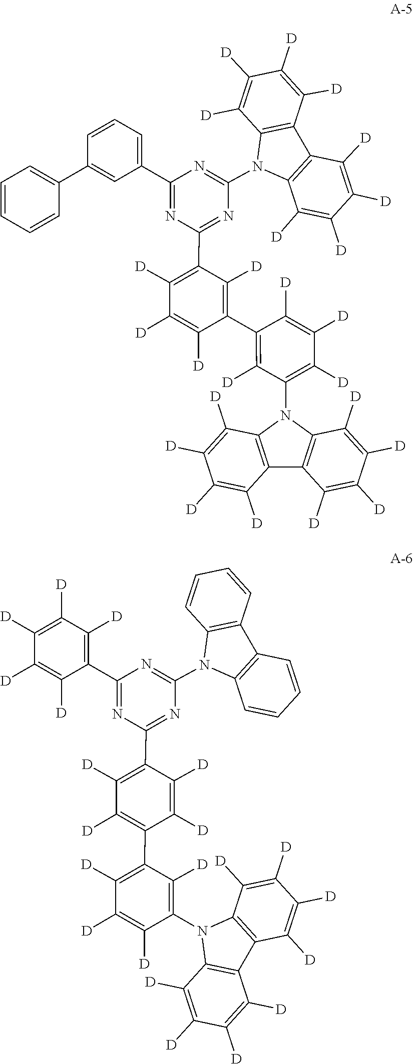 [SMILES: [2H]c1c([2H])c(-c2nc(-c3cccc(-c4ccccc4)c3)nc(-n3c4c([2H])c([2H])c([2H])c([2H])c4c4c([2H])c([2H])c([2H])c([2H])c43)n2)c([2H])c(-c2c([2H])c([2H])c([2H])c(-n3c4c([2H])c([2H])c([2H])c([2H])c4c4c([2H])c([2H])c([2H])c([2H])c43)c2[2H])c1[2H] ; [2H]c1c([2H])c([2H])c(-c2nc(-c3c([2H])c([2H])c(-c4c([2H])c([2H])c([2H])c(-n5c6c([2H])c([2H])c([2H])c([2H])c6c6c([2H])c([2H])c([2H])c([2H])c65)c4[2H])c([2H])c3[2H])nc(-n3c4ccccc4c4ccccc43)n2)c([2H])c1[2H]]

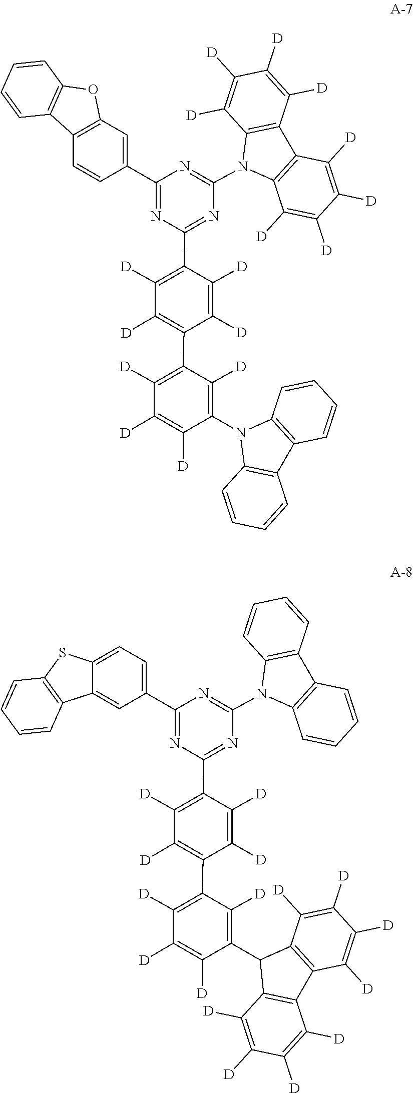 [SMILES: [2H]c1c([2H])c(-c2c([2H])c([2H])c(-c3nc(-c4ccc5c(c4)oc4ccccc45)nc(-n4c5c([2H])c([2H])c([2H])c([2H])c5c5c([2H])c([2H])c([2H])c([2H])c54)n3)c([2H])c2[2H])c([2H])c(-n2c3ccccc3c3ccccc32)c1[2H] ; [2H]c1c([2H])c([2H])c2c(c1[2H])-c1c([2H])c([2H])c([2H])c([2H])c1C2c1c([2H])c([2H])c([2H])c(-c2c([2H])c([2H])c(-c3nc(-c4ccc5sc6ccccc6c5c4)nc(-n4c5ccccc5c5ccccc54)n3)c([2H])c2[2H])c1[2H]]

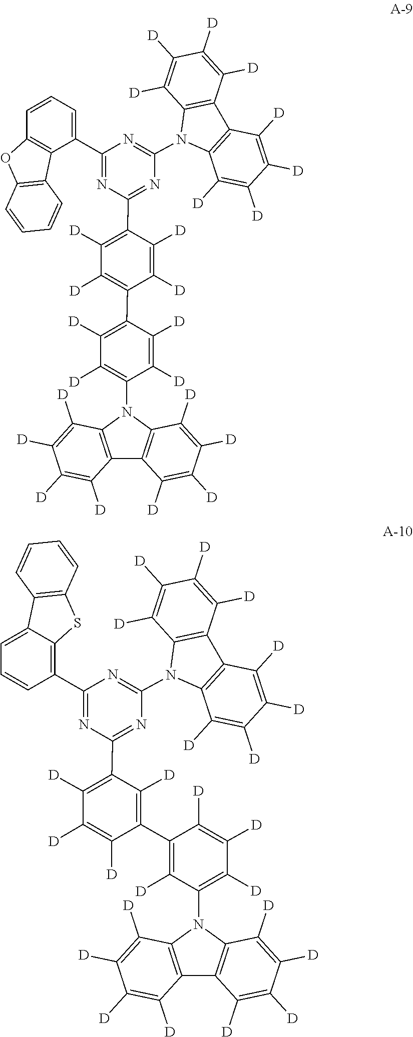 [SMILES: [2H]c1c([2H])c(-c2c([2H])c([2H])c(-n3c4c([2H])c([2H])c([2H])c([2H])c4c4c([2H])c([2H])c([2H])c([2H])c43)c([2H])c2[2H])c([2H])c([2H])c1-c1nc(-c2cccc3oc4ccccc4c23)nc(-n2c3c([2H])c([2H])c([2H])c([2H])c3c3c([2H])c([2H])c([2H])c([2H])c32)n1 ; [2H]c1c([2H])c(-c2nc(-c3cccc4c3sc3ccccc34)nc(-n3c4c([2H])c([2H])c([2H])c([2H])c4c4c([2H])c([2H])c([2H])c([2H])c43)n2)c([2H])c(-c2c([2H])c([2H])c([2H])c(-n3c4c([2H])c([2H])c([2H])c([2H])c4c4c([2H])c([2H])c([2H])c([2H])c43)c2[2H])c1[2H]]

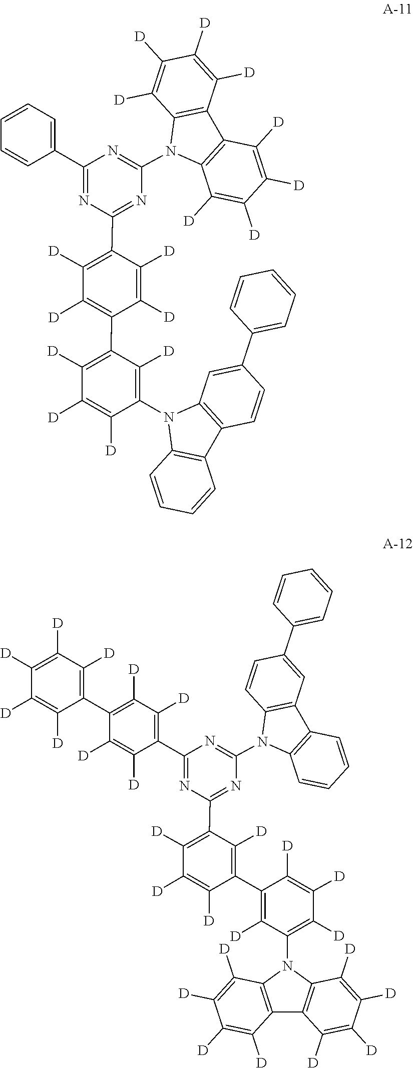 [SMILES: [2H]c1c([2H])c(-c2c([2H])c([2H])c(-c3nc(-c4ccccc4)nc(-n4c5c([2H])c([2H])c([2H])c([2H])c5c5c([2H])c([2H])c([2H])c([2H])c54)n3)c([2H])c2[2H])c([2H])c(-n2c3ccccc3c3ccc(-c4ccccc4)cc32)c1[2H] ; [2H]c1c([2H])c([2H])c(-c2c([2H])c([2H])c(-c3nc(-c4c([2H])c([2H])c([2H])c(-c5c([2H])c([2H])c([2H])c(-n6c7c([2H])c([2H])c([2H])c([2H])c7c7c([2H])c([2H])c([2H])c([2H])c76)c5[2H])c4[2H])nc(-n4c5ccccc5c5cc(-c6ccccc6)ccc54)n3)c([2H])c2[2H])c([2H])c1[2H]]

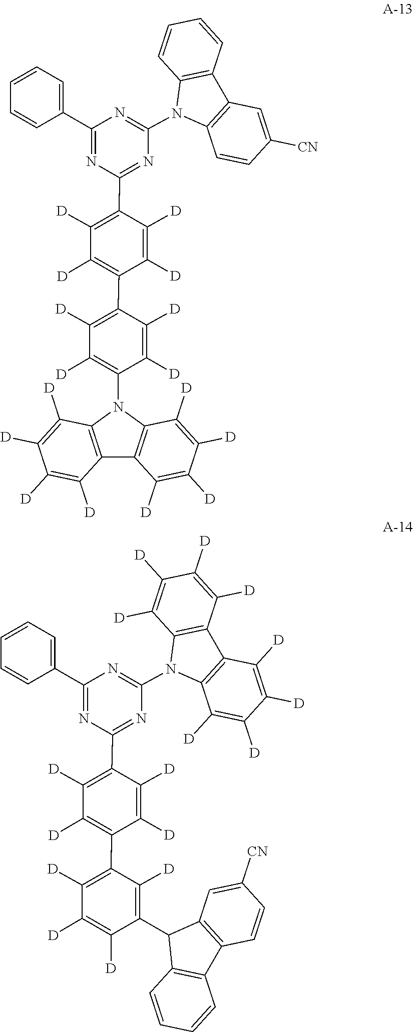

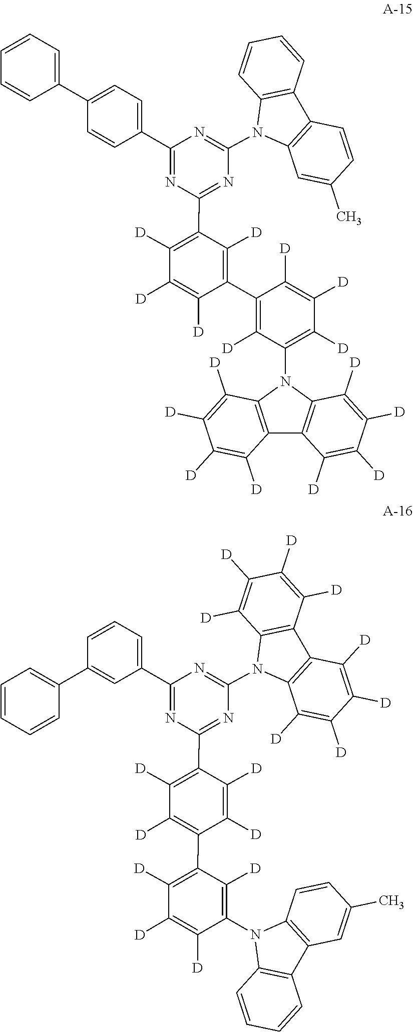 [SMILES: [2H]c1c([2H])c(-c2nc(-c3ccc(-c4ccccc4)cc3)nc(-n3c4ccccc4c4ccc(C)cc43)n2)c([2H])c(-c2c([2H])c([2H])c([2H])c(-n3c4c([2H])c([2H])c([2H])c([2H])c4c4c([2H])c([2H])c([2H])c([2H])c43)c2[2H])c1[2H] ; [2H]c1c([2H])c(-c2c([2H])c([2H])c(-c3nc(-c4cccc(-c5ccccc5)c4)nc(-n4c5c([2H])c([2H])c([2H])c([2H])c5c5c([2H])c([2H])c([2H])c([2H])c54)n3)c([2H])c2[2H])c([2H])c(-n2c3ccccc3c3cc(C)ccc32)c1[2H]]

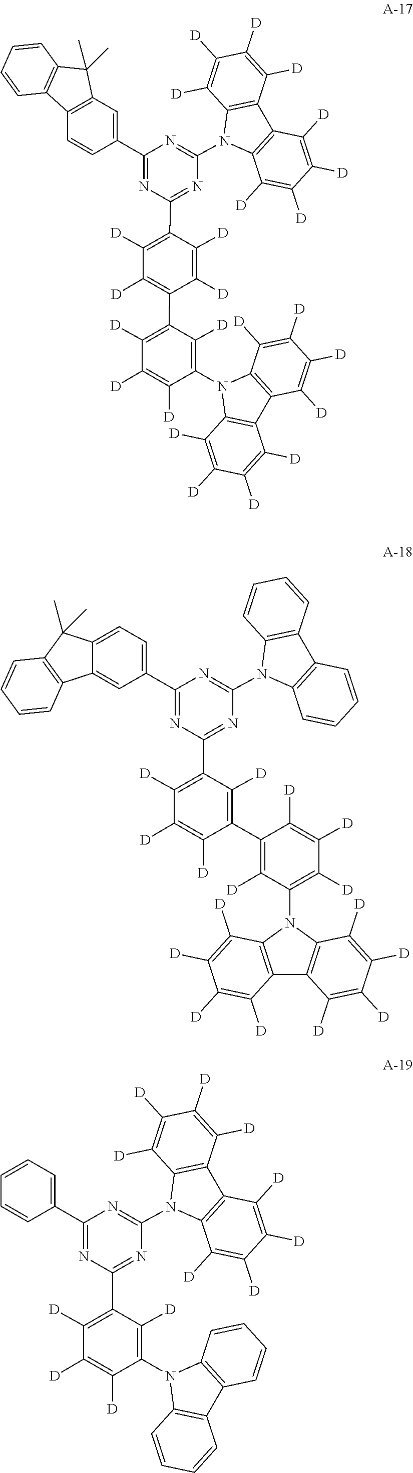

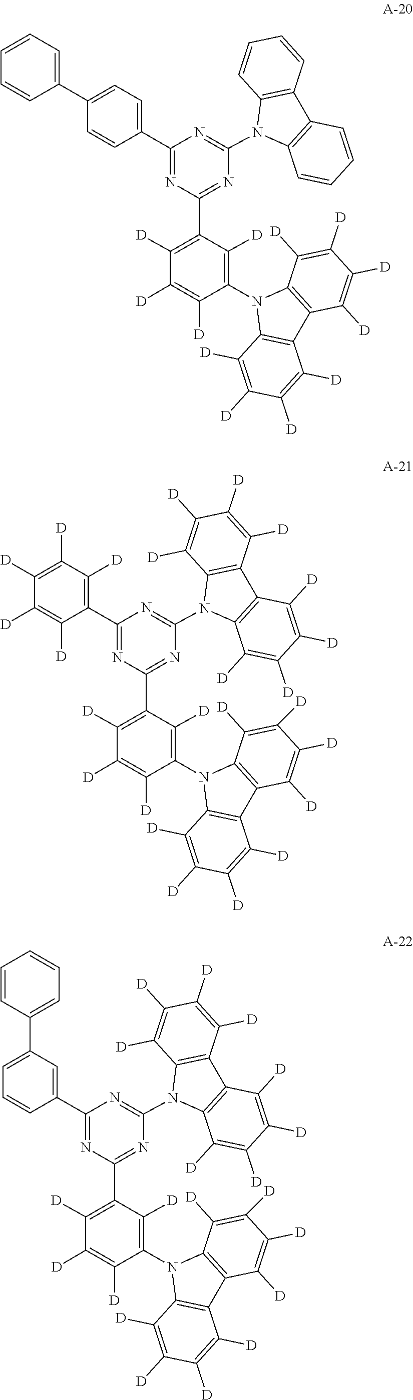

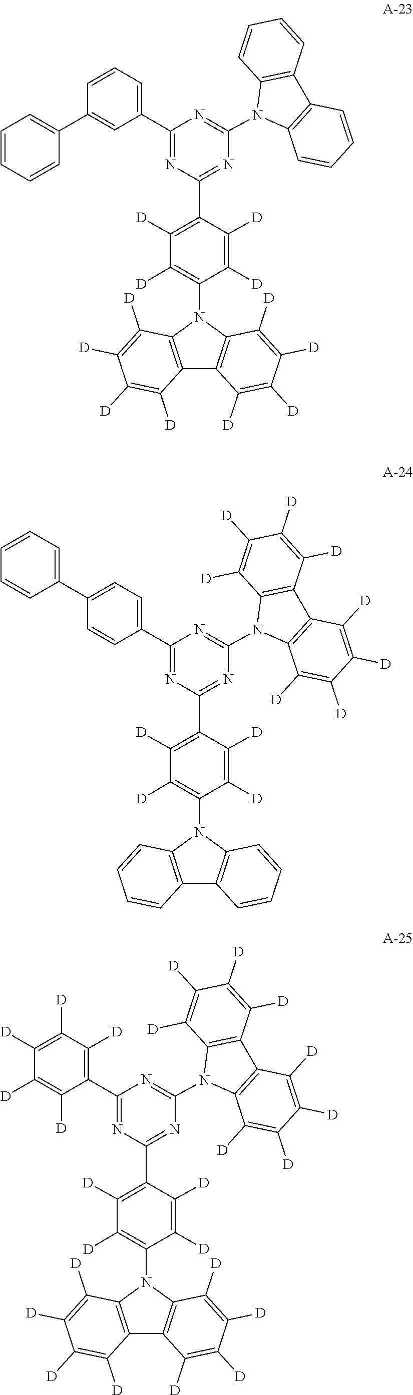

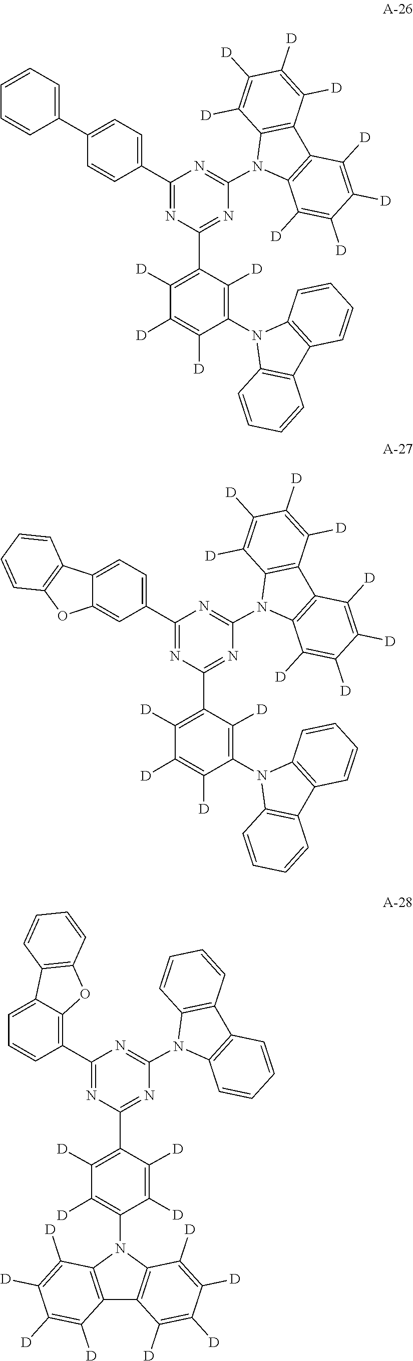 [SMILES: [2H]c1c([2H])c(-c2nc(-c3ccc(-c4ccccc4)cc3)nc(-n3c4c([2H])c([2H])c([2H])c([2H])c4c4c([2H])c([2H])c([2H])c([2H])c43)n2)c([2H])c(-n2c3ccccc3c3ccccc32)c1[2H] ; [2H]c1c([2H])c(-c2nc(-c3ccc4c(c3)oc3ccccc34)nc(-n3c4c([2H])c([2H])c([2H])c([2H])c4c4c([2H])c([2H])c([2H])c([2H])c43)n2)c([2H])c(-n2c3ccccc3c3ccccc32)c1[2H] ; [2H]c1c([2H])c(-n2c3c([2H])c([2H])c([2H])c([2H])c3c3c([2H])c([2H])c([2H])c([2H])c32)c([2H])c([2H])c1-c1nc(-c2cccc3c2oc2ccccc23)nc(-n2c3ccccc3c3ccccc32)n1]

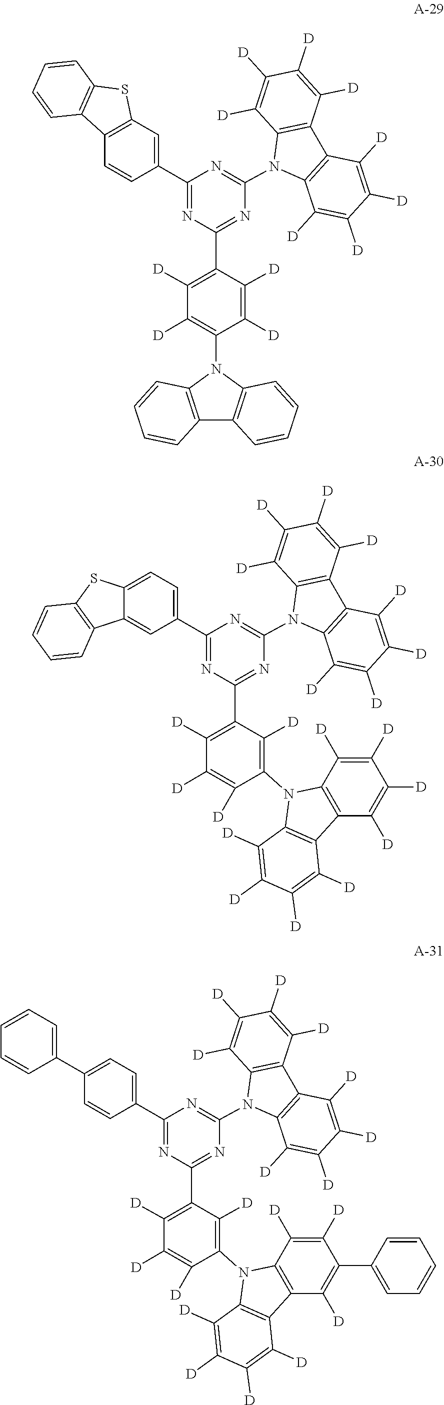

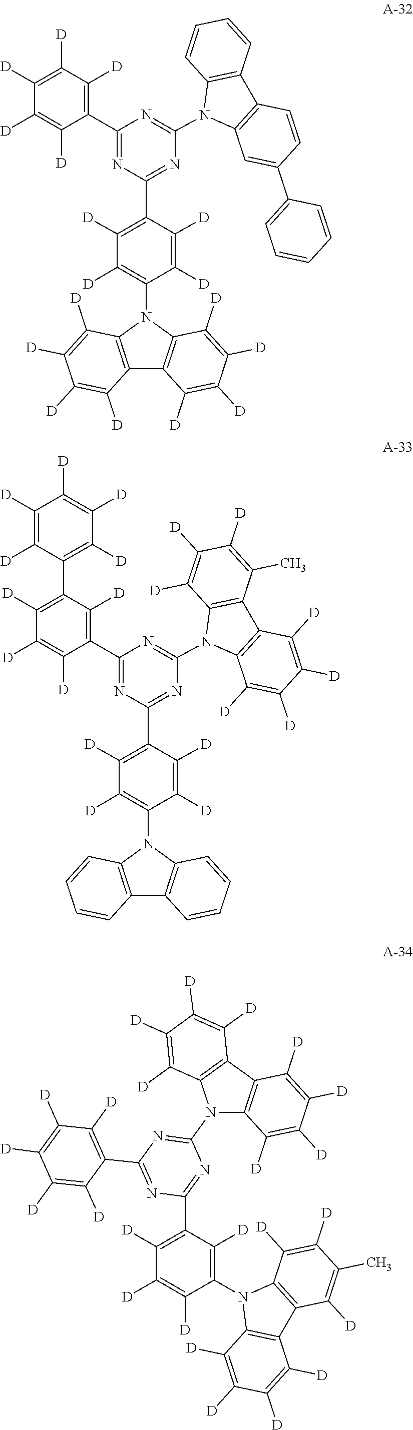

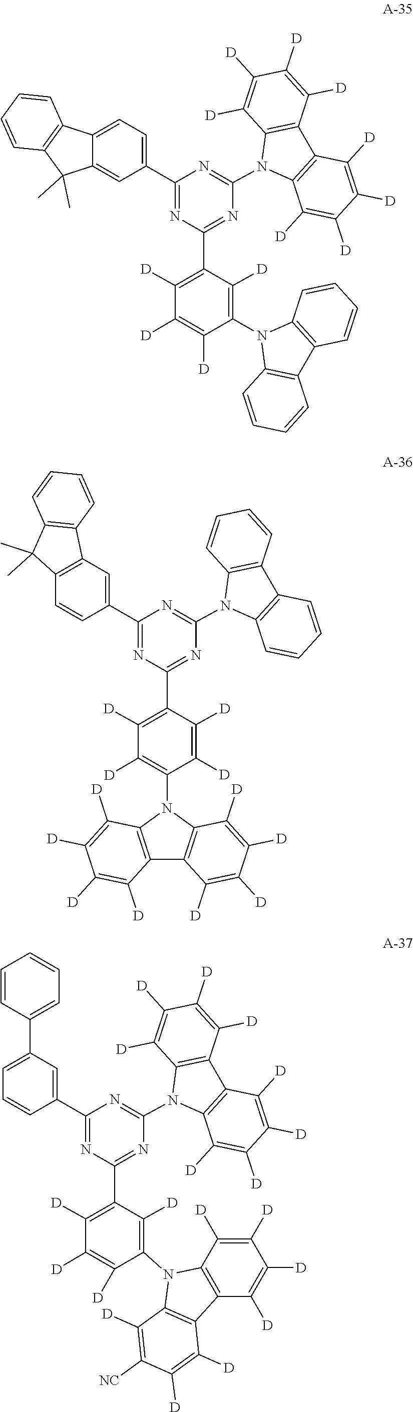

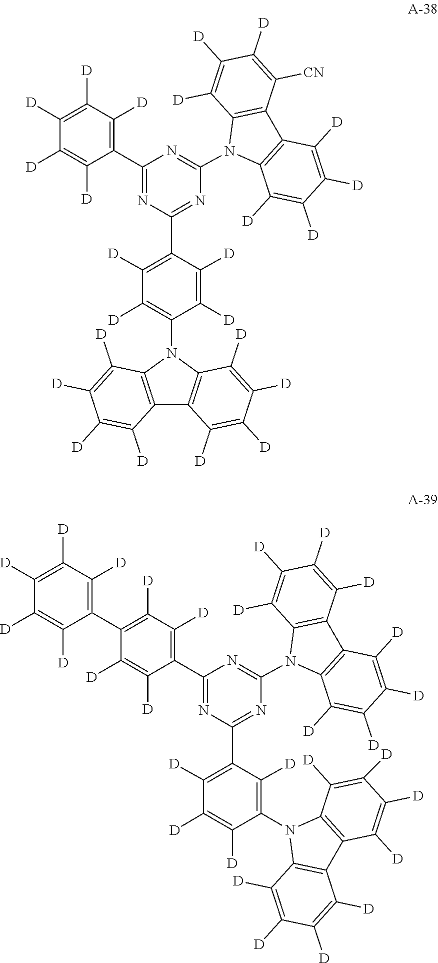 [SMILES: [2H]c1c([2H])c([2H])c(-c2nc(-c3c([2H])c([2H])c(-n4c5c([2H])c([2H])c([2H])c([2H])c5c5c([2H])c([2H])c([2H])c([2H])c54)c([2H])c3[2H])nc(-n3c4c([2H])c([2H])c([2H])c([2H])c4c4c(C#N)c([2H])c([2H])c([2H])c43)n2)c([2H])c1[2H] ; [2H]c1c([2H])c([2H])c(-c2c([2H])c([2H])c(-c3nc(-c4c([2H])c([2H])c([2H])c(-n5c6c([2H])c([2H])c([2H])c([2H])c6c6c([2H])c([2H])c([2H])c([2H])c65)c4[2H])nc(-n4c5c([2H])c([2H])c([2H])c([2H])c5c5c([2H])c([2H])c([2H])c([2H])c54)n3)c([2H])c2[2H])c([2H])c1[2H]]

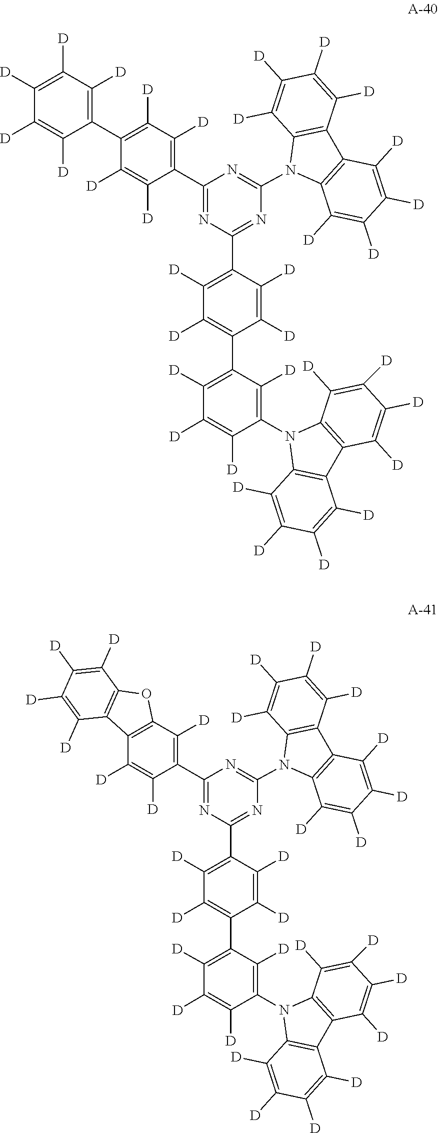

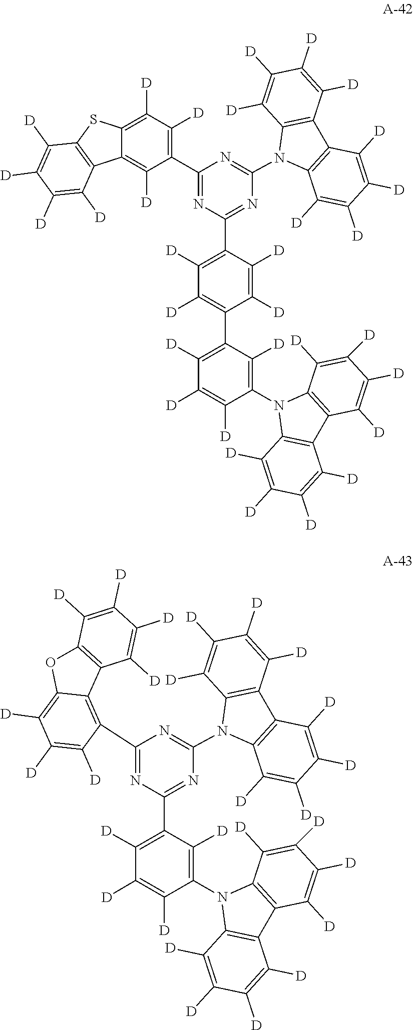

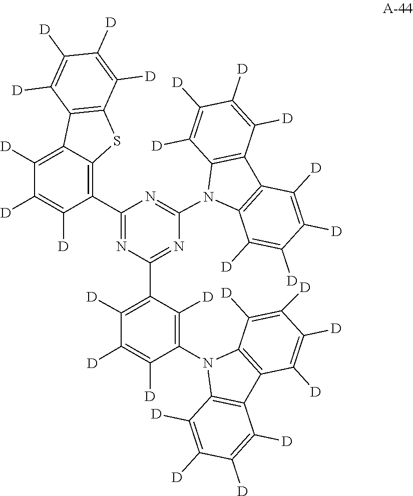 [SMILES: [2H]c1c([2H])c(-c2nc(-c3c([2H])c([2H])c([2H])c4c3sc3c([2H])c([2H])c([2H])c([2H])c34)nc(-n3c4c([2H])c([2H])c([2H])c([2H])c4c4c([2H])c([2H])c([2H])c([2H])c43)n2)c([2H])c(-n2c3c([2H])c([2H])c([2H])c([2H])c3c3c([2H])c([2H])c([2H])c([2H])c32)c1[2H]]

The embodiments may be realized by providing a composition for an organic optoelectronic device, the composition including a first compound; and a second compound, wherein the first compound is the compound for an organic optoelectronic device according to an embodiment, and the second compound is represented by Chemical Formula 2; or a combination of Chemical Formula 3 and Chemical Formula 4,

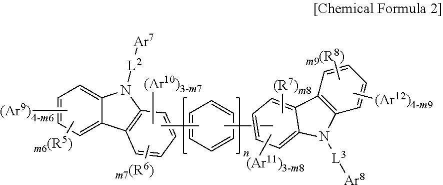

in Chemical Formula 2, R⁵ to R⁹ and Ar⁹ to Ar¹² are each independently hydrogen, deuterium, a cyano group, a halogen, a substituted or unsubstituted amine group, a substituted or unsubstituted C1 to C30 alkyl group, a substituted or unsubstituted C6 to C30 aryl group or a substituted or unsubstituted C2 to C30 heterocyclic group, Ar⁷ and Ar⁸ are each independently a substituted or unsubstituted C6 to C20 aryl group or a substituted or unsubstituted C2 to C30 heterocyclic group, L² and L³ are each independently a single bond or a substituted or unsubstituted C6 to C20 arylene group, m6, m9 and m10 are each independently an integer of 1 to 4, m7 and m8 are each independently an integer of 1 to 3, and n is an integer of 0 to 2,

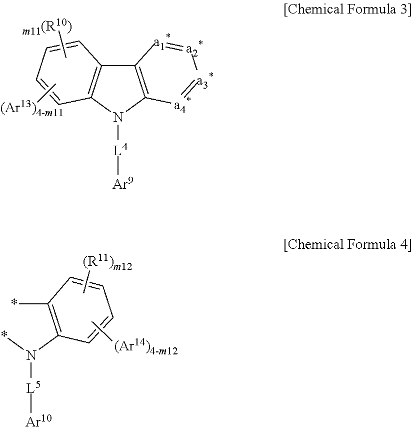

in Chemical Formula 3 and Chemical Formula 4, two adjacent ones of a1* to a4* of Chemical Formula 3 are linking carbons linked at * of Chemical Formula 4, the remaining two of a1* to a4* of Chemical Formula 3, not linked at * of Chemical Formula 4, are C-L^{a}-R^{a}, L^{a}, L⁴, and L⁵ are each independently a single bond or a substituted or unsubstituted C6 to C20 arylene group, R^{a}, R¹⁰, R¹¹, Ar¹³, and Ar¹⁴ are each independently hydrogen, deuterium, a cyano group, a halogen, a substituted or unsubstituted amine group, a substituted or unsubstituted C1 to C30 alkyl group, a substituted or unsubstituted C6 to C30 aryl group or a substituted or unsubstituted C2 to C30 heterocyclic group, Ar⁹ and Ar¹⁰ are each independently a substituted or unsubstituted C6 to C20 aryl group or a substituted or unsubstituted C2 to C30 heterocyclic group, and m11 and m12 are each independently an integer of 1 to 4.

The embodiments may be realized by providing an organic optoelectronic device including an anode and a cathode facing each other, and at least one organic layer between the anode and the cathode, wherein the at least one organic layer includes the compound or the composition for an organic optoelectronic device according to an embodiment.

The embodiments may be realized by providing a display device comprising the organic optoelectronic device according to an embodiment.

### BRIEF DESCRIPTION OF THE DRAWING

Features will be apparent to those of skill in the art by describing in detail exemplary embodiments with reference to the attached drawing in which:

the FIGURE is a cross-sectional view illustrating an organic light emitting diode according to an embodiment.

### DETAILED DESCRIPTION

Example embodiments will now be described more fully hereinafter with reference to the accompanying drawing; however, they may be embodied in different forms and should not be construed as limited to the embodiments set forth herein. Rather, these embodiments are provided so that this disclosure will be thorough and complete, and will fully convey exemplary implementations to those skilled in the art.

In the drawing figure, the dimensions of layers and regions may be exaggerated for clarity of illustration. It will also be understood that when a layer or element is referred to as being “on” another layer or element, it can be directly on the other layer or element, or intervening layers may also be present. In addition, it will also be understood that when a layer is referred to as being “between” two layers, it can be the only layer between the two layers, or one or more intervening layers may also be present. Like reference numerals refer to like elements throughout.

In the present specification, when a definition is not otherwise provided, “substituted” refers to replacement of at least one hydrogen of a substituent or a compound by deuterium, a halogen, a hydroxyl group, an amino group, a substituted or unsubstituted C1 to C30 amine group, a nitro group, a substituted or unsubstituted C1 to C40 silyl group, a C1 to C30 alkyl group, a C1 to C10 alkylsilyl group, a C6 to C30 arylsilyl group, a C3 to C30 cycloalkyl group, a C3 to C30 heterocycloalkyl group, a C6 to C30 aryl group, a C2 to C30 heteroaryl group, a C1 to C20 alkoxy group, a C1 to C10 trifluoroalkyl group, a cyano group, or a combination thereof. As used herein, the term “or” is not necessarily an exclusive term, e.g., “A or B” would include A, B, or A and B.

In one example, the “substituted” refers to replacement of at least one hydrogen of a substituent or a compound by deuterium, a C1 to C30 alkyl group, a C1 to C10 alkylsilyl group, a C6 to C30 arylsilyl group, a C3 to C30 cycloalkyl group, a C3 to C30 heterocycloalkyl group, a C6 to C30 aryl group, a C2 to C30 heteroaryl group, or a cyano group. In specific example, the “substituted” refers to replacement of at least one hydrogen of a substituent or a compound by deuterium, a C1 to C20 alkyl group, a C6 to C30 aryl group, or a cyano group. In specific example, the “substituted” refers to replacement of at least one hydrogen of a substituent or a compound by deuterium, a C1 to C5 alkyl group, a C6 to C18 aryl group, or a cyano group. In specific example, the “substituted” refers to replacement of at least one hydrogen of a substituent or a compound by deuterium, a cyano group, a methyl group, an ethyl group, a propyl group, a butyl group, a phenyl group, a biphenyl group, a terphenyl group, or a naphthyl group.

In this specification, “unsubstituted” means that a hydrogen atom remains as a hydrogen atom without being substituted with another substituent.

In this specification, any hydrogen may naturally include deuterium (-D) or tritium (-T). In this specification, unless defined differently, * is a linking point.

In the present specification, when a definition is not otherwise provided, “hetero” refers to one including one to three heteroatoms selected from N, O, S, P, and Si, and remaining carbons in one functional group.

In the present specification, “an aryl group” refers to a group including at least one hydrocarbon aromatic moiety, and all elements of the hydrocarbon aromatic moiety have p-orbitals which form conjugation, for example a phenyl group, a naphthyl group, and the like, two or more hydrocarbon aromatic moieties may be linked by a sigma bond and may be, for example a biphenyl group, a terphenyl group, a quarterphenyl group, and the like, and two or more hydrocarbon aromatic moieties are fused directly or indirectly to provide a non-aromatic fused ring, for example a fluorenyl group.

The aryl group may include a monocyclic, polycyclic, or fused ring polycyclic (i.e., rings sharing adjacent pairs of carbon atoms) functional group.

In the present specification, “a heterocyclic group” is a generic concept of a heteroaryl group, and may include at least one heteroatom selected from N, O, S, P, and Si instead of carbon (C) in a cyclic compound such as an aryl group, a cycloalkyl group, a fused ring thereof, or a combination thereof. When the heterocyclic group is a fused ring, the entire ring or each ring of the heterocyclic group may include one or more heteroatoms.

For example, “a heteroaryl group” refers to an aryl group including at least one heteroatom selected from N, O, S, P, and Si. Two or more heteroaryl groups are linked by a sigma bond directly, or when the heteroaryl group includes two or more rings, the two or more rings may be fused. When the heteroaryl group is a fused ring, each ring may include one to three heteroatoms.

More specifically, the substituted or unsubstituted C6 to C30 aryl group may be a substituted or unsubstituted phenyl group, a substituted or unsubstituted naphthyl group, a substituted or unsubstituted anthracenyl group, a substituted or unsubstituted phenanthrenyl group, a substituted or unsubstituted naphthacenyl group, a substituted or unsubstituted pyrenyl group, a substituted or unsubstituted biphenyl group, a substituted or unsubstituted p-terphenyl group, a substituted or unsubstituted m-terphenyl group, a substituted or unsubstituted o-terphenyl group, a substituted or unsubstituted chrysenyl group, a substituted or unsubstituted benzophenanthrenyl group, a substituted or unsubstituted triphenylene group, a substituted or unsubstituted perylenyl group, a substituted or unsubstituted fluorenyl group, a substituted or unsubstituted indenyl group, or a combination thereof, but is not limited thereto.

More specifically, the substituted or unsubstituted C2 to C30 heterocyclic group may be a substituted or unsubstituted furanyl group, a substituted or unsubstituted thiophenyl group, a substituted or unsubstituted pyrrolyl group, a substituted or unsubstituted pyrazolyl group, a substituted or unsubstituted imidazolyl group, a substituted or unsubstituted triazolyl group, a substituted or unsubstituted oxazolyl group, a substituted or unsubstituted thiazolyl group, a substituted or unsubstituted oxadiazolyl group, a substituted or unsubstituted thiadiazolyl group, a substituted or unsubstituted pyridyl group, a substituted or unsubstituted pyrimidinyl group, a substituted or unsubstituted pyrazinyl group, a substituted or unsubstituted triazinyl group, a substituted or unsubstituted benzofuranyl group, a substituted or unsubstituted benzothiophenyl group, a substituted or unsubstituted benzimidazolyl group, a substituted or unsubstituted indolyl group, a substituted or unsubstituted quinolinyl group, a substituted or unsubstituted isoquinolinyl group, a substituted or unsubstituted quinazolinyl group, a substituted or unsubstituted quinoxalinyl group, a substituted or unsubstituted naphthyridinyl group, a substituted or unsubstituted benzoxazinyl group, a substituted or unsubstituted benzothiazinyl group, a substituted or unsubstituted acridinyl group, a substituted or unsubstituted phenazinyl group, a substituted or unsubstituted phenothiazinyl group, a substituted or unsubstituted phenoxazinyl group, a substituted or unsubstituted carbazolyl group, a substituted or unsubstituted dibenzofuranyl group, or a substituted or unsubstituted dibenzothiophenyl group, a substituted or unsubstituted benzonaphthofuranyl group, a substituted or unsubstituted benzonaphthothiophenyl group, a substituted or unsubstituted benzofuranofluorenyl group, a substituted or unsubstituted benzothiophenefluorenyl group, or a combination thereof, but are not limited thereto.

In the present specification, hole characteristics refer to an ability to donate an electron to form a hole when an electric field is applied and that a hole formed in the anode may be easily injected into the light emitting layer and transported in the light emitting layer due to conductive characteristics according to a highest occupied molecular orbital (HOMO) level.

In addition, electron characteristics refer to an ability to accept an electron when an electric field is applied and that electron formed in the cathode may be easily injected into the light emitting layer and transported in the light emitting layer due to conductive characteristics according to a lowest unoccupied molecular orbital (LUMO) level.

Hereinafter, a compound for an organic optoelectronic device according to an embodiment is described.

The compound for an organic optoelectronic device according to an embodiment may be represented by, e.g., Chemical Formula 1.

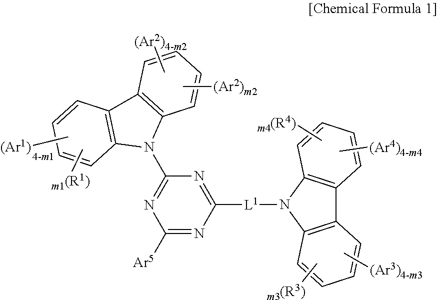

In Chemical Formula 1, R¹ to R⁴ and Ar¹ to Ar⁴ may each independently be or include, e.g., hydrogen, deuterium, a cyano group, a halogen, a substituted or unsubstituted amine group, a substituted or unsubstituted C1 to C30 alkyl group, a substituted or unsubstituted C6 to C30 aryl group, a substituted or unsubstituted C2 to C30 heterocyclic group, or a combination thereof.

Ar⁵ may be or may include, e.g., a substituted or unsubstituted C6 to C30 aryl group, a substituted or unsubstituted dibenzofuranyl group, or a substituted or unsubstituted dibenzothiophenyl group.

In an implementation, at least one of R¹ to R⁴ may be, e.g., deuterium, a C1 to C30 alkyl group substituted with one or more deuterium, a C6 to C30 aryl group substituted with one or more deuterium, or a C2 to C30 heterocyclic group substituted with one or more deuterium,

L¹ may be or may include, e.g., a phenylene group substituted with one or more deuterium or a biphenylene group substituted with one or more deuterium.

m1 to m4 may each independently be, e.g., an integer of 1 to 4.

The compound represented by Chemical Formula 1 may have a structure in which one carbazole group is directly linked to triazine in the N-direction without a linking group, and another carbazole group is linked to triazine in the N-direction through a phenylene or a biphenylene.

One carbazole group may be directly linked to the triazine in the N-direction, e.g., at the 9-position, without a linking group, so that it may have a relatively deep LUMO energy level, which may be advantageous for electron injection and movement.

In addition, another carbazole group may be linked to the triazine in the N-direction, e.g., at the 9-position, the π-bond through the C—N bond may be broken, so that the electron cloud between the HOMO-LUMO may be clearly localized into the hole transport part and the electron transport part. In this localization, the carbazole group may be more effectively separated by being linked to the triazine through phenylene or biphenylene, and thus life-span improvement may be maximized.

In an implementation, in a device including the compound represented by Chemical Formula 1, hole/electron injection and movement may be advantageous, and electron clouds may be effectively localized to implement a structure stable for both electrons and holes, so that it may have more favorable characteristics for life-span.

In an implementation, by including triazine in a central core, fast electron injection and mobility may be ensured, resulting in charge balance with the carbazole moiety having strong hole mobility, which may also greatly contribute to long life-span characteristics.

In an implementation, at least one of R¹ to R⁴ may be, e.g., deuterium, a C1 to C30 alkyl group substituted with one or more deuterium, a C6 to C30 aryl group substituted with one or more deuterium, or a C2 to C30 heterocyclic group substituted with one or more deuterium.

In an implementation, one or more deuterium may be substituted on at least one of R¹ to R⁴ and L¹. In an implementation, when deuterium substitution is performed, a ground state energy may be lowered due to lower zero-point energy and lower vibrational energy compared to compounds substituted with hydrogen, and intermolecular interactions may be reduced. The compound may be made into an amorphous state, and heat resistance may be further improved and life-span of an organic light emitting diode to which it is applied may be more effectively improved. Accordingly, the life-span may be dramatically improved through deuterium substitution.

In Chemical Formula 1, when two or more of R¹, R², R³, R⁴, Ar¹, Ar², Ar³, and Ar⁴ are each substituted, each of R¹, each of R², each of R³, each of R⁴, each of Ar¹, each of Ar², each Ar³, and each Ar⁴ may be the same or different.

In an implementation, at least one of R¹ and R² may be, e.g., deuterium, a C1 to C30 alkyl group substituted with one or more deuterium, a C6 to C30 aryl group substituted with one or more deuterium, or a C2 to C30 heterocyclic group substituted with one or more deuterium.

In an implementation, R¹ and R² may each independently be, e.g., deuterium, a C1 to C30 alkyl group substituted with one or more deuterium, a C6 to C30 aryl group substituted with one or more deuterium, or a C2 to C30 heterocyclic group substituted with one or more deuterium.

In an implementation, at least one of R³ and R⁴ may be deuterium, a C1 to C30 alkyl group substituted with one or more deuterium, a C6 to C30 aryl group substituted with one or more deuterium, or a C2 to C30 heterocyclic group substituted with one or more deuterium.

In an implementation, R³ and R⁴ may each independently be, e.g., deuterium, a C1 to C30 alkyl group substituted with one or more deuterium, a C6 to C30 aryl group substituted with one or more deuterium, or a C2 to C30 heterocyclic group substituted with one or more deuterium.

In an implementation, R¹ to R⁴ may each independently be, e.g., deuterium, a C1 to C30 alkyl group substituted with one or more deuterium, a C6 to C30 aryl group substituted with one or more deuterium, or a C2 to C30 heterocyclic group substituted with one or more deuterium.

In an implementation, L¹ in Chemical Formula 1 may be, e.g., a linking group of Group I.

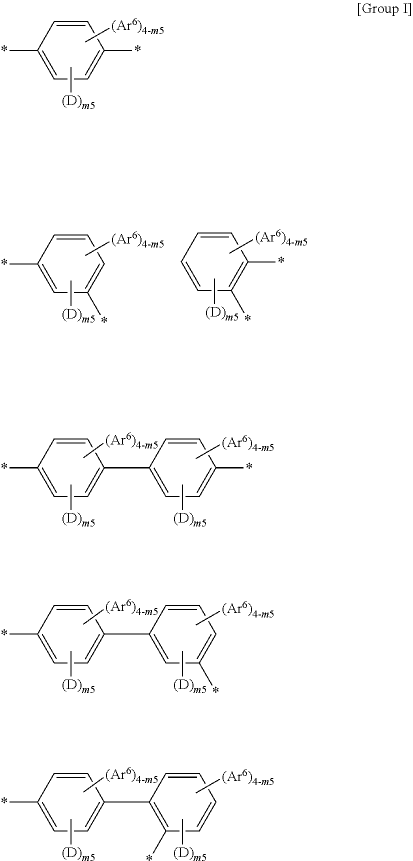

In Group I, D is deuterium.

Each Ar⁶ may independently be, e.g., hydrogen, deuterium, a cyano group, a substituted or unsubstituted C1 to C10 alkyl group, or a substituted or unsubstituted C6 to C12 aryl group.

m5 may be, e.g., an integer of 1 to 4.

In an implementation, m5 may be, e.g., an integer of 2 to 4.

In an implementation, m5 may be, e.g., an integer of 3 or 4.

In an implementation, m5 may be 4.

In an implementation, when L¹ in Chemical Formula 1 is a phenylene group substituted with at least one deuterium, the phenylene may be a meta-phenylene.

In an implementation, Ar⁵ may be, e.g., a substituted or unsubstituted phenyl group, a substituted or unsubstituted biphenyl group, a substituted or unsubstituted fluorenyl group, a substituted or unsubstituted dibenzofuranyl group, or a substituted or unsubstituted dibenzothiophenyl group.

In an implementation, Ar⁵ may be, e.g., a phenyl group substituted with one or more deuterium, a biphenyl group substituted with one or more deuterium, a fluorenyl group substituted with one or more deuterium, a dibenzofuranyl group substituted with one or more deuterium, or a dibenzothiophenyl group substituted with one or more deuterium.

In an implementation, the compound represented by Chemical Formula 1 may be, e.g., a compound of Group 1 below.

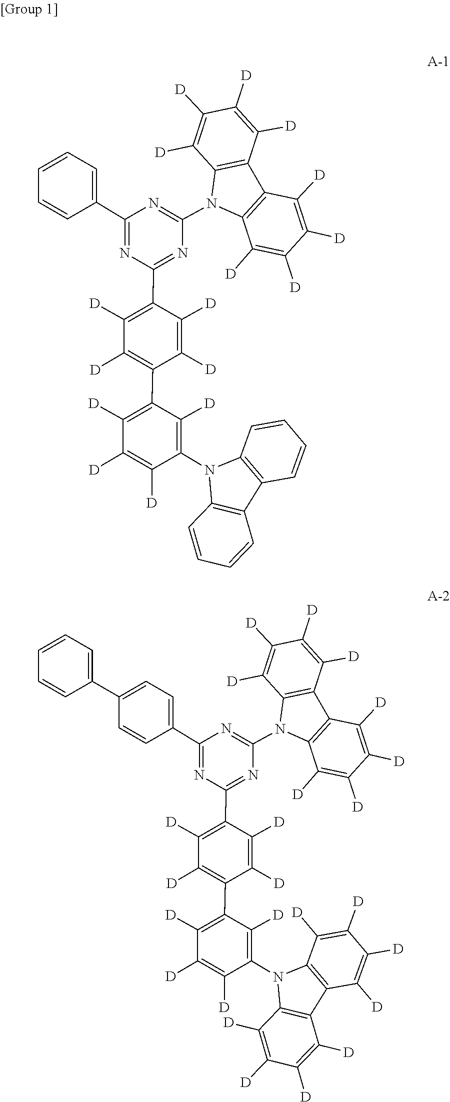 [SMILES: [2H]c1c([2H])c(-c2c([2H])c([2H])c(-c3nc(-c4ccccc4)nc(-n4c5c([2H])c([2H])c([2H])c([2H])c5c5c([2H])c([2H])c([2H])c([2H])c54)n3)c([2H])c2[2H])c([2H])c(-n2c3ccccc3c3ccccc32)c1[2H] ; [2H]c1c([2H])c(-c2c([2H])c([2H])c(-c3nc(-c4ccc(-c5ccccc5)cc4)nc(-n4c5c([2H])c([2H])c([2H])c([2H])c5c5c([2H])c([2H])c([2H])c([2H])c54)n3)c([2H])c2[2H])c([2H])c(-n2c3c([2H])c([2H])c([2H])c([2H])c3c3c([2H])c([2H])c([2H])c([2H])c32)c1[2H]]

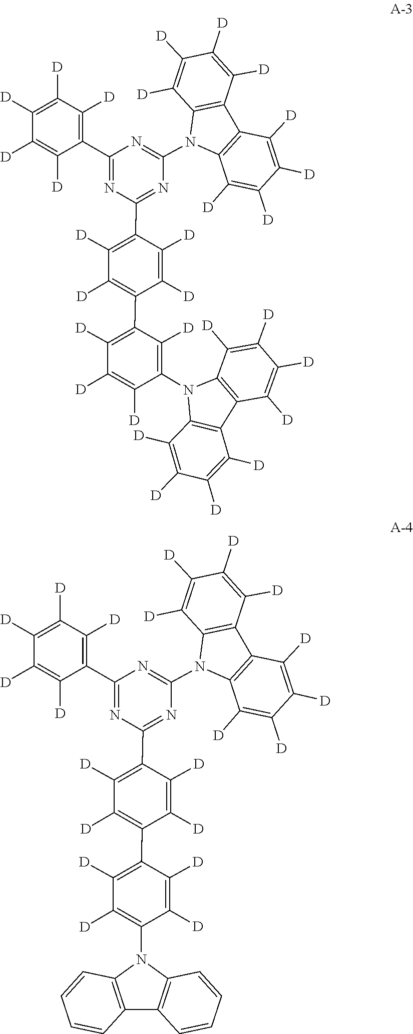

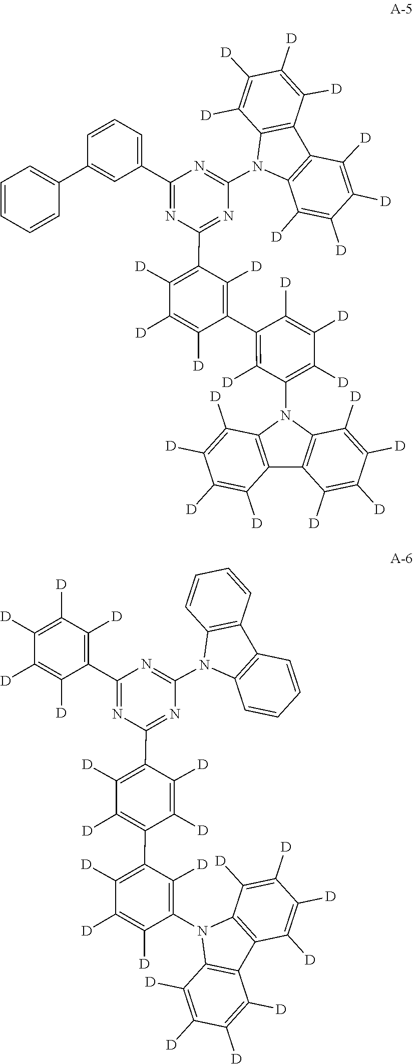 [SMILES: [2H]c1c([2H])c(-c2nc(-c3cccc(-c4ccccc4)c3)nc(-n3c4c([2H])c([2H])c([2H])c([2H])c4c4c([2H])c([2H])c([2H])c([2H])c43)n2)c([2H])c(-c2c([2H])c([2H])c([2H])c(-n3c4c([2H])c([2H])c([2H])c([2H])c4c4c([2H])c([2H])c([2H])c([2H])c43)c2[2H])c1[2H] ; [2H]c1c([2H])c([2H])c(-c2nc(-c3c([2H])c([2H])c(-c4c([2H])c([2H])c([2H])c(-n5c6c([2H])c([2H])c([2H])c([2H])c6c6c([2H])c([2H])c([2H])c([2H])c65)c4[2H])c([2H])c3[2H])nc(-n3c4ccccc4c4ccccc43)n2)c([2H])c1[2H]]

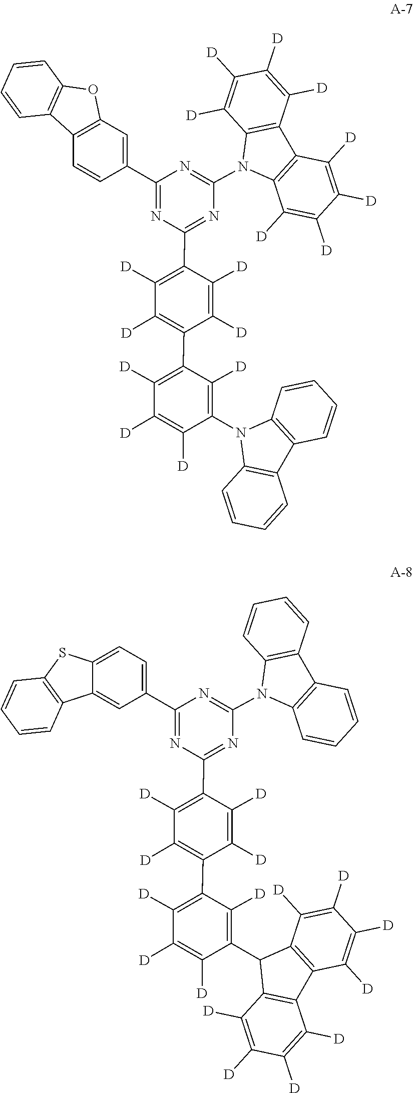 [SMILES: [2H]c1c([2H])c(-c2c([2H])c([2H])c(-c3nc(-c4ccc5c(c4)oc4ccccc45)nc(-n4c5c([2H])c([2H])c([2H])c([2H])c5c5c([2H])c([2H])c([2H])c([2H])c54)n3)c([2H])c2[2H])c([2H])c(-n2c3ccccc3c3ccccc32)c1[2H] ; [2H]c1c([2H])c([2H])c2c(c1[2H])-c1c([2H])c([2H])c([2H])c([2H])c1C2c1c([2H])c([2H])c([2H])c(-c2c([2H])c([2H])c(-c3nc(-c4ccc5sc6ccccc6c5c4)nc(-n4c5ccccc5c5ccccc54)n3)c([2H])c2[2H])c1[2H]]

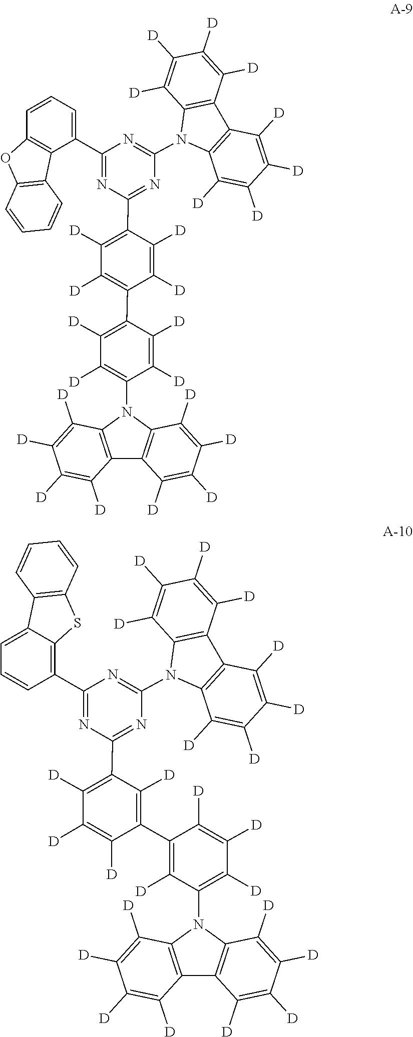 [SMILES: [2H]c1c([2H])c(-c2c([2H])c([2H])c(-n3c4c([2H])c([2H])c([2H])c([2H])c4c4c([2H])c([2H])c([2H])c([2H])c43)c([2H])c2[2H])c([2H])c([2H])c1-c1nc(-c2cccc3oc4ccccc4c23)nc(-n2c3c([2H])c([2H])c([2H])c([2H])c3c3c([2H])c([2H])c([2H])c([2H])c32)n1 ; [2H]c1c([2H])c(-c2nc(-c3cccc4c3sc3ccccc34)nc(-n3c4c([2H])c([2H])c([2H])c([2H])c4c4c([2H])c([2H])c([2H])c([2H])c43)n2)c([2H])c(-c2c([2H])c([2H])c([2H])c(-n3c4c([2H])c([2H])c([2H])c([2H])c4c4c([2H])c([2H])c([2H])c([2H])c43)c2[2H])c1[2H]]

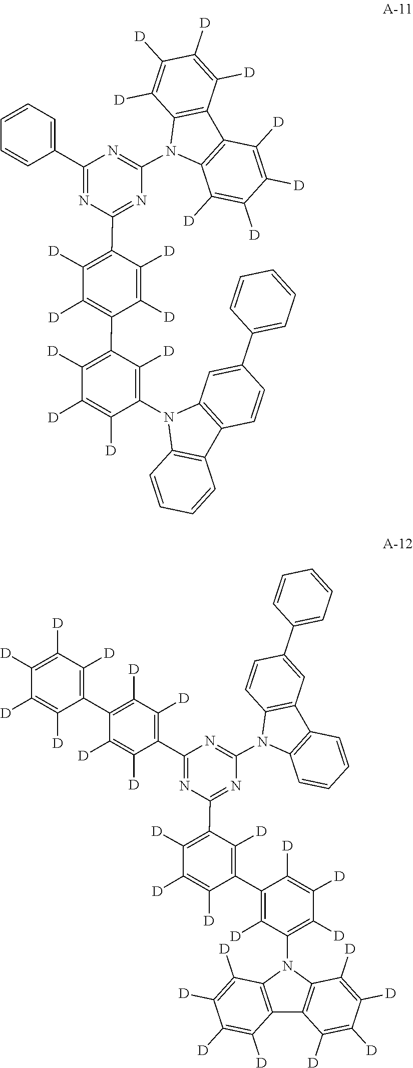 [SMILES: [2H]c1c([2H])c(-c2c([2H])c([2H])c(-c3nc(-c4ccccc4)nc(-n4c5c([2H])c([2H])c([2H])c([2H])c5c5c([2H])c([2H])c([2H])c([2H])c54)n3)c([2H])c2[2H])c([2H])c(-n2c3ccccc3c3ccc(-c4ccccc4)cc32)c1[2H] ; [2H]c1c([2H])c([2H])c(-c2c([2H])c([2H])c(-c3nc(-c4c([2H])c([2H])c([2H])c(-c5c([2H])c([2H])c([2H])c(-n6c7c([2H])c([2H])c([2H])c([2H])c7c7c([2H])c([2H])c([2H])c([2H])c76)c5[2H])c4[2H])nc(-n4c5ccccc5c5cc(-c6ccccc6)ccc54)n3)c([2H])c2[2H])c([2H])c1[2H]]

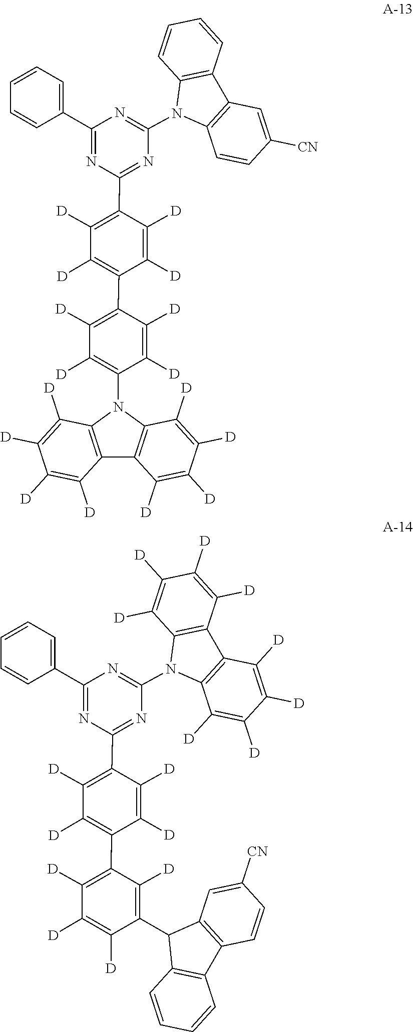

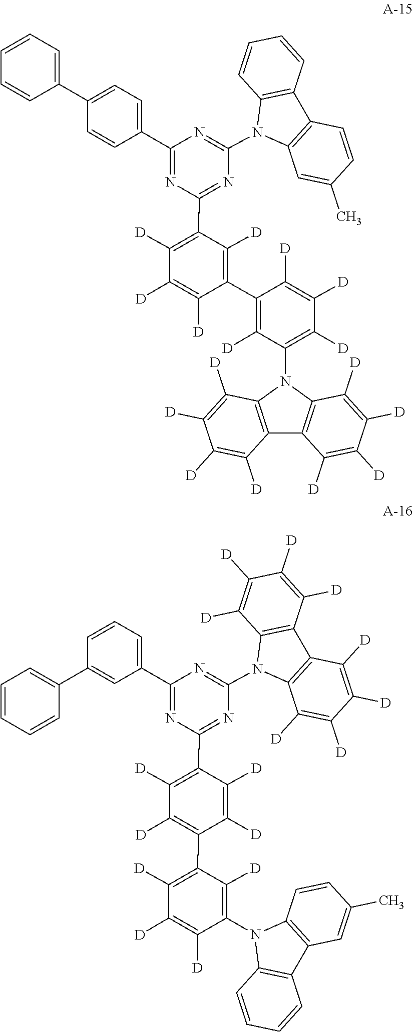 [SMILES: [2H]c1c([2H])c(-c2nc(-c3ccc(-c4ccccc4)cc3)nc(-n3c4ccccc4c4ccc(C)cc43)n2)c([2H])c(-c2c([2H])c([2H])c([2H])c(-n3c4c([2H])c([2H])c([2H])c([2H])c4c4c([2H])c([2H])c([2H])c([2H])c43)c2[2H])c1[2H] ; [2H]c1c([2H])c(-c2c([2H])c([2H])c(-c3nc(-c4cccc(-c5ccccc5)c4)nc(-n4c5c([2H])c([2H])c([2H])c([2H])c5c5c([2H])c([2H])c([2H])c([2H])c54)n3)c([2H])c2[2H])c([2H])c(-n2c3ccccc3c3cc(C)ccc32)c1[2H]]

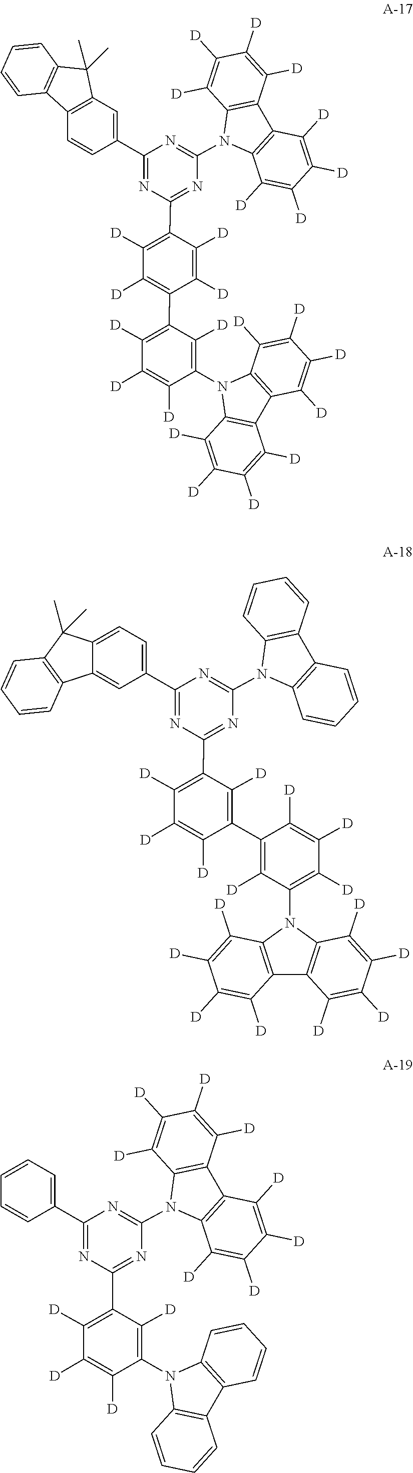

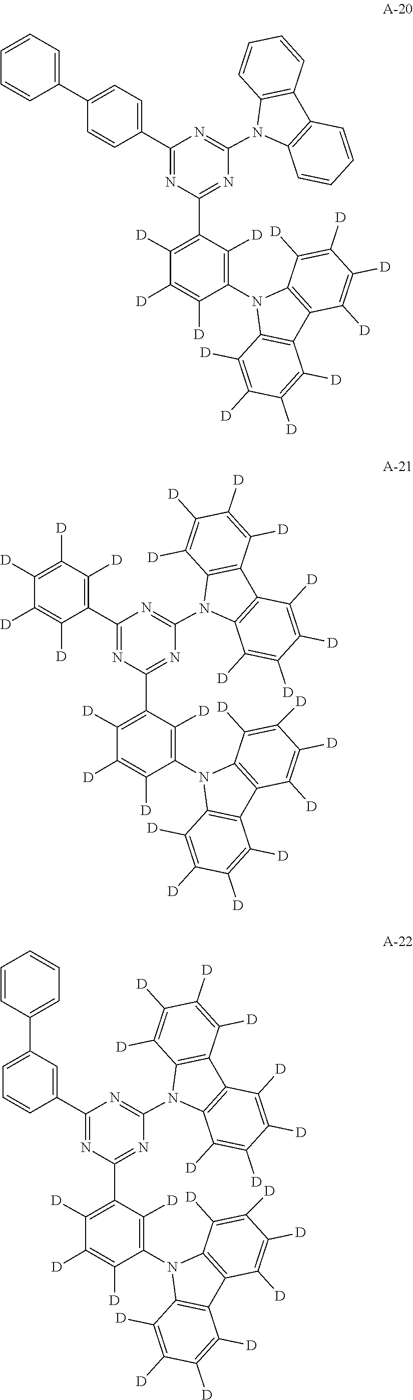

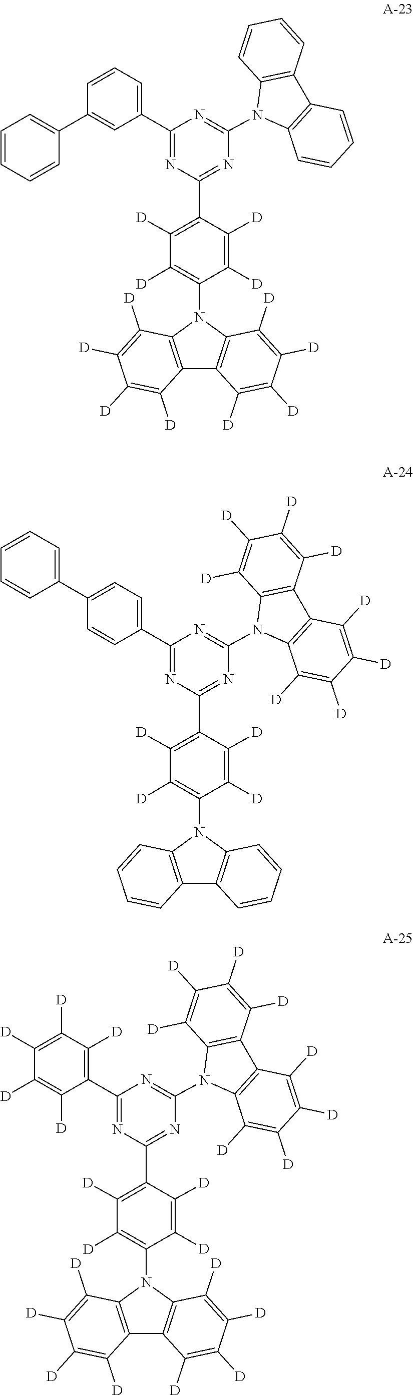

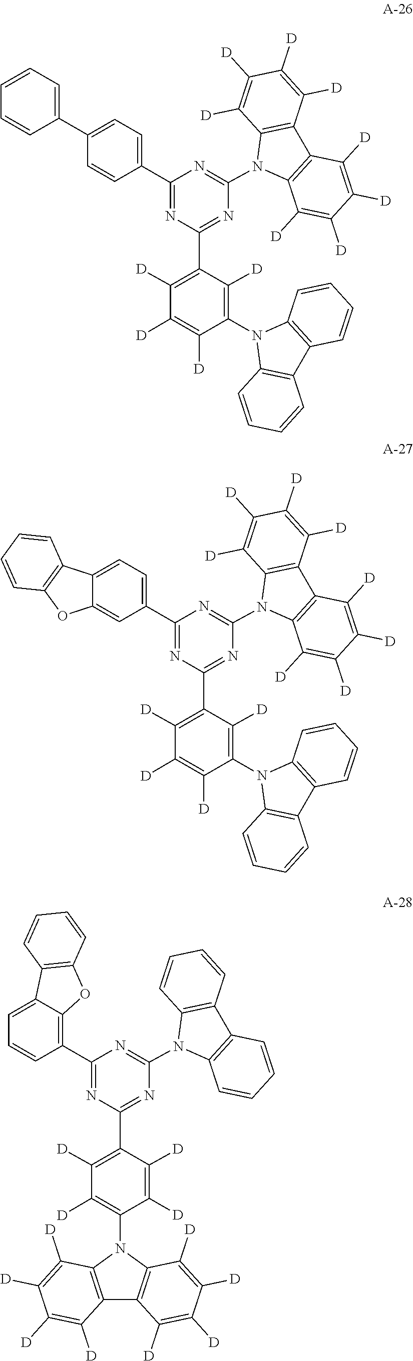 [SMILES: [2H]c1c([2H])c(-c2nc(-c3ccc(-c4ccccc4)cc3)nc(-n3c4c([2H])c([2H])c([2H])c([2H])c4c4c([2H])c([2H])c([2H])c([2H])c43)n2)c([2H])c(-n2c3ccccc3c3ccccc32)c1[2H] ; [2H]c1c([2H])c(-c2nc(-c3ccc4c(c3)oc3ccccc34)nc(-n3c4c([2H])c([2H])c([2H])c([2H])c4c4c([2H])c([2H])c([2H])c([2H])c43)n2)c([2H])c(-n2c3ccccc3c3ccccc32)c1[2H] ; [2H]c1c([2H])c(-n2c3c([2H])c([2H])c([2H])c([2H])c3c3c([2H])c([2H])c([2H])c([2H])c32)c([2H])c([2H])c1-c1nc(-c2cccc3c2oc2ccccc23)nc(-n2c3ccccc3c3ccccc32)n1]

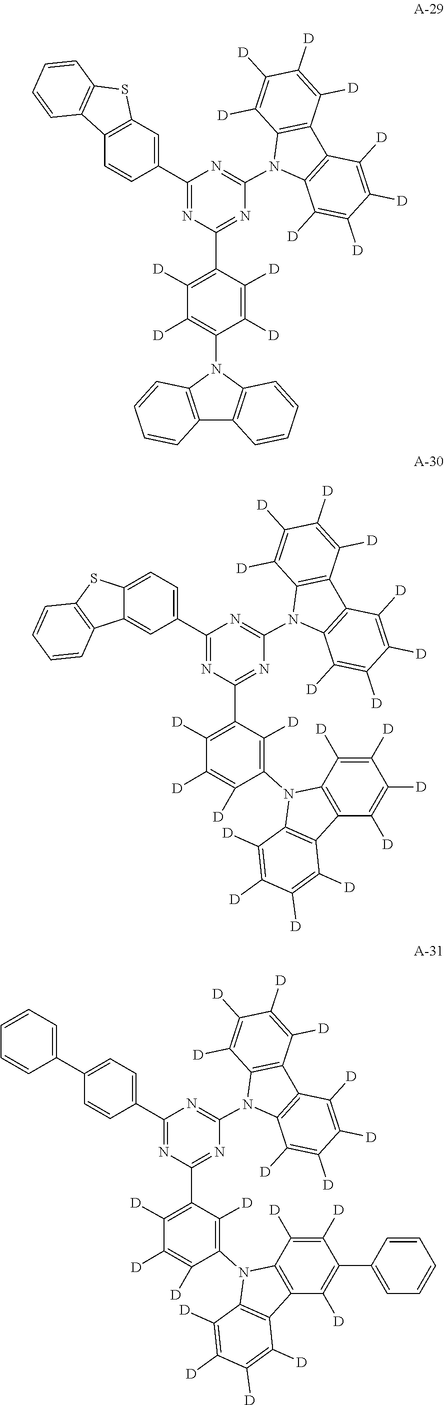

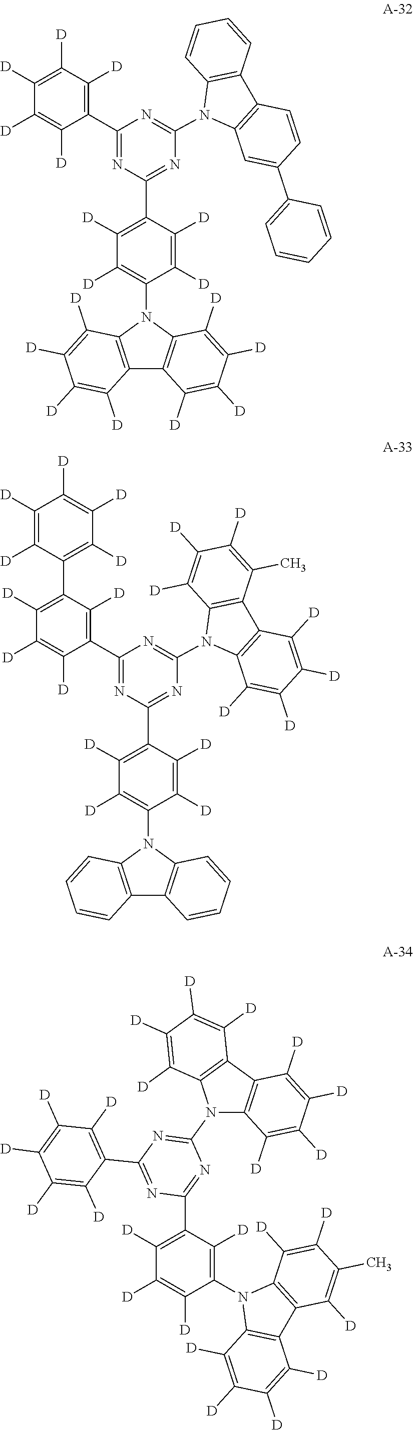

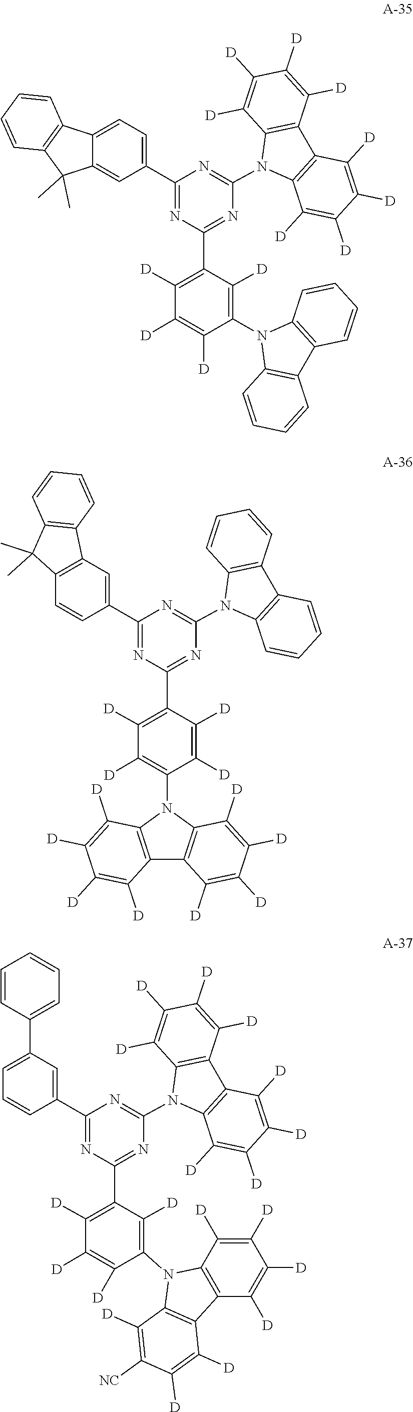

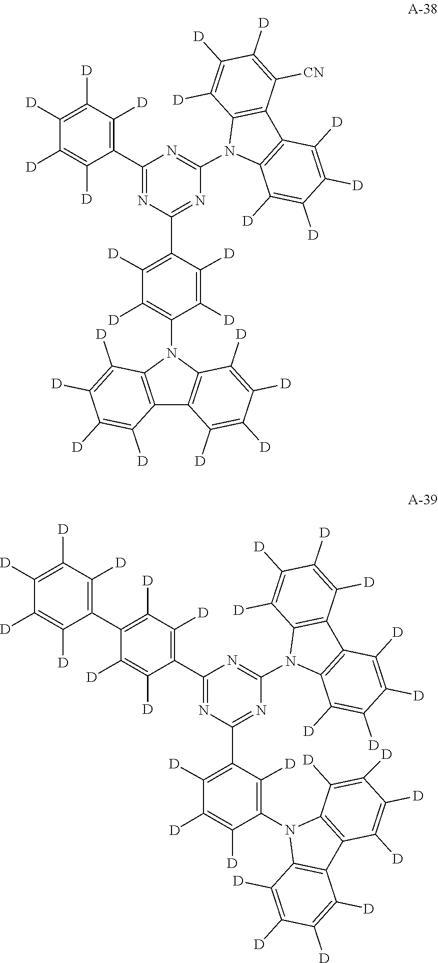 [SMILES: [2H]c1c([2H])c([2H])c(-c2nc(-c3c([2H])c([2H])c(-n4c5c([2H])c([2H])c([2H])c([2H])c5c5c([2H])c([2H])c([2H])c([2H])c54)c([2H])c3[2H])nc(-n3c4c([2H])c([2H])c([2H])c([2H])c4c4c(C#N)c([2H])c([2H])c([2H])c43)n2)c([2H])c1[2H] ; [2H]c1c([2H])c([2H])c(-c2c([2H])c([2H])c(-c3nc(-c4c([2H])c([2H])c([2H])c(-n5c6c([2H])c([2H])c([2H])c([2H])c6c6c([2H])c([2H])c([2H])c([2H])c65)c4[2H])nc(-n4c5c([2H])c([2H])c([2H])c([2H])c5c5c([2H])c([2H])c([2H])c([2H])c54)n3)c([2H])c2[2H])c([2H])c1[2H]]

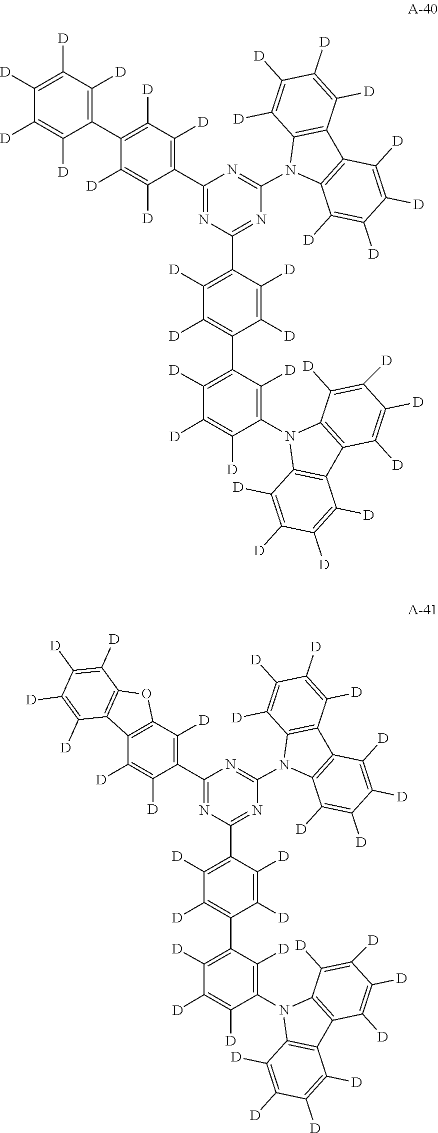

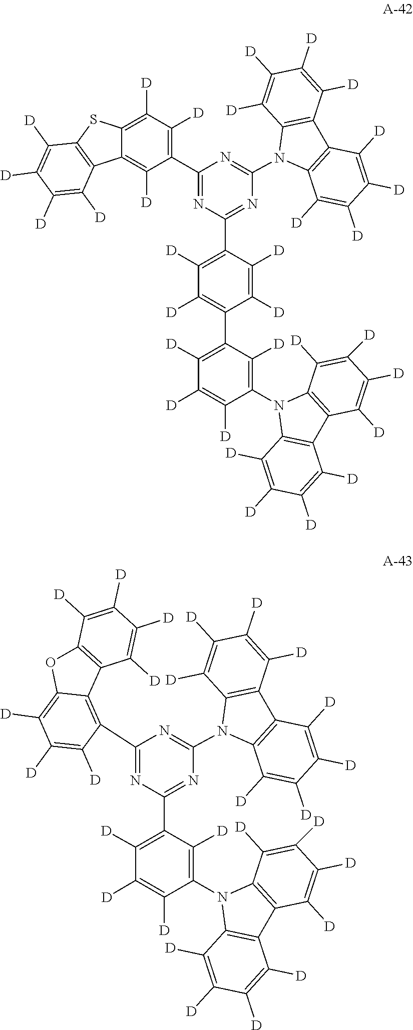

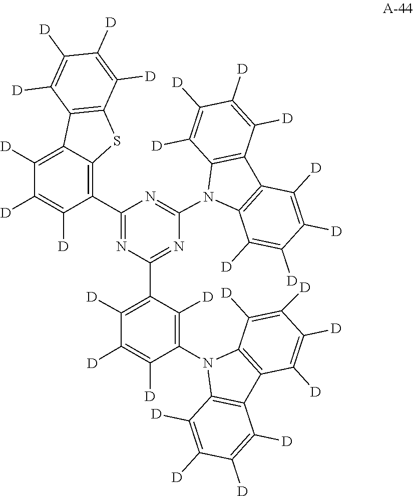 [SMILES: [2H]c1c([2H])c(-c2nc(-c3c([2H])c([2H])c([2H])c4c3sc3c([2H])c([2H])c([2H])c([2H])c34)nc(-n3c4c([2H])c([2H])c([2H])c([2H])c4c4c([2H])c([2H])c([2H])c([2H])c43)n2)c([2H])c(-n2c3c([2H])c([2H])c([2H])c([2H])c3c3c([2H])c([2H])c([2H])c([2H])c32)c1[2H]]

A composition for an organic optoelectronic device according to another embodiment may include a first compound and a second compound. In an implementation, the first compound may be, e.g., the aforementioned compound for an organic optoelectronic device, and the second compound may be represented by, e.g., Chemical Formula 2; or a combination of Chemical Formula 3 and Chemical Formula 4.

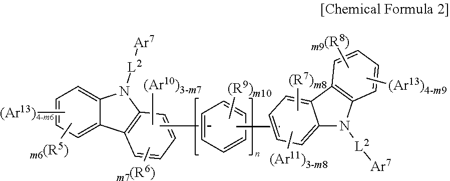

In Chemical Formula 2, R⁵ to R⁹ and Ar⁹ to Ar¹² may each independently be or include, e.g., hydrogen, deuterium, a cyano group, a halogen, a substituted or unsubstituted amine group, a substituted or unsubstituted C1 to C30 alkyl group, a substituted or unsubstituted C6 to C30 aryl group or a substituted or unsubstituted C2 to C30 heterocyclic group.

Ar⁷ and Ar⁸ may each independently be or include, e.g., a substituted or unsubstituted C6 to C20 aryl group or a substituted or unsubstituted C2 to C30 heterocyclic group.

L² and L³ may each independently be or include, e.g., a single bond or a substituted or unsubstituted C6 to C20 arylene group.

m6, m9, and m10 may each independently be, e.g., an integer of 1 to 4.

m7 and m8 may each independently be, e.g., an integer of 1 to 3.

n may be, e.g., an integer of 0 to 2.

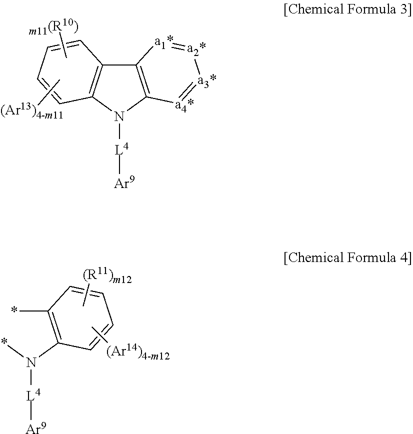

In Chemical Formula 3 and Chemical Formula 4, two adjacent ones of a1* to a4* of Chemical Formula 3 are linking carbons linked at * of Chemical Formula 4, the remaining two of a1* to a4* of Chemical Formula 3, not linked at * of Chemical Formula 4, may be, e.g., C-L^{a}-R^{a}. As used herein, the term “linking carbon” refers to a shared carbon at which fused rings are linked.

L^{a}, L⁴, and L⁵ may each independently be or include, e.g., a single bond or a substituted or unsubstituted C6 to C20 arylene group.

R^{a}, R¹⁰, R¹¹, Ar¹³, and Ar¹⁴ may each independently be or include, e.g., hydrogen, deuterium, a cyano group, a halogen, a substituted or unsubstituted amine group, a substituted or unsubstituted C1 to C30 alkyl group, a substituted or unsubstituted C6 to C30 aryl group or a substituted or unsubstituted C2 to C30 heterocyclic group.

Ar⁹ and Ar¹⁰ may each independently be or include, e.g., a substituted or unsubstituted C6 to C20 aryl group or a substituted or unsubstituted C2 to C30 heterocyclic group.

m11 and m12 may each independently be, e.g., an integer of 1 to 4.

The second compound may be used (e.g., mixed) together with the first compound in the light emitting layer to help increase charge mobility and stability, thereby improving luminous efficiency and life-span characteristics.

In an implementation, Ar⁷ and Ar⁸ in Chemical Formula 2 may each independently be, e.g., a substituted or unsubstituted phenyl group, a substituted or unsubstituted biphenyl group, a substituted or unsubstituted terphenyl group, a substituted or unsubstituted naphthyl group, a substituted or unsubstituted anthracenyl group, a substituted or unsubstituted triphenylenyl group, a substituted or unsubstituted carbazolyl group, a substituted or unsubstituted dibenzothiophenyl group, a substituted or unsubstituted dibenzofuranyl group, or a substituted or unsubstituted fluorenyl group.

In an implementation, L² and L³ in Chemical Formula 2 may each independently be, e.g., a single bond, a substituted or unsubstituted phenylene group, or a substituted or unsubstituted biphenylene group.

In an implementation, R⁵ to R⁸ and Ar⁹ to Ar¹² in Chemical Formula 2 may each independently be, e.g., hydrogen, deuterium, or a substituted or unsubstituted C6 to C12 aryl group.

In an implementation, n may be, e.g., 0 or 1.

In an implementation, “substituted” in Chemical Formula 2 means that at least one hydrogen is substituted with deuterium, a C1 to C4 alkyl group, a C6 to C18 aryl group, or a C2 to C30 heteroaryl group.

In an implementation, Chemical Formula 2 may be represented by one of Chemical Formula 2-1 to Chemical Formula 2-15.

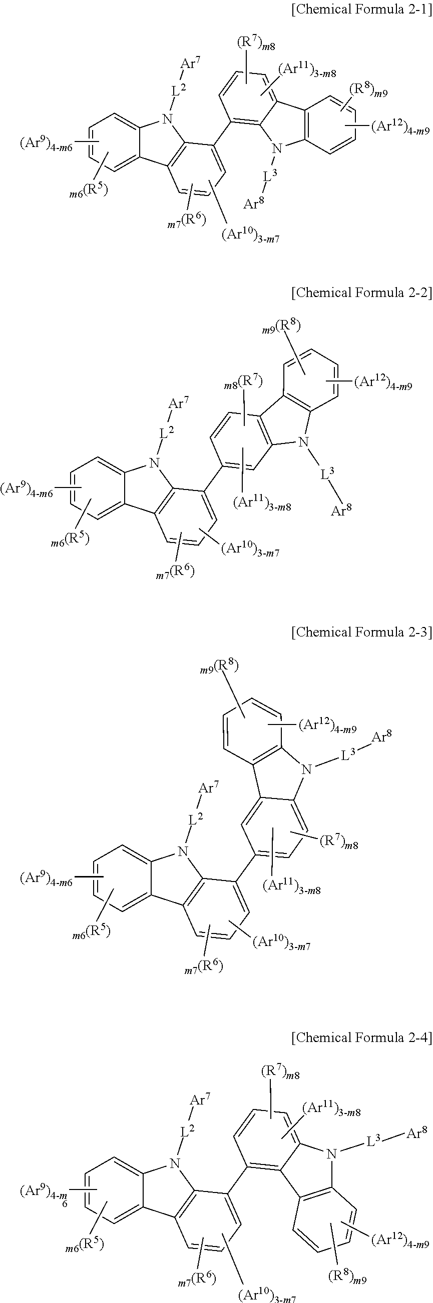

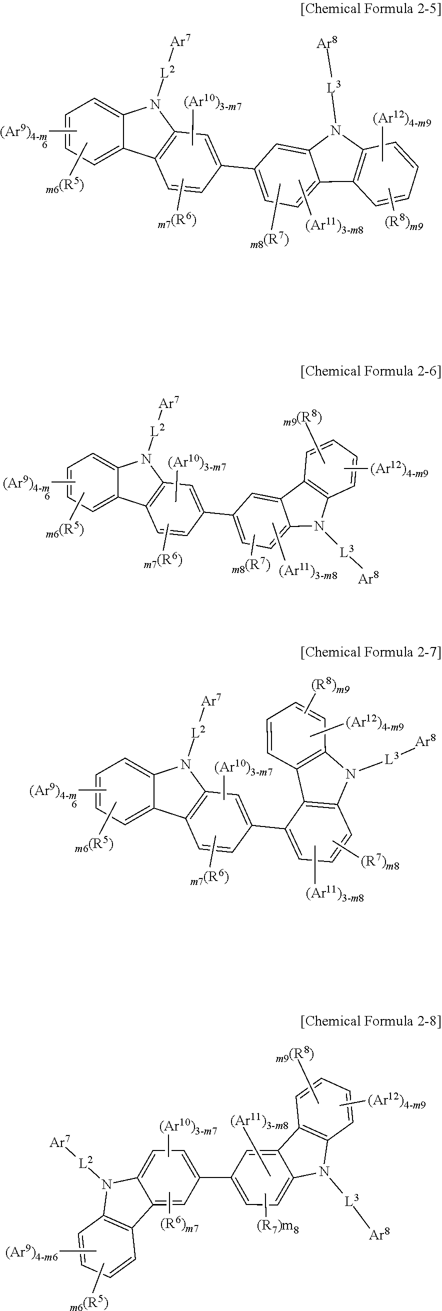

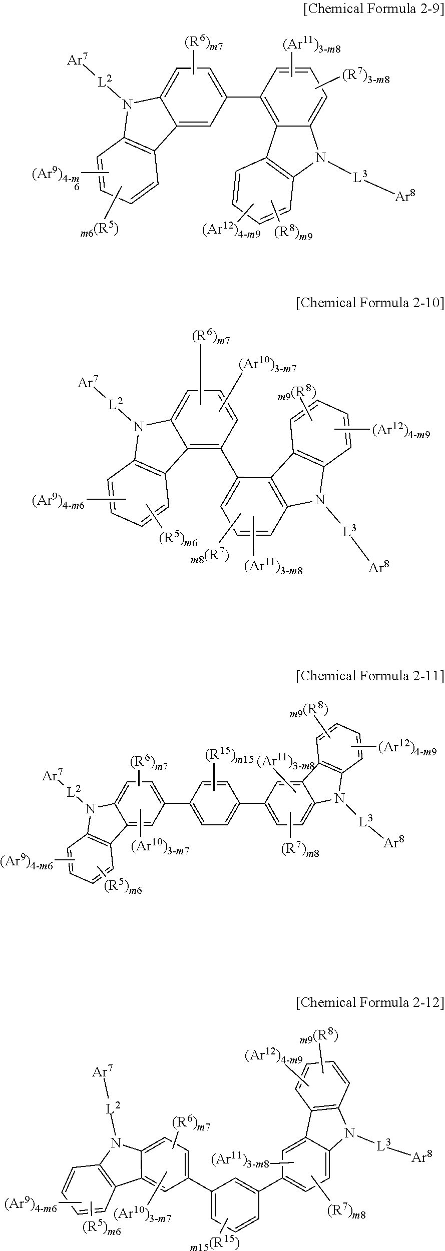

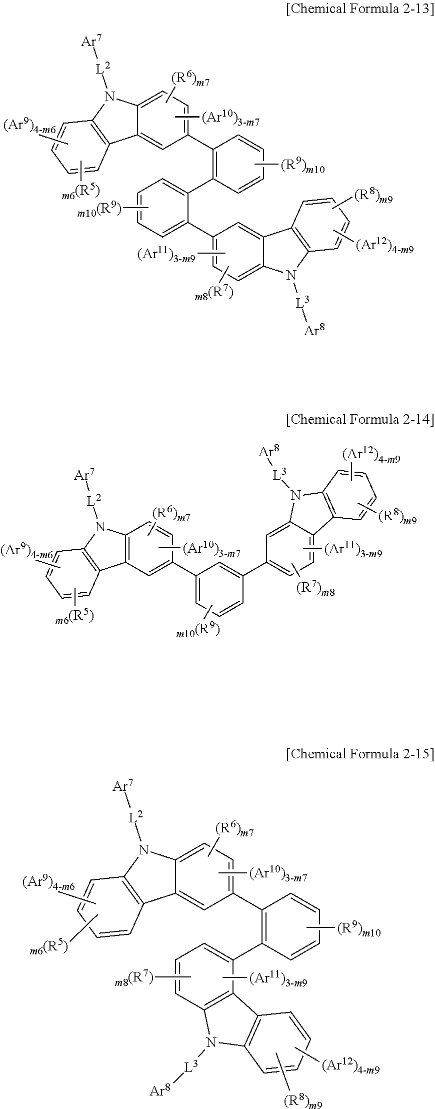

In Chemical Formula 2-1 to Chemical Formula 2-15, R⁵ to R⁹ and Ar⁹ to Ar¹² may each independently be, e.g., hydrogen, deuterium, or a substituted or unsubstituted C6 to C12 aryl group, and moieties *-L²-Ar⁷ and *-L³-Ar⁸ may each independently be, e.g., a moiety of Group II.

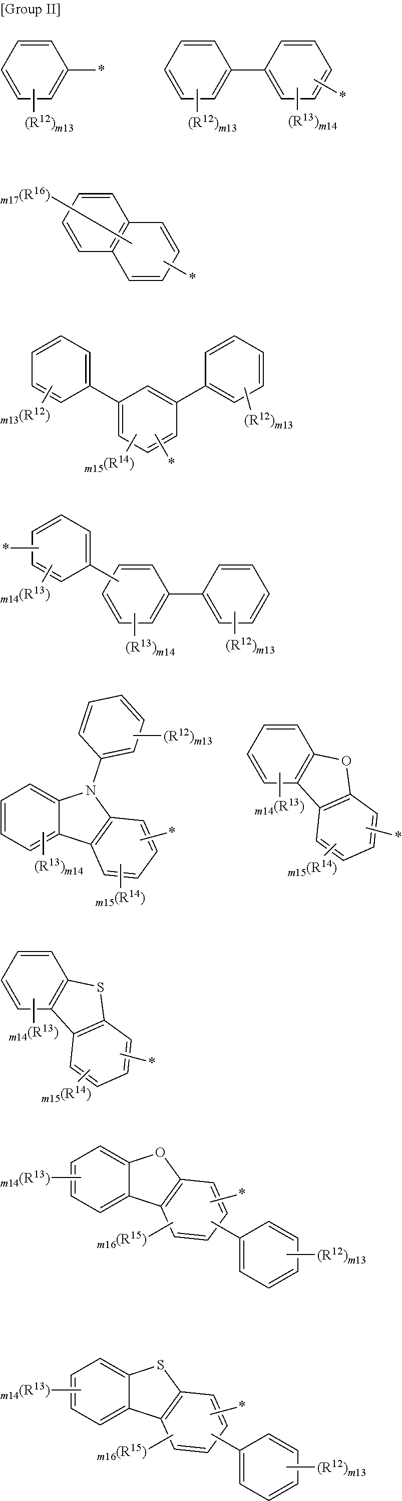

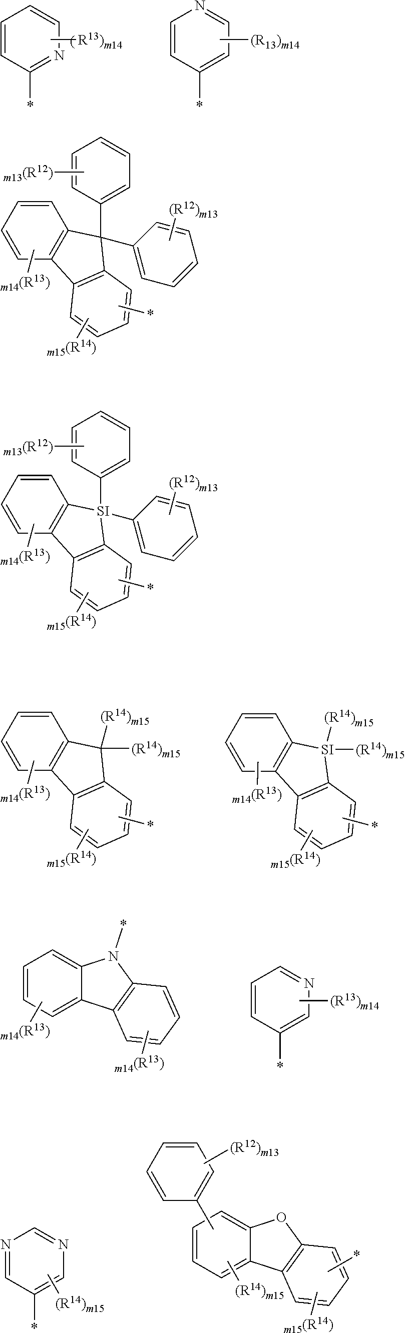

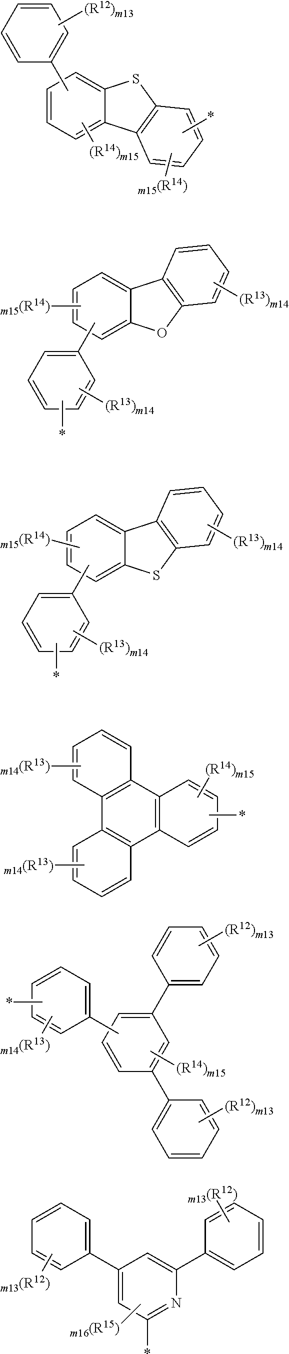

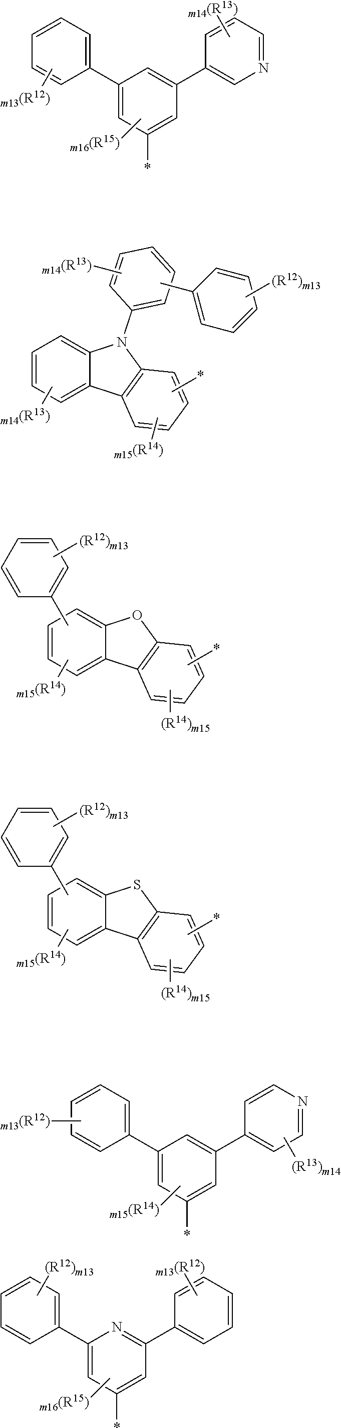

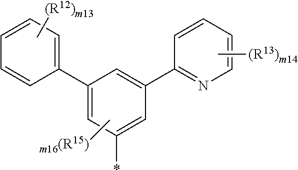

In Group II, R¹² to R¹⁶ may each independently be, e.g., hydrogen, deuterium, a cyano group, a substituted or unsubstituted C1 to C10 alkyl group, or a substituted or unsubstituted C6 to C12 aryl group.

m13 may be, e.g., an integer of 1 to 5.

m14 may be, e.g., an integer of 1 to 4.

m15 may be, e.g., an integer of 1 to 3.

m16 may be, e.g., 1 or 2.

m17 may be, e.g., an integer 1 to 7.

* is a linking point.

In an implementation, Chemical Formula 2 may be, e.g., represented by Chemical Formula 2-8.

In an implementation, moieties *-L²-Ar⁷ and *-L³-Ar⁸ in Chemical Formula 2-8 may each independently be, e.g., a moiety of Group II.

In an implementation, the second compound represented by the combination of Chemical Formula 3 and Chemical Formula 4 may be represented by, e.g., Chemical Formula 3A, Chemical Formula 3B, Chemical Formula 3C, Chemical Formula 3D, or Chemical Formula 3E.

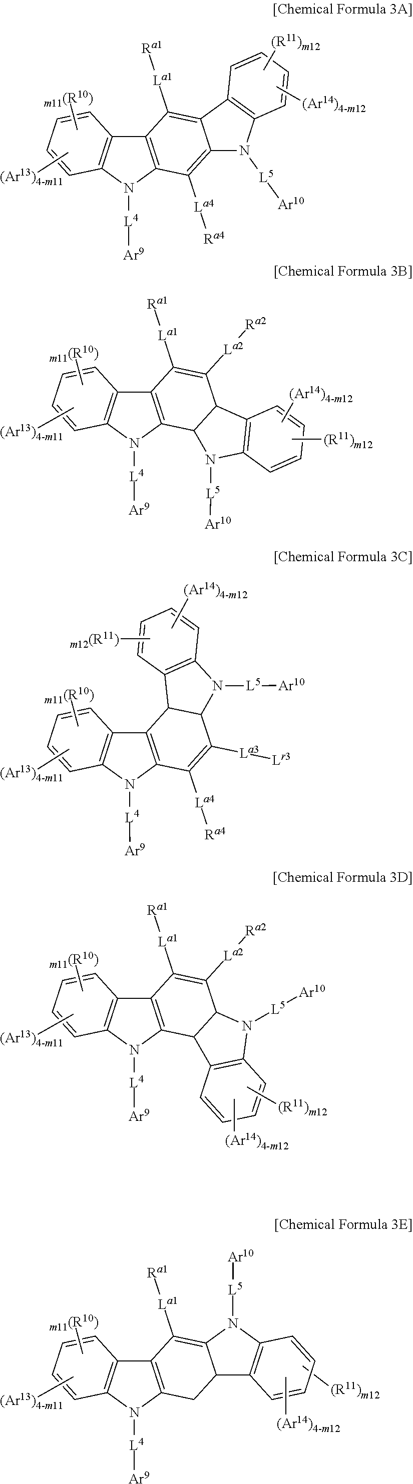

In Chemical Formula 3A to Chemical Formula 3E, Ar¹³, Ar¹⁴, L⁴, L⁵, Ar⁹, Ar¹⁰, R¹⁰, and R¹¹ may be defined the same as those described above.

L^{a1} to L^{a4} may be defined the same as L⁴ and L⁵ described above.

R^{a1} to R^{a4} may be defined the same as Ar¹³, Ar¹⁴, R¹⁰, and R¹¹.

In an implementation, Ar⁹ and Ar¹⁰ in Chemical Formulas 3 and 4 may each independently be, e.g., a substituted or unsubstituted phenyl group, a substituted or unsubstituted biphenyl group, a substituted or unsubstituted pyridinyl group, a substituted or unsubstituted carbazolyl group, a substituted or unsubstituted dibenzofuranyl group, or a substituted or unsubstituted dibenzothiophenyl group.

In an implementation, R^{a1} to R^{a4}, Ar¹³, Ar¹⁴, R¹⁰, and R¹¹ may each independently be, e.g., hydrogen, deuterium, a cyano group, a substituted or unsubstituted phenyl group, a substituted or unsubstituted biphenyl group, a substituted or unsubstituted pyridinyl group, a substituted or unsubstituted carbazolyl group, a substituted or unsubstituted dibenzofuranyl group, or a substituted or unsubstituted dibenzothiophenyl group.

In an implementation, moieties *-L⁴-Ar⁹ and *-L⁵-Ar¹⁰ in Chemical Formulas 3 and 4 may each independently be, e.g., a moiety of Group II.

In an implementation, R^{a1} to R^{a4}, Ar¹³, Ar¹⁴, R¹⁰, and R¹¹ may each independently be, e.g., hydrogen, deuterium, a cyano group, a substituted or unsubstituted phenyl group, a substituted or unsubstituted biphenyl group, a substituted or unsubstituted pyridinyl group, a substituted or unsubstituted carbazolyl group, a substituted or unsubstituted dibenzofuranyl group, or a substituted or unsubstituted dibenzothiophenyl group.

In an implementation, R^{a1} to R^{a4}, Ar¹³, Ar¹⁴, R¹⁰, and R¹¹ may each independently be, e.g., hydrogen, deuterium, a cyano group, or a substituted or unsubstituted phenyl group.

In an implementation, R^{a1} to R^{a4}, Ar¹³, Ar¹⁴, R¹⁰, and R¹¹ may each independently be hydrogen, deuterium, or a substituted or unsubstituted phenyl group.

In an implementation, the second compound may be represented by Chemical Formula 2-8, and in Chemical Formula 2-8, Ar⁷ and Ar⁸ may each independently be, e.g., a substituted or unsubstituted phenyl group, a substituted or unsubstituted biphenyl group, a substituted or unsubstituted pyridinyl group, a substituted or unsubstituted carbazolyl group, a substituted or unsubstituted dibenzofuranyl group, or a substituted or unsubstituted dibenzothiophenyl group, L² and L³ may each independently be, e.g., a single bond, or a substituted or unsubstituted C6 to C20 arylene group, and Ar⁹ to Ar¹² and R⁵ to R⁸ may each independently be, e.g., hydrogen, deuterium, a cyano group, a substituted or unsubstituted phenyl group, a substituted or unsubstituted biphenyl group, a substituted or unsubstituted pyridinyl group, a substituted or unsubstituted carbazolyl group, a substituted or unsubstituted dibenzofuranyl group, or a substituted or unsubstituted dibenzothiophenyl group.

In an implementation, the second compound may be represented by Chemical Formula 3C, and in Chemical Formula 3C, L^{a3} and L^{a4} may be, e.g., a single bond, L⁴ and L⁵ may each independently be, e.g., a single bond or a substituted or unsubstituted C6 to C12 arylene group, Ar¹³, Ar¹⁴, R¹⁰, R¹¹, R^{a3}, and R^{a4} may each independently be, e.g., hydrogen, deuterium or a substituted or unsubstituted phenyl group, and Ar⁹ and Ar¹⁰ may each independently be, e.g., a substituted or unsubstituted phenyl group, a substituted or unsubstituted biphenyl group, or a substituted or unsubstituted terphenyl group.

In an implementation, the second compound for an organic optoelectronic device may be, e.g., a compound of Group 2.

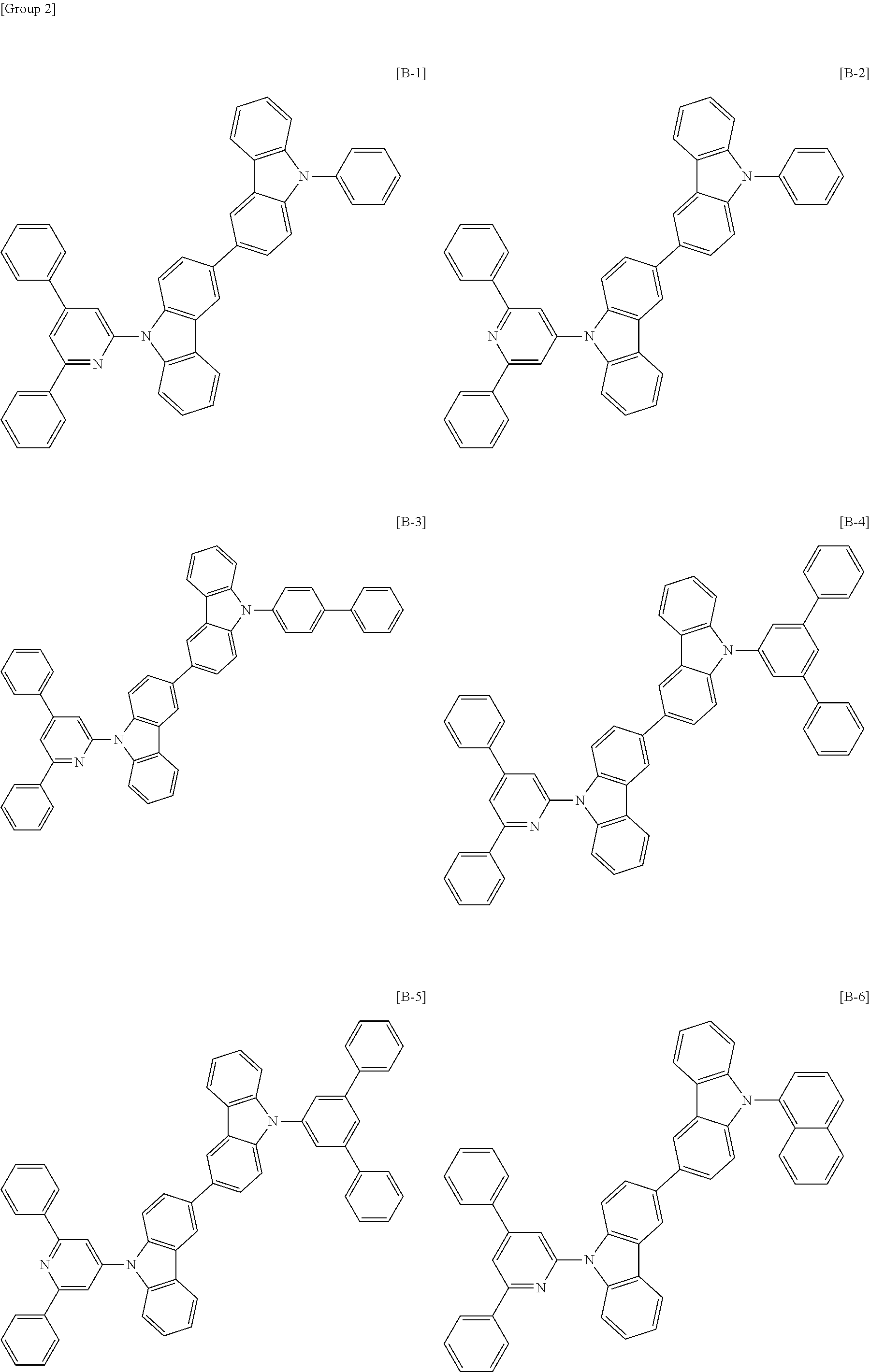

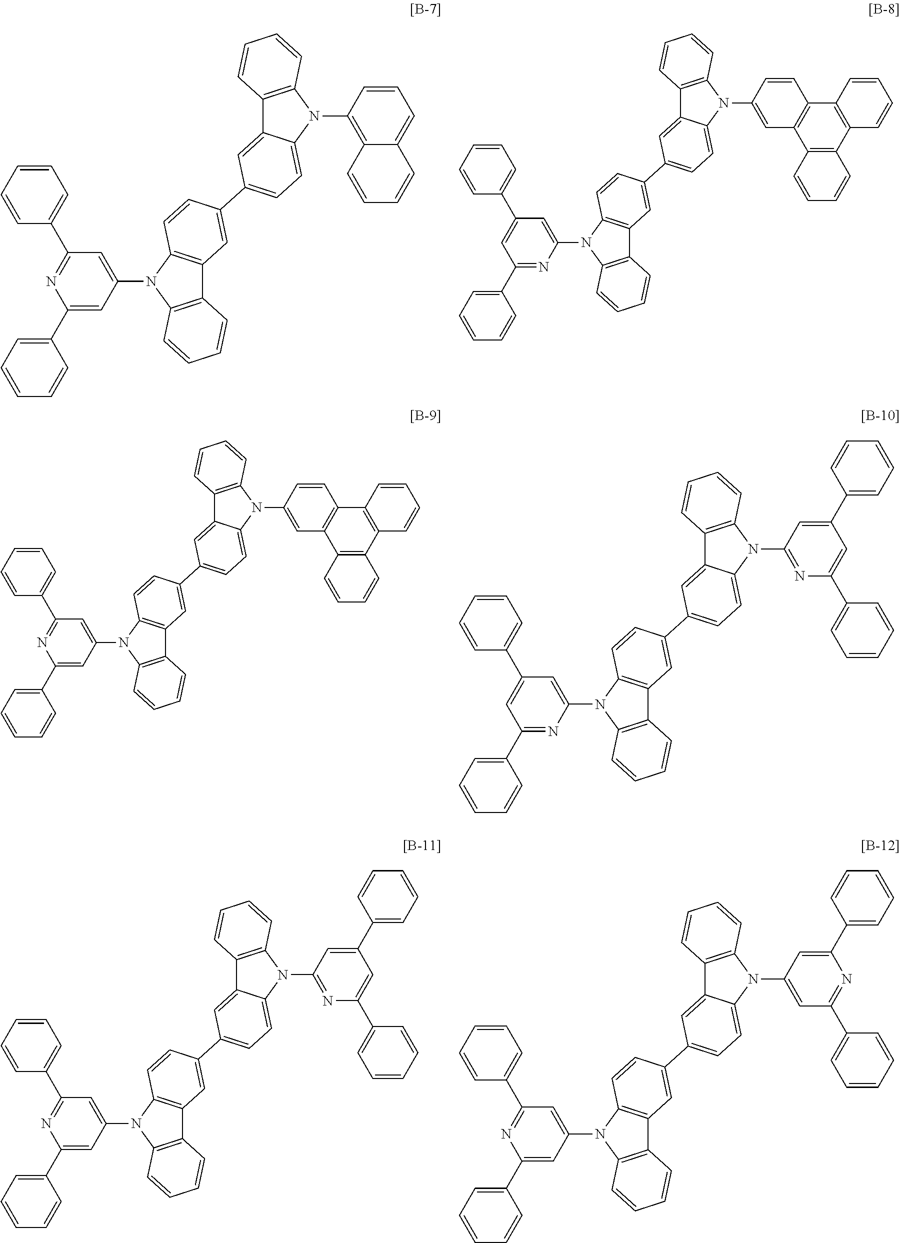

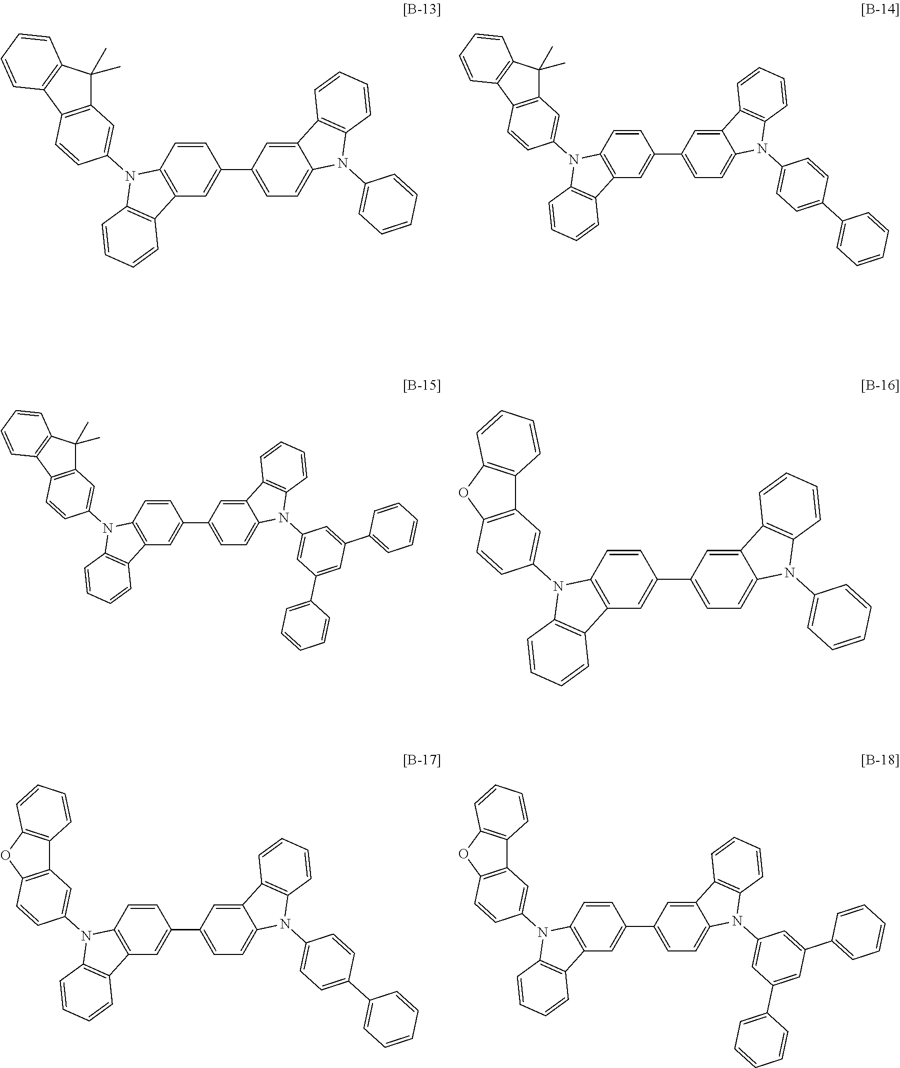

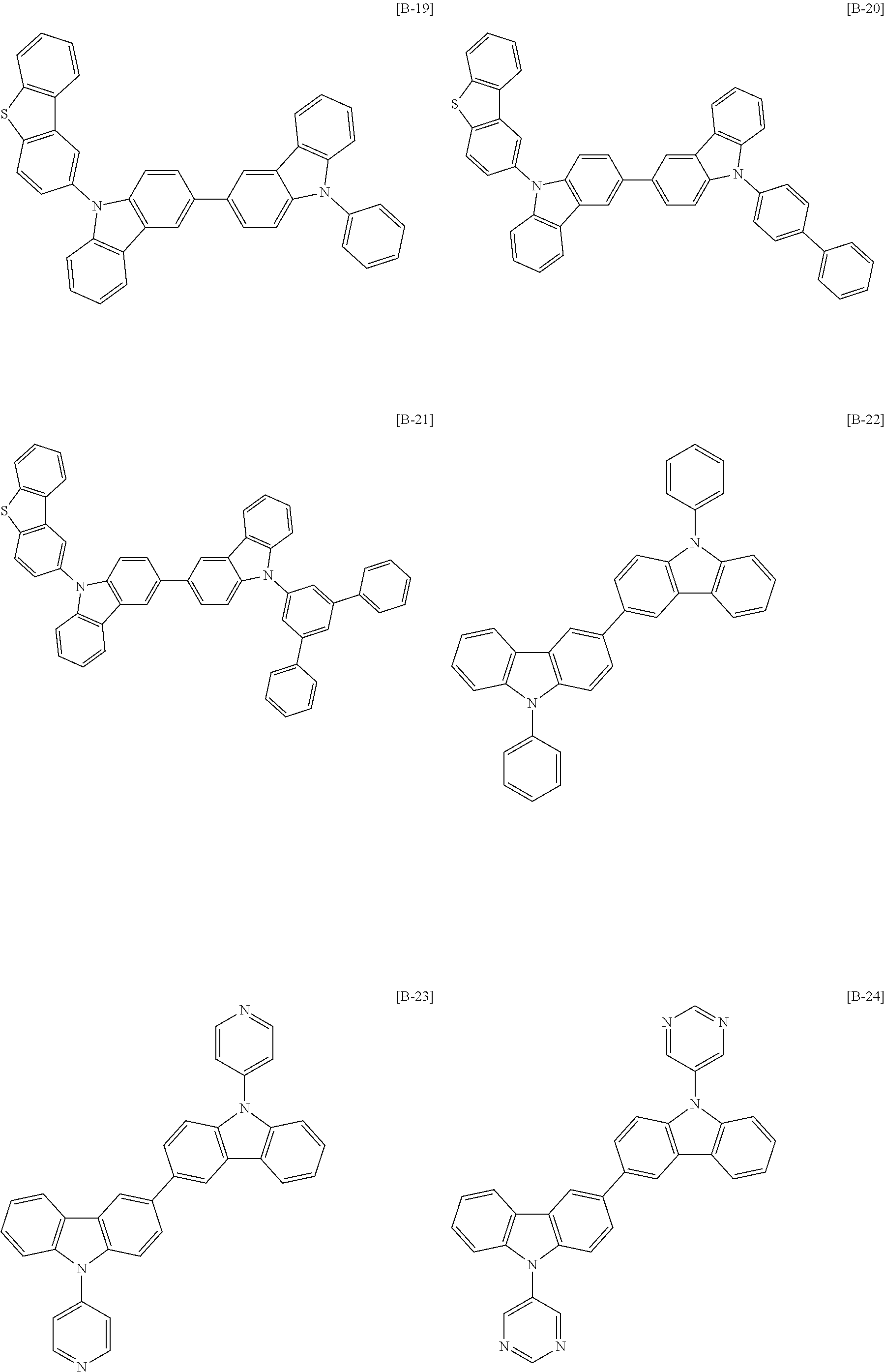 [SMILES: c1ccc(-n2c3ccccc3c3cc(-c4ccc5c(c4)c4ccccc4n5-c4ccc5sc6ccccc6c5c4)ccc32)cc1 ; c1ccc(-c2ccc(-n3c4ccccc4c4cc(-c5ccc6c(c5)c5ccccc5n6-c5ccc6sc7ccccc7c6c5)ccc43)cc2)cc1 ; c1ccc(-c2cc(-c3ccccc3)cc(-n3c4ccccc4c4cc(-c5ccc6c(c5)c5ccccc5n6-c5ccc6sc7ccccc7c6c5)ccc43)c2)cc1 ; c1ccc(-n2c3ccccc3c3cc(-c4ccc5c(c4)c4ccccc4n5-c4ccccc4)ccc32)cc1 ; c1ccc2c(c1)c1cc(-c3ccc4c(c3)c3ccccc3n4-c3ccncc3)ccc1n2-c1ccncc1 ; c1ccc2c(c1)c1cc(-c3ccc4c(c3)c3ccccc3n4-c3cncnc3)ccc1n2-c1cncnc1]

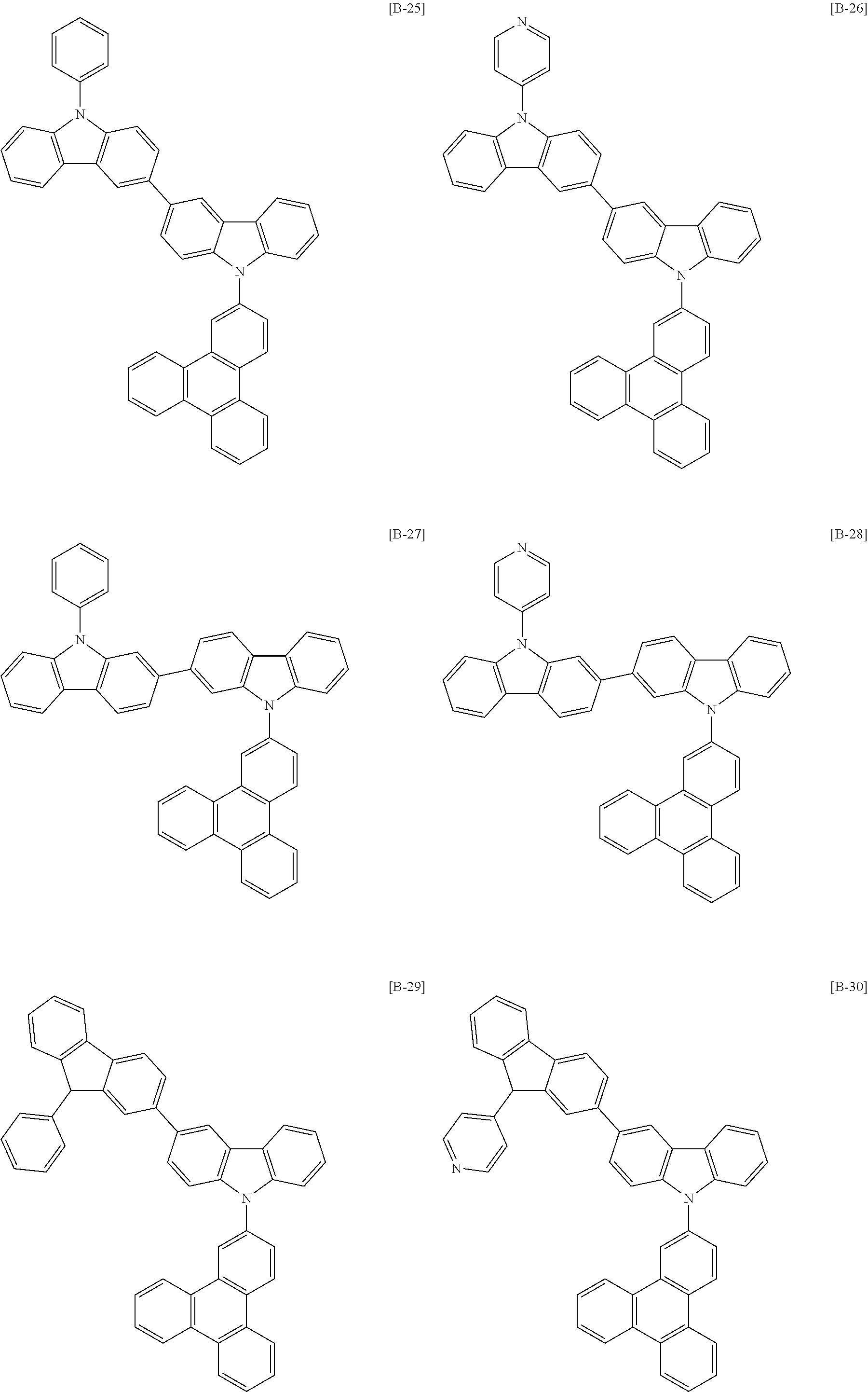

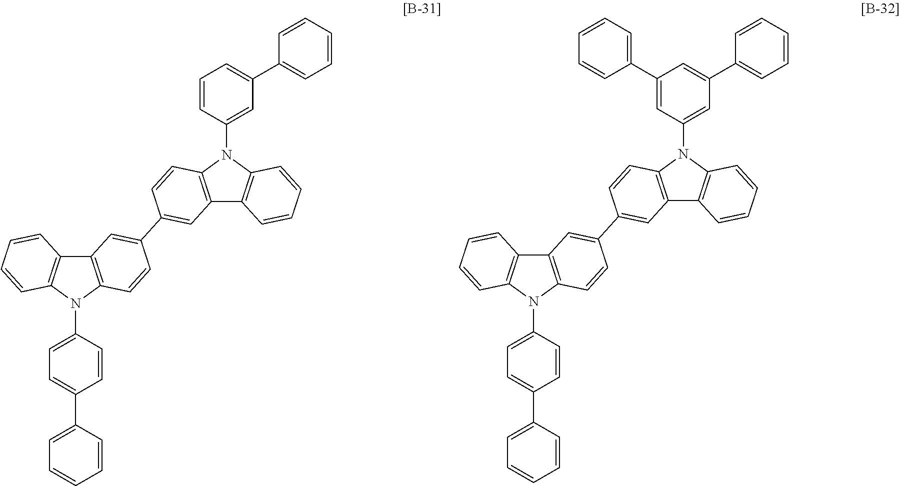 [SMILES: c1ccc(-c2ccc(-n3c4ccccc4c4cc(-c5ccc6c(c5)c5ccccc5n6-c5cccc(-c6ccccc6)c5)ccc43)cc2)cc1 ; c1ccc(-c2ccc(-n3c4ccccc4c4cc(-c5ccc6c(c5)c5ccccc5n6-c5cc(-c6ccccc6)cc(-c6ccccc6)c5)ccc43)cc2)cc1]

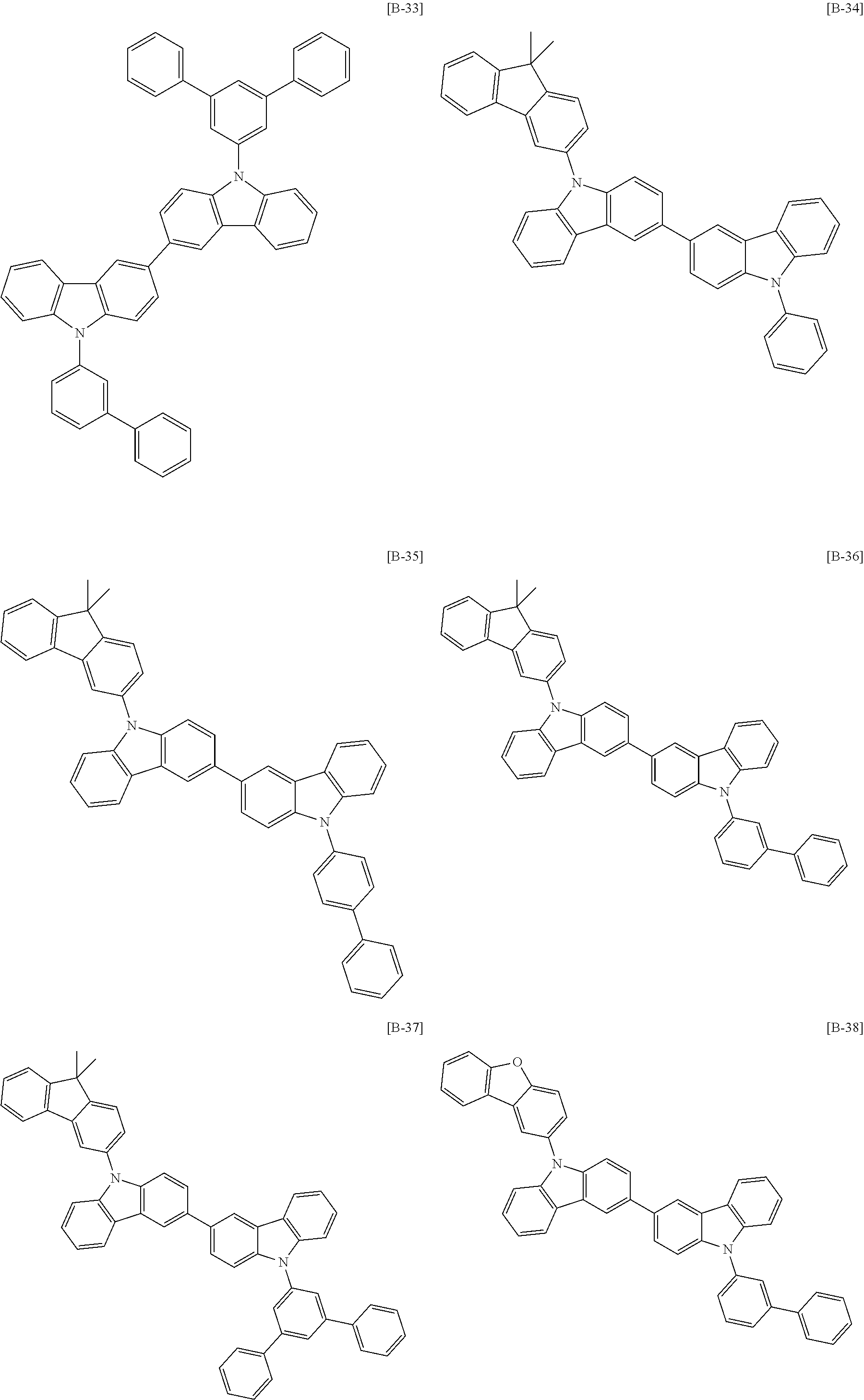

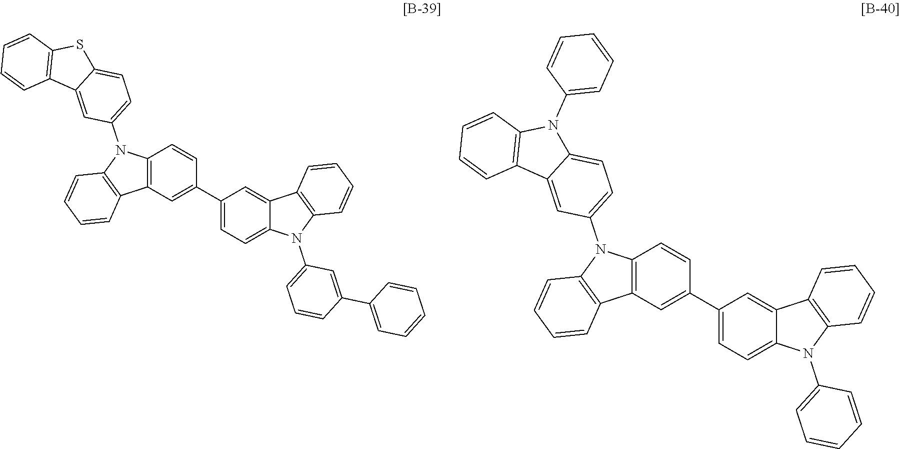 [SMILES: c1ccc(-c2cccc(-n3c4ccccc4c4cc(-c5ccc6c(c5)c5ccccc5n6-c5ccc6sc7ccccc7c6c5)ccc43)c2)cc1 ; c1ccc(-n2c3ccccc3c3cc(-c4ccc5c(c4)c4ccccc4n5-c4ccc5c(c4)c4ccccc4n5-c4ccccc4)ccc32)cc1]

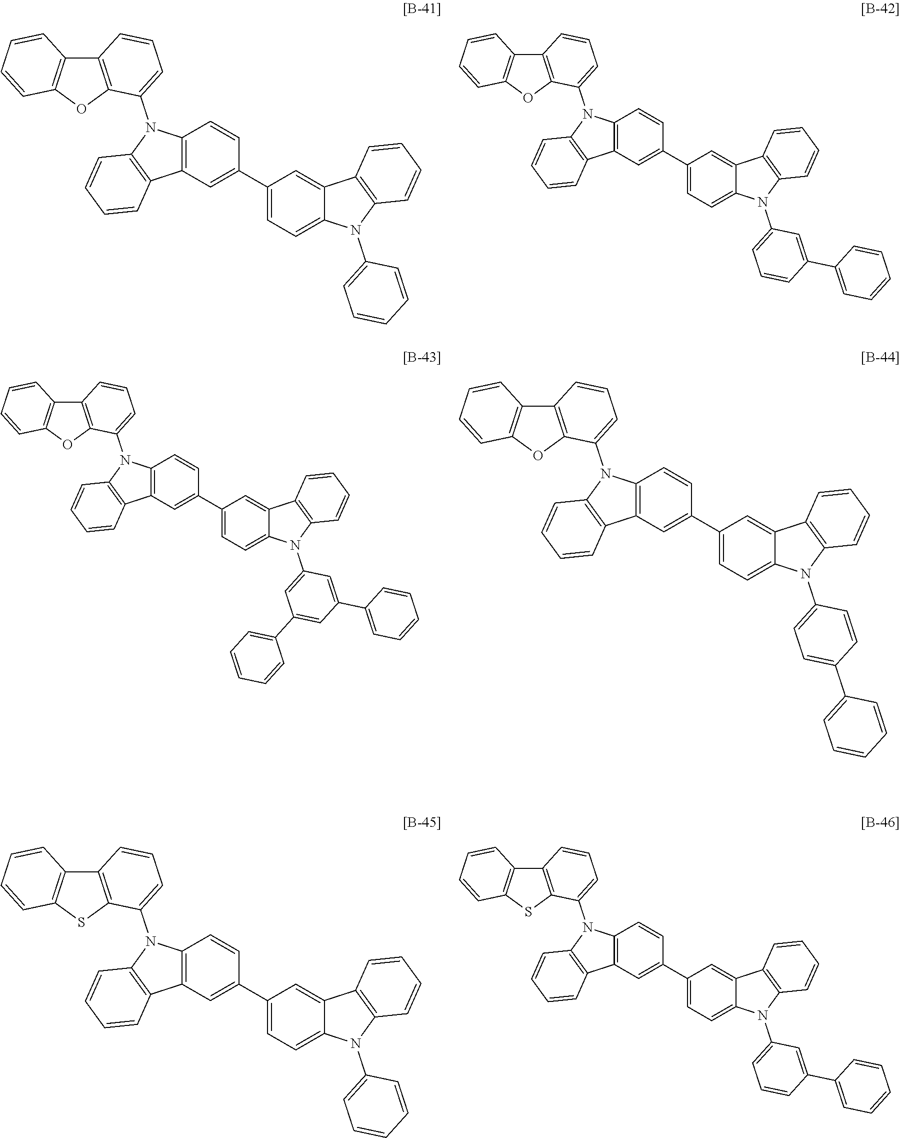

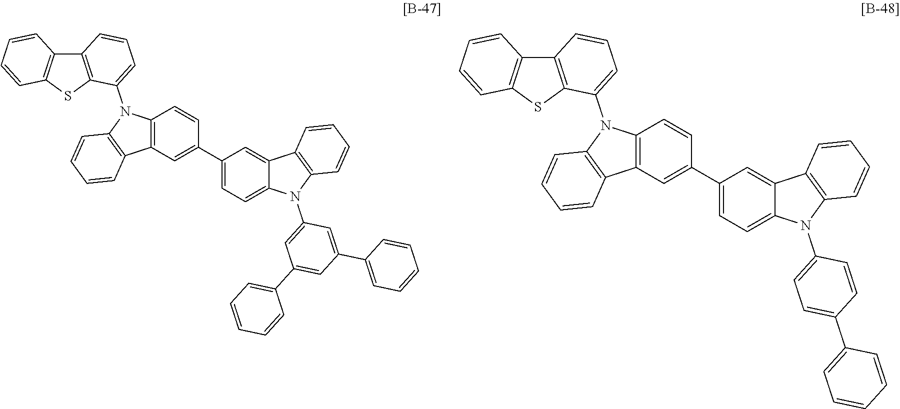 [SMILES: c1ccc(-c2cc(-c3ccccc3)cc(-n3c4ccccc4c4cc(-c5ccc6c(c5)c5ccccc5n6-c5cccc6c5sc5ccccc56)ccc43)c2)cc1 ; c1ccc(-c2ccc(-n3c4ccccc4c4cc(-c5ccc6c(c5)c5ccccc5n6-c5cccc6c5sc5ccccc56)ccc43)cc2)cc1]

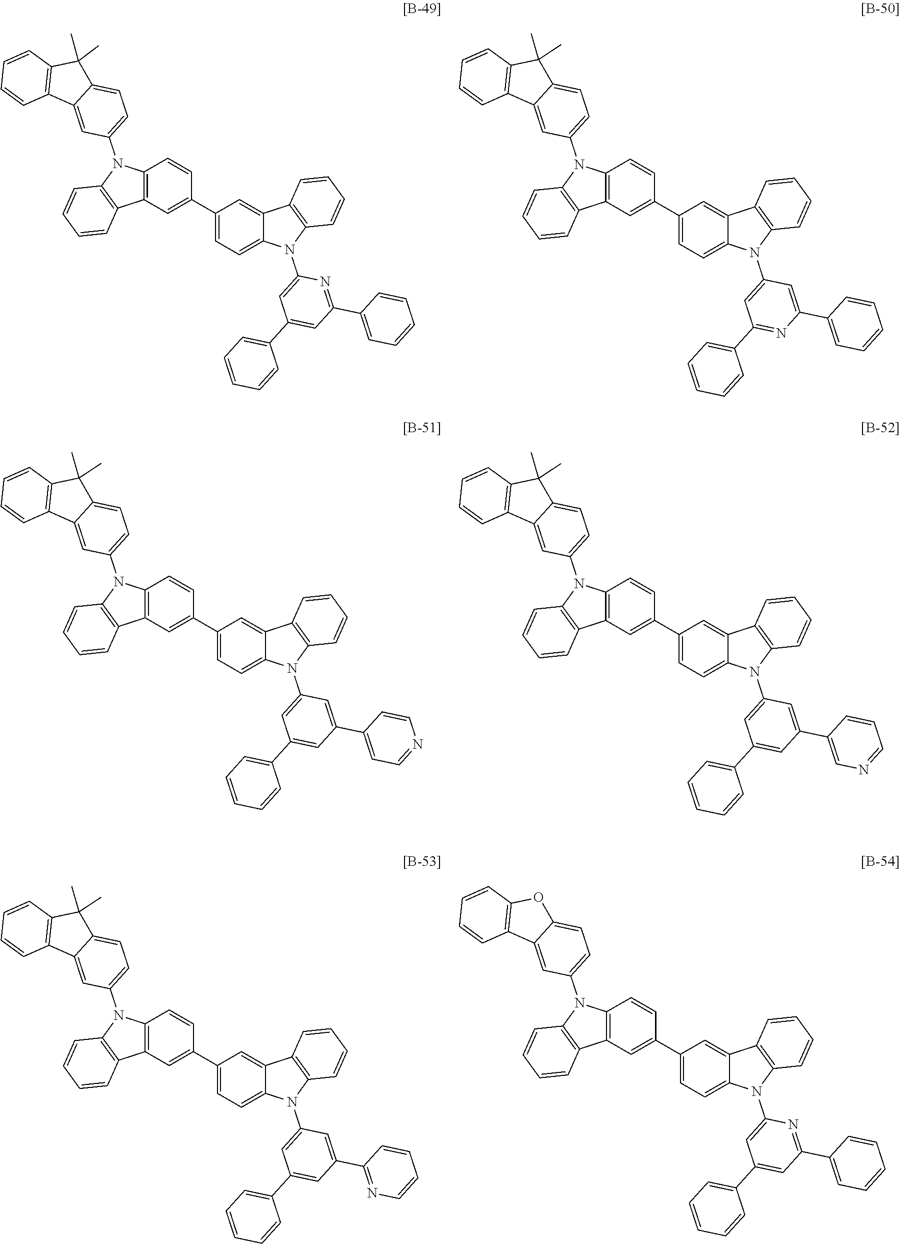

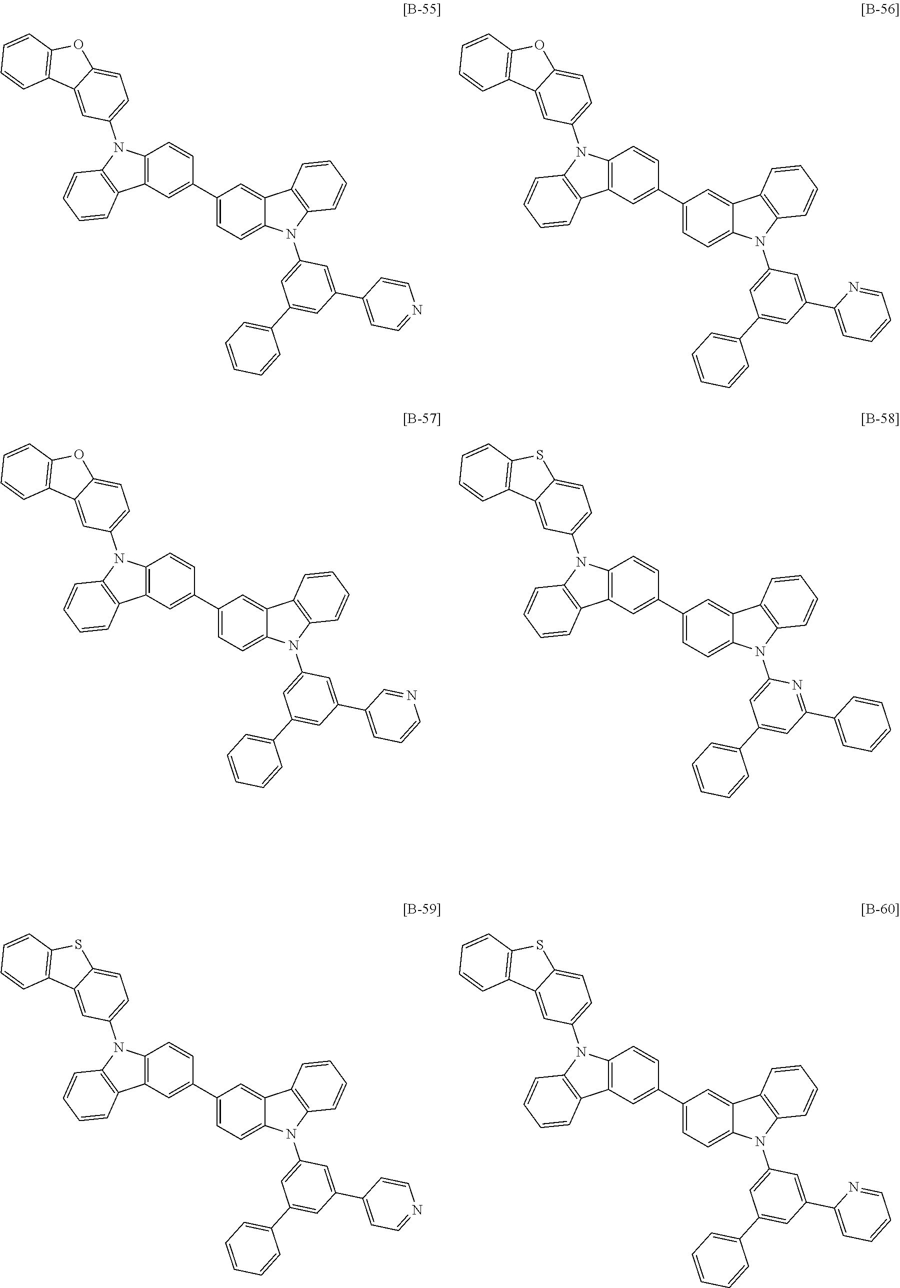

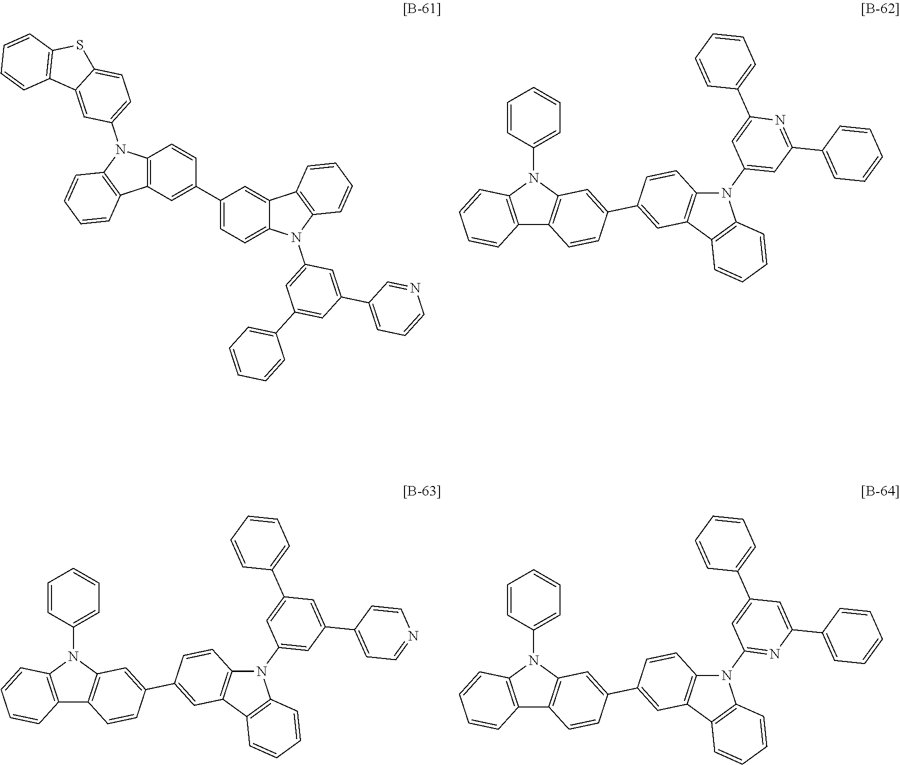 [SMILES: c1ccc(-c2cc(-c3cccnc3)cc(-n3c4ccccc4c4cc(-c5ccc6c(c5)c5ccccc5n6-c5ccc6sc7ccccc7c6c5)ccc43)c2)cc1 ; c1ccc(-c2cc(-n3c4ccccc4c4cc(-c5ccc6c7ccccc7n(-c7ccccc7)c6c5)ccc43)cc(-c3ccccc3)n2)cc1 ; c1ccc(-c2cc(-c3ccncc3)cc(-n3c4ccccc4c4cc(-c5ccc6c7ccccc7n(-c7ccccc7)c6c5)ccc43)c2)cc1 ; c1ccc(-c2cc(-c3ccccc3)nc(-n3c4ccccc4c4cc(-c5ccc6c7ccccc7n(-c7ccccc7)c6c5)ccc43)c2)cc1]

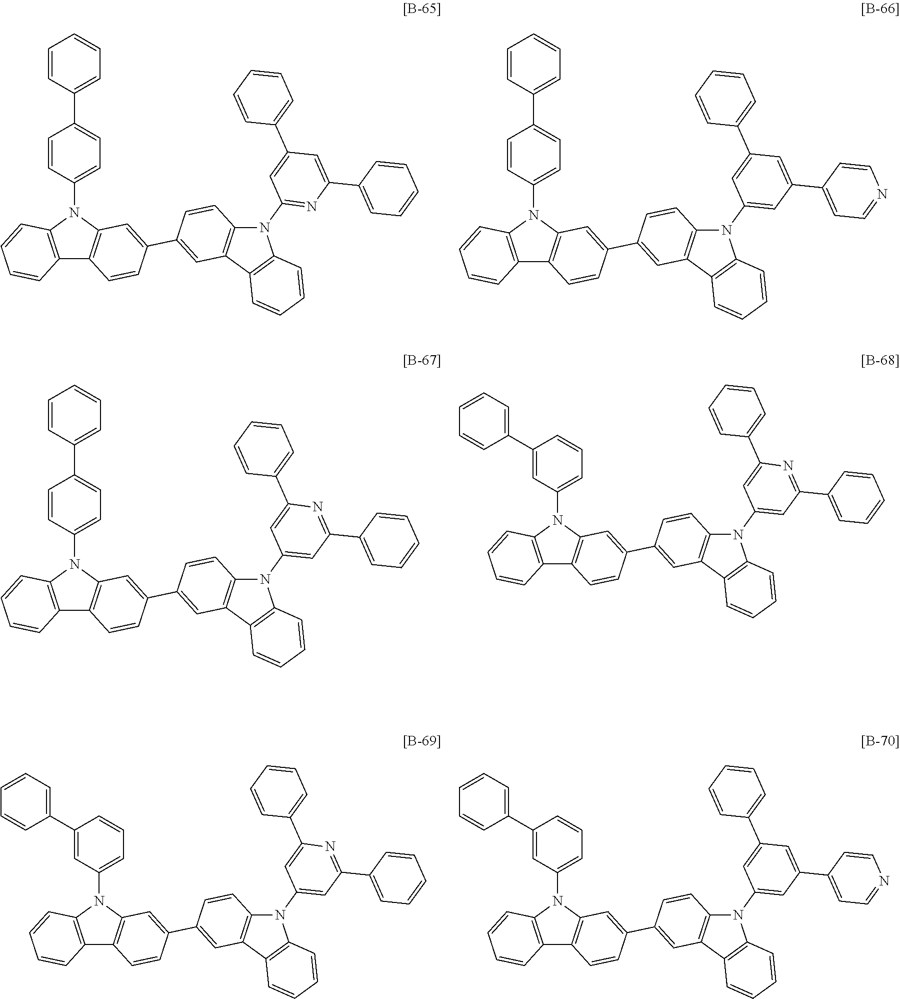

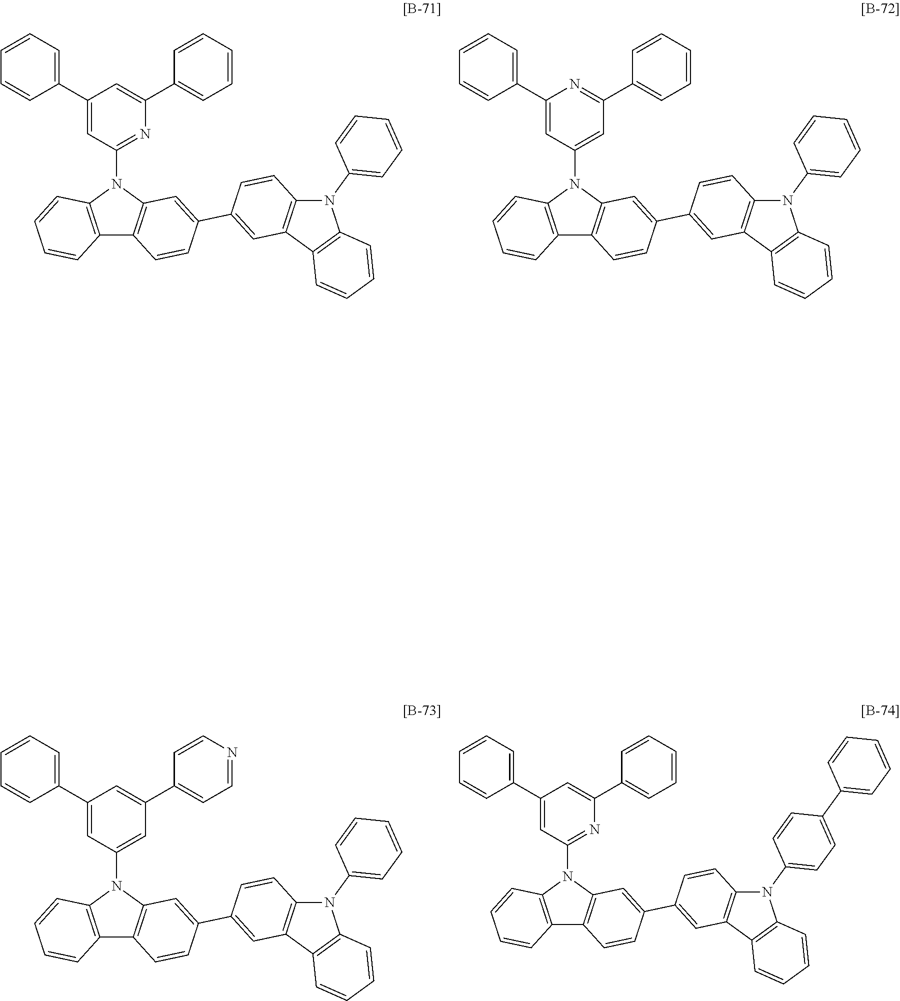 [SMILES: c1ccc(-c2cc(-c3ccccc3)nc(-n3c4ccccc4c4ccc(-c5ccc6c(c5)c5ccccc5n6-c5ccccc5)cc43)c2)cc1 ; c1ccc(-c2cc(-n3c4ccccc4c4ccc(-c5ccc6c(c5)c5ccccc5n6-c5ccccc5)cc43)cc(-c3ccccc3)n2)cc1 ; c1ccc(-c2cc(-c3ccncc3)cc(-n3c4ccccc4c4ccc(-c5ccc6c(c5)c5ccccc5n6-c5ccccc5)cc43)c2)cc1 ; c1ccc(-c2ccc(-n3c4ccccc4c4cc(-c5ccc6c7ccccc7n(-c7cc(-c8ccccc8)cc(-c8ccccc8)n7)c6c5)ccc43)cc2)cc1]

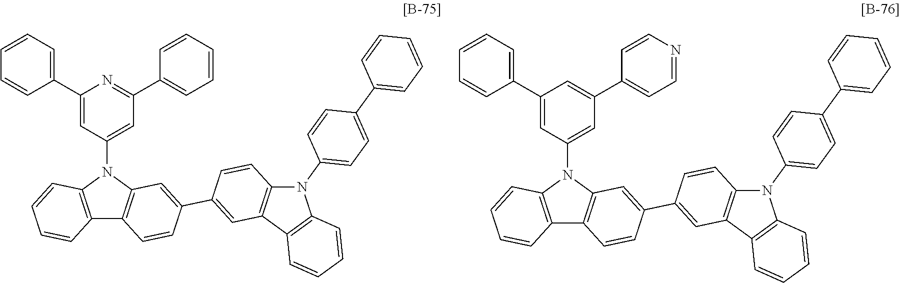

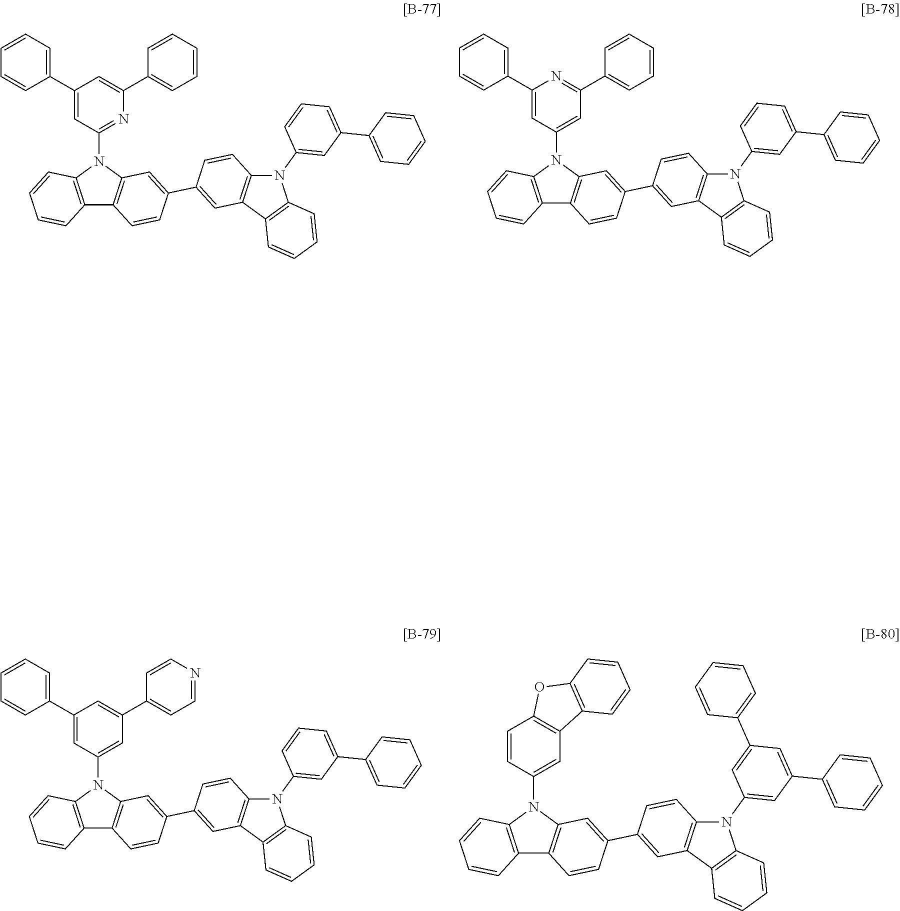 [SMILES: c1ccc(-c2cccc(-n3c4ccccc4c4cc(-c5ccc6c7ccccc7n(-c7cc(-c8ccccc8)cc(-c8ccccc8)n7)c6c5)ccc43)c2)cc1 ; c1ccc(-c2cccc(-n3c4ccccc4c4cc(-c5ccc6c7ccccc7n(-c7cc(-c8ccccc8)nc(-c8ccccc8)c7)c6c5)ccc43)c2)cc1 ; c1ccc(-c2cccc(-n3c4ccccc4c4cc(-c5ccc6c7ccccc7n(-c7cc(-c8ccccc8)cc(-c8ccncc8)c7)c6c5)ccc43)c2)cc1 ; c1ccc(-c2cc(-c3ccccc3)cc(-n3c4ccccc4c4cc(-c5ccc6c7ccccc7n(-c7ccc8oc9ccccc9c8c7)c6c5)ccc43)c2)cc1]

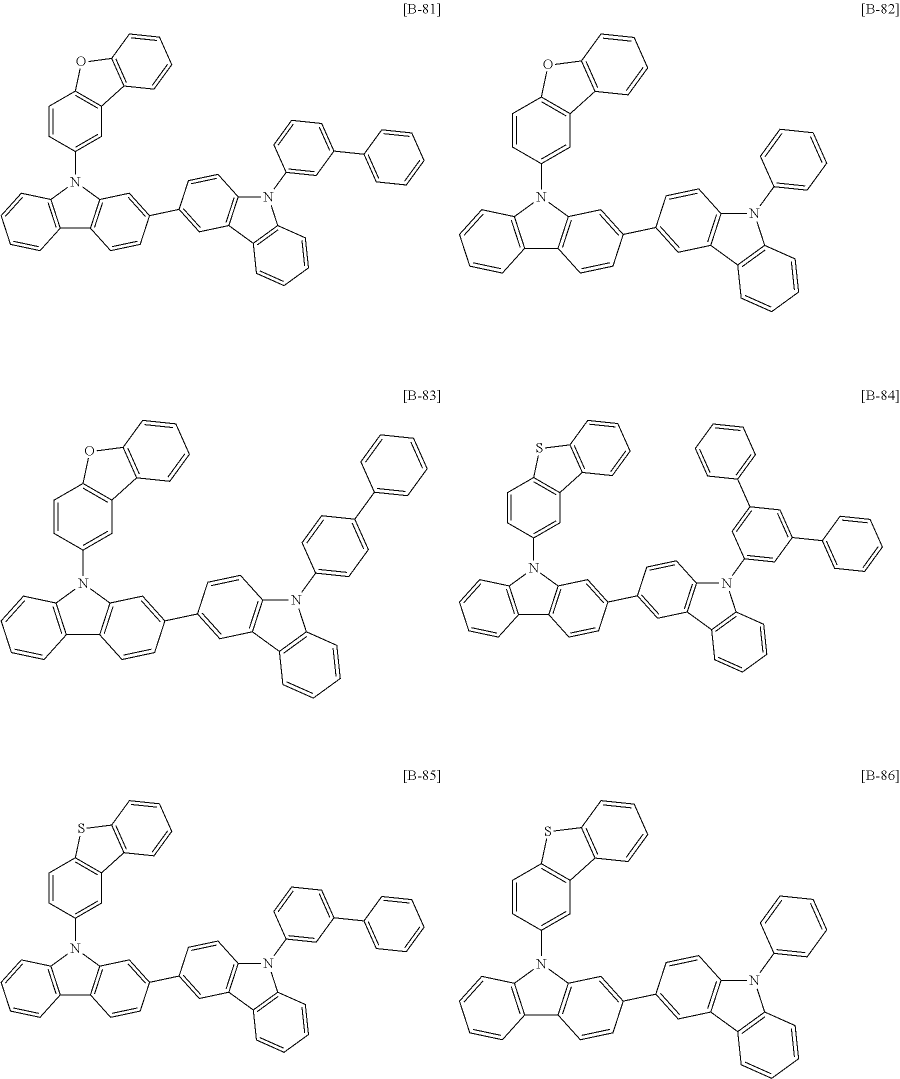

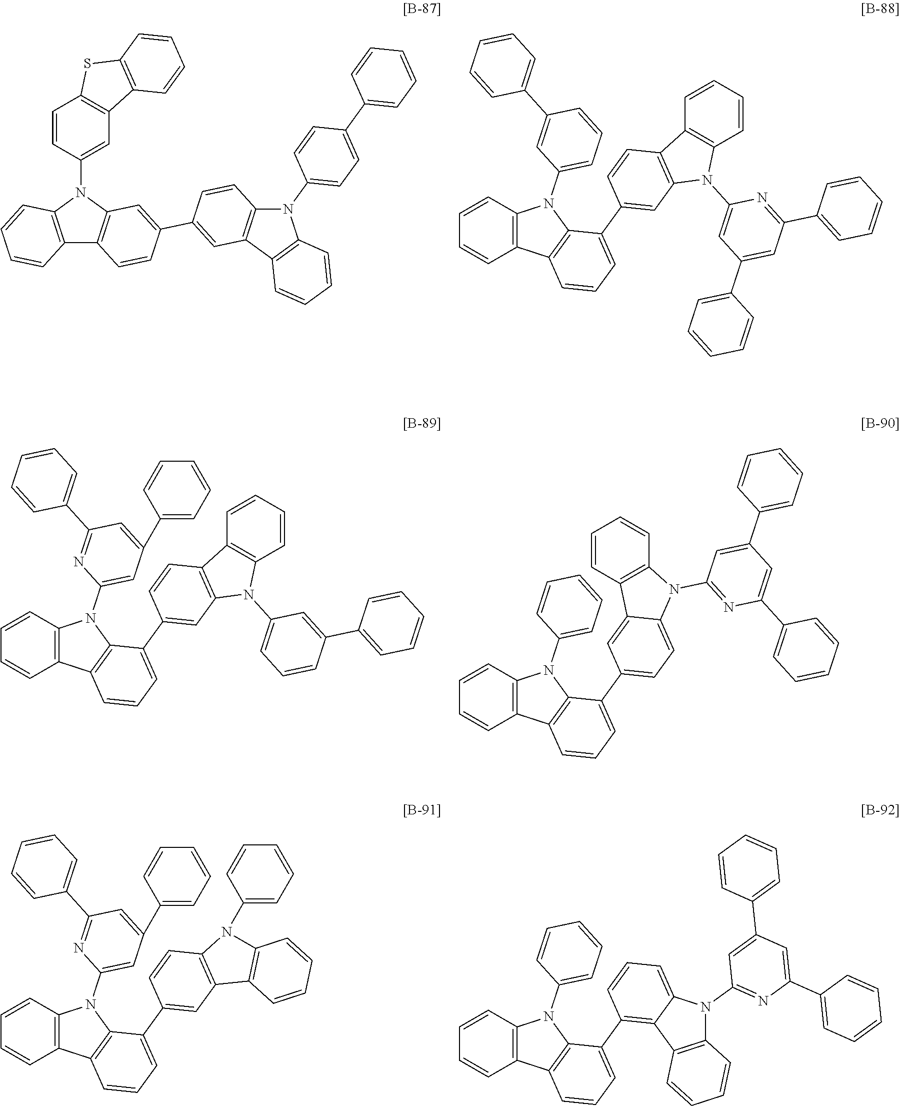

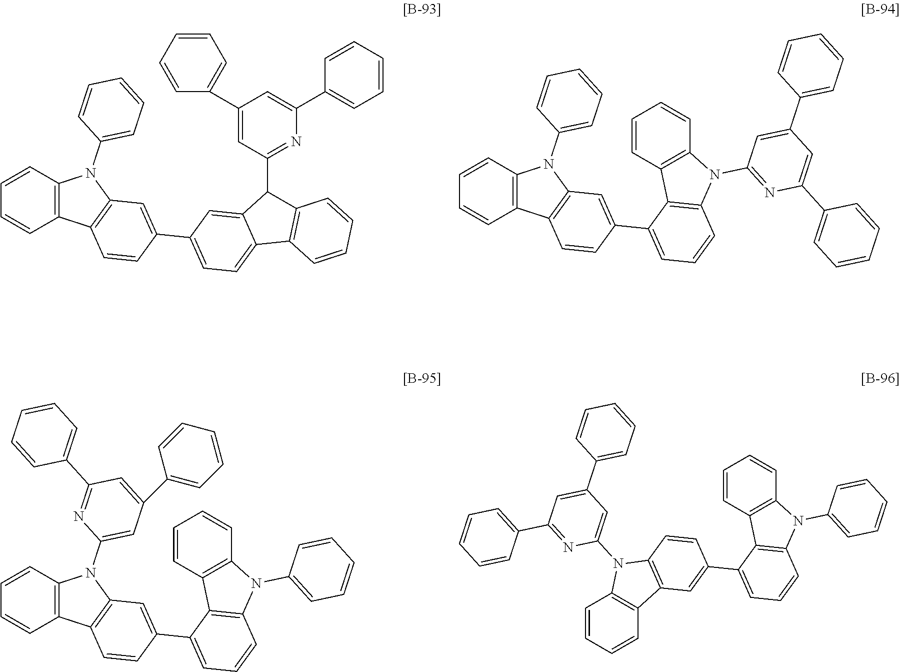

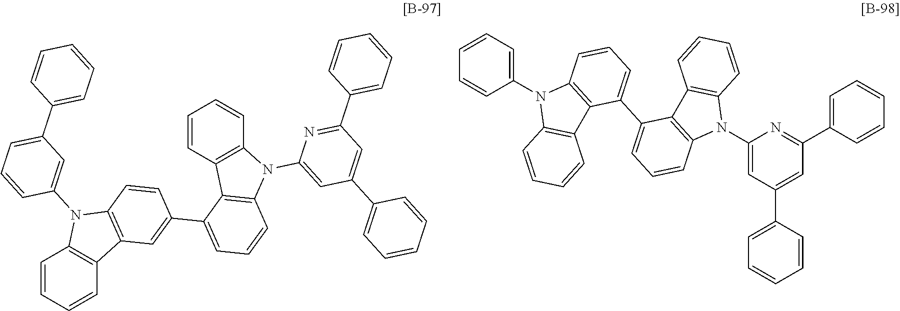 [SMILES: c1ccc(-c2cccc(-n3c4ccccc4c4cc(-c5cccc6c5c5ccccc5n6-c5cc(-c6ccccc6)cc(-c6ccccc6)n5)ccc43)c2)cc1 ; c1ccc(-c2cc(-c3ccccc3)nc(-n3c4ccccc4c4c(-c5cccc6c5c5ccccc5n6-c5ccccc5)cccc43)c2)cc1]

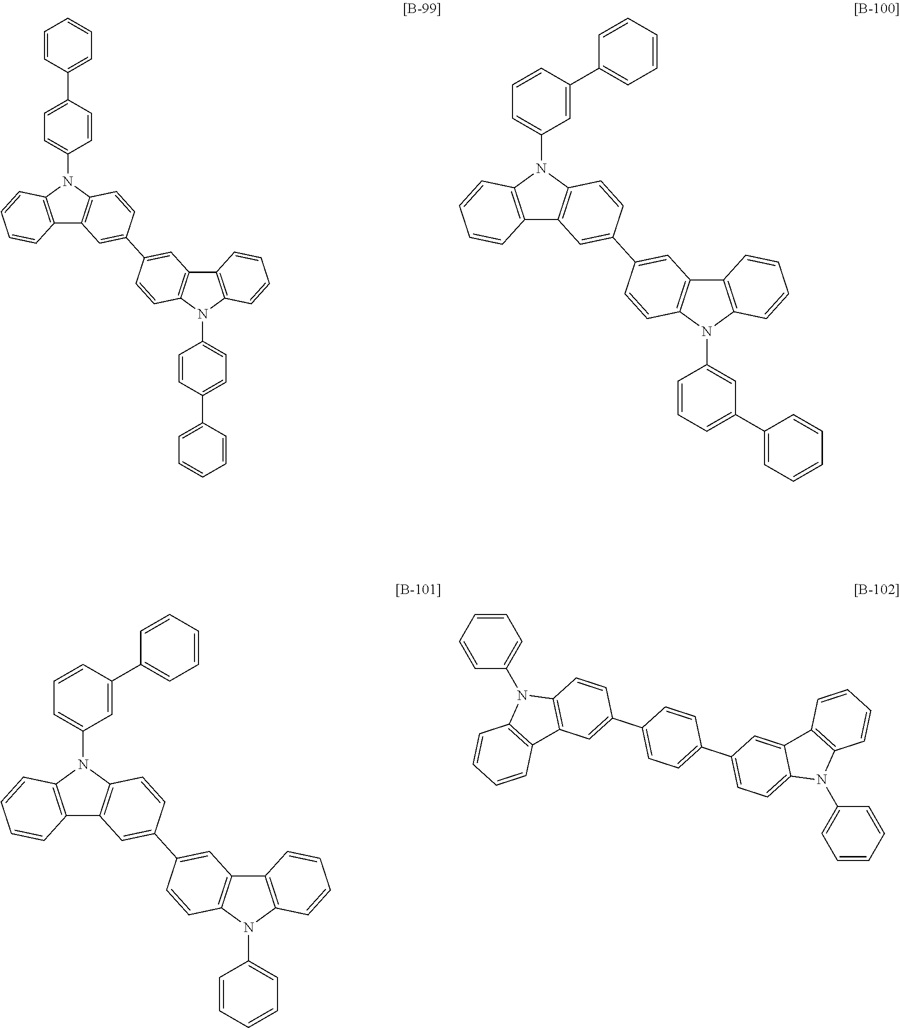 [SMILES: c1ccc(-c2ccc(-n3c4ccccc4c4cc(-c5ccc6c(c5)c5ccccc5n6-c5ccc(-c6ccccc6)cc5)ccc43)cc2)cc1 ; c1ccc(-c2cccc(-n3c4ccccc4c4cc(-c5ccc6c(c5)c5ccccc5n6-c5cccc(-c6ccccc6)c5)ccc43)c2)cc1 ; c1ccc(-c2cccc(-n3c4ccccc4c4cc(-c5ccc6c(c5)c5ccccc5n6-c5ccccc5)ccc43)c2)cc1 ; c1ccc(-n2c3ccccc3c3cc(-c4ccc(-c5ccc6c(c5)c5ccccc5n6-c5ccccc5)cc4)ccc32)cc1]

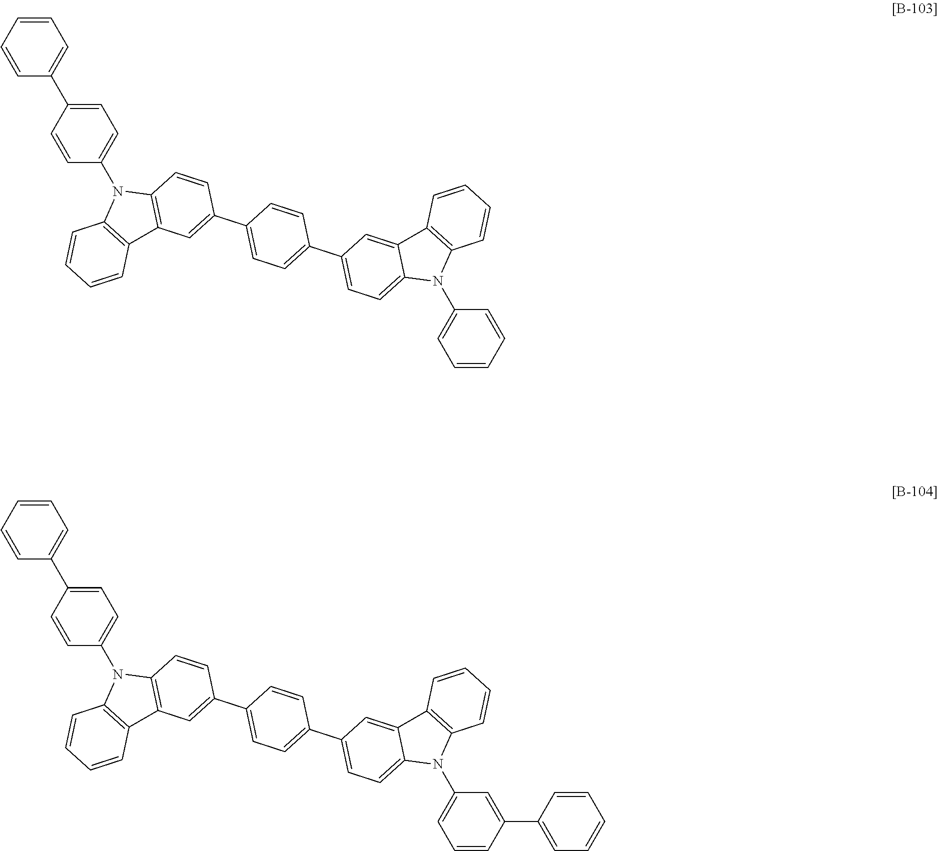 [SMILES: c1ccc(-c2ccc(-n3c4ccccc4c4cc(-c5ccc(-c6ccc7c(c6)c6ccccc6n7-c6ccccc6)cc5)ccc43)cc2)cc1 ; c1ccc(-c2ccc(-n3c4ccccc4c4cc(-c5ccc(-c6ccc7c(c6)c6ccccc6n7-c6cccc(-c7ccccc7)c6)cc5)ccc43)cc2)cc1]

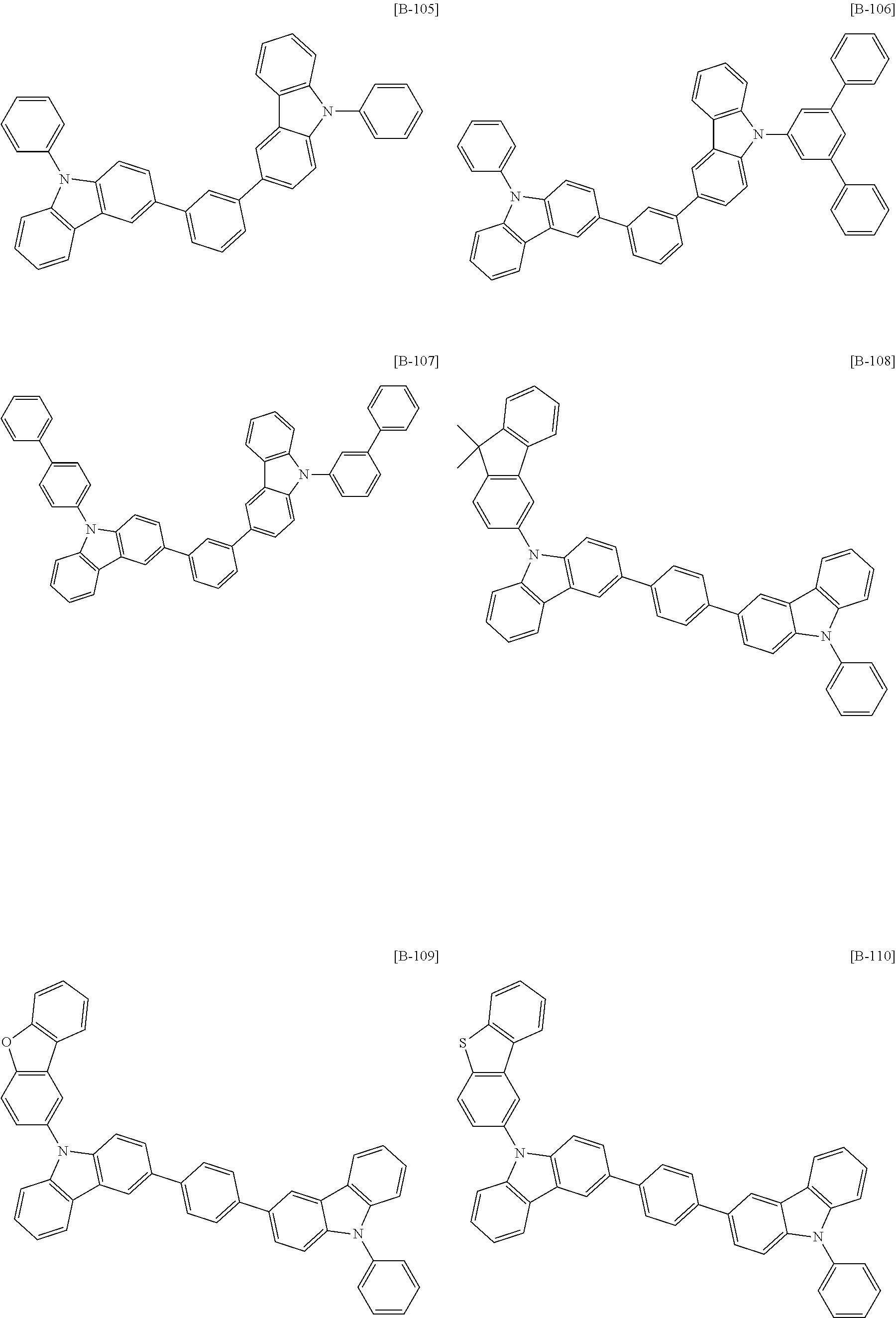

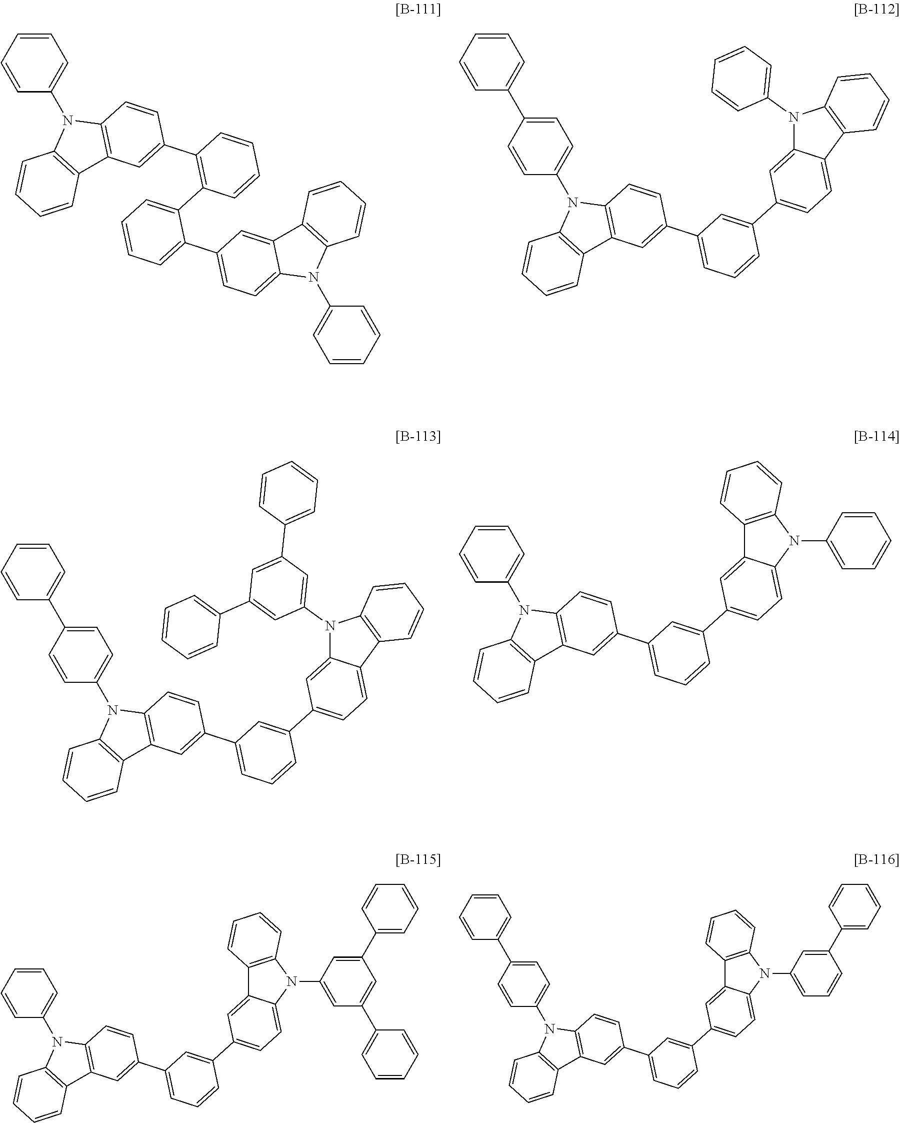

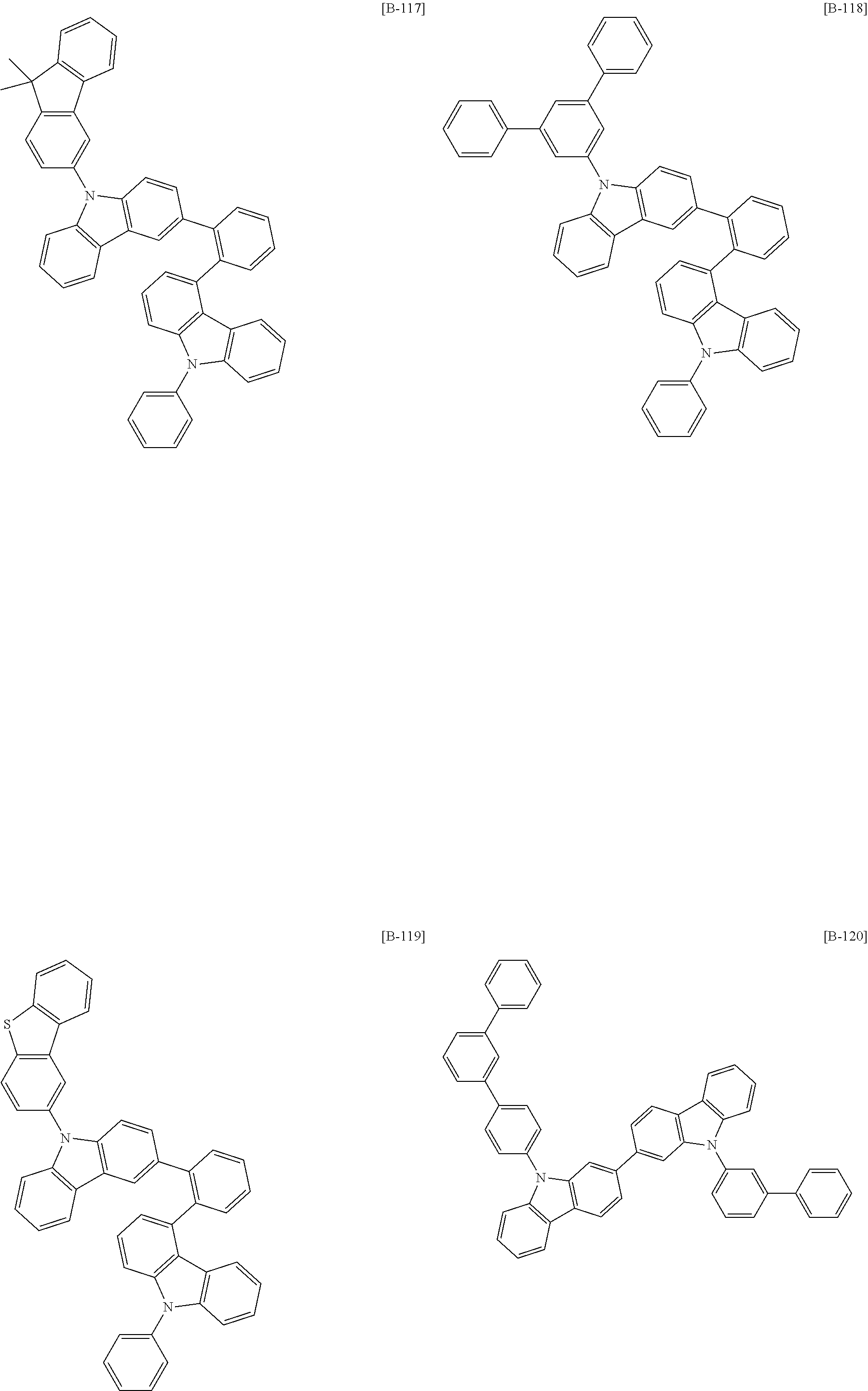 [SMILES: CC1(C)c2ccccc2-c2cc(-n3c4ccccc4c4cc(-c5ccccc5-c5cccc6c5c5ccccc5n6-c5ccccc5)ccc43)ccc21 ; c1ccc(-c2cc(-c3ccccc3)cc(-n3c4ccccc4c4cc(-c5ccccc5-c5cccc6c5c5ccccc5n6-c5ccccc5)ccc43)c2)cc1 ; c1ccc(-n2c3ccccc3c3c(-c4ccccc4-c4ccc5c(c4)c4ccccc4n5-c4ccc5sc6ccccc6c5c4)cccc32)cc1 ; c1ccc(-c2cccc(-c3ccc(-n4c5ccccc5c5ccc(-c6ccc7c8ccccc8n(-c8cccc(-c9ccccc9)c8)c7c6)cc54)cc3)c2)cc1]

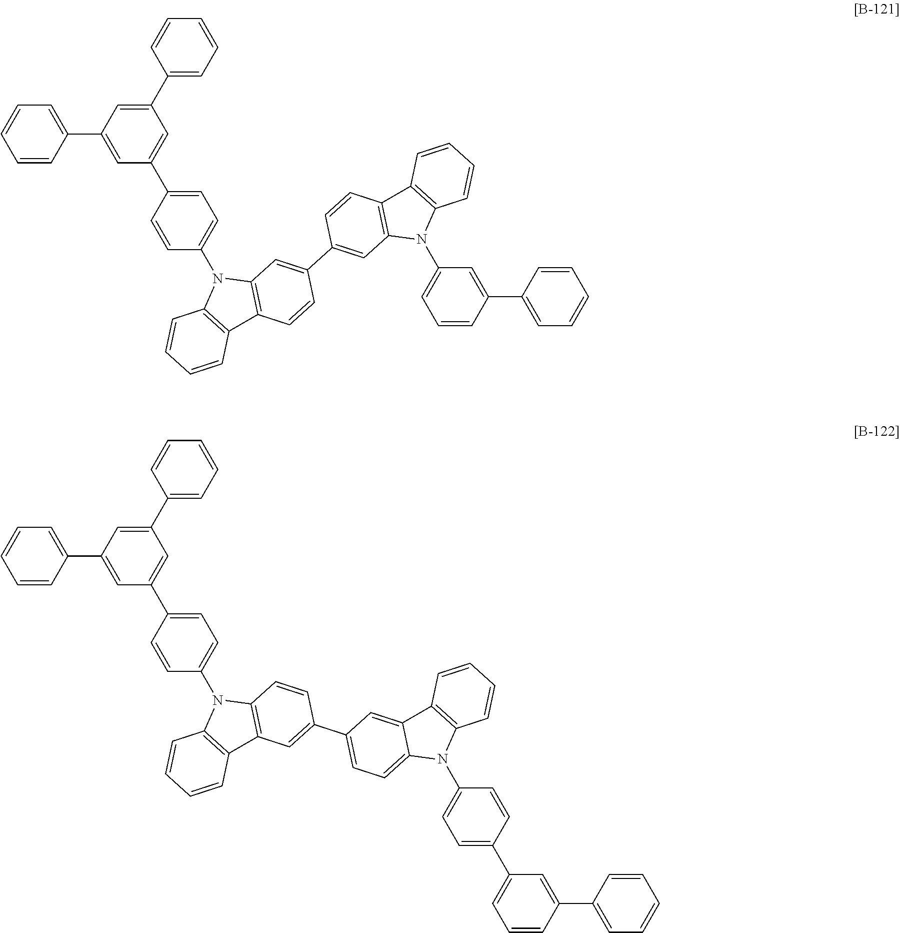 [SMILES: c1ccc(-c2cc(-c3ccccc3)cc(-c3ccc(-n4c5ccccc5c5ccc(-c6ccc7c8ccccc8n(-c8cccc(-c9ccccc9)c8)c7c6)cc54)cc3)c2)cc1 ; c1ccc(-c2cccc(-c3ccc(-n4c5ccccc5c5cc(-c6ccc7c(c6)c6ccccc6n7-c6ccc(-c7cc(-c8ccccc8)cc(-c8ccccc8)c7)cc6)ccc54)cc3)c2)cc1]

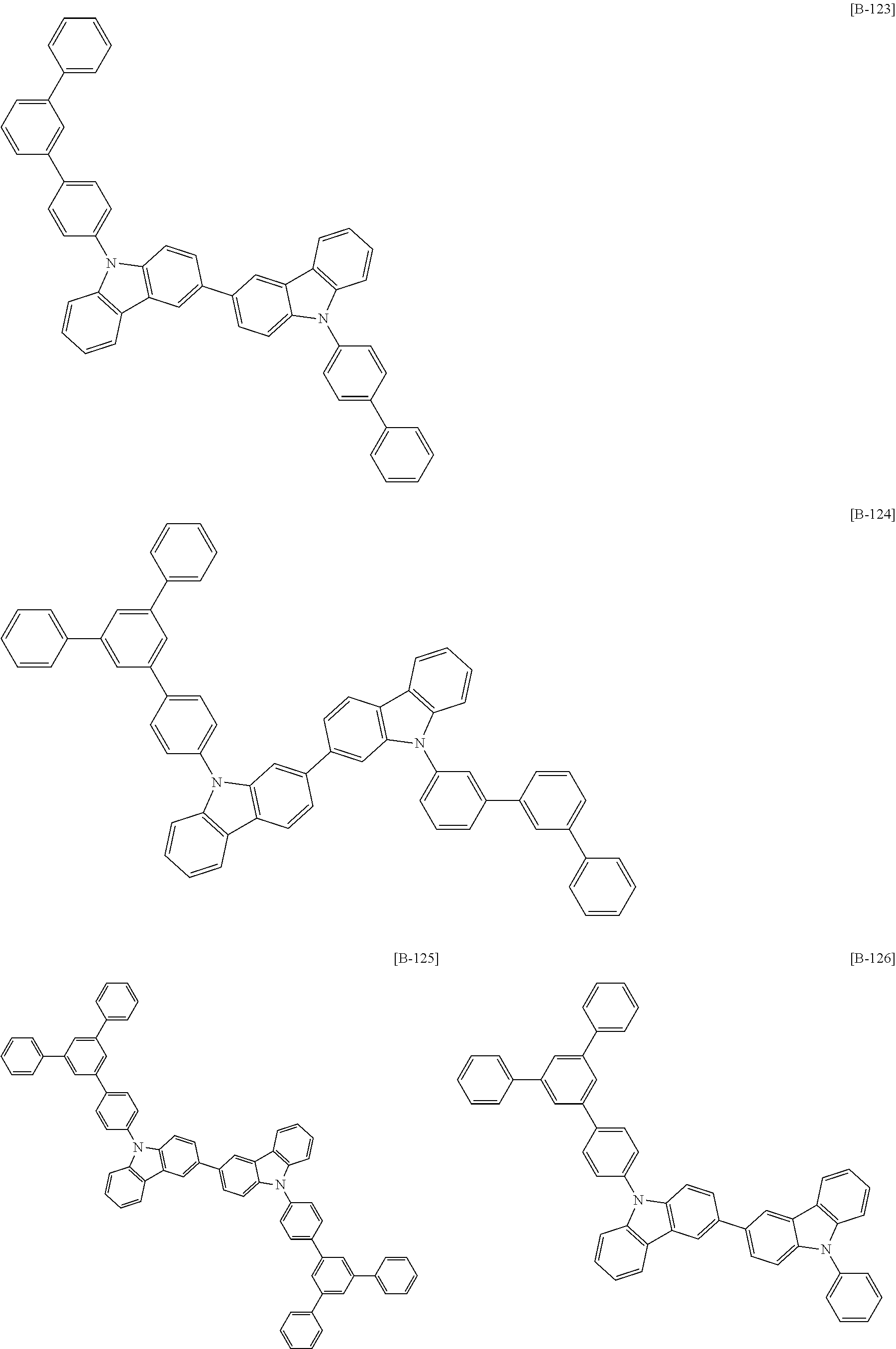 [SMILES: c1ccc(-c2ccc(-n3c4ccccc4c4cc(-c5ccc6c(c5)c5ccccc5n6-c5ccc(-c6cccc(-c7ccccc7)c6)cc5)ccc43)cc2)cc1 ; c1ccc(-c2cccc(-c3cccc(-n4c5ccccc5c5ccc(-c6ccc7c8ccccc8n(-c8ccc(-c9cc(-c%10ccccc%10)cc(-c%10ccccc%10)c9)cc8)c7c6)cc54)c3)c2)cc1 ; c1ccc(-c2cc(-c3ccccc3)cc(-c3ccc(-n4c5ccccc5c5cc(-c6ccc7c(c6)c6ccccc6n7-c6ccc(-c7cc(-c8ccccc8)cc(-c8ccccc8)c7)cc6)ccc54)cc3)c2)cc1 ; c1ccc(-c2cc(-c3ccccc3)cc(-c3ccc(-n4c5ccccc5c5cc(-c6ccc7c(c6)c6ccccc6n7-c6ccccc6)ccc54)cc3)c2)cc1]

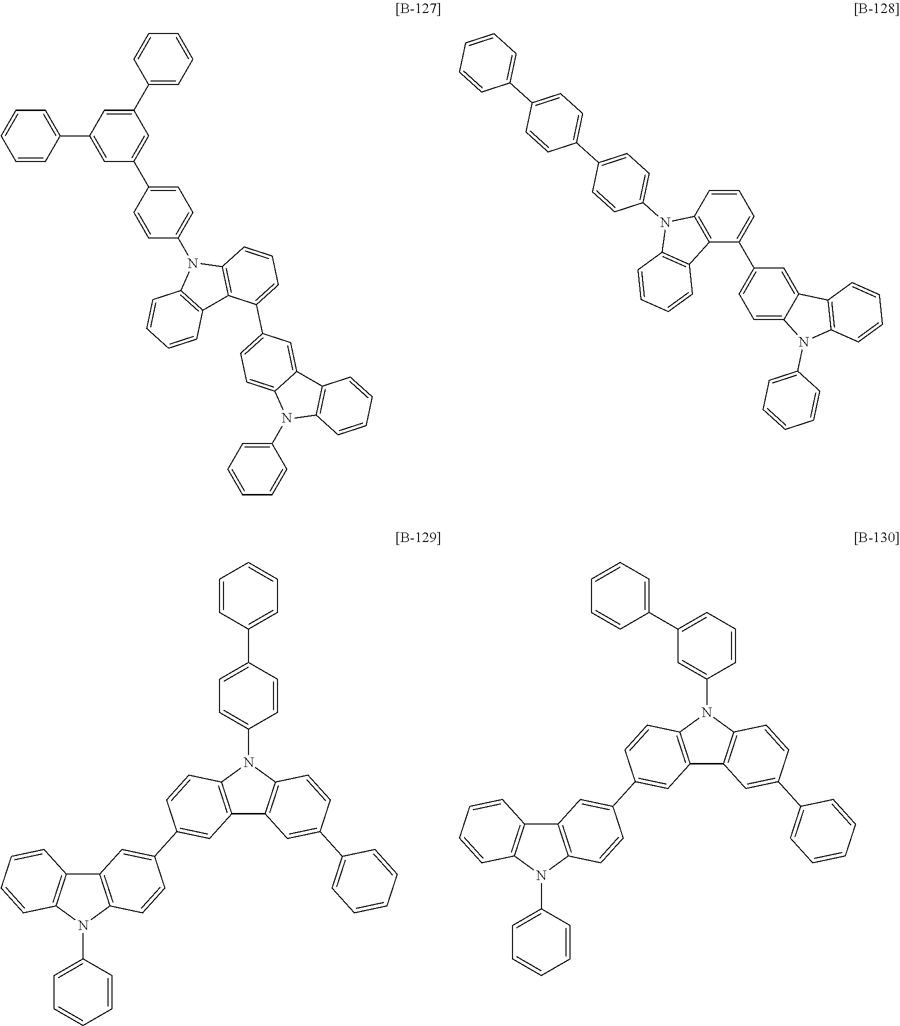 [SMILES: c1ccc(-c2cc(-c3ccccc3)cc(-c3ccc(-n4c5ccccc5c5c(-c6ccc7c(c6)c6ccccc6n7-c6ccccc6)cccc54)cc3)c2)cc1 ; c1ccc(-c2ccc(-c3ccc(-n4c5ccccc5c5c(-c6ccc7c(c6)c6ccccc6n7-c6ccccc6)cccc54)cc3)cc2)cc1 ; c1ccc(-c2ccc(-n3c4ccc(-c5ccccc5)cc4c4cc(-c5ccc6c(c5)c5ccccc5n6-c5ccccc5)ccc43)cc2)cc1 ; c1ccc(-c2cccc(-n3c4ccc(-c5ccccc5)cc4c4cc(-c5ccc6c(c5)c5ccccc5n6-c5ccccc5)ccc43)c2)cc1]

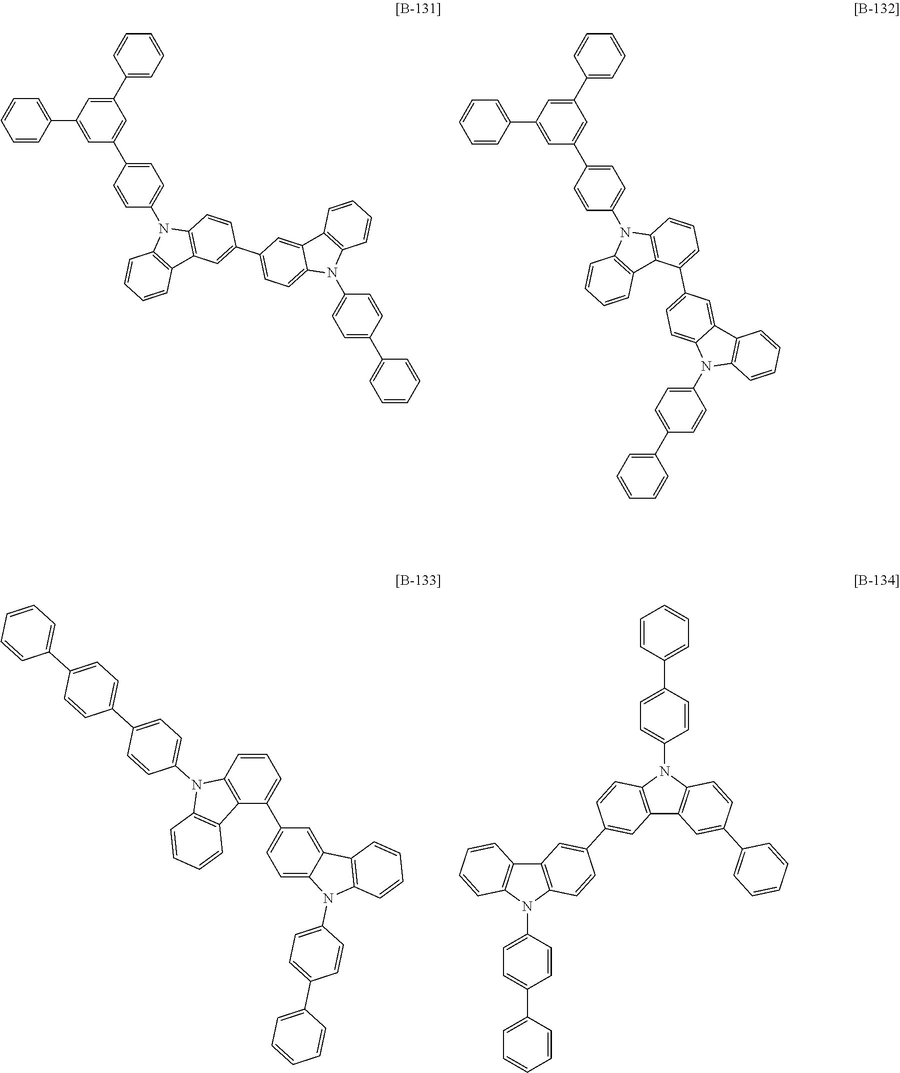 [SMILES: c1ccc(-c2ccc(-n3c4ccccc4c4cc(-c5ccc6c(c5)c5ccccc5n6-c5ccc(-c6cc(-c7ccccc7)cc(-c7ccccc7)c6)cc5)ccc43)cc2)cc1 ; c1ccc(-c2ccc(-n3c4ccccc4c4cc(-c5cccc6c5c5ccccc5n6-c5ccc(-c6cc(-c7ccccc7)cc(-c7ccccc7)c6)cc5)ccc43)cc2)cc1 ; c1ccc(-c2ccc(-c3ccc(-n4c5ccccc5c5c(-c6ccc7c(c6)c6ccccc6n7-c6ccc(-c7ccccc7)cc6)cccc54)cc3)cc2)cc1 ; c1ccc(-c2ccc(-n3c4ccccc4c4cc(-c5ccc6c(c5)c5cc(-c7ccccc7)ccc5n6-c5ccc(-c6ccccc6)cc5)ccc43)cc2)cc1]

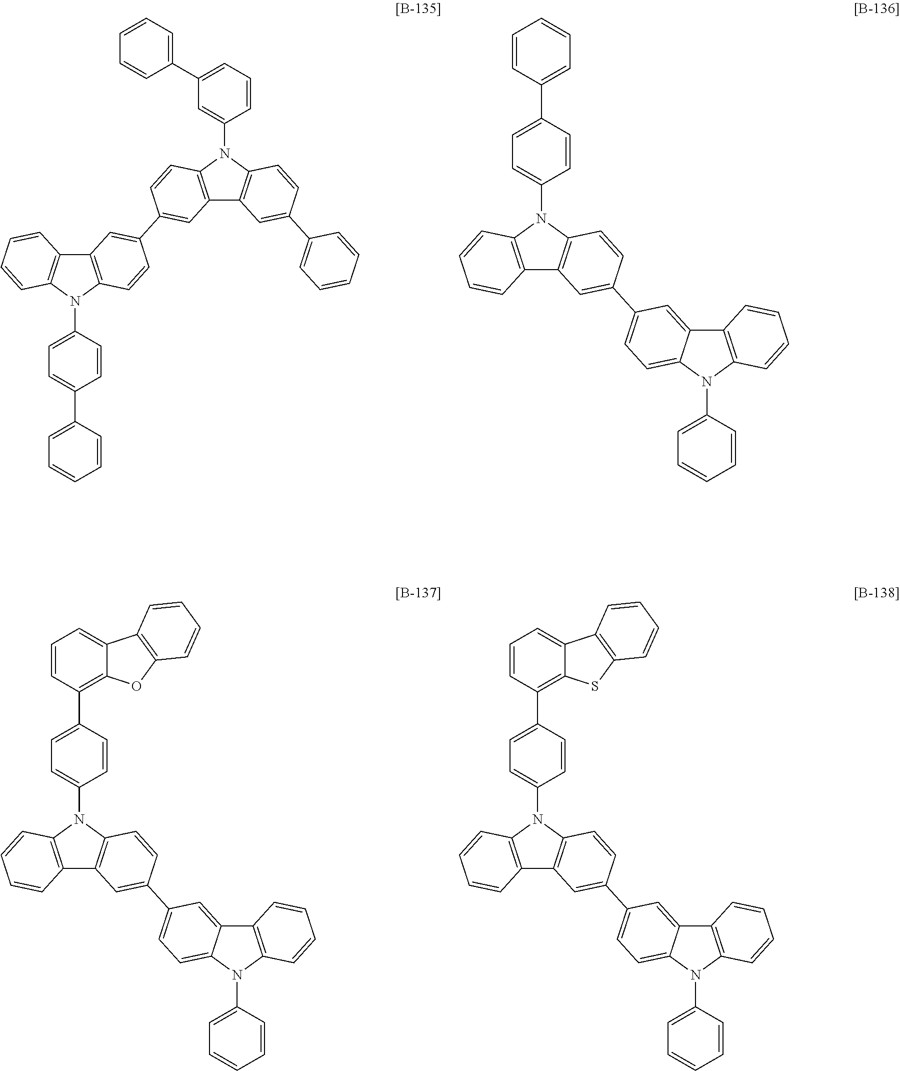 [SMILES: c1ccc(-c2ccc(-n3c4ccccc4c4cc(-c5ccc6c(c5)c5cc(-c7ccccc7)ccc5n6-c5cccc(-c6ccccc6)c5)ccc43)cc2)cc1 ; c1ccc(-c2ccc(-n3c4ccccc4c4cc(-c5ccc6c(c5)c5ccccc5n6-c5ccccc5)ccc43)cc2)cc1 ; c1ccc(-n2c3ccccc3c3cc(-c4ccc5c(c4)c4ccccc4n5-c4ccc(-c5cccc6c5oc5ccccc56)cc4)ccc32)cc1 ; c1ccc(-n2c3ccccc3c3cc(-c4ccc5c(c4)c4ccccc4n5-c4ccc(-c5cccc6c5sc5ccccc56)cc4)ccc32)cc1]

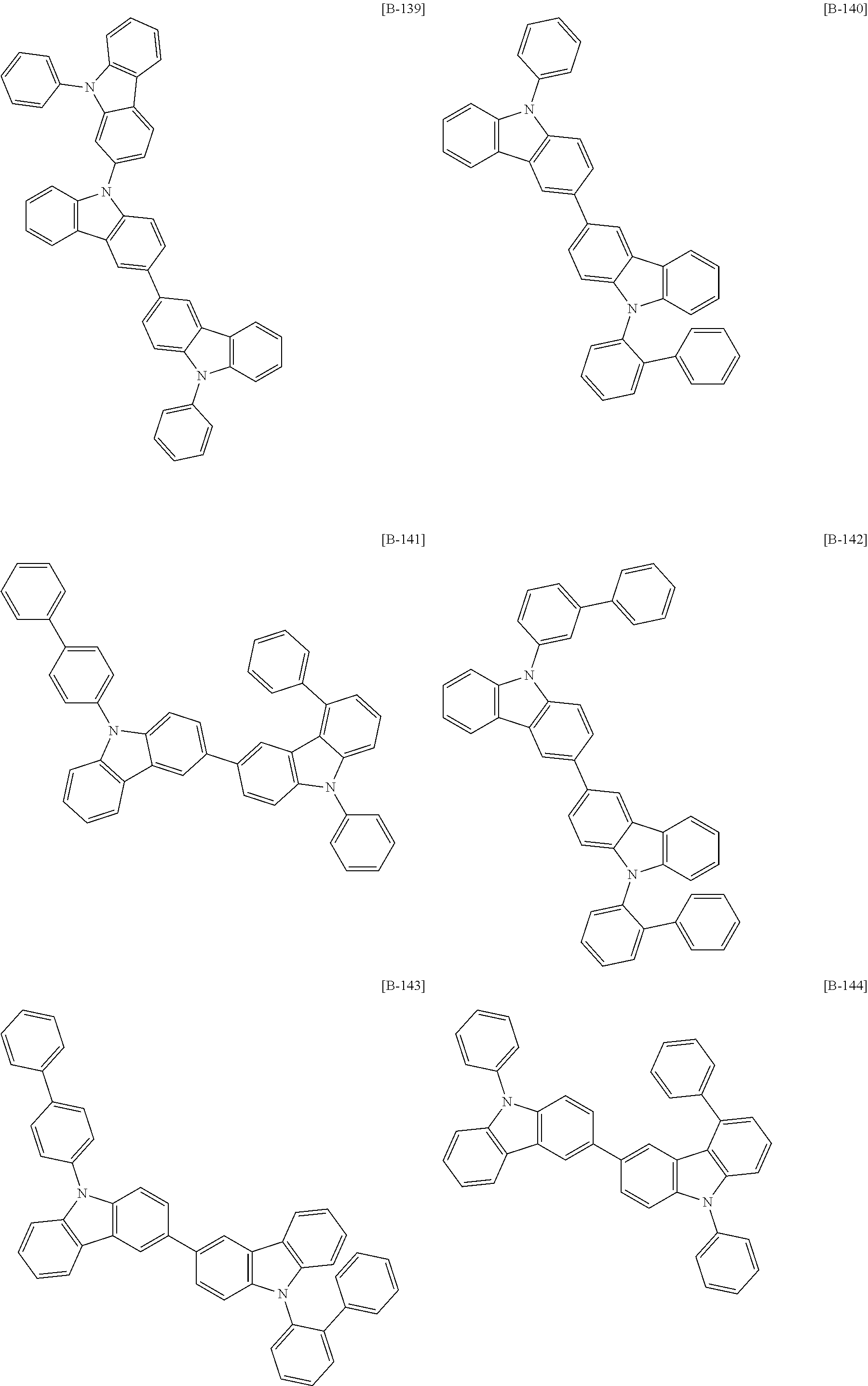 [SMILES: c1ccc(-n2c3ccccc3c3cc(-c4ccc5c(c4)c4ccccc4n5-c4ccc5c6ccccc6n(-c6ccccc6)c5c4)ccc32)cc1 ; c1ccc(-c2ccccc2-n2c3ccccc3c3cc(-c4ccc5c(c4)c4ccccc4n5-c4ccccc4)ccc32)cc1 ; c1ccc(-c2ccc(-n3c4ccccc4c4cc(-c5ccc6c(c5)c5c(-c7ccccc7)cccc5n6-c5ccccc5)ccc43)cc2)cc1 ; c1ccc(-c2cccc(-n3c4ccccc4c4cc(-c5ccc6c(c5)c5ccccc5n6-c5ccccc5-c5ccccc5)ccc43)c2)cc1 ; c1ccc(-c2ccc(-n3c4ccccc4c4cc(-c5ccc6c(c5)c5ccccc5n6-c5ccccc5-c5ccccc5)ccc43)cc2)cc1 ; c1ccc(-c2cccc3c2c2cc(-c4ccc5c(c4)c4ccccc4n5-c4ccccc4)ccc2n3-c2ccccc2)cc1]

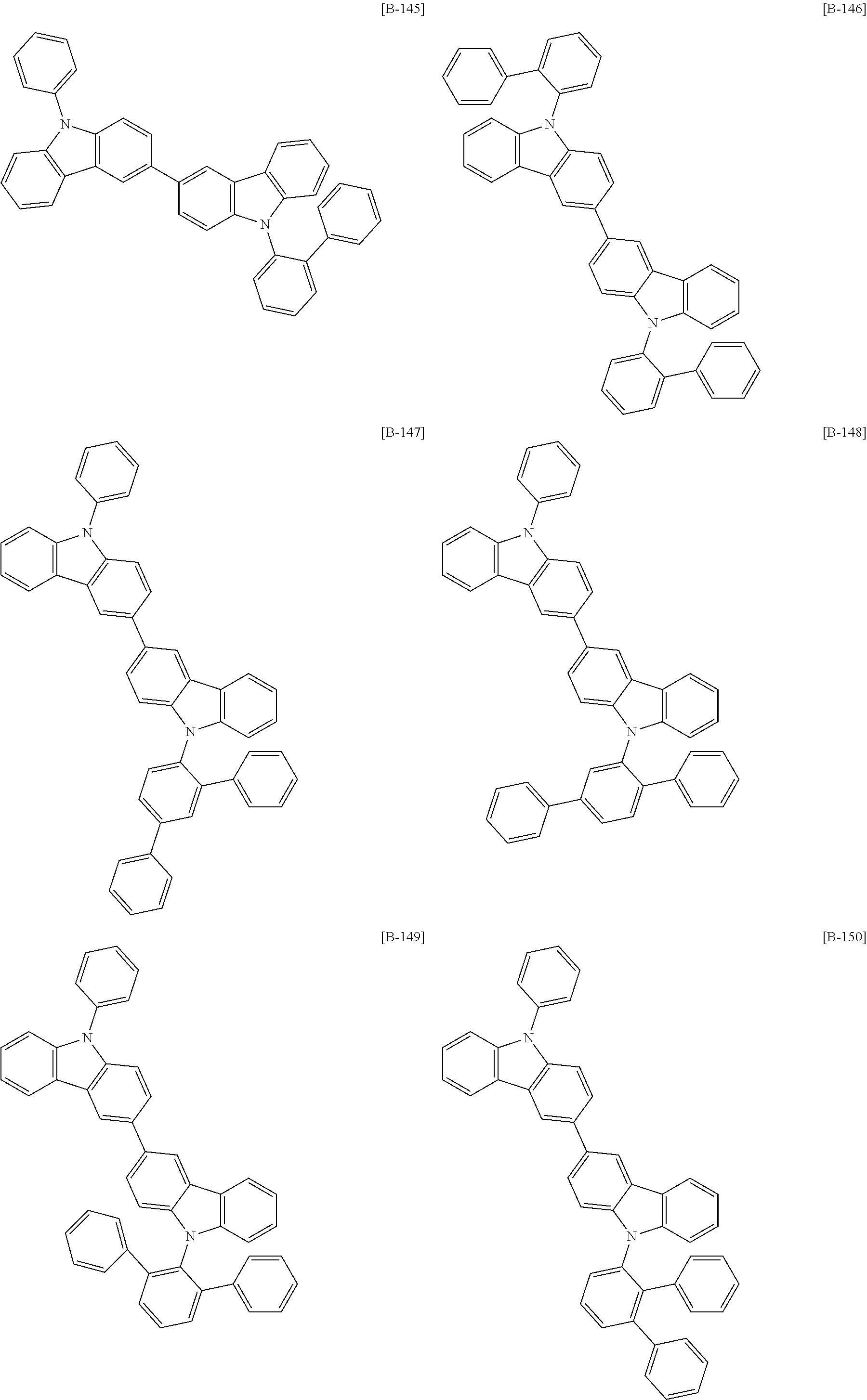

In addition, examples of Compound B-1 to Compound B-150 listed in Group 2 in which at least one hydrogen is substituted with deuterium are shown below.

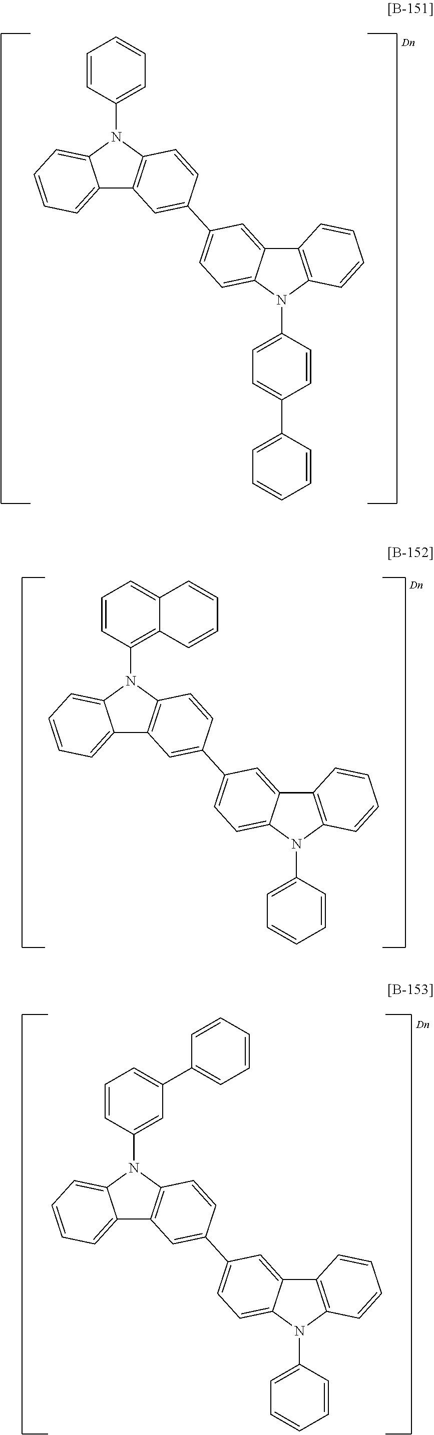

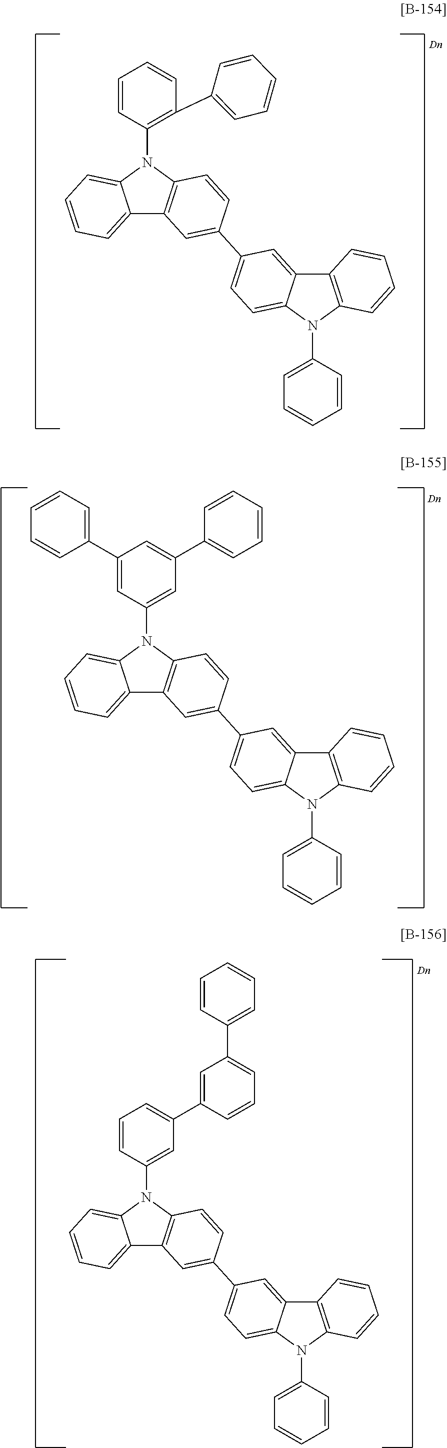

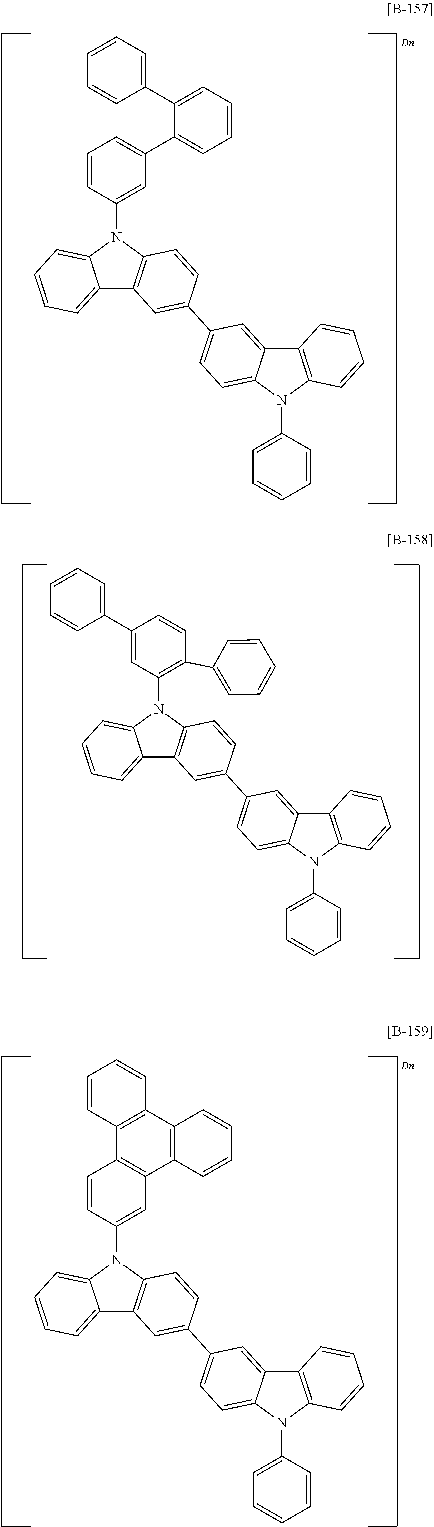

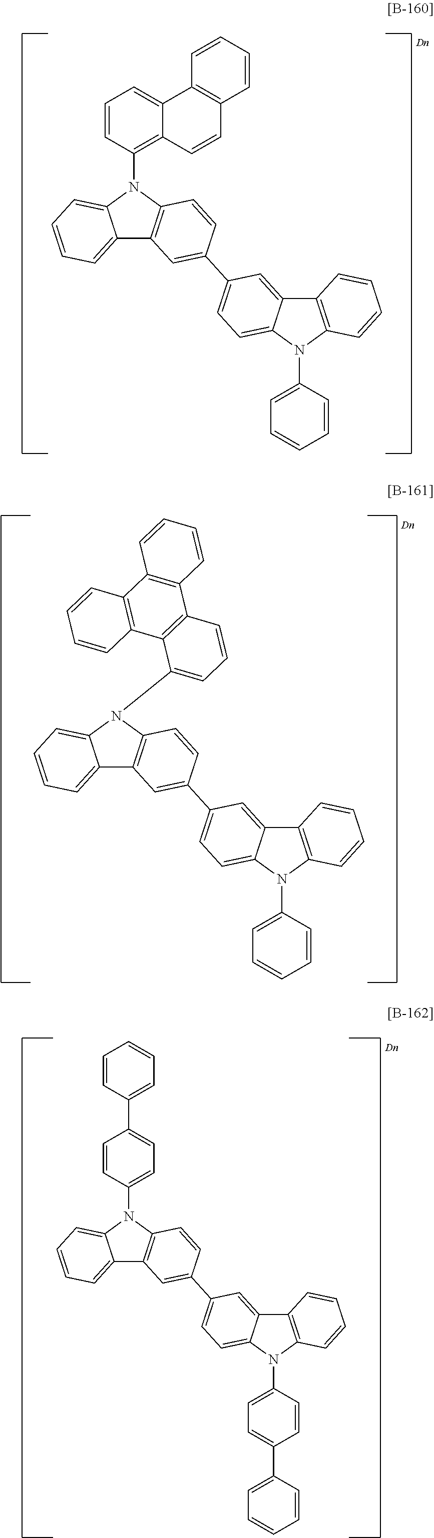

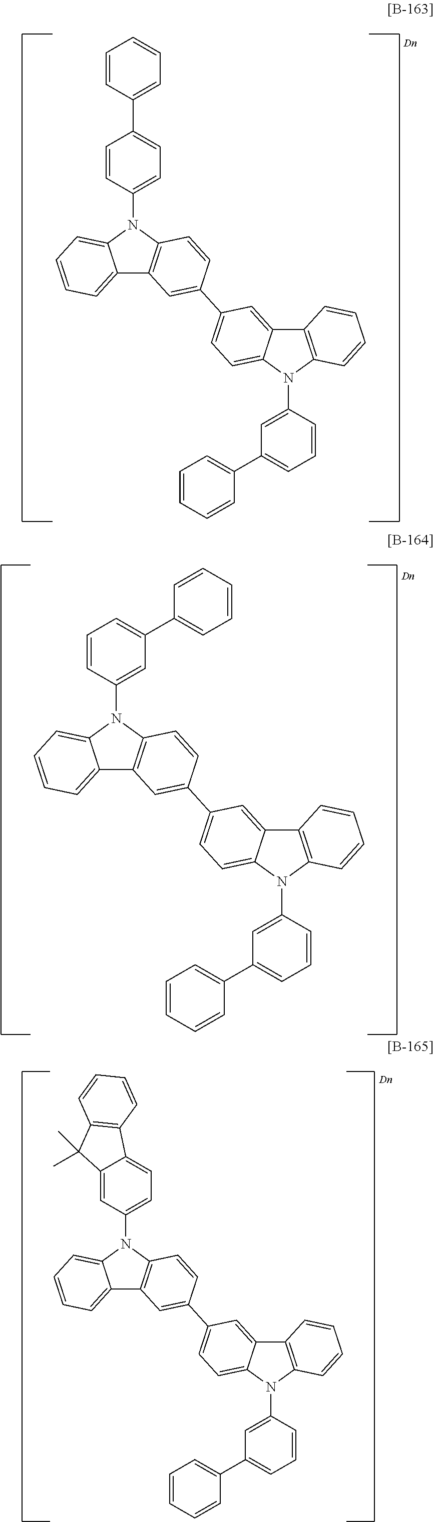

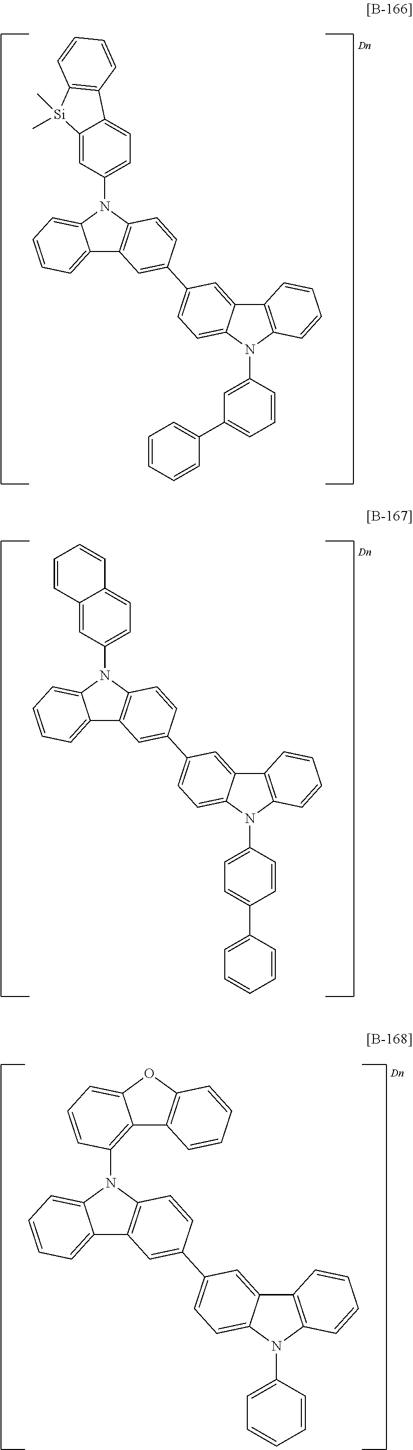

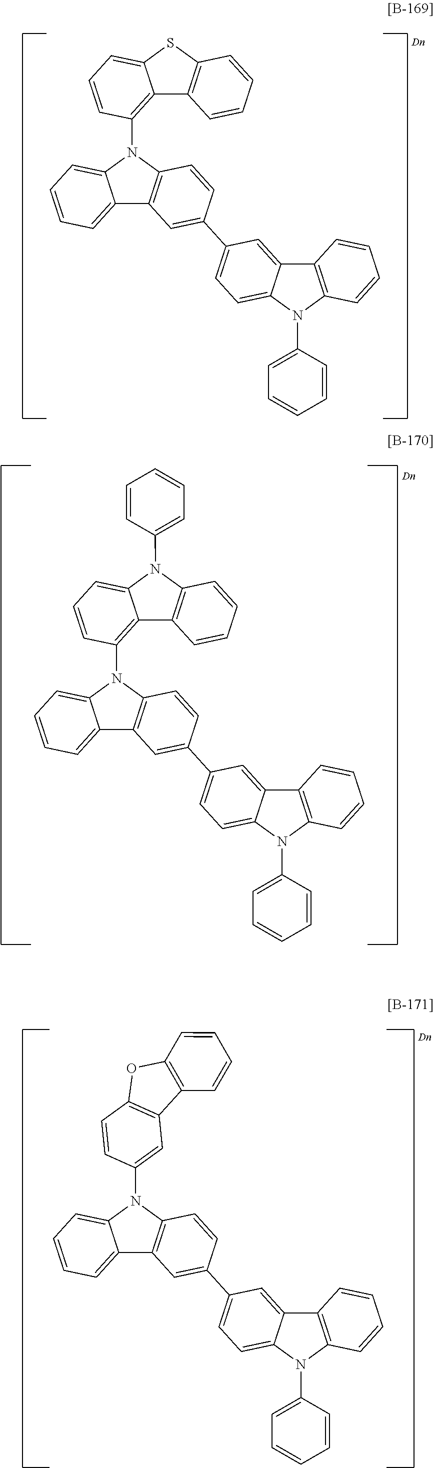

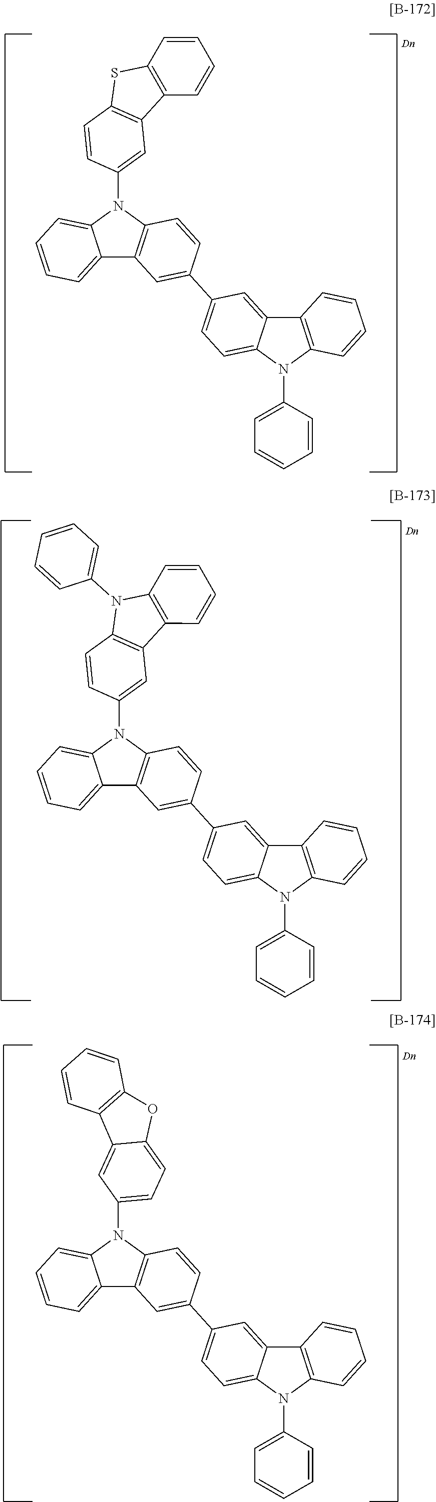

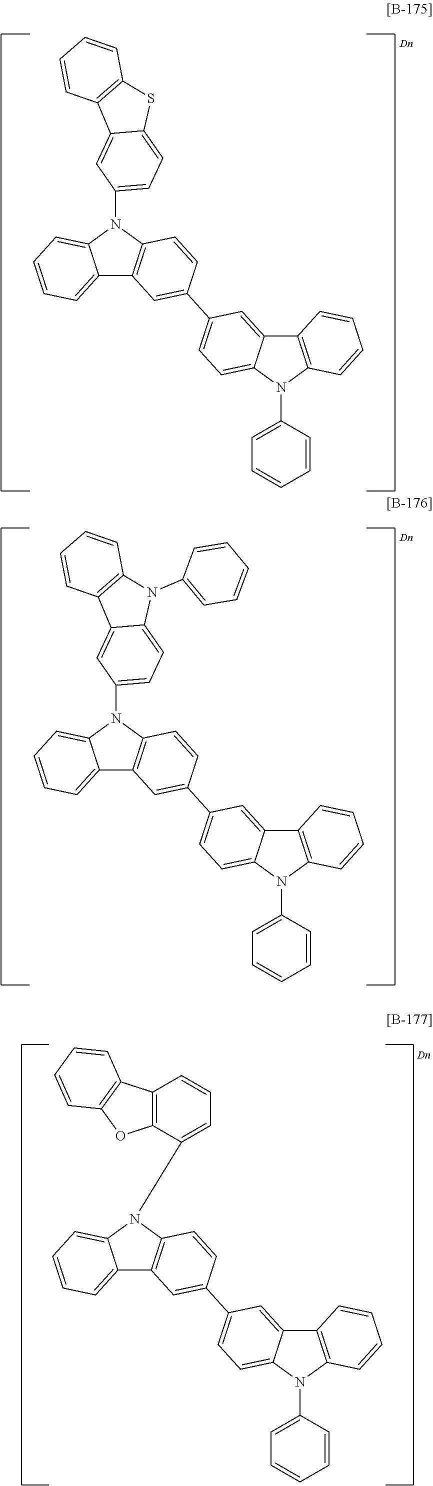

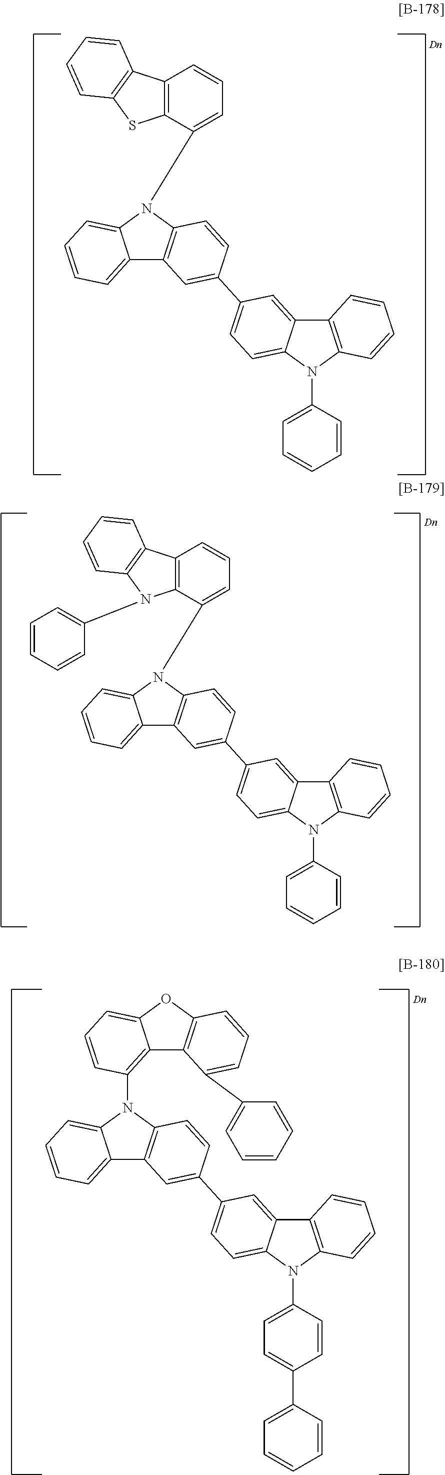

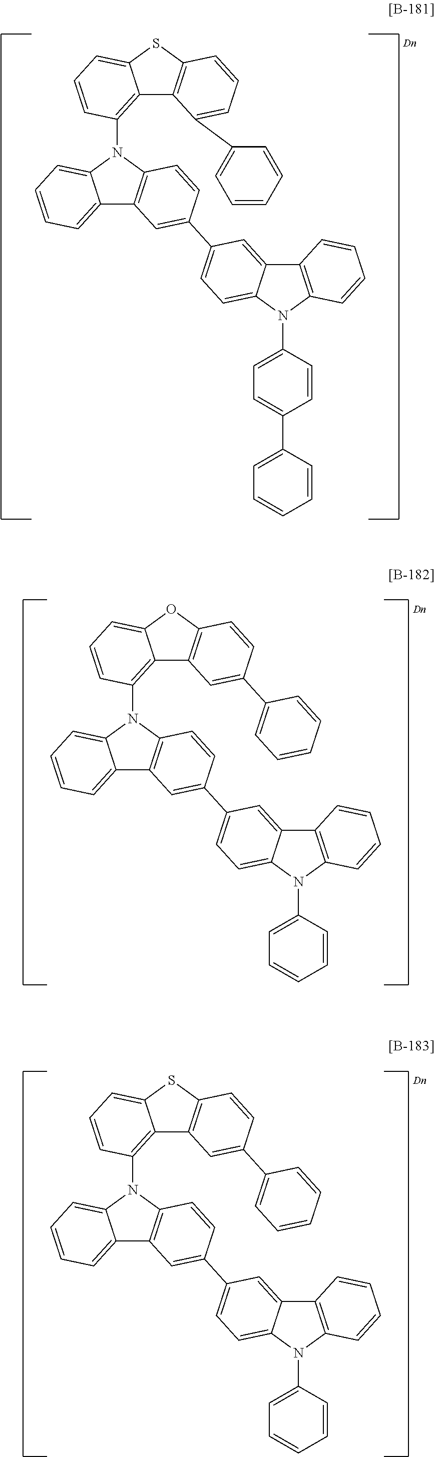

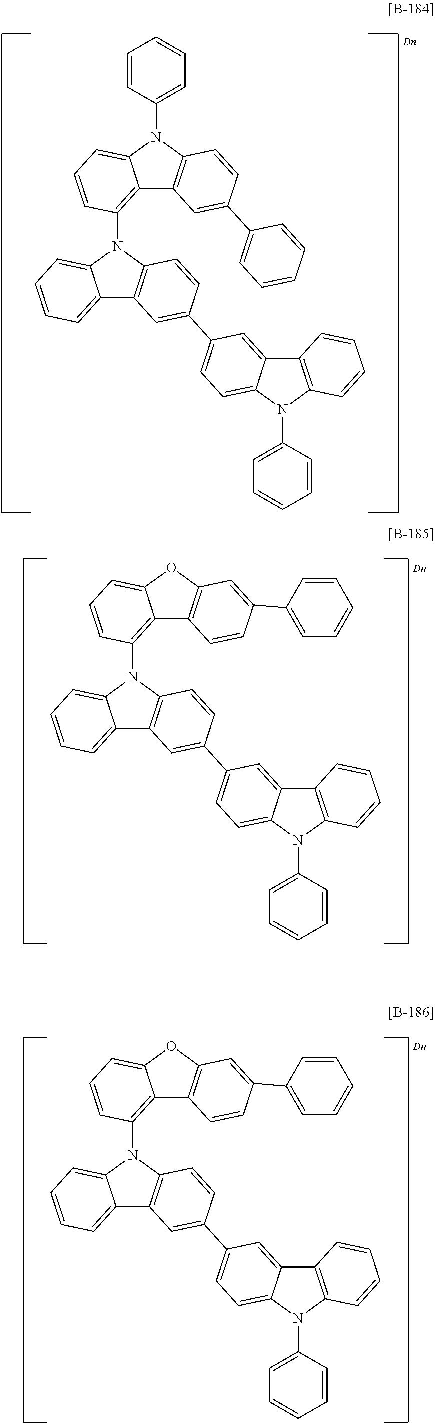

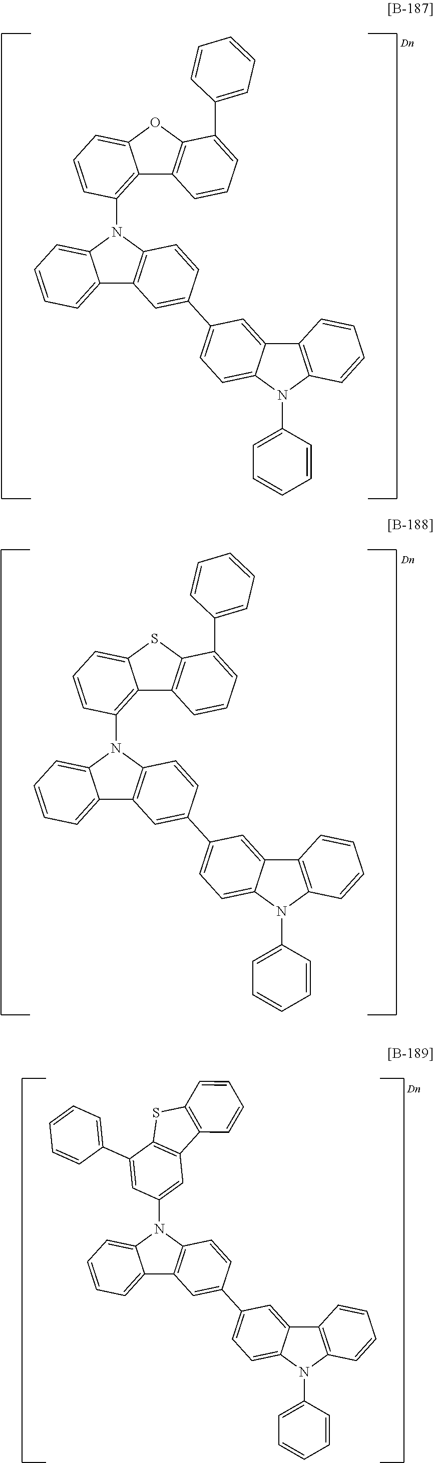

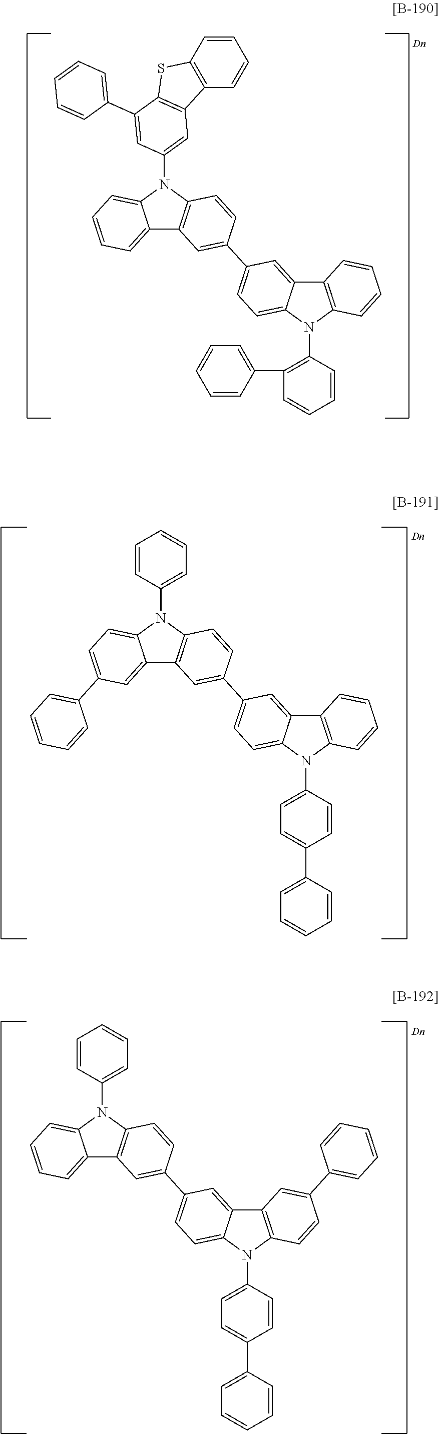

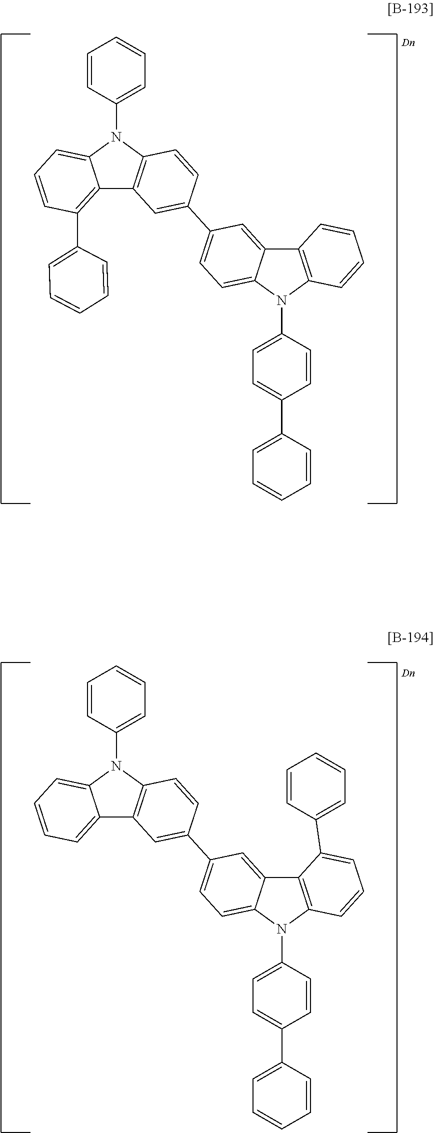

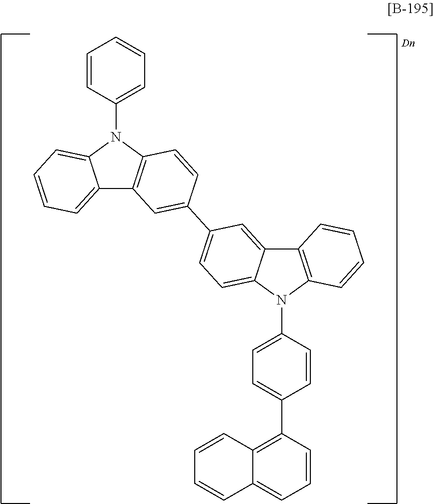

(Dn means the number of deuterium substituted, and represents a structure substituted with one or more deuterium)

For Compound B-151 to Compound B-195 of Group 2, the most specific structures according to the deuterium substitution position and substitution ratio are exemplarily shown below.

When deuterium is substituted, it is not limited to the compounds exemplified herein, and the deuterium substitution position and deuterium substitution ratio may include all changeable ranges within the ranges of Compound B-1 to Compound B-195.

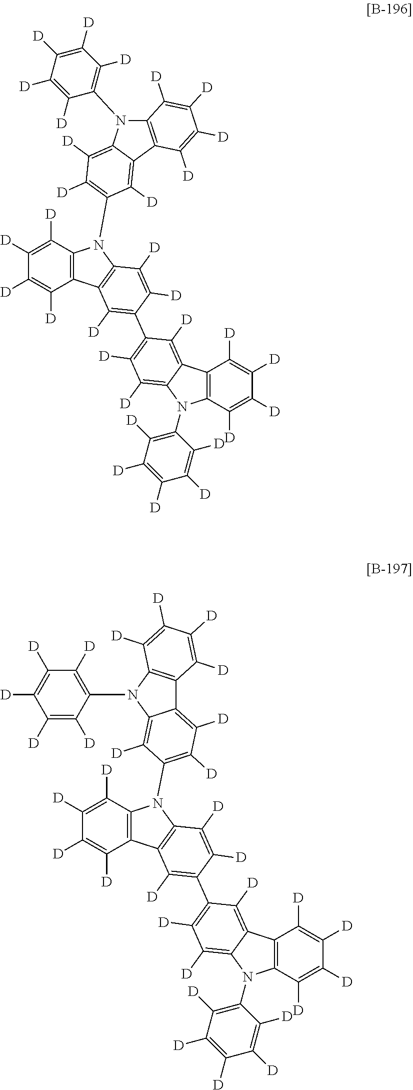

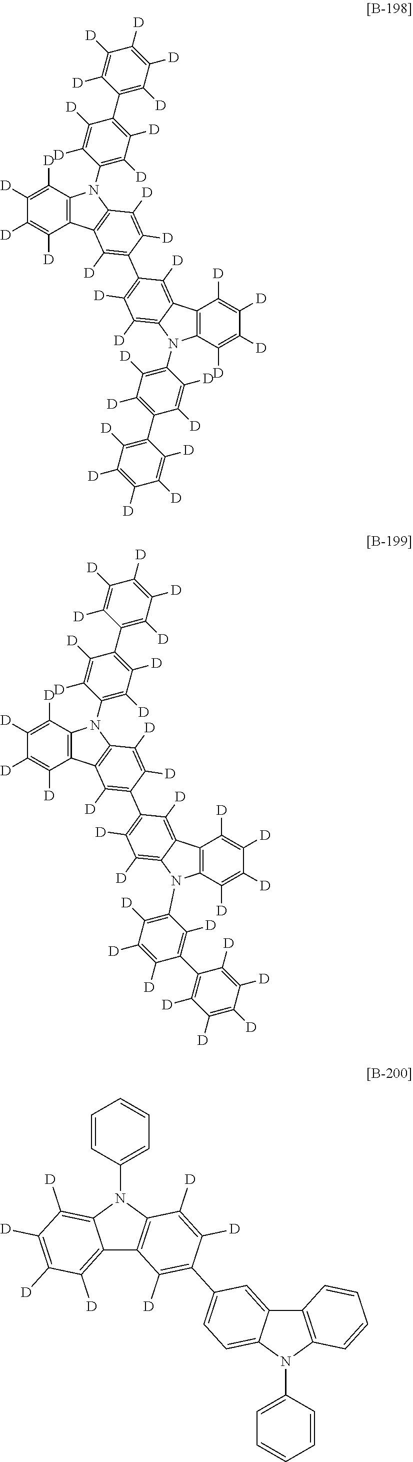

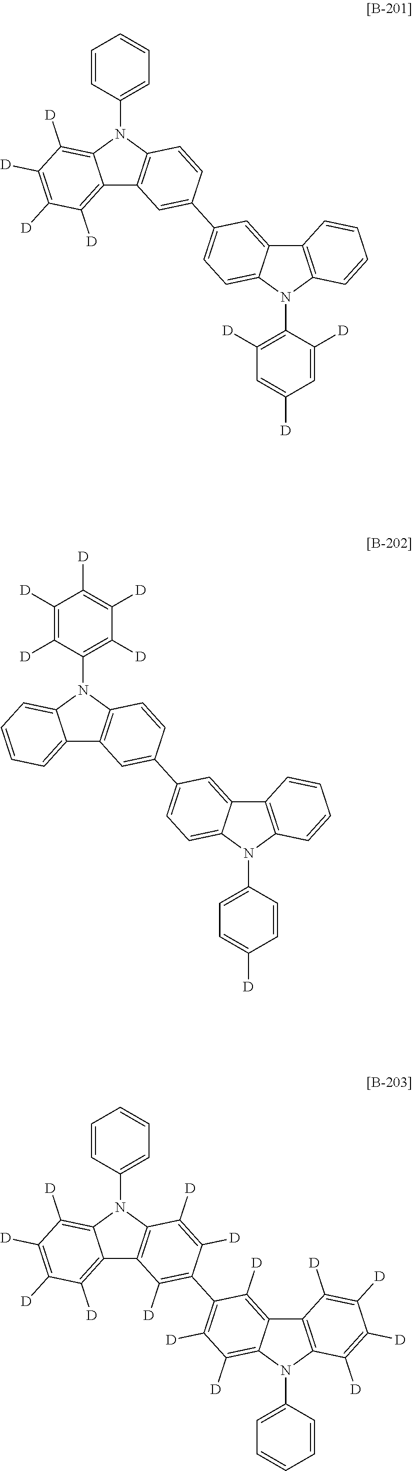 [SMILES: [2H]c1cc([2H])c(-n2c3ccccc3c3cc(-c4ccc5c(c4)c4c([2H])c([2H])c([2H])c([2H])c4n5-c4ccccc4)ccc32)c([2H])c1 ; [2H]c1ccc(-n2c3ccccc3c3cc(-c4ccc5c(c4)c4ccccc4n5-c4c([2H])c([2H])c([2H])c([2H])c4[2H])ccc32)cc1 ; [2H]c1c([2H])c([2H])c2c(c1[2H])c1c([2H])c(-c3c([2H])c([2H])c4c(c3[2H])c3c([2H])c([2H])c([2H])c([2H])c3n4-c3ccccc3)c([2H])c([2H])c1n2-c1ccccc1]

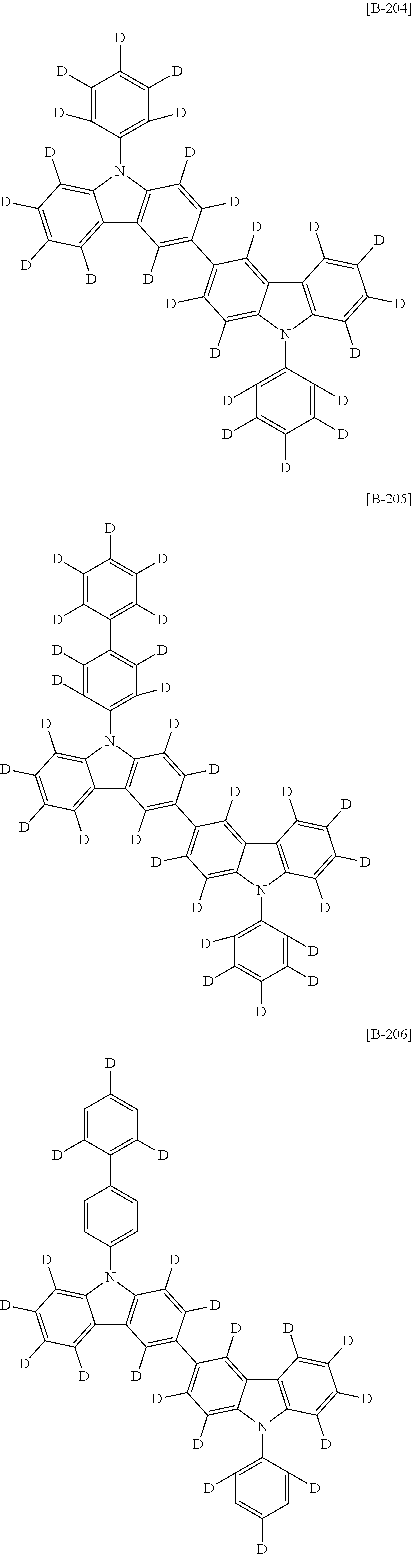

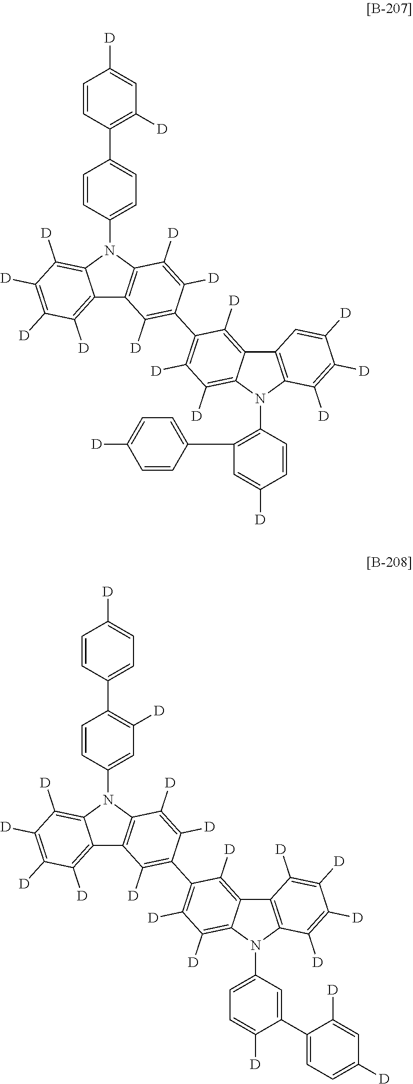 [SMILES: [2H]c1ccc(-c2cc([2H])ccc2-n2c3c([2H])c([2H])c([2H])cc3c3c([2H])c(-c4c([2H])c([2H])c5c(c4[2H])c4c([2H])c([2H])c([2H])c([2H])c4n5-c4ccc(-c5ccc([2H])cc5[2H])cc4)c([2H])c([2H])c32)cc1 ; [2H]c1ccc(-c2ccc(-n3c4c([2H])c([2H])c([2H])c([2H])c4c4c([2H])c(-c5c([2H])c([2H])c6c(c5[2H])c5c([2H])c([2H])c([2H])c([2H])c5n6-c5ccc([2H])c(-c6ccc([2H])cc6[2H])c5)c([2H])c([2H])c43)cc2[2H])cc1]

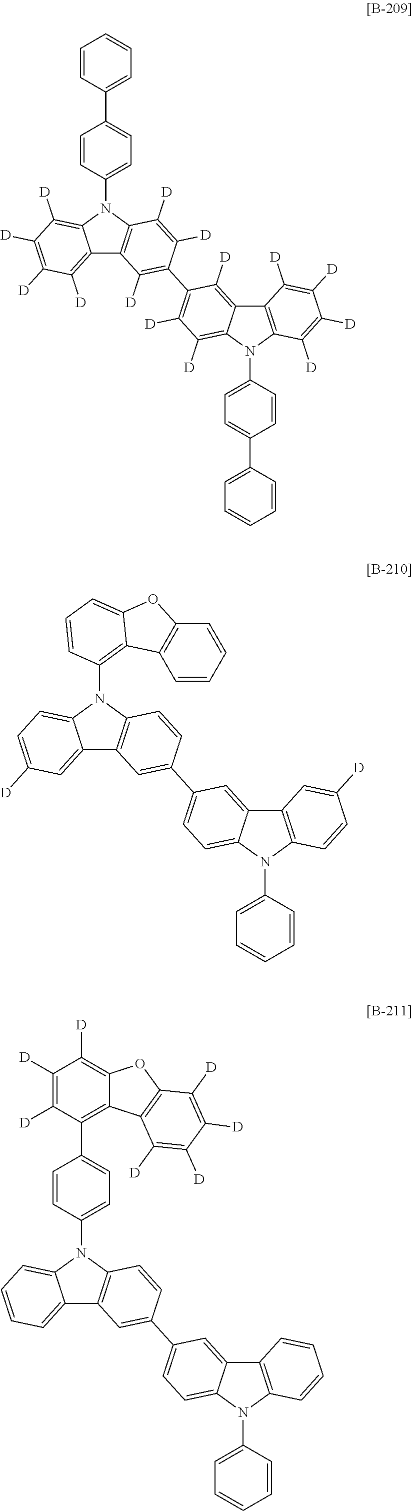 [SMILES: [2H]c1c([2H])c([2H])c2c(c1[2H])c1c([2H])c(-c3c([2H])c([2H])c4c(c3[2H])c3c([2H])c([2H])c([2H])c([2H])c3n4-c3ccc(-c4ccccc4)cc3)c([2H])c([2H])c1n2-c1ccc(-c2ccccc2)cc1 ; [2H]c1ccc2c(c1)c1cc(-c3ccc4c(c3)c3cc([2H])ccc3n4-c3cccc4oc5ccccc5c34)ccc1n2-c1ccccc1 ; [2H]c1c([2H])c([2H])c2c(oc3c([2H])c([2H])c([2H])c(-c4ccc(-n5c6ccccc6c6cc(-c7ccc8c(c7)c7ccccc7n8-c7ccccc7)ccc65)cc4)c32)c1[2H]]

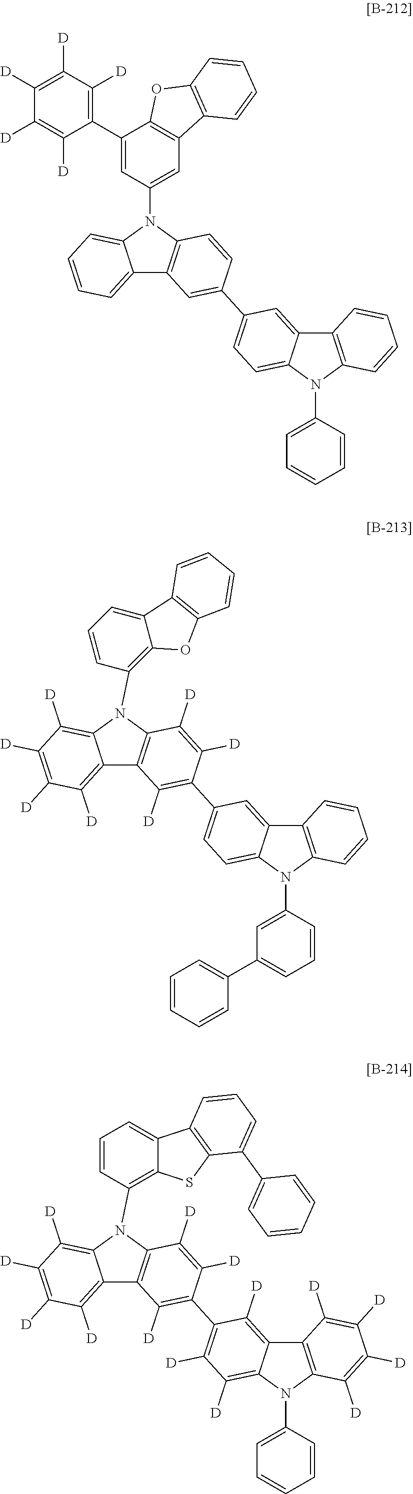 [SMILES: [2H]c1c([2H])c([2H])c(-c2cc(-n3c4ccccc4c4cc(-c5ccc6c(c5)c5ccccc5n6-c5ccccc5)ccc43)cc3c2oc2ccccc23)c([2H])c1[2H] ; [2H]c1c([2H])c([2H])c2c(c1[2H])c1c([2H])c(-c3ccc4c(c3)c3ccccc3n4-c3cccc(-c4ccccc4)c3)c([2H])c([2H])c1n2-c1cccc2c1oc1ccccc12 ; [2H]c1c([2H])c([2H])c2c(c1[2H])c1c([2H])c(-c3c([2H])c([2H])c4c(c3[2H])c3c([2H])c([2H])c([2H])c([2H])c3n4-c3cccc4c3sc3c(-c5ccccc5)cccc34)c([2H])c([2H])c1n2-c1ccccc1]

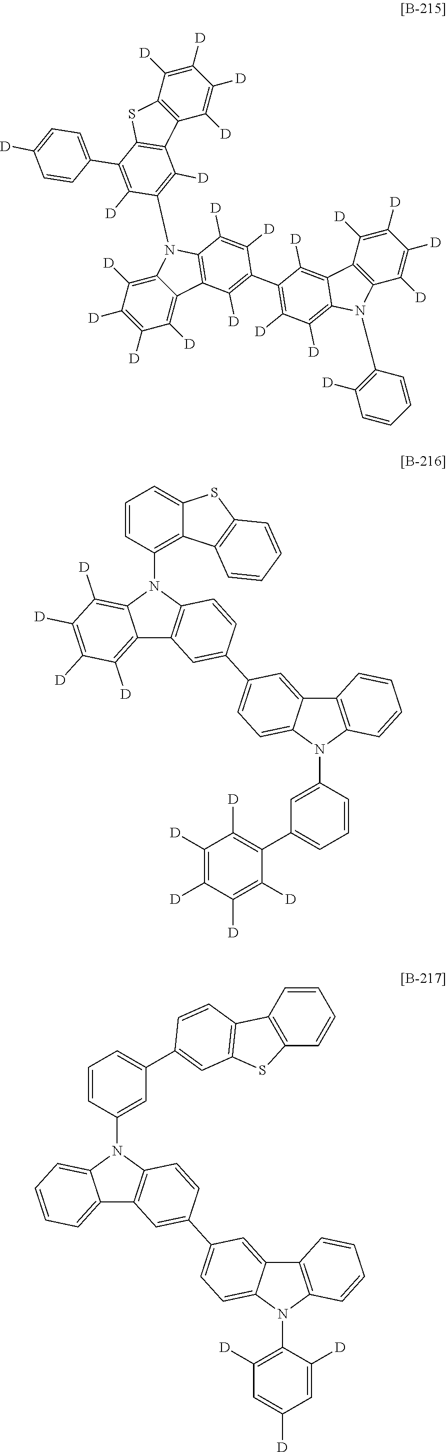 [SMILES: [2H]c1ccc(-c2c([2H])c(-n3c4c([2H])c([2H])c([2H])c([2H])c4c4c([2H])c(-c5c([2H])c([2H])c6c(c5[2H])c5c([2H])c([2H])c([2H])c([2H])c5n6-c5ccccc5[2H])c([2H])c([2H])c43)c([2H])c3c2sc2c([2H])c([2H])c([2H])c([2H])c23)cc1 ; [2H]c1c([2H])c([2H])c(-c2cccc(-n3c4ccccc4c4cc(-c5ccc6c(c5)c5c([2H])c([2H])c([2H])c([2H])c5n6-c5cccc6sc7ccccc7c56)ccc43)c2)c([2H])c1[2H] ; [2H]c1cc([2H])c(-n2c3ccccc3c3cc(-c4ccc5c(c4)c4ccccc4n5-c4cccc(-c5ccc6c(c5)sc5ccccc56)c4)ccc32)c([2H])c1]

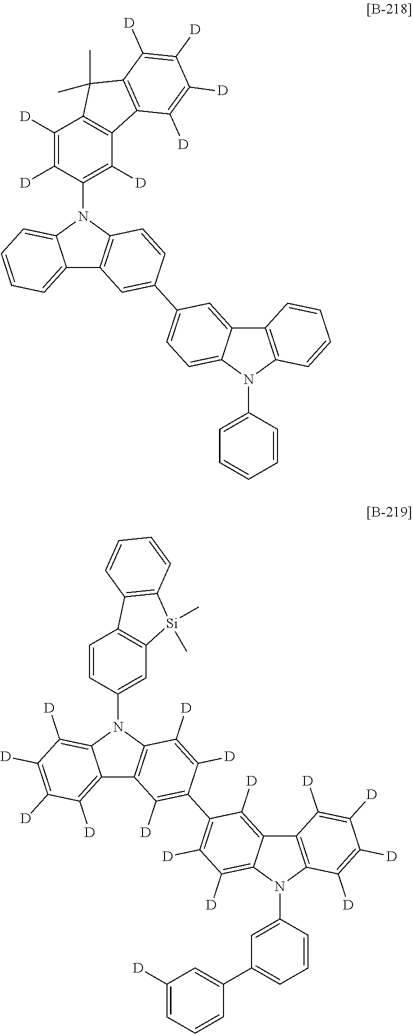 [SMILES: [2H]c1c([2H])c([2H])c2c(c1[2H])-c1c([2H])c(-n3c4ccccc4c4cc(-c5ccc6c(c5)c5ccccc5n6-c5ccccc5)ccc43)c([2H])c([2H])c1C2(C)C ; [2H]c1cccc(-c2cccc(-n3c4c([2H])c([2H])c([2H])c([2H])c4c4c([2H])c(-c5c([2H])c([2H])c6c(c5[2H])c5c([2H])c([2H])c([2H])c([2H])c5n6-c5ccc6c(c5)[Si](C)(C)c5ccccc5-6)c([2H])c([2H])c43)c2)c1]

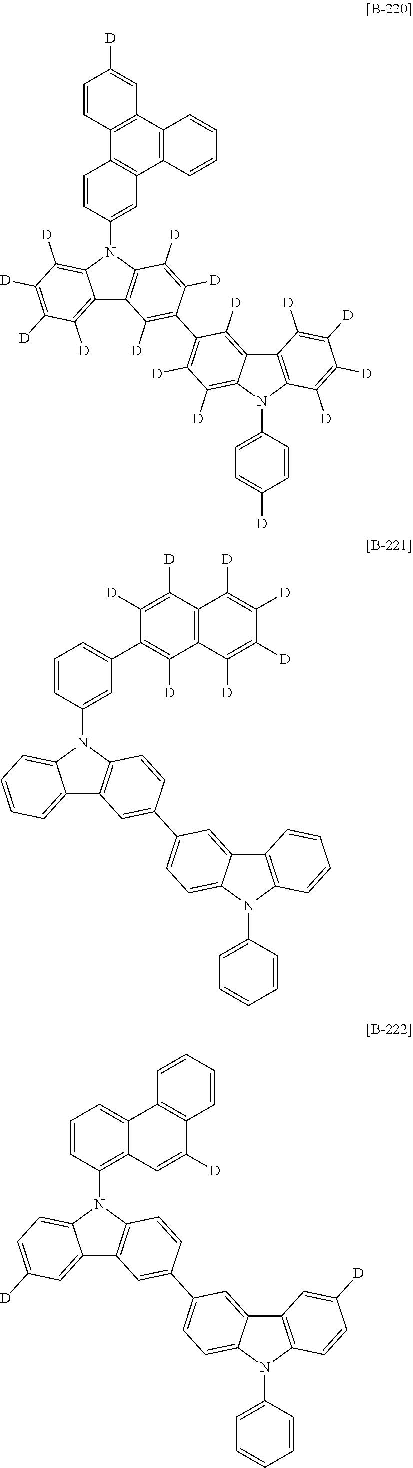 [SMILES: [2H]c1ccc(-n2c3c([2H])c([2H])c([2H])c([2H])c3c3c([2H])c(-c4c([2H])c([2H])c5c(c4[2H])c4c([2H])c([2H])c([2H])c([2H])c4n5-c4ccc5c6ccc([2H])cc6c6ccccc6c5c4)c([2H])c([2H])c32)cc1 ; [2H]c1c([2H])c([2H])c2c([2H])c(-c3cccc(-n4c5ccccc5c5cc(-c6ccc7c(c6)c6ccccc6n7-c6ccccc6)ccc54)c3)c([2H])c([2H])c2c1[2H] ; [2H]c1ccc2c(c1)c1cc(-c3ccc4c(c3)c3cc([2H])ccc3n4-c3cccc4c3cc([2H])c3ccccc34)ccc1n2-c1ccccc1]

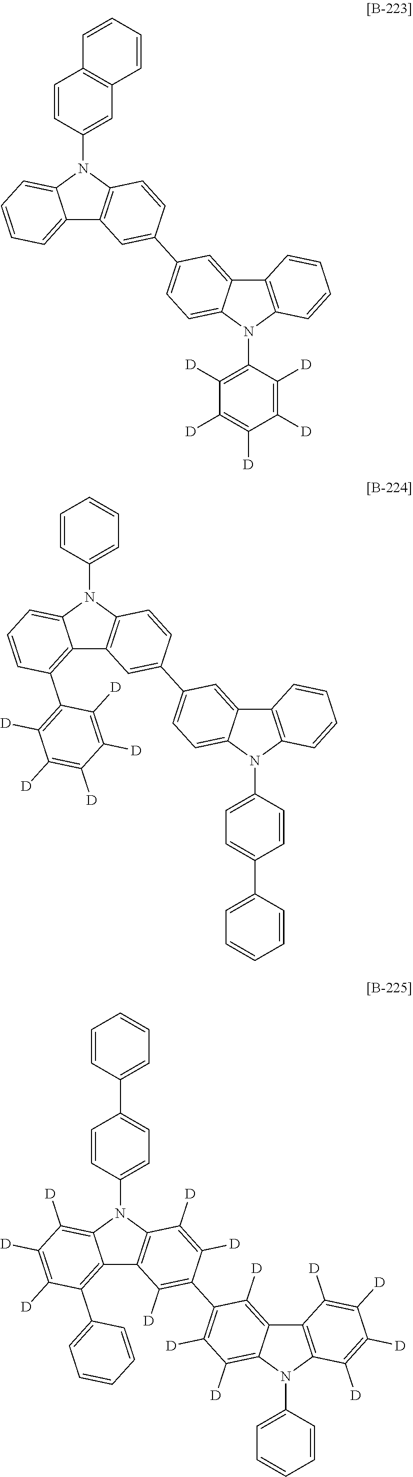 [SMILES: [2H]c1c([2H])c([2H])c(-n2c3ccccc3c3cc(-c4ccc5c(c4)c4ccccc4n5-c4ccc5ccccc5c4)ccc32)c([2H])c1[2H] ; [2H]c1c([2H])c([2H])c(-c2cccc3c2c2cc(-c4ccc5c(c4)c4ccccc4n5-c4ccc(-c5ccccc5)cc4)ccc2n3-c2ccccc2)c([2H])c1[2H] ; [2H]c1c([2H])c([2H])c2c(c1[2H])c1c([2H])c(-c3c([2H])c([2H])c4c(c3[2H])c3c(-c5ccccc5)c([2H])c([2H])c([2H])c3n4-c3ccc(-c4ccccc4)cc3)c([2H])c([2H])c1n2-c1ccccc1]

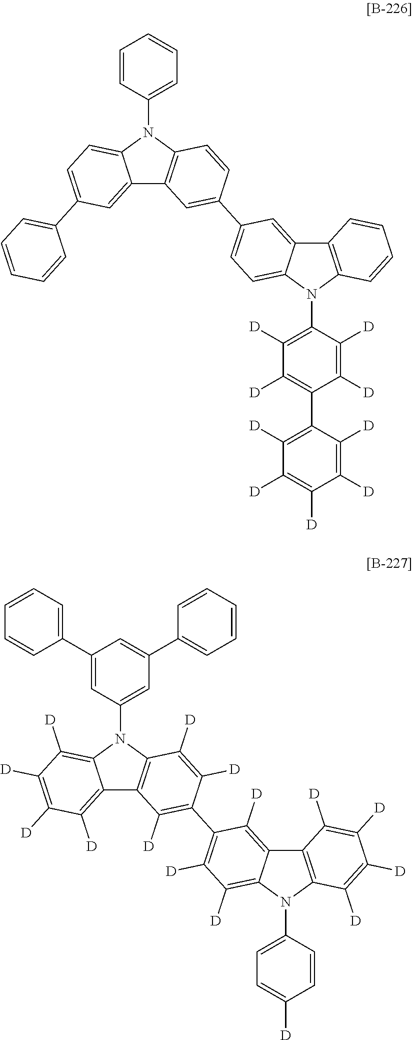 [SMILES: [2H]c1c([2H])c([2H])c(-c2c([2H])c([2H])c(-n3c4ccccc4c4cc(-c5ccc6c(c5)c5cc(-c7ccccc7)ccc5n6-c5ccccc5)ccc43)c([2H])c2[2H])c([2H])c1[2H] ; [2H]c1ccc(-n2c3c([2H])c([2H])c([2H])c([2H])c3c3c([2H])c(-c4c([2H])c([2H])c5c(c4[2H])c4c([2H])c([2H])c([2H])c([2H])c4n5-c4cc(-c5ccccc5)cc(-c5ccccc5)c4)c([2H])c([2H])c32)cc1]

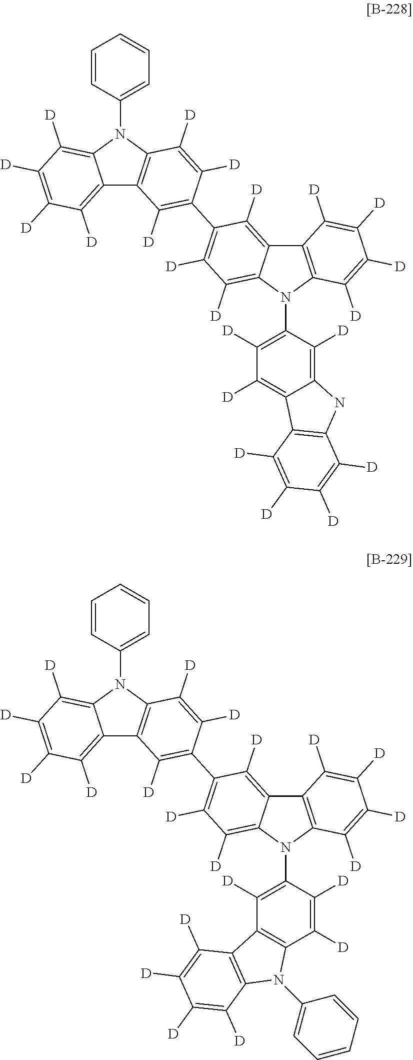 [SMILES: [2H]c1c([2H])c([2H])c2c([nH]c3c([2H])c(-n4c5c([2H])c([2H])c([2H])c([2H])c5c5c([2H])c(-c6c([2H])c([2H])c7c(c6[2H])c6c([2H])c([2H])c([2H])c([2H])c6n7-c6ccccc6)c([2H])c([2H])c54)c([2H])c([2H])c32)c1[2H] ; [2H]c1c([2H])c([2H])c2c(c1[2H])c1c([2H])c(-c3c([2H])c([2H])c4c(c3[2H])c3c([2H])c([2H])c([2H])c([2H])c3n4-c3c([2H])c([2H])c4c(c3[2H])c3c([2H])c([2H])c([2H])c([2H])c3n4-c3ccccc3)c([2H])c([2H])c1n2-c1ccccc1]

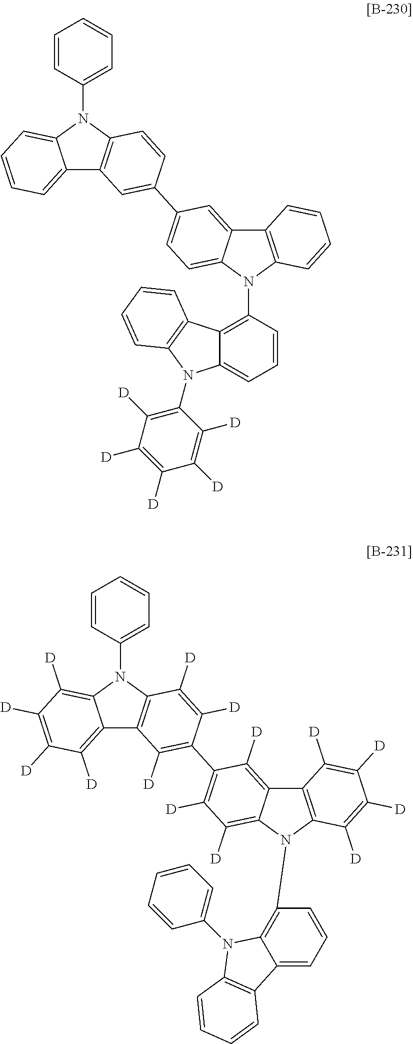 [SMILES: [2H]c1c([2H])c([2H])c(-n2c3ccccc3c3c(-n4c5ccccc5c5cc(-c6ccc7c(c6)c6ccccc6n7-c6ccccc6)ccc54)cccc32)c([2H])c1[2H] ; [2H]c1c([2H])c([2H])c2c(c1[2H])c1c([2H])c(-c3c([2H])c([2H])c4c(c3[2H])c3c([2H])c([2H])c([2H])c([2H])c3n4-c3cccc4c5ccccc5n(-c5ccccc5)c34)c([2H])c([2H])c1n2-c1ccccc1]

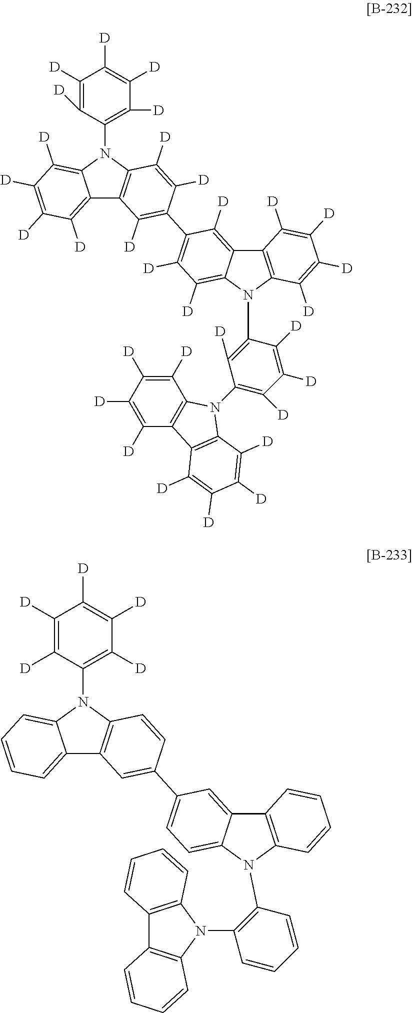 [SMILES: [2H]c1c([2H])c([2H])c(-n2c3c([2H])c([2H])c([2H])c([2H])c3c3c([2H])c(-c4c([2H])c([2H])c5c(c4[2H])c4c([2H])c([2H])c([2H])c([2H])c4n5-c4c([2H])c([2H])c([2H])c(-n5c6c([2H])c([2H])c([2H])c([2H])c6c6c([2H])c([2H])c([2H])c([2H])c65)c4[2H])c([2H])c([2H])c32)c([2H])c1[2H] ; [2H]c1c([2H])c([2H])c(-n2c3ccccc3c3cc(-c4ccc5c(c4)c4ccccc4n5-c4ccccc4-n4c5ccccc5c5ccccc54)ccc32)c([2H])c1[2H]]

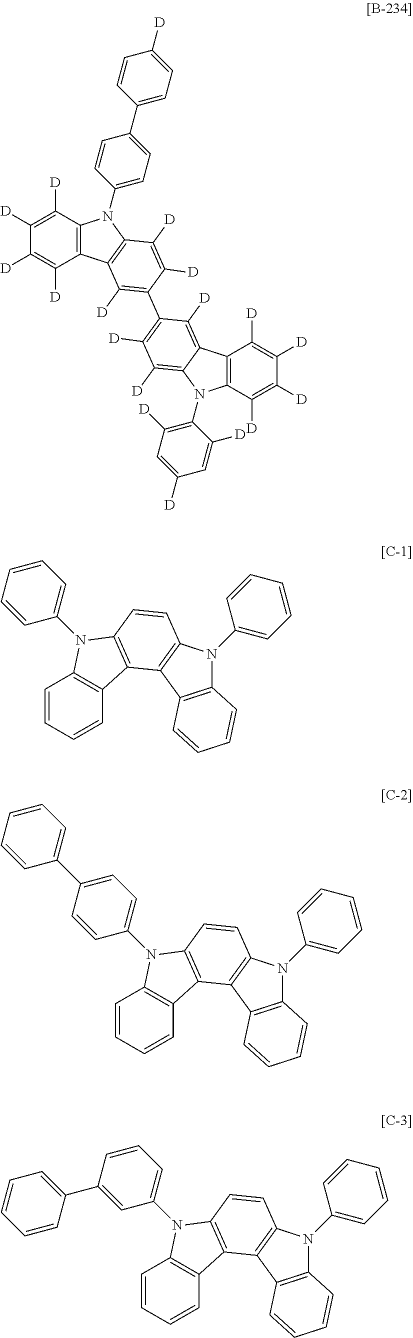 [SMILES: [2H]c1ccc(-c2ccc(-n3c4c([2H])c([2H])c([2H])c([2H])c4c4c([2H])c(-c5c([2H])c([2H])c6c(c5[2H])c5c([2H])c([2H])c([2H])c([2H])c5n6-c5c([2H])cc([2H])cc5[2H])c([2H])c([2H])c43)cc2)cc1 ; c1ccc(-n2c3ccccc3c3c4c5ccccc5n(-c5ccccc5)c4ccc32)cc1 ; c1ccc(-c2ccc(-n3c4ccccc4c4c5c6ccccc6n(-c6ccccc6)c5ccc43)cc2)cc1 ; c1ccc(-c2cccc(-n3c4ccccc4c4c5c6ccccc6n(-c6ccccc6)c5ccc43)c2)cc1]

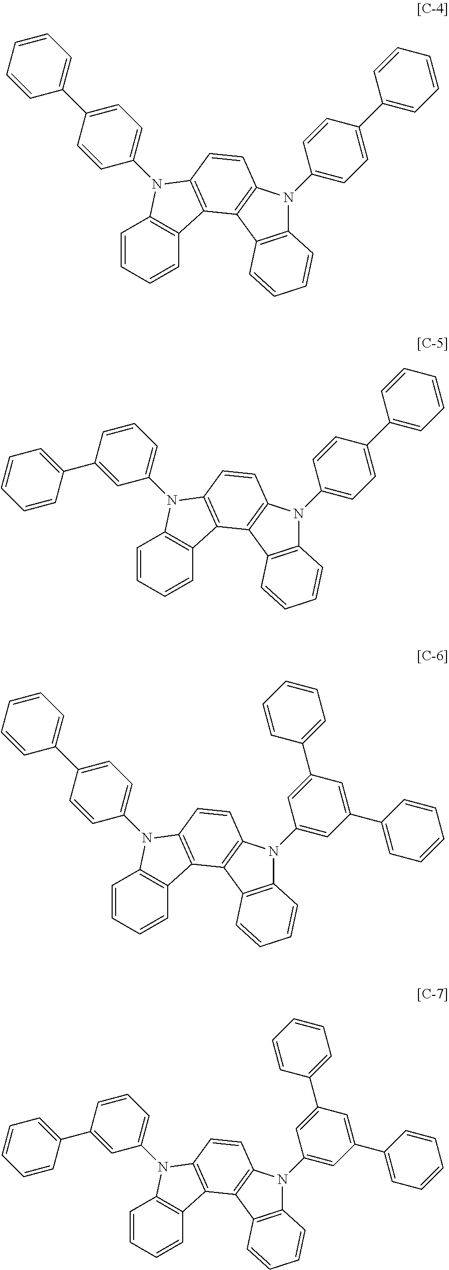 [SMILES: c1ccc(-c2ccc(-n3c4ccccc4c4c5c6ccccc6n(-c6ccc(-c7ccccc7)cc6)c5ccc43)cc2)cc1 ; c1ccc(-c2ccc(-n3c4ccccc4c4c5c6ccccc6n(-c6cccc(-c7ccccc7)c6)c5ccc43)cc2)cc1 ; c1ccc(-c2ccc(-n3c4ccccc4c4c5c6ccccc6n(-c6cc(-c7ccccc7)cc(-c7ccccc7)c6)c5ccc43)cc2)cc1 ; c1ccc(-c2cccc(-n3c4ccccc4c4c5c6ccccc6n(-c6cc(-c7ccccc7)cc(-c7ccccc7)c6)c5ccc43)c2)cc1]

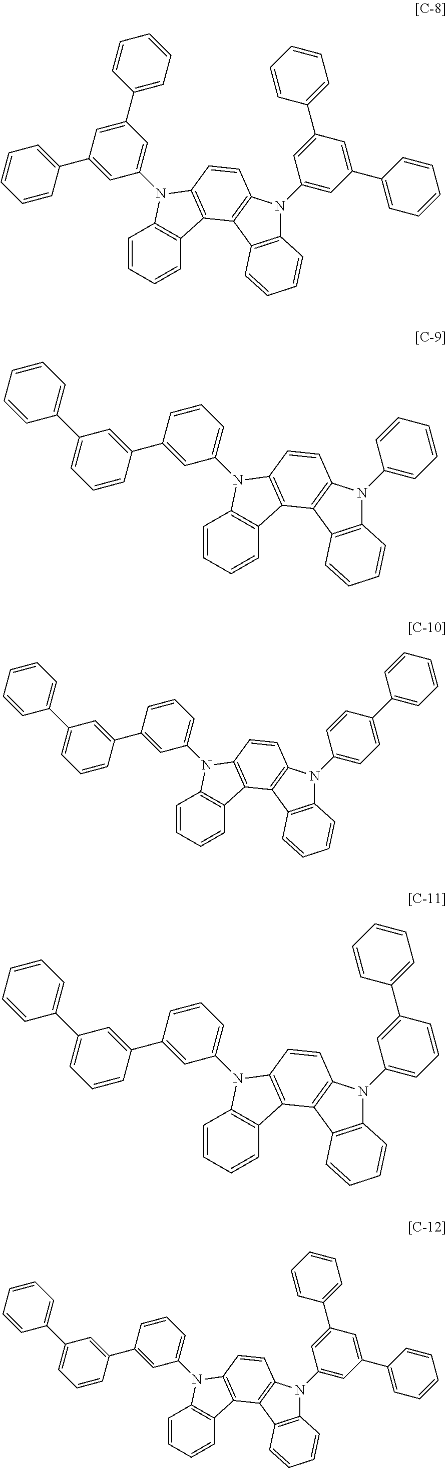 [SMILES: c1ccc(-c2cc(-c3ccccc3)cc(-n3c4ccccc4c4c5c6ccccc6n(-c6cc(-c7ccccc7)cc(-c7ccccc7)c6)c5ccc43)c2)cc1 ; c1ccc(-c2cccc(-c3cccc(-n4c5ccccc5c5c6c7ccccc7n(-c7ccccc7)c6ccc54)c3)c2)cc1 ; c1ccc(-c2ccc(-n3c4ccccc4c4c5c6ccccc6n(-c6cccc(-c7cccc(-c8ccccc8)c7)c6)c5ccc43)cc2)cc1 ; c1ccc(-c2cccc(-c3cccc(-n4c5ccccc5c5c6c7ccccc7n(-c7cccc(-c8ccccc8)c7)c6ccc54)c3)c2)cc1 ; c1ccc(-c2cccc(-c3cccc(-n4c5ccccc5c5c6c7ccccc7n(-c7cc(-c8ccccc8)cc(-c8ccccc8)c7)c6ccc54)c3)c2)cc1]

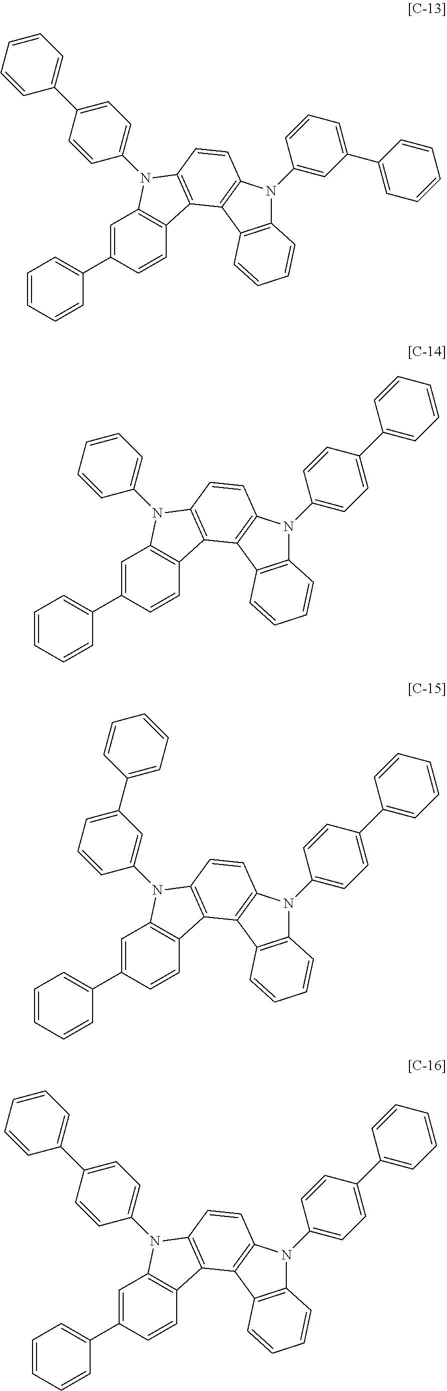 [SMILES: c1ccc(-c2ccc(-n3c4cc(-c5ccccc5)ccc4c4c5c6ccccc6n(-c6cccc(-c7ccccc7)c6)c5ccc43)cc2)cc1 ; c1ccc(-c2ccc(-n3c4ccccc4c4c5c6ccc(-c7ccccc7)cc6n(-c6ccccc6)c5ccc43)cc2)cc1 ; c1ccc(-c2ccc(-n3c4ccccc4c4c5c6ccc(-c7ccccc7)cc6n(-c6cccc(-c7ccccc7)c6)c5ccc43)cc2)cc1 ; c1ccc(-c2ccc(-n3c4ccccc4c4c5c6ccc(-c7ccccc7)cc6n(-c6ccc(-c7ccccc7)cc6)c5ccc43)cc2)cc1]

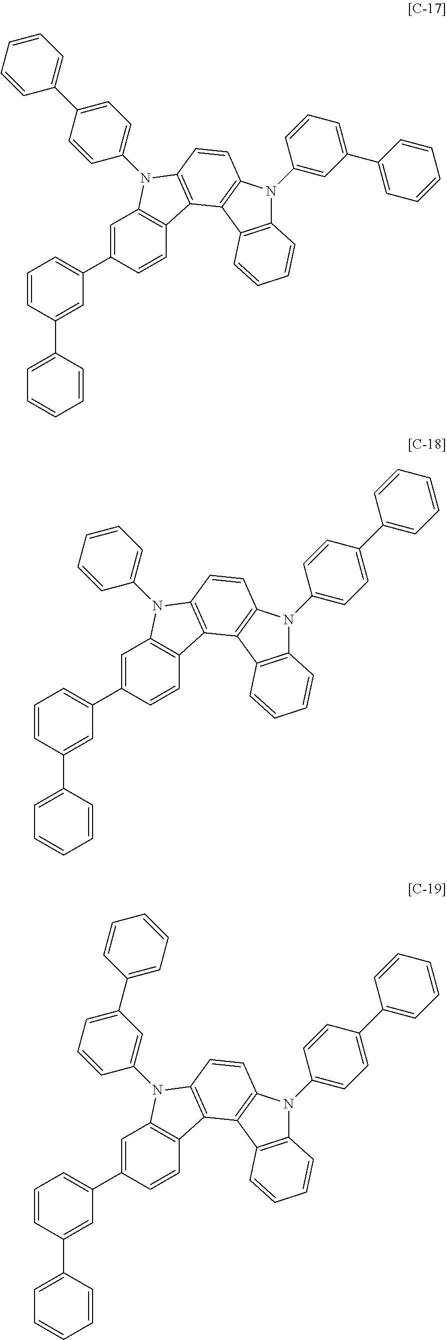 [SMILES: c1ccc(-c2ccc(-n3c4cc(-c5cccc(-c6ccccc6)c5)ccc4c4c5c6ccccc6n(-c6cccc(-c7ccccc7)c6)c5ccc43)cc2)cc1 ; c1ccc(-c2ccc(-n3c4ccccc4c4c5c6ccc(-c7cccc(-c8ccccc8)c7)cc6n(-c6ccccc6)c5ccc43)cc2)cc1 ; c1ccc(-c2ccc(-n3c4ccccc4c4c5c6ccc(-c7cccc(-c8ccccc8)c7)cc6n(-c6cccc(-c7ccccc7)c6)c5ccc43)cc2)cc1]

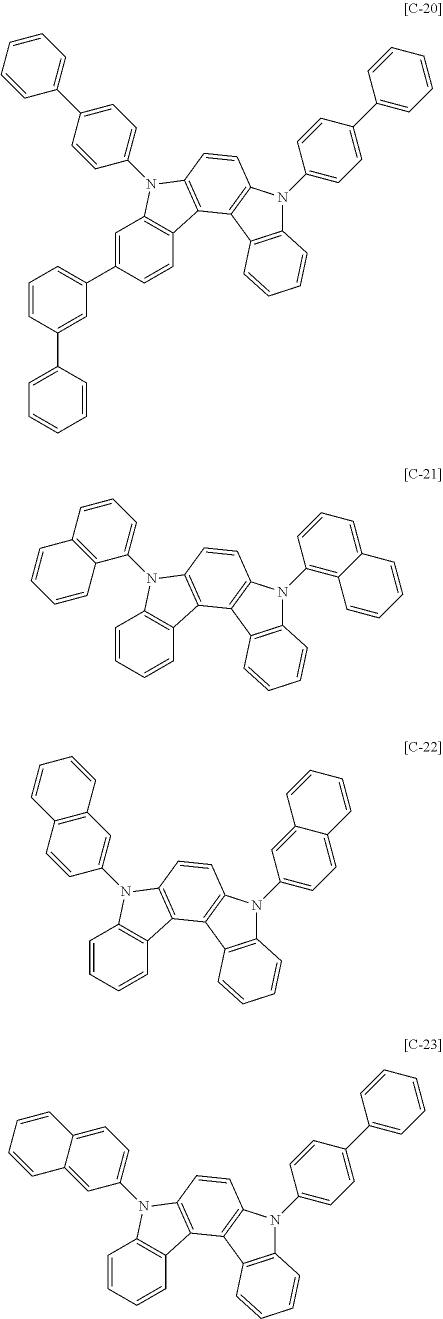 [SMILES: c1ccc(-c2ccc(-n3c4ccccc4c4c5c6ccc(-c7cccc(-c8ccccc8)c7)cc6n(-c6ccc(-c7ccccc7)cc6)c5ccc43)cc2)cc1 ; c1ccc2c(-n3c4ccccc4c4c5c6ccccc6n(-c6cccc7ccccc67)c5ccc43)cccc2c1 ; c1ccc2cc(-n3c4ccccc4c4c5c6ccccc6n(-c6ccc7ccccc7c6)c5ccc43)ccc2c1 ; c1ccc(-c2ccc(-n3c4ccccc4c4c5c6ccccc6n(-c6ccc7ccccc7c6)c5ccc43)cc2)cc1]

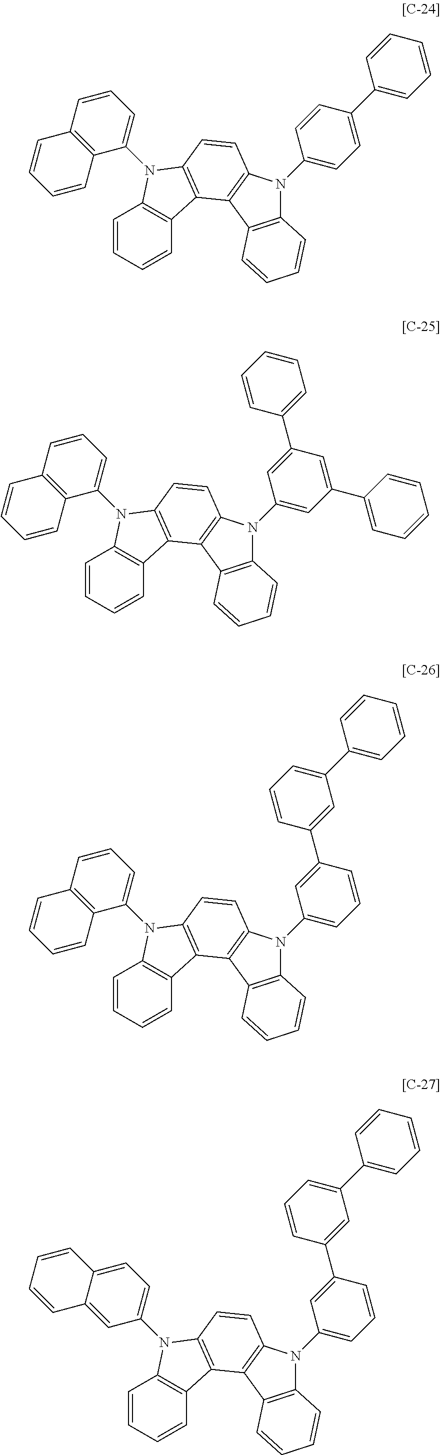 [SMILES: c1ccc(-c2ccc(-n3c4ccccc4c4c5c6ccccc6n(-c6cccc7ccccc67)c5ccc43)cc2)cc1 ; c1ccc(-c2cc(-c3ccccc3)cc(-n3c4ccccc4c4c5c6ccccc6n(-c6cccc7ccccc67)c5ccc43)c2)cc1 ; c1ccc(-c2cccc(-c3cccc(-n4c5ccccc5c5c6c7ccccc7n(-c7cccc8ccccc78)c6ccc54)c3)c2)cc1 ; c1ccc(-c2cccc(-c3cccc(-n4c5ccccc5c5c6c7ccccc7n(-c7ccc8ccccc8c7)c6ccc54)c3)c2)cc1]

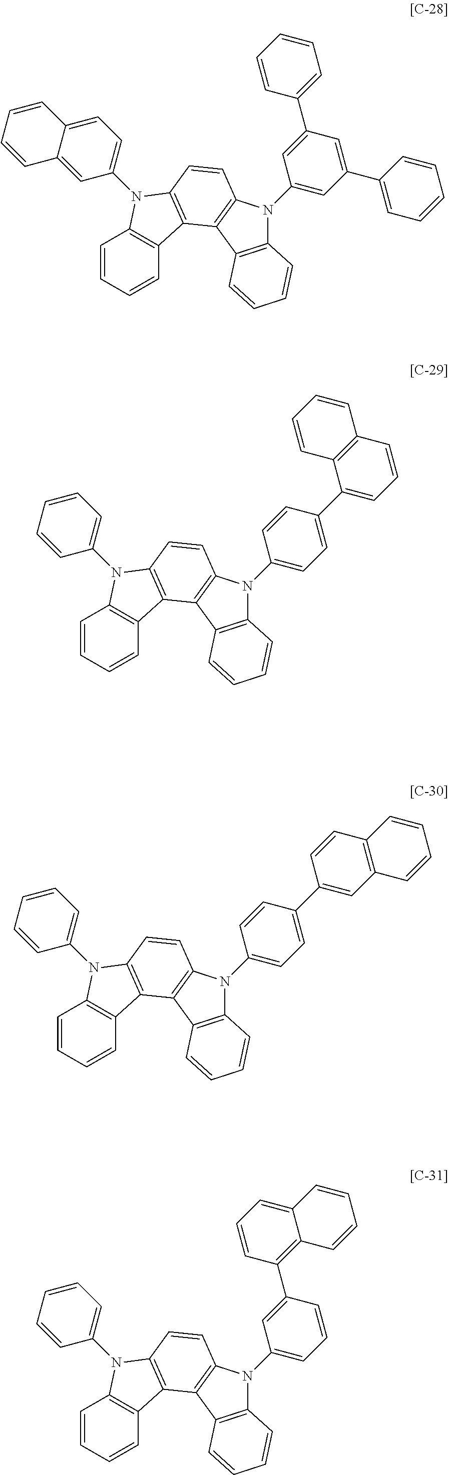 [SMILES: c1ccc(-c2cc(-c3ccccc3)cc(-n3c4ccccc4c4c5c6ccccc6n(-c6ccc7ccccc7c6)c5ccc43)c2)cc1 ; c1ccc(-n2c3ccccc3c3c4c5ccccc5n(-c5ccc(-c6cccc7ccccc67)cc5)c4ccc32)cc1 ; c1ccc(-n2c3ccccc3c3c4c5ccccc5n(-c5ccc(-c6ccc7ccccc7c6)cc5)c4ccc32)cc1 ; c1ccc(-n2c3ccccc3c3c4c5ccccc5n(-c5cccc(-c6cccc7ccccc67)c5)c4ccc32)cc1]

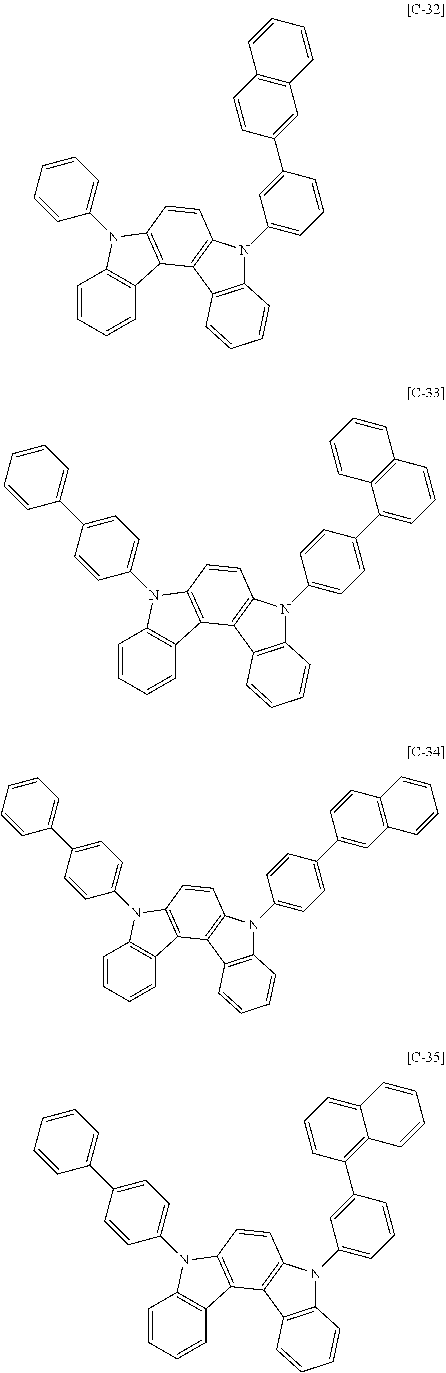 [SMILES: c1ccc(-n2c3ccccc3c3c4c5ccccc5n(-c5cccc(-c6ccc7ccccc7c6)c5)c4ccc32)cc1 ; c1ccc(-c2ccc(-n3c4ccccc4c4c5c6ccccc6n(-c6ccc(-c7cccc8ccccc78)cc6)c5ccc43)cc2)cc1 ; c1ccc(-c2ccc(-n3c4ccccc4c4c5c6ccccc6n(-c6ccc(-c7ccc8ccccc8c7)cc6)c5ccc43)cc2)cc1 ; c1ccc(-c2ccc(-n3c4ccccc4c4c5c6ccccc6n(-c6cccc(-c7cccc8ccccc78)c6)c5ccc43)cc2)cc1]

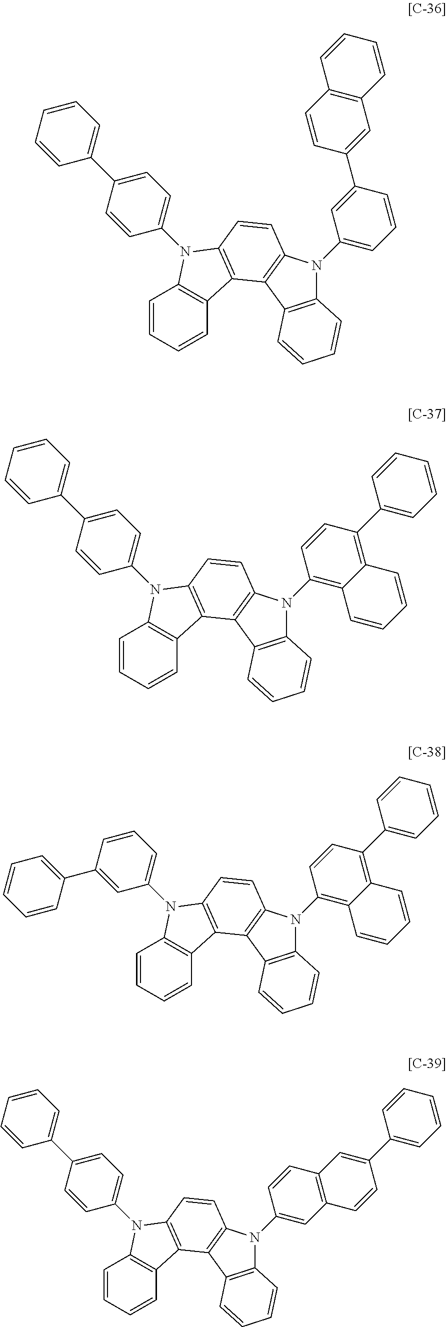 [SMILES: c1ccc(-c2ccc(-n3c4ccccc4c4c5c6ccccc6n(-c6cccc(-c7ccc8ccccc8c7)c6)c5ccc43)cc2)cc1 ; c1ccc(-c2ccc(-n3c4ccccc4c4c5c6ccccc6n(-c6ccc(-c7ccccc7)c7ccccc67)c5ccc43)cc2)cc1 ; c1ccc(-c2cccc(-n3c4ccccc4c4c5c6ccccc6n(-c6ccc(-c7ccccc7)c7ccccc67)c5ccc43)c2)cc1 ; c1ccc(-c2ccc(-n3c4ccccc4c4c5c6ccccc6n(-c6ccc7cc(-c8ccccc8)ccc7c6)c5ccc43)cc2)cc1]

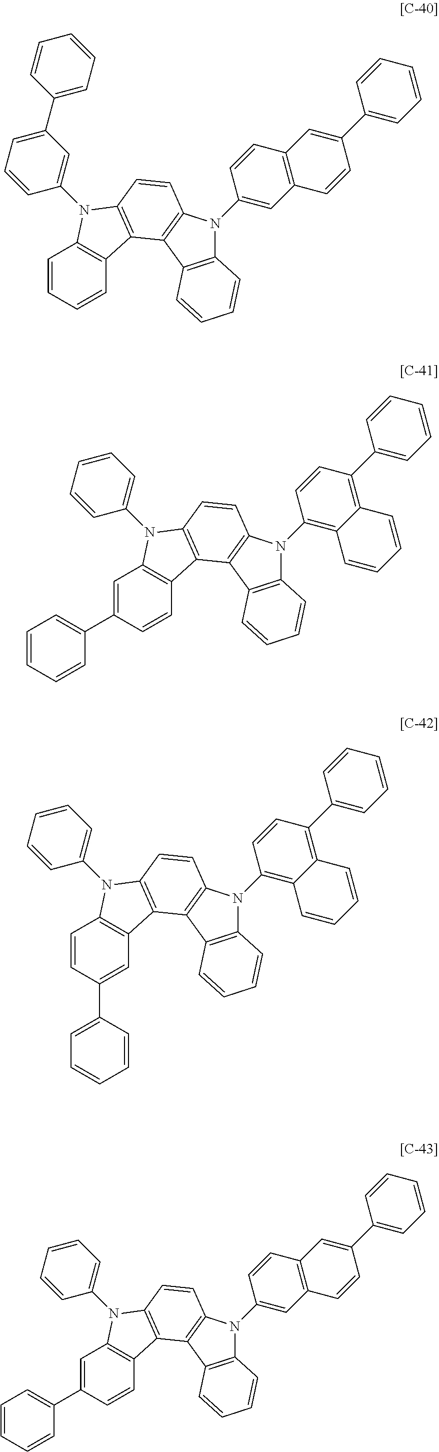 [SMILES: c1ccc(-c2cccc(-n3c4ccccc4c4c5c6ccccc6n(-c6ccc7cc(-c8ccccc8)ccc7c6)c5ccc43)c2)cc1 ; c1ccc(-c2ccc3c4c5c6ccccc6n(-c6ccc(-c7ccccc7)c7ccccc67)c5ccc4n(-c4ccccc4)c3c2)cc1 ; c1ccc(-c2ccc3c(c2)c2c4c5ccccc5n(-c5ccc(-c6ccccc6)c6ccccc56)c4ccc2n3-c2ccccc2)cc1 ; c1ccc(-c2ccc3cc(-n4c5ccccc5c5c6c7ccc(-c8ccccc8)cc7n(-c7ccccc7)c6ccc54)ccc3c2)cc1]

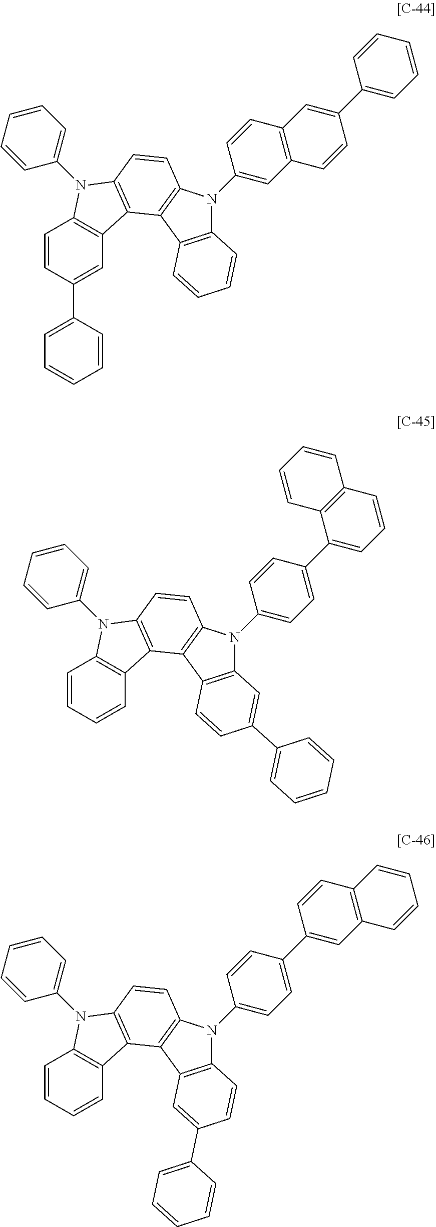 [SMILES: c1ccc(-c2ccc3cc(-n4c5ccccc5c5c6c7cc(-c8ccccc8)ccc7n(-c7ccccc7)c6ccc54)ccc3c2)cc1 ; c1ccc(-c2ccc3c4c5c6ccccc6n(-c6ccccc6)c5ccc4n(-c4ccc(-c5cccc6ccccc56)cc4)c3c2)cc1 ; c1ccc(-c2ccc3c(c2)c2c4c5ccccc5n(-c5ccccc5)c4ccc2n3-c2ccc(-c3ccc4ccccc4c3)cc2)cc1]

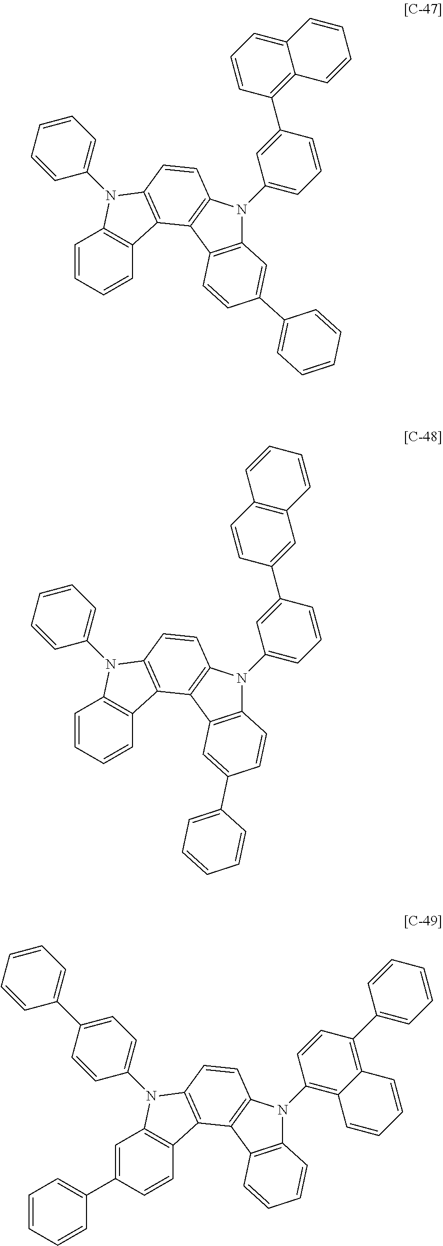 [SMILES: c1ccc(-c2ccc3c4c5c6ccccc6n(-c6ccccc6)c5ccc4n(-c4cccc(-c5cccc6ccccc56)c4)c3c2)cc1 ; c1ccc(-c2ccc3c(c2)c2c4c5ccccc5n(-c5ccccc5)c4ccc2n3-c2cccc(-c3ccc4ccccc4c3)c2)cc1 ; c1ccc(-c2ccc(-n3c4cc(-c5ccccc5)ccc4c4c5c6ccccc6n(-c6ccc(-c7ccccc7)c7ccccc67)c5ccc43)cc2)cc1]

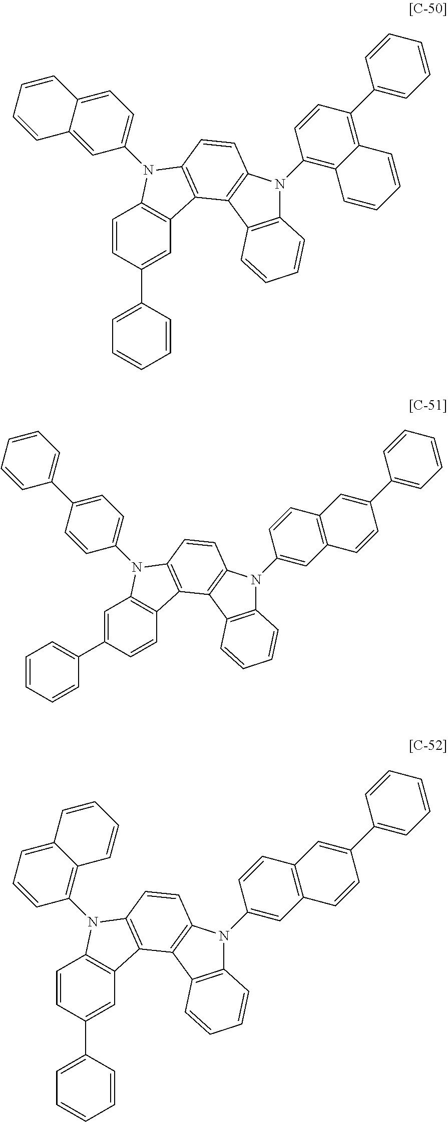 [SMILES: c1ccc(-c2ccc3c(c2)c2c4c5ccccc5n(-c5ccc(-c6ccccc6)c6ccccc56)c4ccc2n3-c2ccc3ccccc3c2)cc1 ; c1ccc(-c2ccc(-n3c4cc(-c5ccccc5)ccc4c4c5c6ccccc6n(-c6ccc7cc(-c8ccccc8)ccc7c6)c5ccc43)cc2)cc1 ; c1ccc(-c2ccc3cc(-n4c5ccccc5c5c6c7cc(-c8ccccc8)ccc7n(-c7cccc8ccccc78)c6ccc54)ccc3c2)cc1]

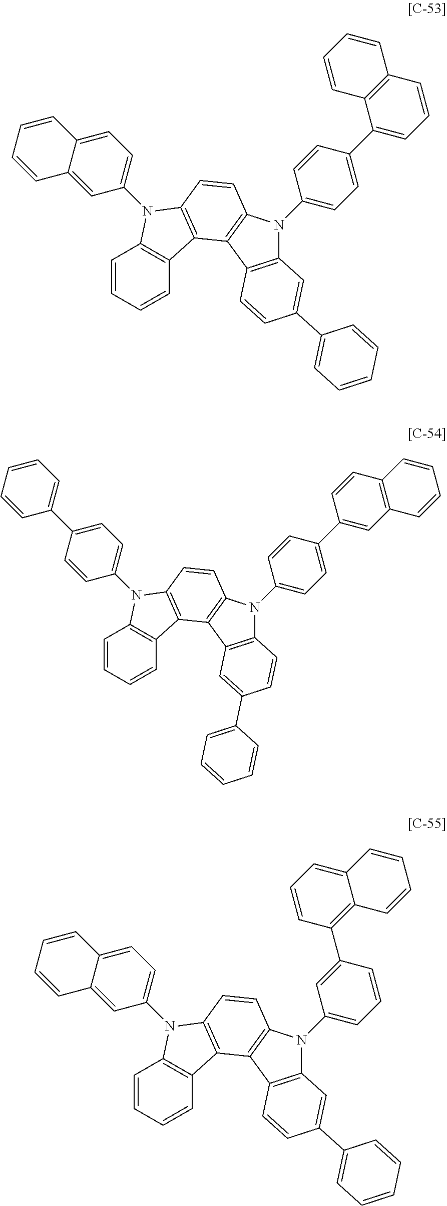 [SMILES: c1ccc(-c2ccc3c4c5c6ccccc6n(-c6ccc7ccccc7c6)c5ccc4n(-c4ccc(-c5cccc6ccccc56)cc4)c3c2)cc1 ; c1ccc(-c2ccc(-n3c4ccccc4c4c5c6cc(-c7ccccc7)ccc6n(-c6ccc(-c7ccc8ccccc8c7)cc6)c5ccc43)cc2)cc1 ; c1ccc(-c2ccc3c4c5c6ccccc6n(-c6ccc7ccccc7c6)c5ccc4n(-c4cccc(-c5cccc6ccccc56)c4)c3c2)cc1]

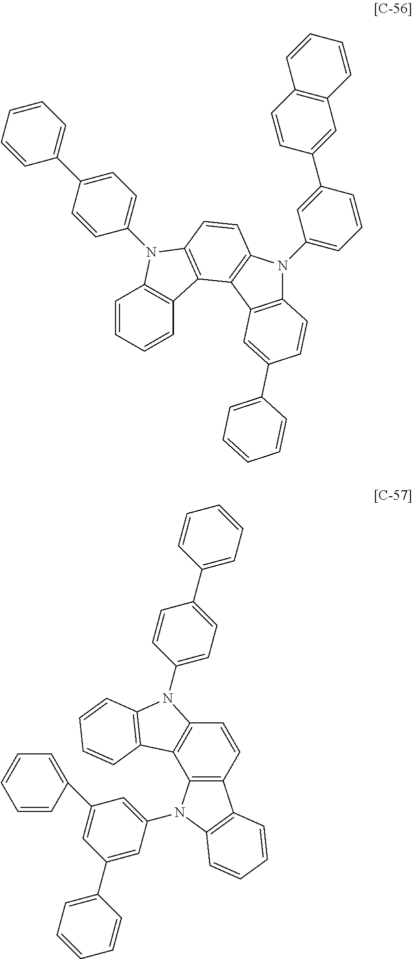 [SMILES: c1ccc(-c2ccc(-n3c4ccccc4c4c5c6cc(-c7ccccc7)ccc6n(-c6cccc(-c7ccc8ccccc8c7)c6)c5ccc43)cc2)cc1 ; c1ccc(-c2ccc(-n3c4ccccc4c4c3ccc3c5ccccc5n(-c5cc(-c6ccccc6)cc(-c6ccccc6)c5)c34)cc2)cc1]

In addition, examples of Compound C-1 to Compound C-57 listed in Group 2 in which at least one hydrogen is substituted with deuterium are shown below.

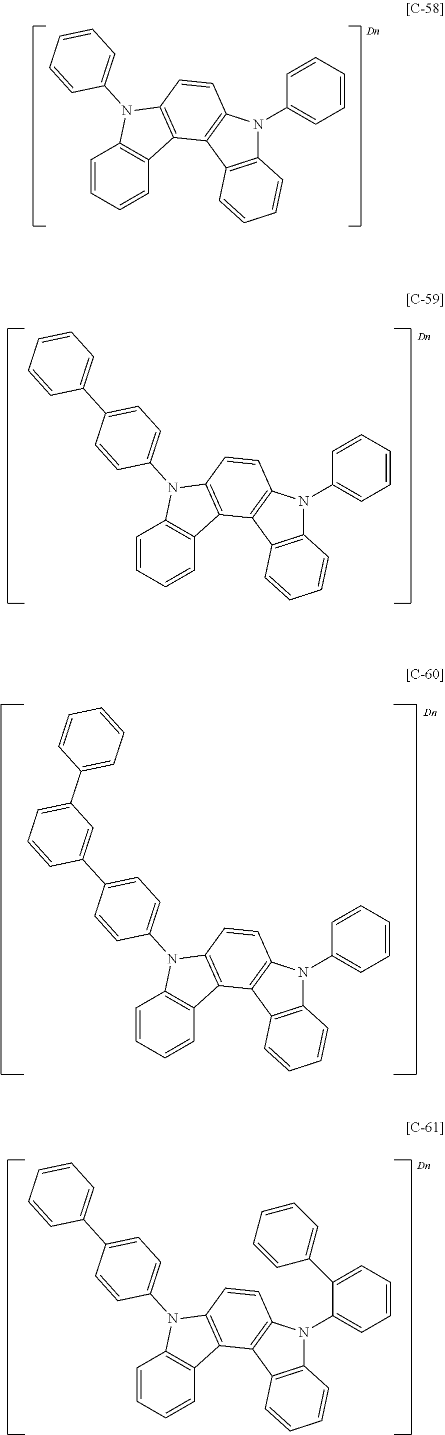

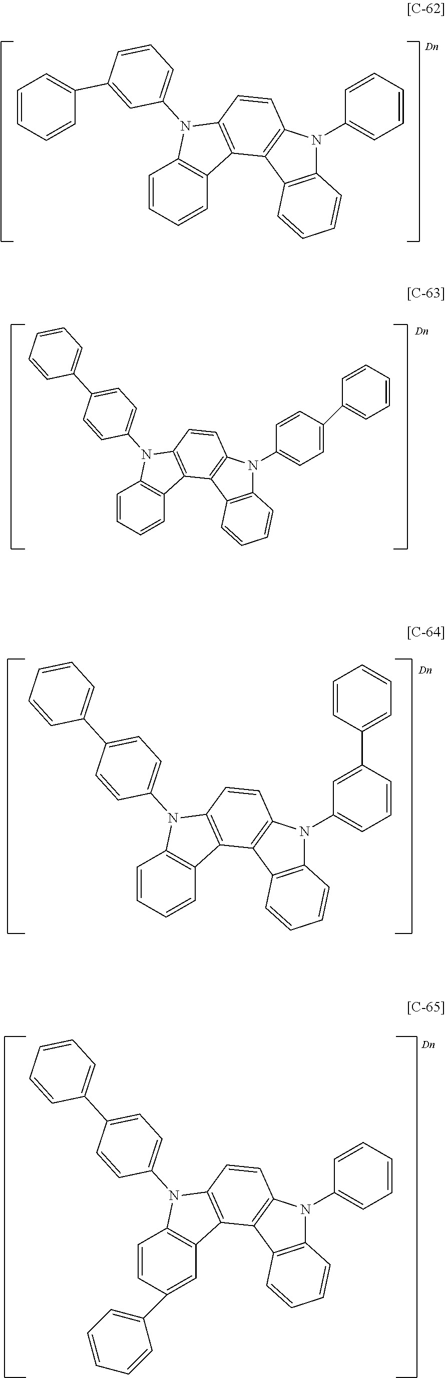

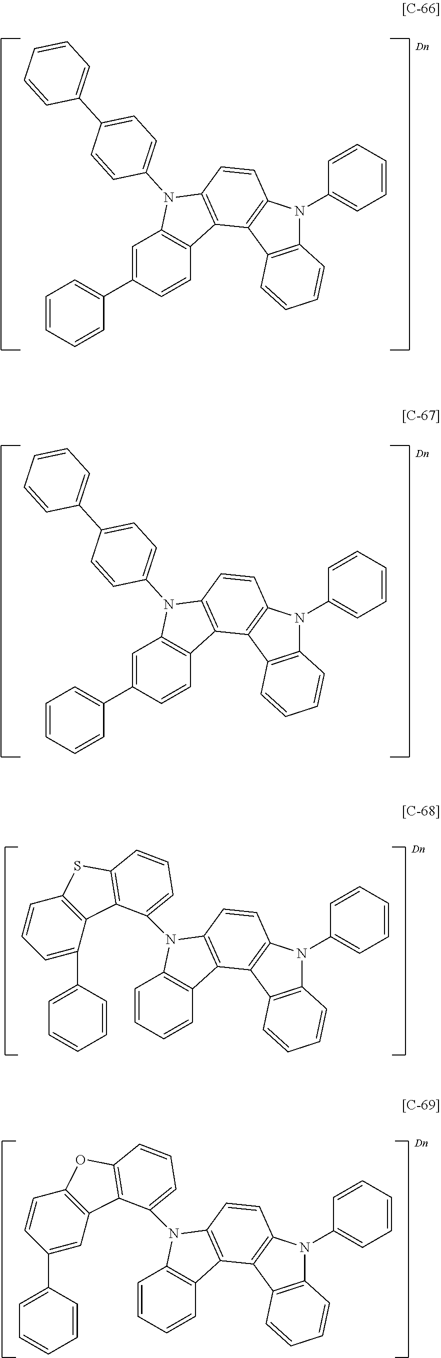

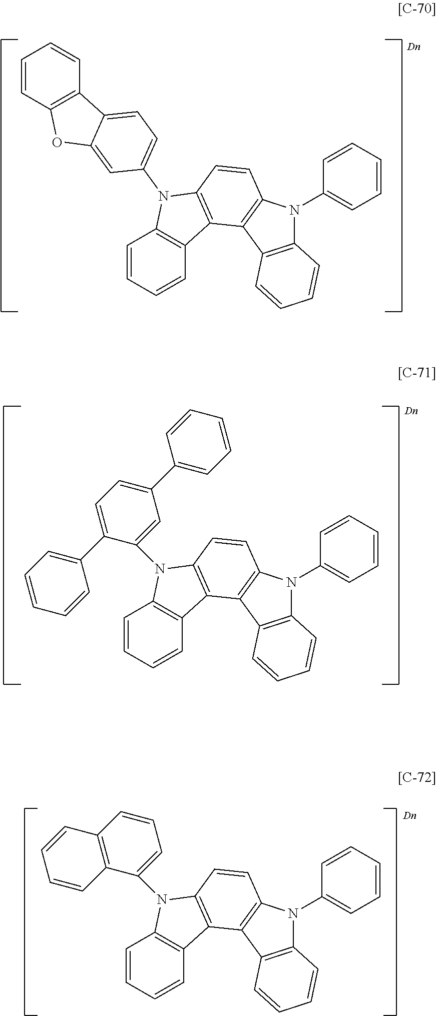

(Dn means the number of deuterium substituted, and represents a structure substituted with one or more deuterium)

For Compound C-58 to Compound C-72 of Group 2, the most specific structures according to the deuterium substitution position and substitution ratio are exemplarily shown.

When deuterium is substituted, it is not limited to the compounds exemplified below, and the deuterium substitution position and deuterium substitution ratio may include all changeable ranges within the ranges of Compound C-1 to Compound C-72.

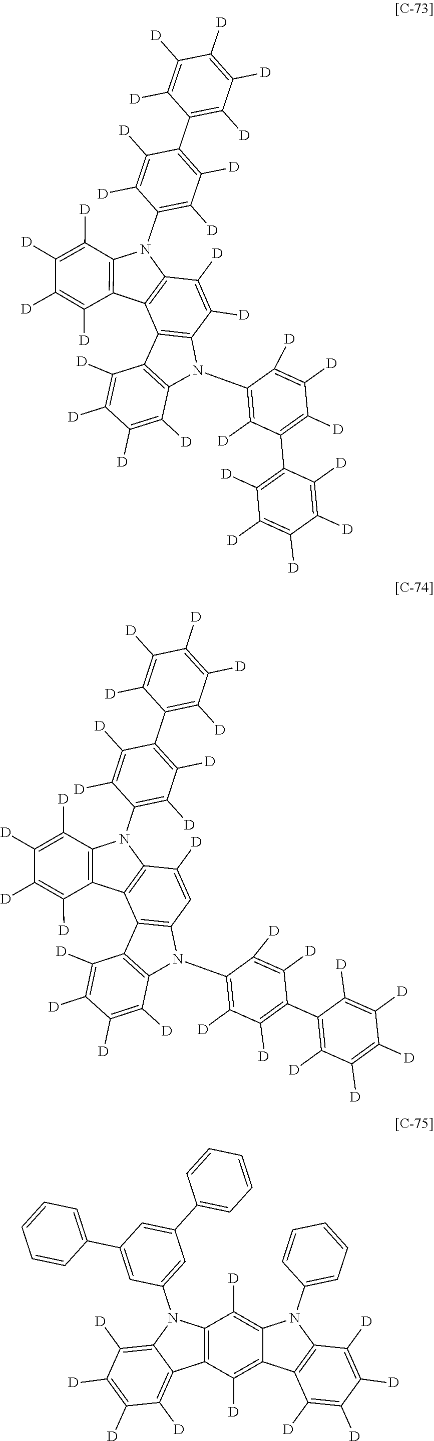

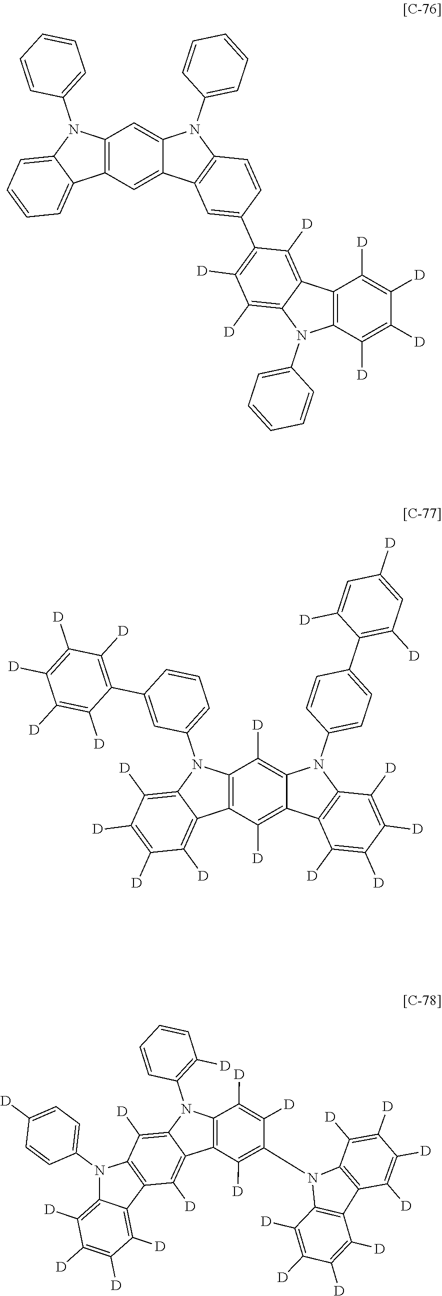 [SMILES: [2H]c1c([2H])c([2H])c2c(c1[2H])c1c([2H])c(-c3ccc4c(c3)c3cc5c6ccccc6n(-c6ccccc6)c5cc3n4-c3ccccc3)c([2H])c([2H])c1n2-c1ccccc1 ; [2H]c1cc([2H])c(-c2ccc(-n3c4c([2H])c([2H])c([2H])c([2H])c4c4c([2H])c5c6c([2H])c([2H])c([2H])c([2H])c6n(-c6cccc(-c7c([2H])c([2H])c([2H])c([2H])c7[2H])c6)c5c([2H])c43)cc2)c([2H])c1 ; [2H]c1ccc(-n2c3c([2H])c([2H])c([2H])c([2H])c3c3c([2H])c4c5c([2H])c(-n6c7c([2H])c([2H])c([2H])c([2H])c7c7c([2H])c([2H])c([2H])c([2H])c76)c([2H])c([2H])c5n(-c5ccccc5[2H])c4c([2H])c32)cc1]

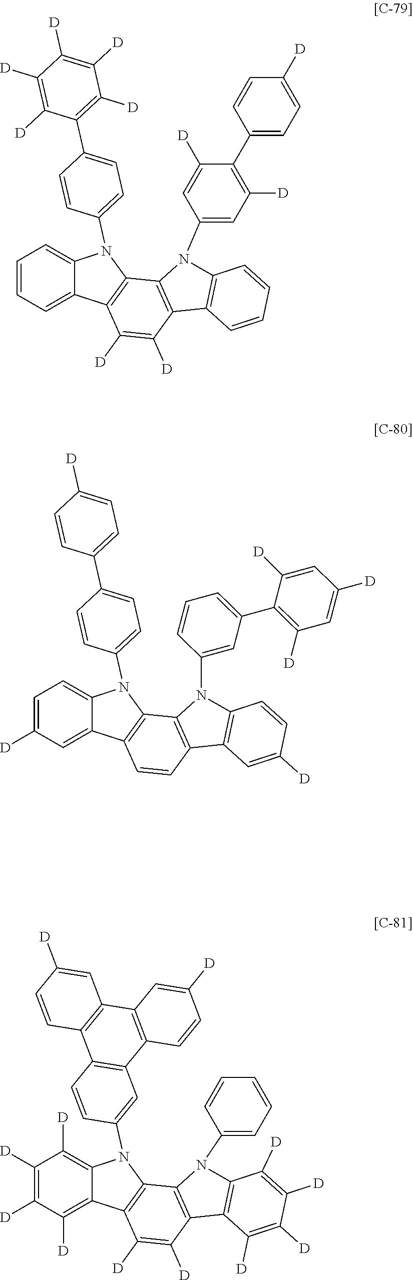 [SMILES: [2H]c1ccc(-c2c([2H])cc(-n3c4ccccc4c4c([2H])c([2H])c5c6ccccc6n(-c6ccc(-c7c([2H])c([2H])c([2H])c([2H])c7[2H])cc6)c5c43)cc2[2H])cc1 ; [2H]c1ccc(-c2ccc(-n3c4ccc([2H])cc4c4ccc5c6cc([2H])ccc6n(-c6cccc(-c7c([2H])cc([2H])cc7[2H])c6)c5c43)cc2)cc1 ; [2H]c1ccc2c3ccc(-n4c5c([2H])c([2H])c([2H])c([2H])c5c5c([2H])c([2H])c6c7c([2H])c([2H])c([2H])c([2H])c7n(-c7ccccc7)c6c54)cc3c3ccc([2H])cc3c2c1]

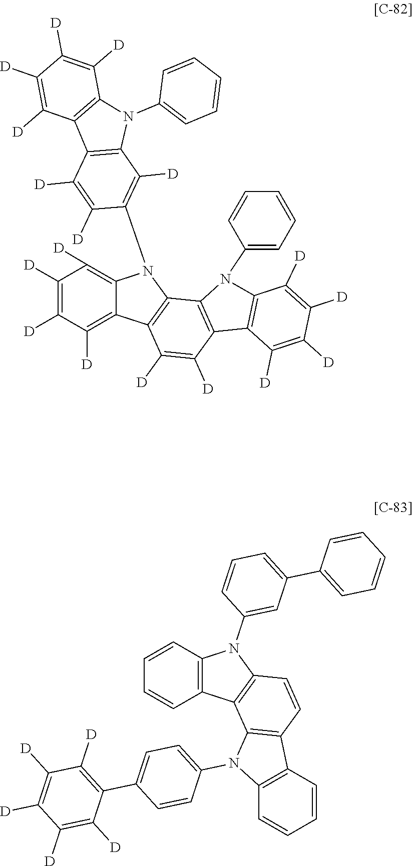 [SMILES: [2H]c1c([2H])c([2H])c2c(c1[2H])c1c([2H])c([2H])c(-n3c4c([2H])c([2H])c([2H])c([2H])c4c4c([2H])c([2H])c5c6c([2H])c([2H])c([2H])c([2H])c6n(-c6ccccc6)c5c43)c([2H])c1n2-c1ccccc1 ; [2H]c1c([2H])c([2H])c(-c2ccc(-n3c4ccccc4c4ccc5c(c6ccccc6n5-c5cccc(-c6ccccc6)c5)c43)cc2)c([2H])c1[2H]]

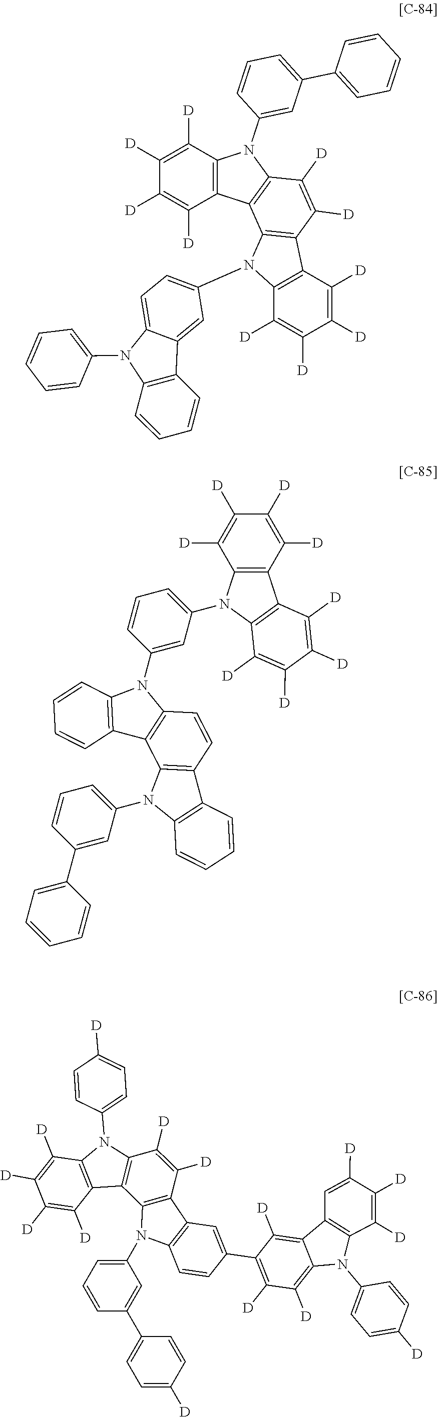 [SMILES: [2H]c1c([2H])c([2H])c2c(c1[2H])c1c(c([2H])c([2H])c3c4c([2H])c([2H])c([2H])c([2H])c4n(-c4ccc5c(c4)c4ccccc4n5-c4ccccc4)c31)n2-c1cccc(-c2ccccc2)c1 ; [2H]c1c([2H])c([2H])c2c(c1[2H])c1c([2H])c([2H])c([2H])c([2H])c1n2-c1cccc(-n2c3ccccc3c3c2ccc2c4ccccc4n(-c4cccc(-c5ccccc5)c4)c23)c1 ; [2H]c1ccc(-c2cccc(-n3c4ccc(-c5c([2H])c([2H])c6c(c5[2H])c5cc([2H])c([2H])c([2H])c5n6-c5ccc([2H])cc5)cc4c4c([2H])c([2H])c5c(c6c([2H])c([2H])c([2H])c([2H])c6n5-c5ccc([2H])cc5)c43)c2)cc1]

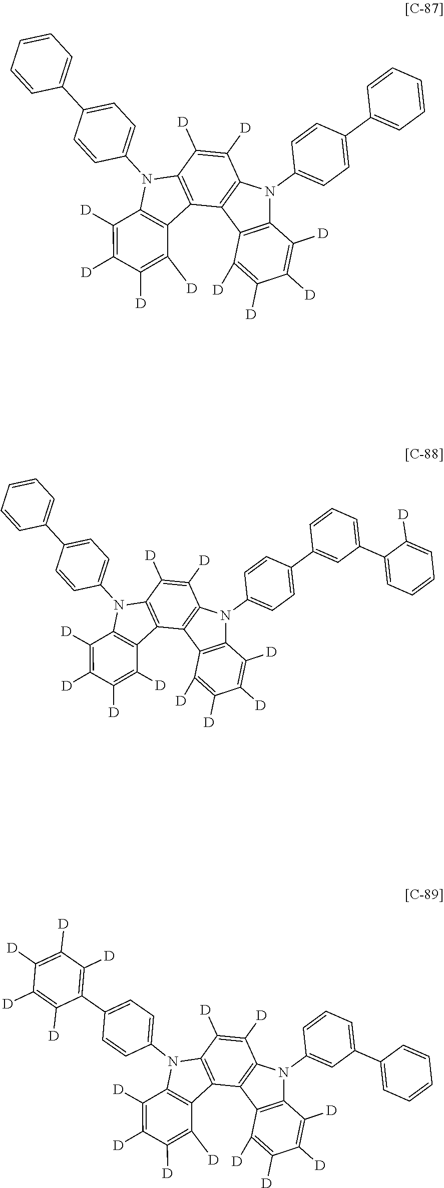 [SMILES: [2H]c1c([2H])c([2H])c2c(c1[2H])c1c3c4c([2H])c([2H])c([2H])c([2H])c4n(-c4ccc(-c5ccccc5)cc4)c3c([2H])c([2H])c1n2-c1ccc(-c2ccccc2)cc1 ; [2H]c1ccccc1-c1cccc(-c2ccc(-n3c4c([2H])c([2H])c([2H])c([2H])c4c4c5c6c([2H])c([2H])c([2H])c([2H])c6n(-c6ccc(-c7ccccc7)cc6)c5c([2H])c([2H])c43)cc2)c1 ; [2H]c1c([2H])c([2H])c(-c2ccc(-n3c4c([2H])c([2H])c([2H])c([2H])c4c4c5c6c([2H])c([2H])c([2H])c([2H])c6n(-c6cccc(-c7ccccc7)c6)c5c([2H])c([2H])c43)cc2)c([2H])c1[2H]]

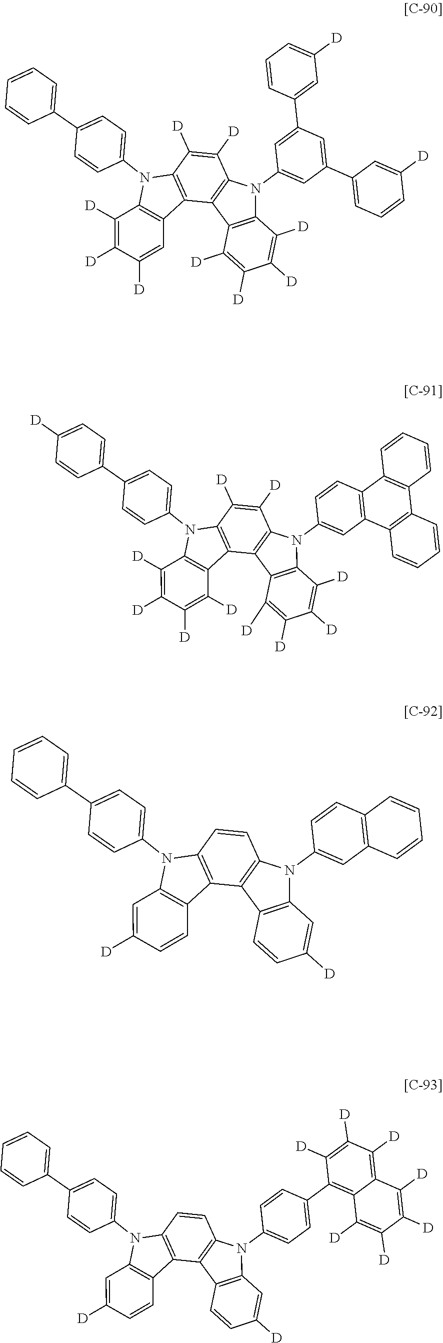

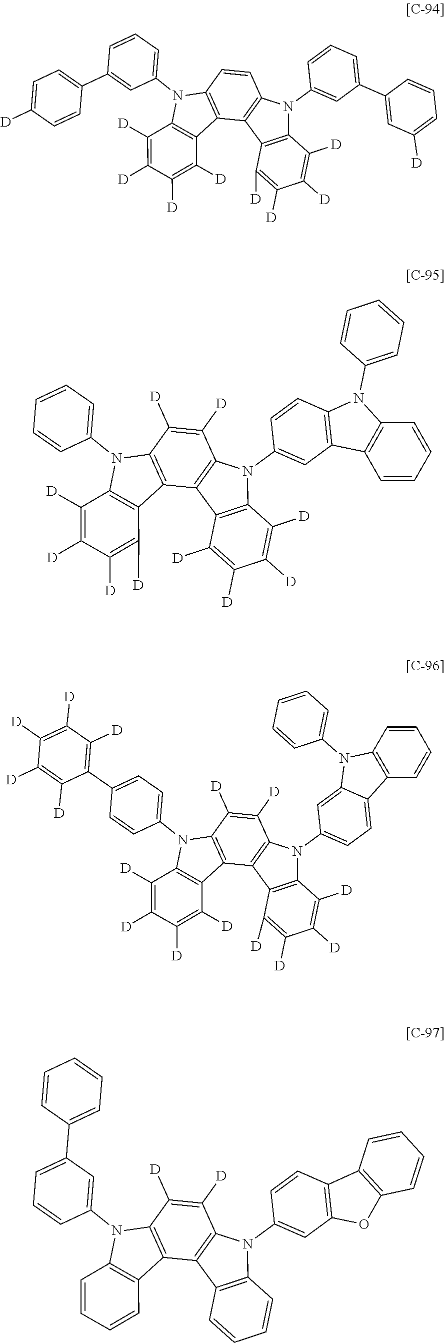

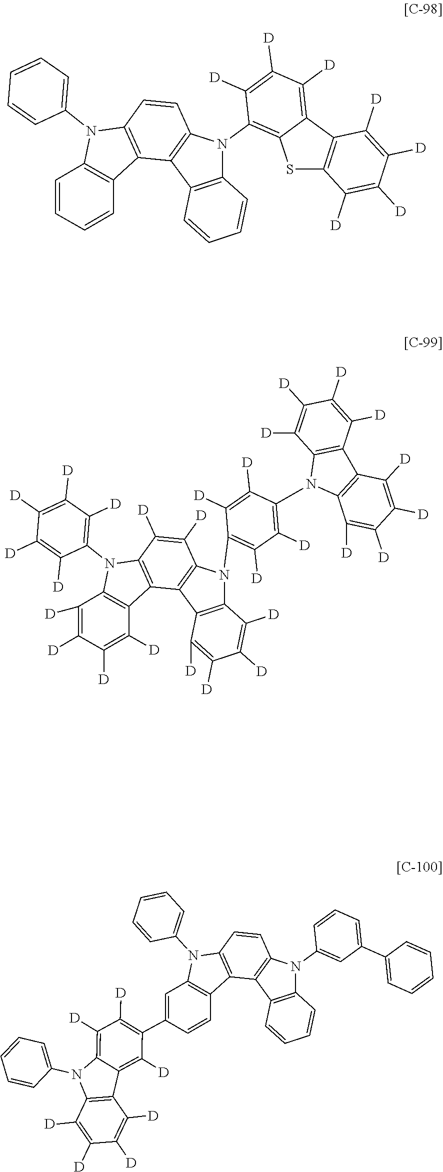 [SMILES: [2H]c1c([2H])c([2H])c2c(sc3c(-n4c5ccccc5c5c6c7ccccc7n(-c7ccccc7)c6ccc54)c([2H])c([2H])c([2H])c32)c1[2H] ; [2H]c1c([2H])c([2H])c(-n2c3c([2H])c([2H])c([2H])c([2H])c3c3c4c5c([2H])c([2H])c([2H])c([2H])c5n(-c5c([2H])c([2H])c(-n6c7c([2H])c([2H])c([2H])c([2H])c7c7c([2H])c([2H])c([2H])c([2H])c76)c([2H])c5[2H])c4c([2H])c([2H])c32)c([2H])c1[2H] ; [2H]c1c([2H])c([2H])c2c(c1[2H])c1c([2H])c(-c3ccc4c5c6c7ccccc7n(-c7cccc(-c8ccccc8)c7)c6ccc5n(-c5ccccc5)c4c3)c([2H])c([2H])c1n2-c1ccccc1]

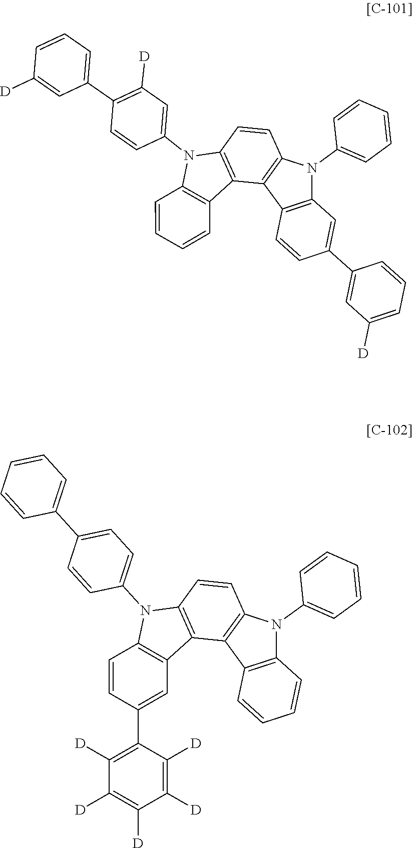 [SMILES: [2H]c1cccc(-c2ccc3c4c5c6ccccc6n(-c6ccc(-c7cccc([2H])c7)c([2H])c6)c5ccc4n(-c4ccccc4)c3c2)c1 ; [2H]c1c([2H])c([2H])c(-c2ccc3c(c2)c2c4c5ccccc5n(-c5ccccc5)c4ccc2n3-c2ccc(-c3ccccc3)cc2)c([2H])c1[2H]]

The first compound and the second compound may be included in a weight ratio of, e.g., about 1:99 to 99:1. Within the above range, bipolar properties may be implemented by matching an appropriate weight ratio using electron transport capability of the first compound and the hole transport capability of the second compound, to improve efficiency and life-span. Within this range, e.g., they may be included in a weight ratio of about 10:90 to 90:10, about 20:80 to 80:20, about 20:80 to about 70:30, about 20:80 to about 60:40, or about 30:70 to about 60:40. In an implementation, they may be included in a weight ratio of about 40:60, about 50:50, or about 60:40.

In addition to the aforementioned first compound and second compound, one or more compounds may be further included.

The aforementioned compound for an organic optoelectronic device or composition for an organic optoelectronic device may be a composition that further includes a dopant.

The dopant may be, e.g., a phosphorescent dopant, e.g., a red, green, or blue phosphorescent dopant, and may be, e.g., a red or green phosphorescent dopant.

The dopant is a material mixed with the compound or composition for an organic optoelectronic device in a small amount to cause light emission, and may be generally a material such as a metal complex that emits light by multiple excitation into a triplet or more. The dopant may be, e.g., an inorganic, organic, or organic/inorganic compound, and one or more types thereof may be used.

Examples of the dopant may include a phosphorescent dopant and examples of the phosphorescent dopant may be an organometal compound including Ir, Pt, Os, Ti, Zr, Hf, Eu, Tb, Tm, Fe, Co, Ni, Ru, Rh, Pd, or a combination thereof. The phosphorescent dopant may be, e.g., a compound represented by Chemical Formula Z.

L⁶MX [Chemical Formula Z]

In Chemical Formula Z, M may be, e.g., a metal, L⁶ and X may each independently be, e.g., a ligand to form a complex compound with M.

The M may be, e.g., Ir, Pt, Os, Ti, Zr, Hf, Eu, Tb, Tm, Fe, Co, Ni, Ru, Rh, Pd, or a combination thereof and L⁶ and X may be, e.g., a bidentate ligand.

Examples of ligands represented by L⁶ and X may be ligands of Group A.

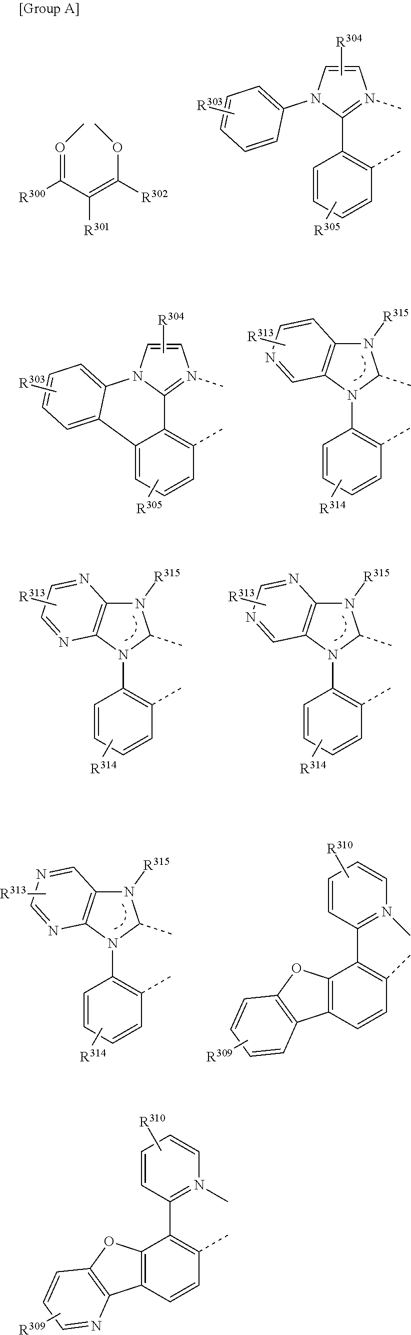

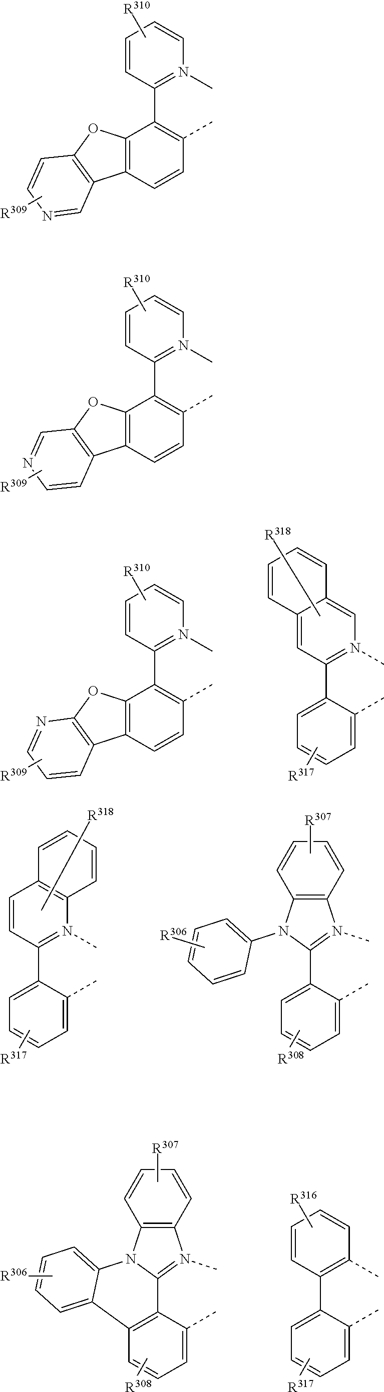

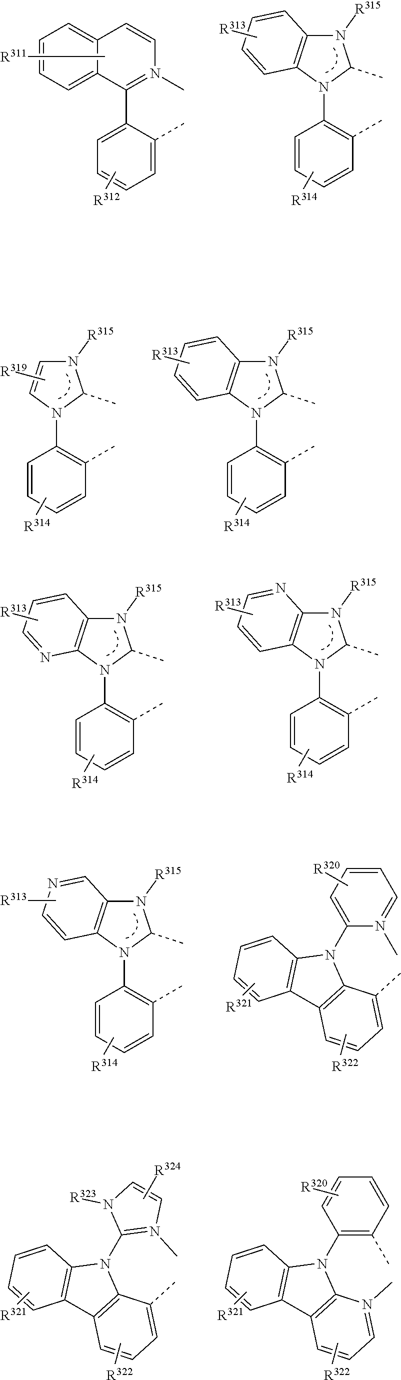

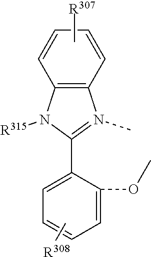

In Group A, R³⁰ to R³⁰² may each independently be, e.g., hydrogen, deuterium, a C1 to C30 alkyl group that is substituted or unsubstituted with a halogen, a C6 to C30 aryl group that is substituted or unsubstituted with a C1 to C30 alkyl, or a halogen.

R³³ to R³²⁴ may each independently be, e.g., hydrogen, deuterium, halogen, a substituted or unsubstituted C1 to C30 alkyl group, a substituted or unsubstituted C1 to C30 alkoxy Group, a substituted or unsubstituted C3 to C30 cycloalkyl group, a substituted or unsubstituted C2 to C30 alkenyl group, a substituted or unsubstituted C6 to C30 aryl group, a substituted or unsubstituted C1 to C30 heteroaryl group, a substituted or unsubstituted C1 to C30 amino group, a substituted or unsubstituted C6 to C30 arylamino group, SF₅, a trialkylsilyl group having a substituted or unsubstituted C1 to C30 alkyl group, a dialkylarylsilyl group having a substituted or unsubstituted C1 to C30 alkyl group and a C6 to C30 aryl group, or a triarylsilyl group having a substituted or unsubstituted C6 to C30 aryl group.

In an implementation, the dopant may be represented by Chemical Formula V.

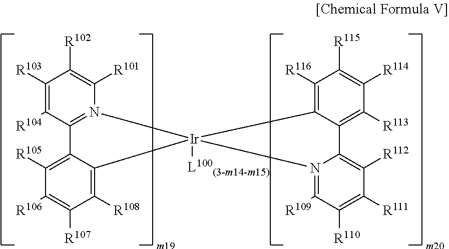

In Chemical Formula V, R¹⁰¹ to R¹¹⁶ may each independently be, e.g., hydrogen, deuterium, a substituted or unsubstituted C1 to C10 alkyl group, a substituted or unsubstituted C6 to C20 aryl group, or —SiR¹³²R¹³³R¹³⁴.

R¹³² to R¹³⁴ may each independently be, e.g., a substituted or unsubstituted C1 to C6 alkyl group.

In an implementation, at least one of R¹⁰¹ to R¹¹⁶ may be a functional group represented by Chemical Formula V-1.

L¹⁰⁰ may be, e.g., a bidentate ligand of a monovalent anion, and may be a ligand that coordinates to iridium through a lone pair of carbons or heteroatoms.

m19 and m20 may each independently be, e.g., an integer of 0 to 3, and m19+m20 may be an integer of 1 to 3.

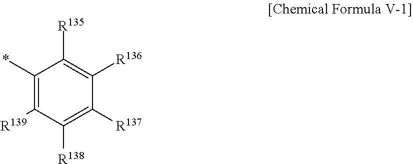

In Chemical Formula V-1, R¹³⁵ to R¹³⁹ may each independently be, e.g., hydrogen, deuterium, a substituted or unsubstituted C1 to C10 alkyl group, a substituted or unsubstituted C6 to C20 aryl group, or —SiR¹³²R¹³³R¹³⁴.

* means a portion linked to a carbon atom.

In an implementation, a dopant represented by Chemical Formula Z-1 may be included.

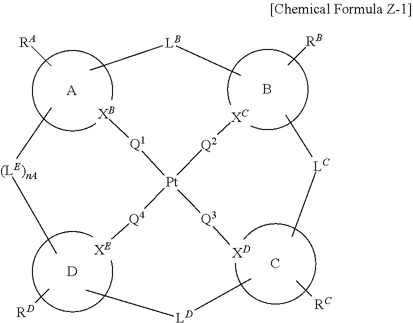

In Chemical Formula Z-1, rings A, B, C, and D may each independently be, e.g., a 5-membered or 6-membered carbocyclic or heterocyclic ring.

R^{A}, R^{B}, R^{C}, and R^{D} may each independently represent mono-, di-, tri-, or tetra-substitution, or unsubstitution.

L^{B}, L^{C}, and L^{D} may each independently be, e.g., a direct bond, BR, NR, PR, 0, S, Se, C═O, S═O, SO₂, CRR′, SiRR′, GeRR′, or a combination thereof.

In an implementation, when nA is 1, L^{E} may be selected from a direct bond, BR, NR, PR, O, S, Se, C═O, S═O, SO₂, CRR′, SiRR′, GeRR′, or a combination thereof, when nA is 0, L^{E} does not exist; and

R^{A}, R^{B}, R^{C}, R^{D}, R, and R′ may each independently be, e.g., hydrogen, deuterium, a halogen, an alkyl group, a cycloalkyl group, a heteroalkyl group, an arylalkyl group, an alkoxy group, an aryloxy group, an amino group, a silyl group, an alkenyl group, a cycloalkenyl group, a heteroalkenyl group, an alkynyl group, an aryl group, a heteroaryl group, an acyl group, a carbonyl group, a carboxylic acid group, an ester group, a nitrile group, an isonitrile group, a sulfanyl group, a sulfinyl group, a sulfonyl group, a phosphino group, or a combination thereof, any adjacent R^{A}, R^{B}, R^{C}, R^{D}, R, and R′ may be optionally linked to each other to provide a ring; X^{B}, X^{C}, X^{D}, and X^{E} may each independently be, e.g., carbon or nitrogen; and Q¹, Q², Q³, and Q⁴ may each independently be, e.g., oxygen or a direct bond.

The dopant according to an embodiment may be a platinum complex, and may be, e.g., represented by Chemical Formula VI.

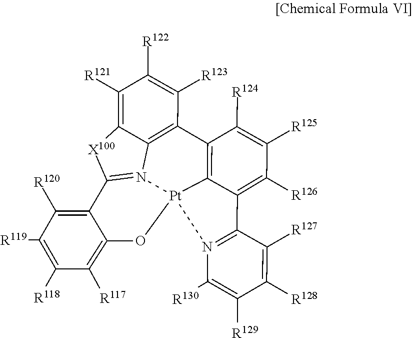

In Chemical Formula VI, X¹⁰⁰ may be, e.g., O, S, or NR¹³¹

R¹¹⁷ to R¹³¹ may each independently be, e.g., hydrogen, deuterium, a substituted or unsubstituted C1 to C10 alkyl group, a substituted or unsubstituted C6 to C20 aryl group, or —SiR¹³²R¹³³R¹³⁴.

R¹³² to R¹³⁴ may each independently be, e.g., a substituted or unsubstituted C1 to C6 alkyl group.

In an implementation, at least one of R¹¹⁷ to R¹³¹ may be, e.g., —SiR¹³²R¹³³R¹³⁴ or a tert-butyl group.

R¹³² to R¹³⁴ may each independently be, e.g., a substituted or unsubstituted C1 to C6 alkyl group.

Hereinafter, an organic optoelectronic device including the aforementioned compound for an organic optoelectronic device or composition for an organic optoelectronic device is described.

The organic optoelectronic device may be a suitable device to convert electrical energy into photoenergy and vice versa, and may be, e.g., an organic photoelectric device, an organic light emitting diode, an organic solar cell, or an organic photoconductor drum.

Herein, an organic light emitting diode as one example of an organic optoelectronic device is described referring to the drawing.

The FIGURE a cross-sectional view illustrating an organic light emitting diode according to an embodiment.

Referring to the FIGURE, an organic light emitting diode 100 according to an embodiment may include an anode 120 and a cathode 110 facing each other and an organic layer 105 between the anode 120 and cathode 110.

The anode 120 may be made of a conductor having a large work function to help hole injection, and may be e.g., a metal, a metal oxide, or a conductive polymer. The anode 120 may be, e.g., a metal such as nickel, platinum, vanadium, chromium, copper, zinc, gold, or the like or an alloy thereof; a metal oxide such as zinc oxide, indium oxide, indium tin oxide (ITO), indium zinc oxide (IZO), or the like; a combination of a metal and an oxide such as ZnO and Al or SnO₂ and Sb; or a conductive polymer such as poly(3-methylthiophene), poly(3,4-(ethylene-1,2-dioxy)thiophene) (PEDOT), polypyrrole, or polyaniline.

The cathode 110 may be made of a conductor having a small work function to help electron injection, and may be e.g., a metal, a metal oxide, or a conductive polymer. The cathode 110 may be, e.g., a metal such as magnesium, calcium, sodium, potassium, titanium, indium, yttrium, lithium, gadolinium, aluminum silver, tin, lead, cesium, barium, or the like, or an alloy thereof; or a multi-layer structure material such as LiF/Al, LiO₂/Al, LiF/Ca, or BaF₂/Ca.

The organic layer 105 may include the aforementioned compound for an organic optoelectronic device or composition for an organic optoelectronic device.

The organic layer 105 may include the light emitting layer 130 and the light emitting layer 130 may include the aforementioned compound for an organic optoelectronic device or composition for an organic optoelectronic device.

The composition for an organic optoelectronic device further including a dopant may be, e.g., a green light emitting composition.

The light emitting layer 130 may include, e.g., the aforementioned compound for an organic optoelectronic device or composition for an organic optoelectronic device as a phosphorescent host.

The organic layer may further include a charge transport region in addition to the light emitting layer.

The charge transport region may be, e.g., a hole transport region 140.

The hole transport region 140 may further increase hole injection and/or hole mobility and block electrons between the anode 120 and the light emitting layer 130.

In an implementation, the hole transport region 140 may include a hole transport layer between the anode 120 and the light emitting layer 130, and a hole transport auxiliary layer between the light emitting layer 130 and the hole transport layer, and a compound of Group B may be included in at least one of the hole transport layer and the hole transport auxiliary layer.

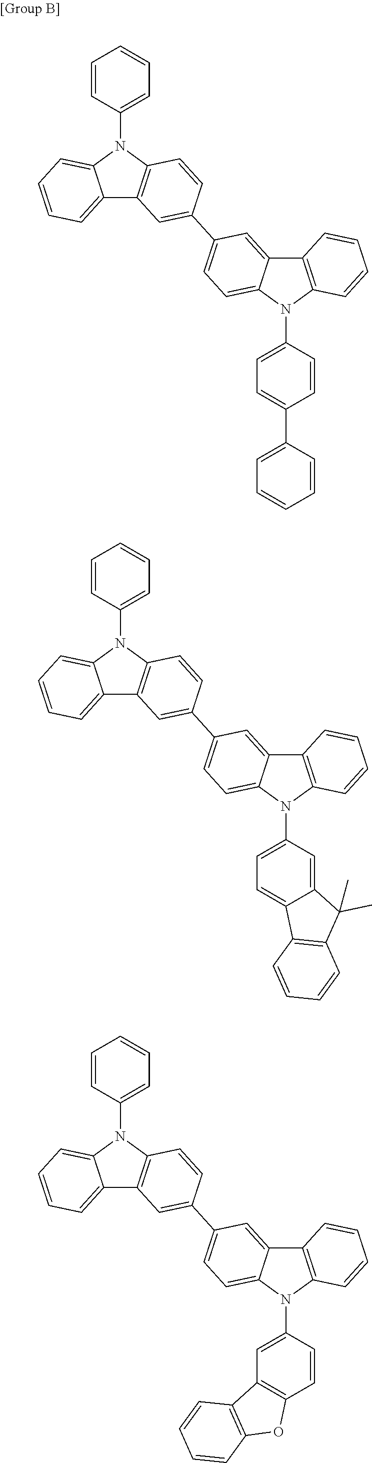 [SMILES: c1ccc(-c2ccc(-n3c4ccccc4c4cc(-c5ccc6c(c5)c5ccccc5n6-c5ccccc5)ccc43)cc2)cc1 ; CC1(C)c2ccccc2-c2ccc(-n3c4ccccc4c4cc(-c5ccc6c(c5)c5ccccc5n6-c5ccccc5)ccc43)cc21 ; c1ccc(-n2c3ccccc3c3cc(-c4ccc5c(c4)c4ccccc4n5-c4ccc5oc6ccccc6c5c4)ccc32)cc1]

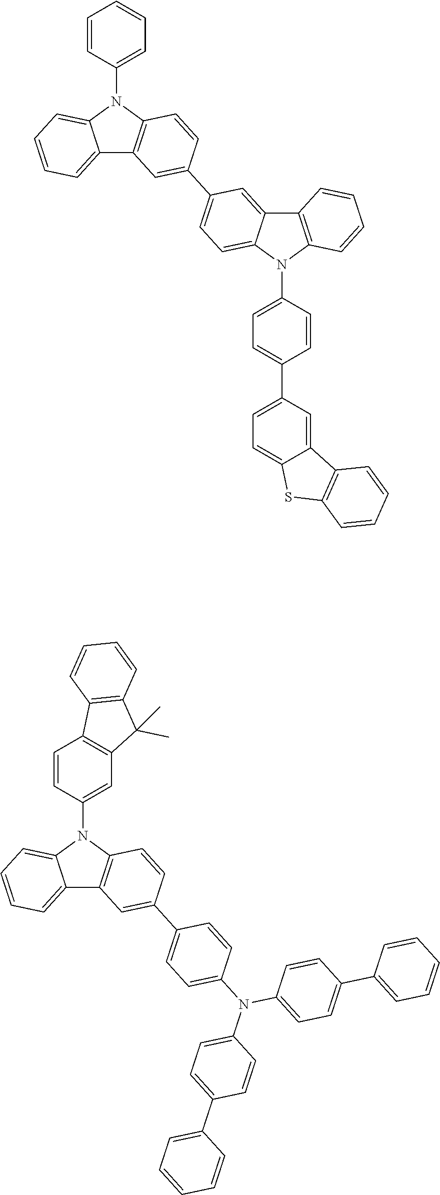 [SMILES: c1ccc(-n2c3ccccc3c3cc(-c4ccc5c(c4)c4ccccc4n5-c4ccc(-c5ccc6sc7ccccc7c6c5)cc4)ccc32)cc1 ; CC1(C)c2ccccc2-c2ccc(-n3c4ccccc4c4cc(-c5ccc(N(c6ccc(-c7ccccc7)cc6)c6ccc(-c7ccccc7)cc6)cc5)ccc43)cc21]

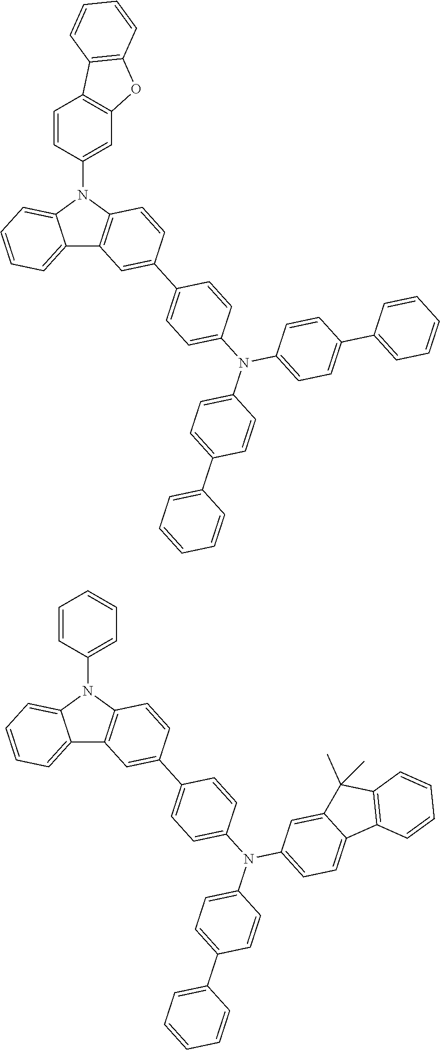 [SMILES: c1ccc(-c2ccc(N(c3ccc(-c4ccccc4)cc3)c3ccc(-c4ccc5c(c4)c4ccccc4n5-c4ccc5c(c4)oc4ccccc45)cc3)cc2)cc1 ; CC1(C)c2ccccc2-c2ccc(N(c3ccc(-c4ccccc4)cc3)c3ccc(-c4ccc5c(c4)c4ccccc4n5-c4ccccc4)cc3)cc21]

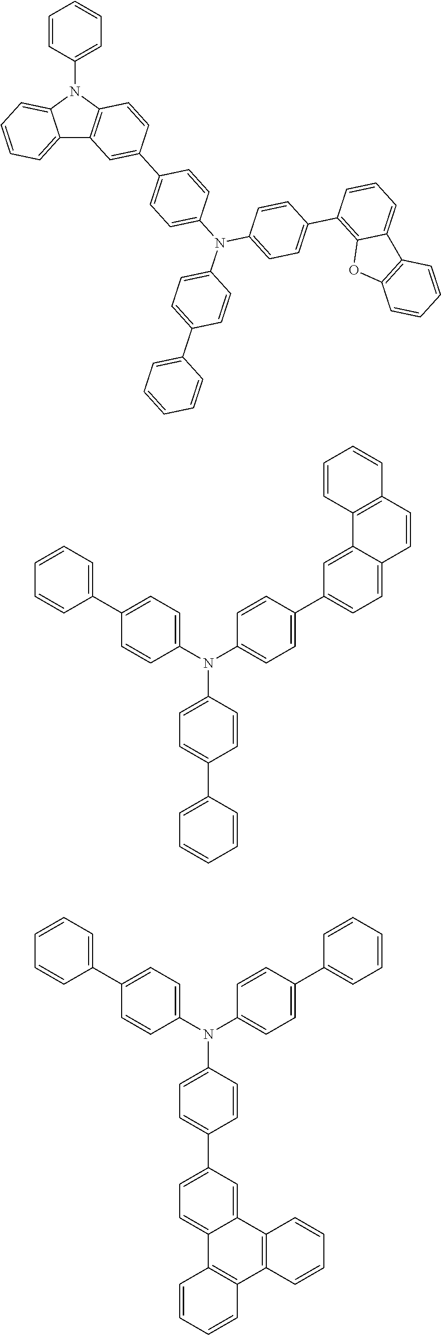 [SMILES: c1ccc(-c2ccc(N(c3ccc(-c4ccc5c(c4)c4ccccc4n5-c4ccccc4)cc3)c3ccc(-c4cccc5c4oc4ccccc45)cc3)cc2)cc1 ; c1ccc(-c2ccc(N(c3ccc(-c4ccccc4)cc3)c3ccc(-c4ccc5ccc6ccccc6c5c4)cc3)cc2)cc1 ; c1ccc(-c2ccc(N(c3ccc(-c4ccccc4)cc3)c3ccc(-c4ccc5c6ccccc6c6ccccc6c5c4)cc3)cc2)cc1]

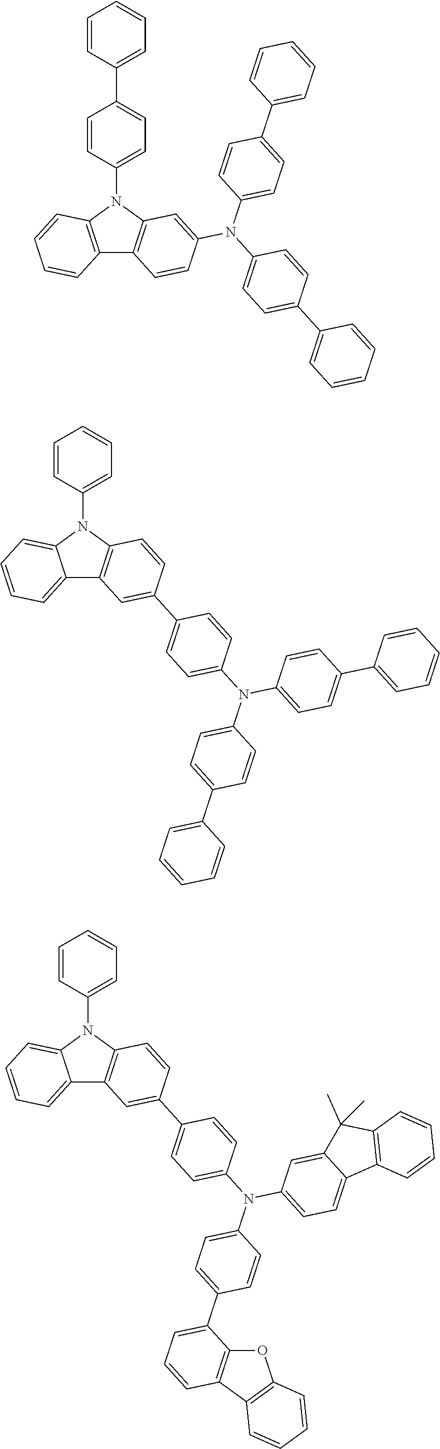 [SMILES: c1ccc(-c2ccc(N(c3ccc(-c4ccccc4)cc3)c3ccc4c5ccccc5n(-c5ccc(-c6ccccc6)cc5)c4c3)cc2)cc1 ; c1ccc(-c2ccc(N(c3ccc(-c4ccccc4)cc3)c3ccc(-c4ccc5c(c4)c4ccccc4n5-c4ccccc4)cc3)cc2)cc1 ; CC1(C)c2ccccc2-c2ccc(N(c3ccc(-c4ccc5c(c4)c4ccccc4n5-c4ccccc4)cc3)c3ccc(-c4cccc5c4oc4ccccc45)cc3)cc21]

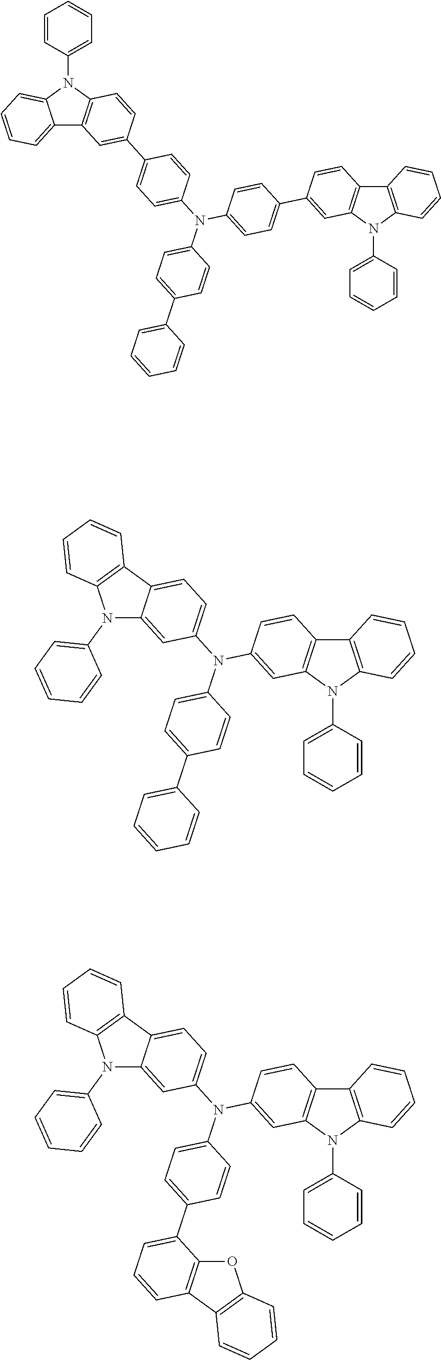 [SMILES: c1ccc(-c2ccc(N(c3ccc(-c4ccc5c(c4)c4ccccc4n5-c4ccccc4)cc3)c3ccc(-c4ccc5c6ccccc6n(-c6ccccc6)c5c4)cc3)cc2)cc1 ; c1ccc(-c2ccc(N(c3ccc4c5ccccc5n(-c5ccccc5)c4c3)c3ccc4c5ccccc5n(-c5ccccc5)c4c3)cc2)cc1 ; c1ccc(-n2c3ccccc3c3ccc(N(c4ccc(-c5cccc6c5oc5ccccc56)cc4)c4ccc5c6ccccc6n(-c6ccccc6)c5c4)cc32)cc1]

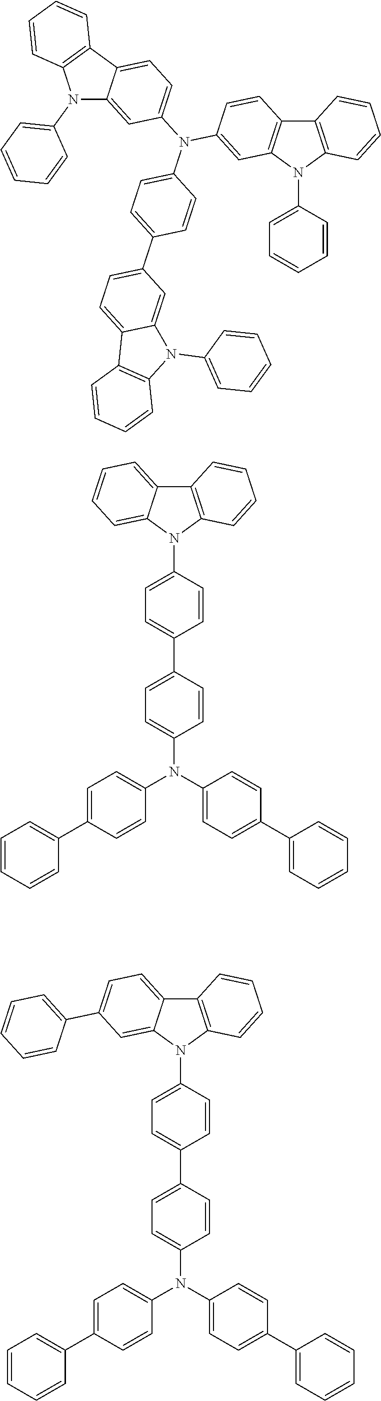

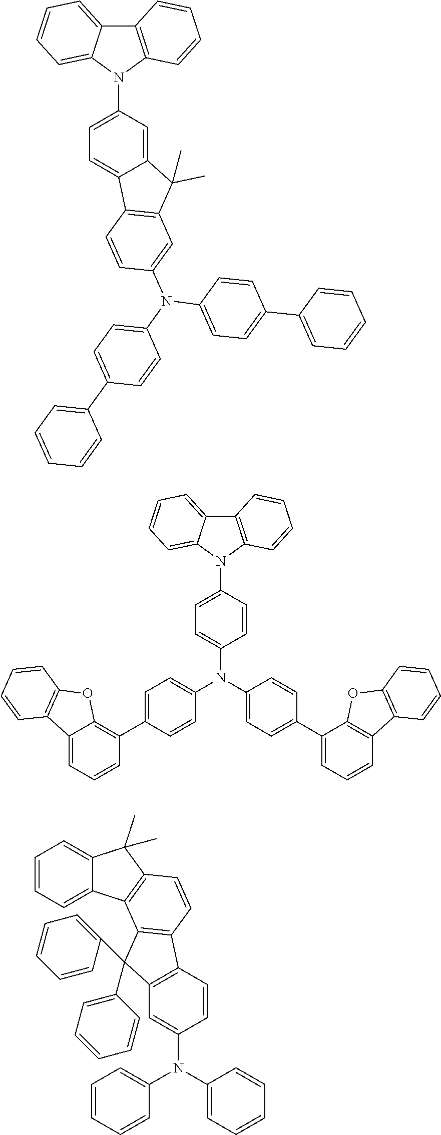 [SMILES: CC1(C)c2cc(N(c3ccc(-c4ccccc4)cc3)c3ccc(-c4ccccc4)cc3)ccc2-c2ccc(-n3c4ccccc4c4ccccc43)cc21 ; c1ccc2c(c1)oc1c(-c3ccc(N(c4ccc(-c5cccc6c5oc5ccccc56)cc4)c4ccc(-n5c6ccccc6c6ccccc65)cc4)cc3)cccc12 ; CC1(C)c2ccccc2-c2c1ccc1c2C(c2ccccc2)(c2ccccc2)c2cc(N(c3ccccc3)c3ccccc3)ccc2-1]

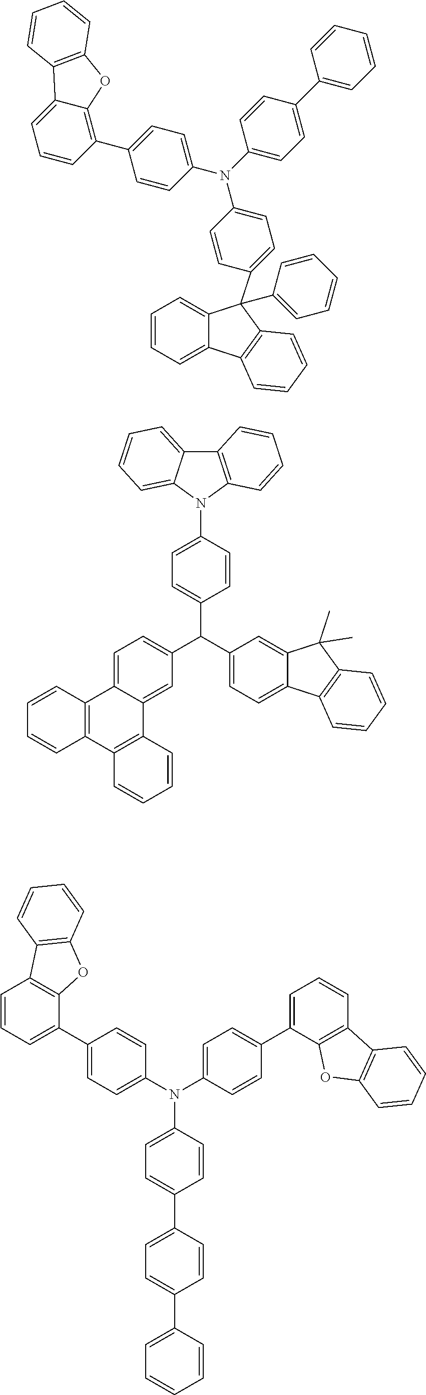

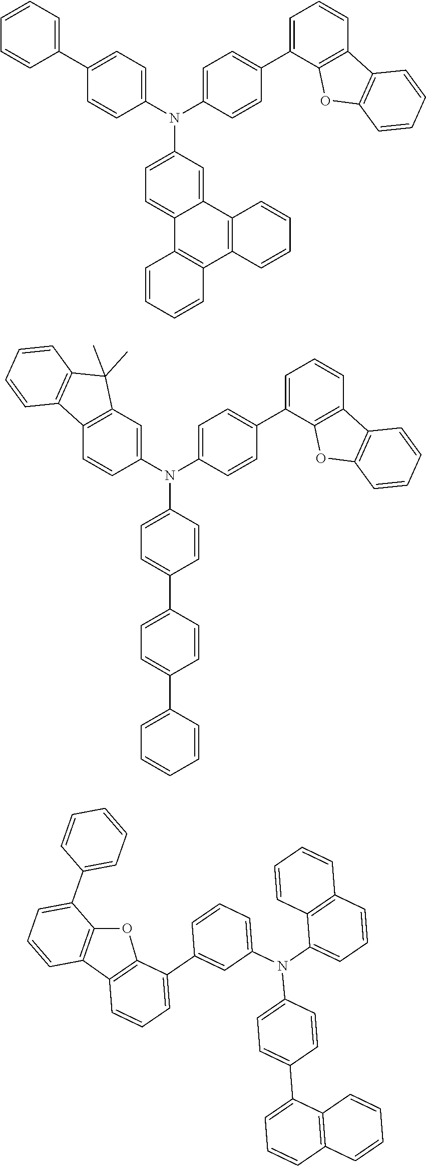 [SMILES: c1ccc(-c2ccc(N(c3ccc(-c4cccc5c4oc4ccccc45)cc3)c3ccc4c5ccccc5c5ccccc5c4c3)cc2)cc1 ; CC1(C)c2ccccc2-c2ccc(N(c3ccc(-c4ccc(-c5ccccc5)cc4)cc3)c3ccc(-c4cccc5c4oc4ccccc45)cc3)cc21 ; c1ccc(-c2cccc3c2oc2c(-c4cccc(N(c5ccc(-c6cccc7ccccc67)cc5)c5cccc6ccccc56)c4)cccc23)cc1]

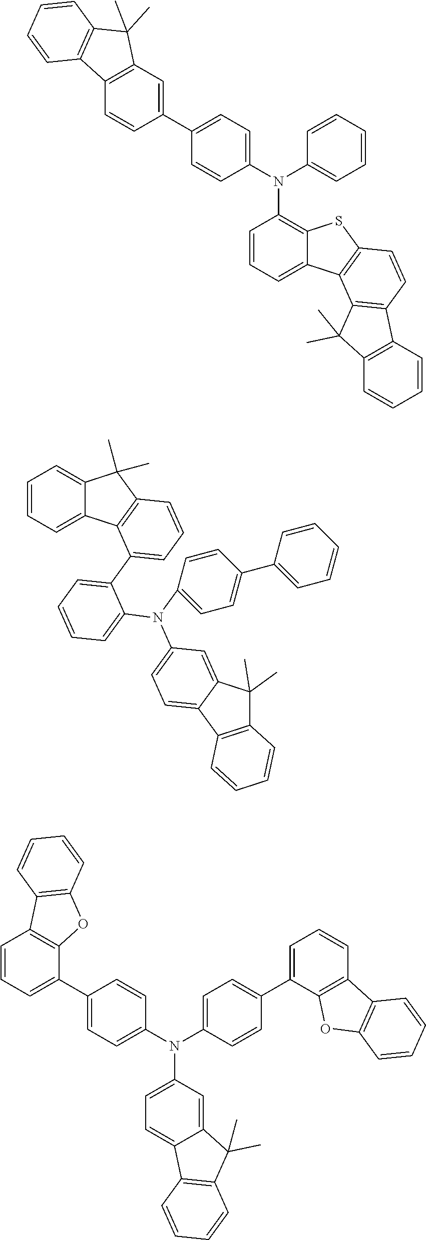 [SMILES: CC1(C)c2ccccc2-c2ccc(-c3ccc(N(c4ccccc4)c4cccc5c4sc4ccc6c(c45)C(C)(C)c4ccccc4-6)cc3)cc21 ; CC1(C)c2ccccc2-c2ccc(N(c3ccc(-c4ccccc4)cc3)c3ccccc3-c3cccc4c3-c3ccccc3C4(C)C)cc21 ; CC1(C)c2ccccc2-c2ccc(N(c3ccc(-c4cccc5c4oc4ccccc45)cc3)c3ccc(-c4cccc5c4oc4ccccc45)cc3)cc21]

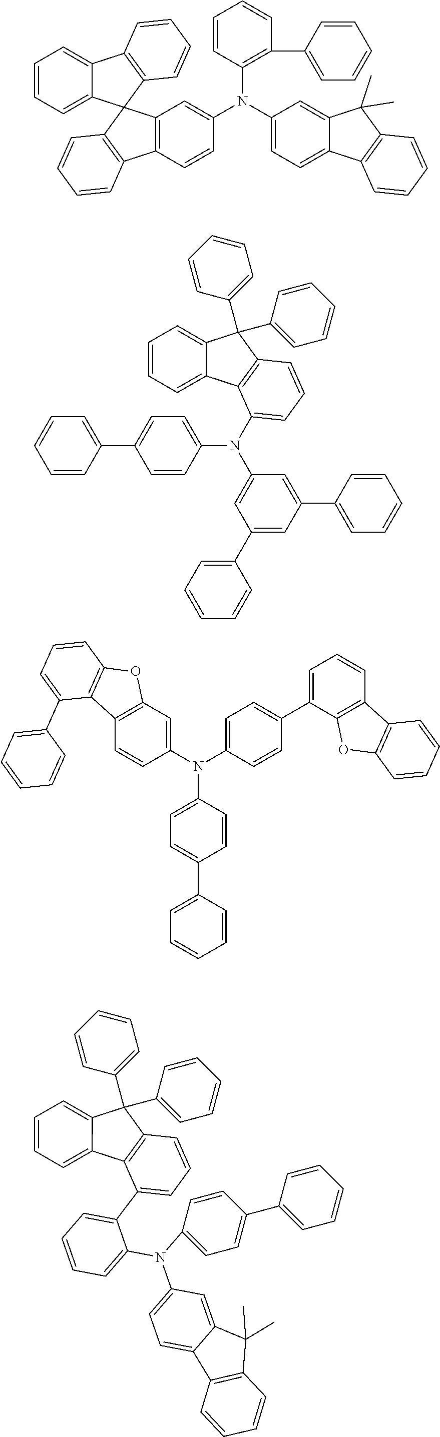

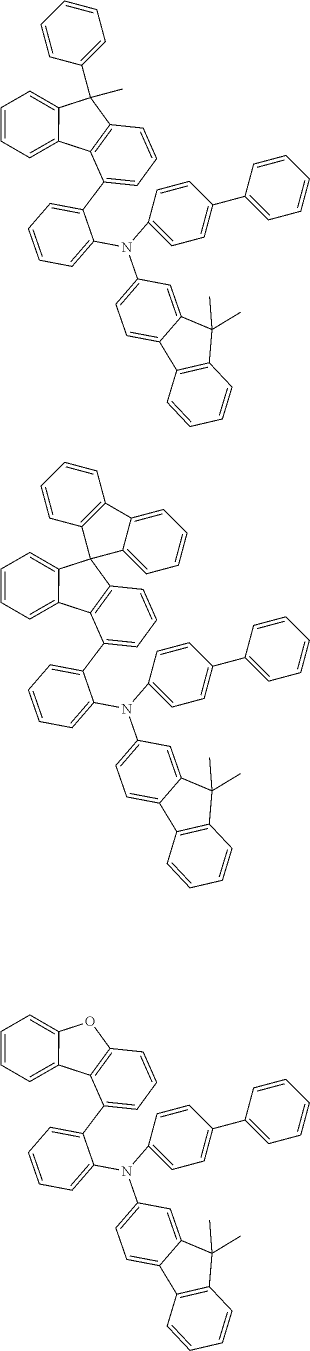 [SMILES: CC1(C)c2ccccc2-c2ccc(N(c3ccc(-c4ccccc4)cc3)c3ccccc3-c3cccc4c3-c3ccccc3C4(C)c3ccccc3)cc21 ; CC1(C)c2ccccc2-c2ccc(N(c3ccc(-c4ccccc4)cc3)c3ccccc3-c3cccc4c3-c3ccccc3C43c4ccccc4-c4ccccc43)cc21 ; CC1(C)c2ccccc2-c2ccc(N(c3ccc(-c4ccccc4)cc3)c3ccccc3-c3cccc4oc5ccccc5c34)cc21]

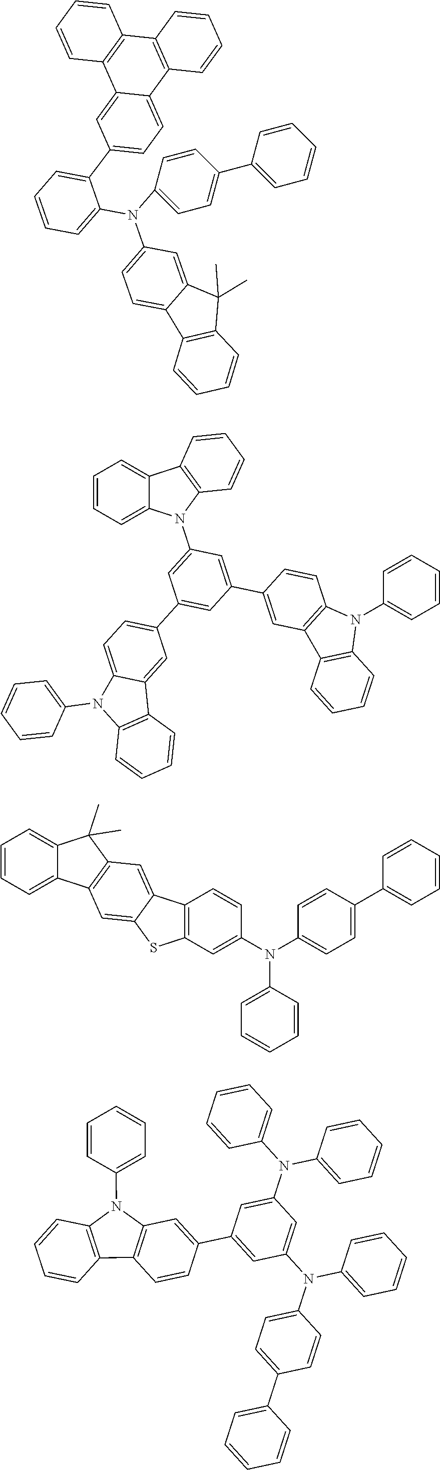

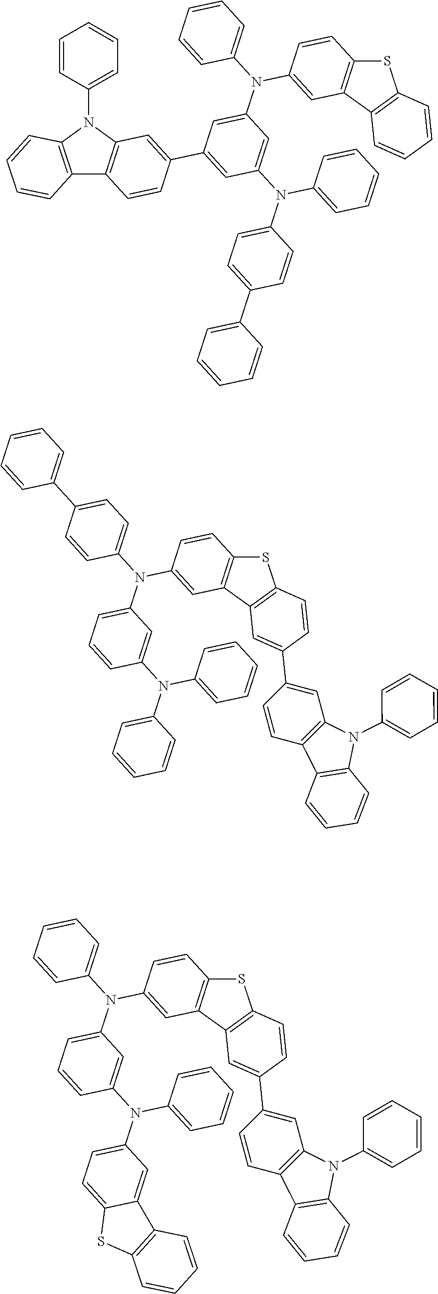 [SMILES: c1ccc(-c2ccc(N(c3ccccc3)c3cc(-c4ccc5c6ccccc6n(-c6ccccc6)c5c4)cc(N(c4ccccc4)c4ccc5sc6ccccc6c5c4)c3)cc2)cc1 ; c1ccc(-c2ccc(N(c3cccc(N(c4ccccc4)c4ccccc4)c3)c3ccc4sc5ccc(-c6ccc7c8ccccc8n(-c8ccccc8)c7c6)cc5c4c3)cc2)cc1 ; c1ccc(N(c2cccc(N(c3ccccc3)c3ccc4sc5ccc(-c6ccc7c8ccccc8n(-c8ccccc8)c7c6)cc5c4c3)c2)c2ccc3sc4ccccc4c3c2)cc1]

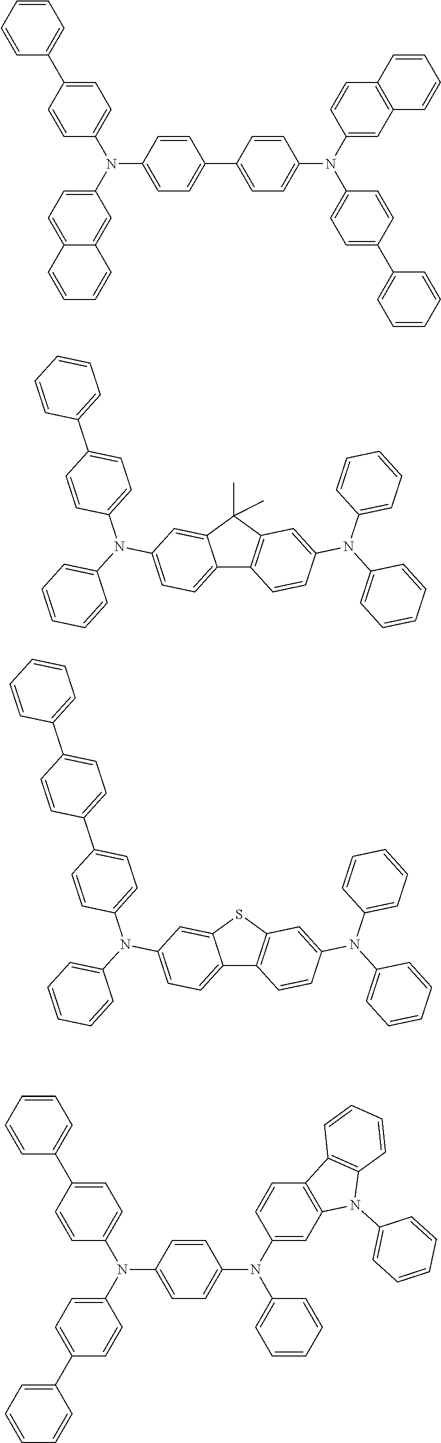

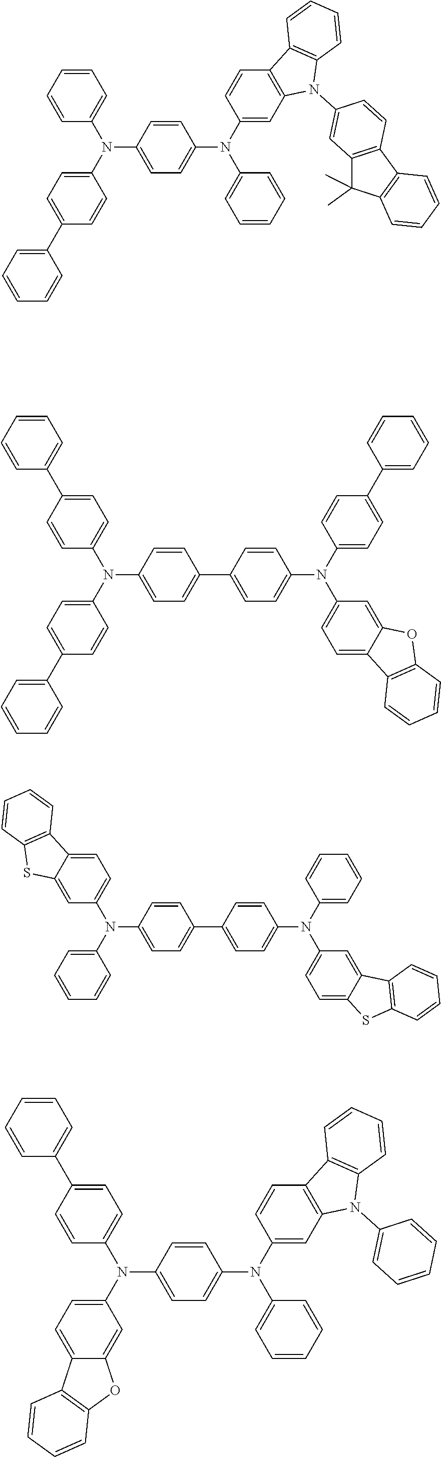 [SMILES: CC1(C)c2ccccc2-c2ccc(-n3c4ccccc4c4ccc(N(c5ccccc5)c5ccc(N(c6ccccc6)c6ccc(-c7ccccc7)cc6)cc5)cc43)cc21 ; c1ccc(-c2ccc(N(c3ccc(-c4ccccc4)cc3)c3ccc(-c4ccc(N(c5ccc(-c6ccccc6)cc5)c5ccc6c(c5)oc5ccccc56)cc4)cc3)cc2)cc1 ; c1ccc(N(c2ccc(-c3ccc(N(c4ccccc4)c4ccc5sc6ccccc6c5c4)cc3)cc2)c2ccc3c(c2)sc2ccccc23)cc1 ; c1ccc(-c2ccc(N(c3ccc(N(c4ccccc4)c4ccc5c6ccccc6n(-c6ccccc6)c5c4)cc3)c3ccc4c(c3)oc3ccccc34)cc2)cc1]

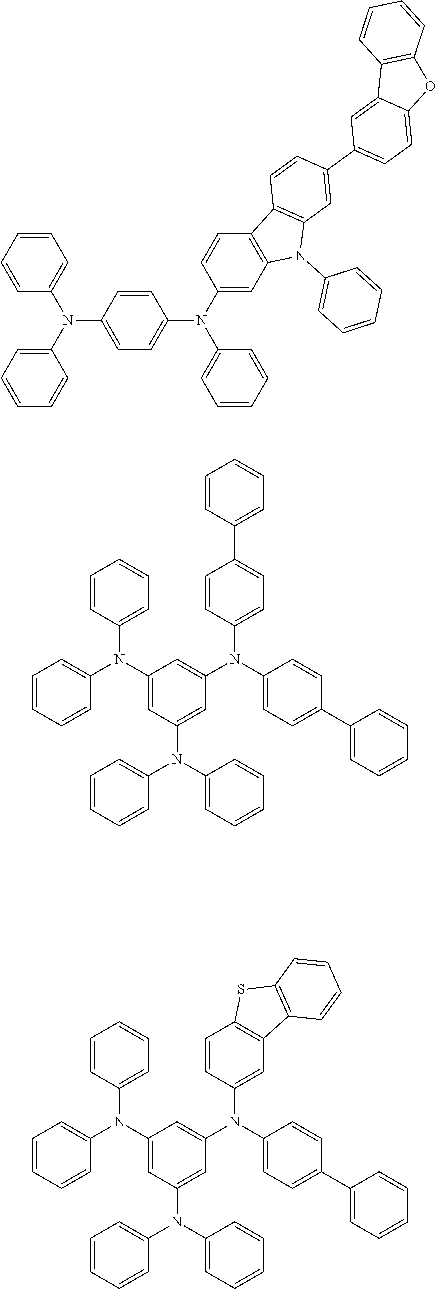 [SMILES: c1ccc(N(c2ccccc2)c2ccc(N(c3ccccc3)c3ccc4c5ccc(-c6ccc7oc8ccccc8c7c6)cc5n(-c5ccccc5)c4c3)cc2)cc1 ; c1ccc(-c2ccc(N(c3ccc(-c4ccccc4)cc3)c3cc(N(c4ccccc4)c4ccccc4)cc(N(c4ccccc4)c4ccccc4)c3)cc2)cc1 ; c1ccc(-c2ccc(N(c3cc(N(c4ccccc4)c4ccccc4)cc(N(c4ccccc4)c4ccccc4)c3)c3ccc4sc5ccccc5c4c3)cc2)cc1]

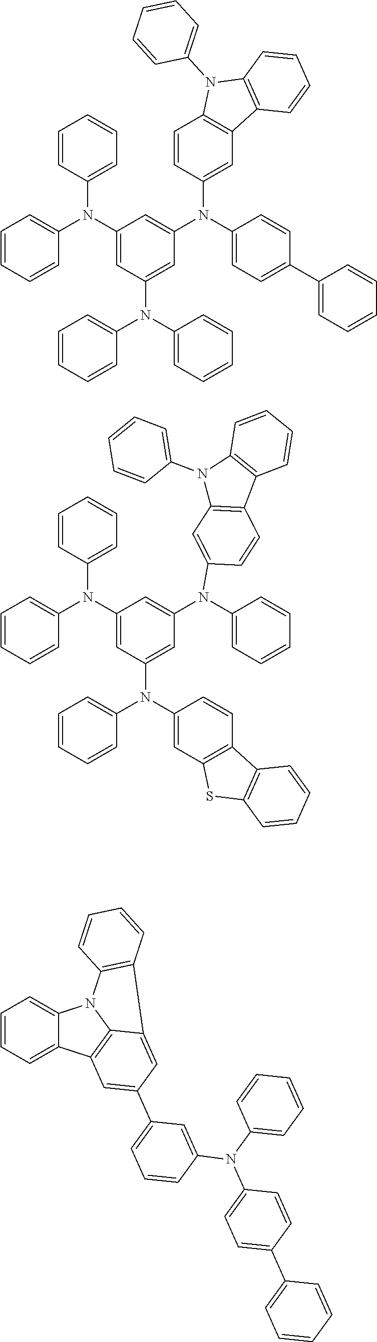 [SMILES: c1ccc(-c2ccc(N(c3cc(N(c4ccccc4)c4ccccc4)cc(N(c4ccccc4)c4ccccc4)c3)c3ccc4c(c3)c3ccccc3n4-c3ccccc3)cc2)cc1 ; c1ccc(N(c2ccccc2)c2cc(N(c3ccccc3)c3ccc4c(c3)sc3ccccc34)cc(N(c3ccccc3)c3ccc4c5ccccc5n(-c5ccccc5)c4c3)c2)cc1 ; c1ccc(-c2ccc(N(c3ccccc3)c3cccc(-c4cc5c6ccccc6n6c7ccccc7c(c4)c56)c3)cc2)cc1]

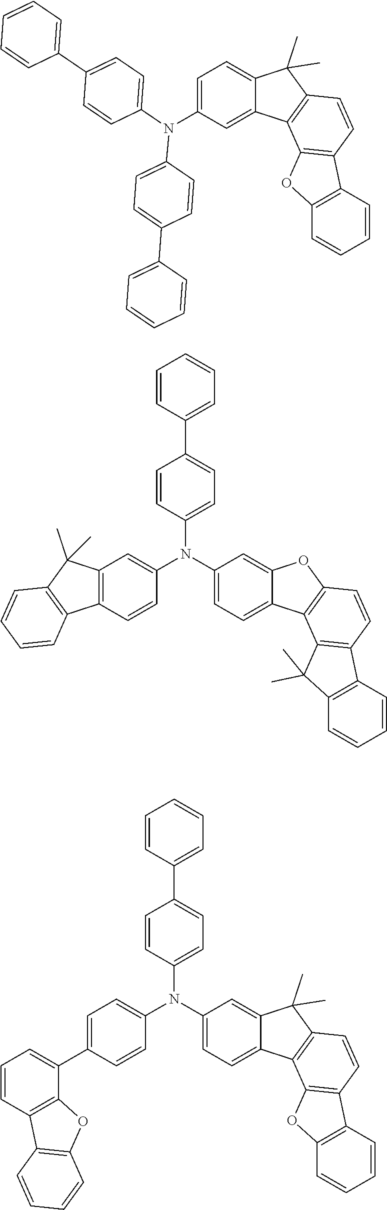

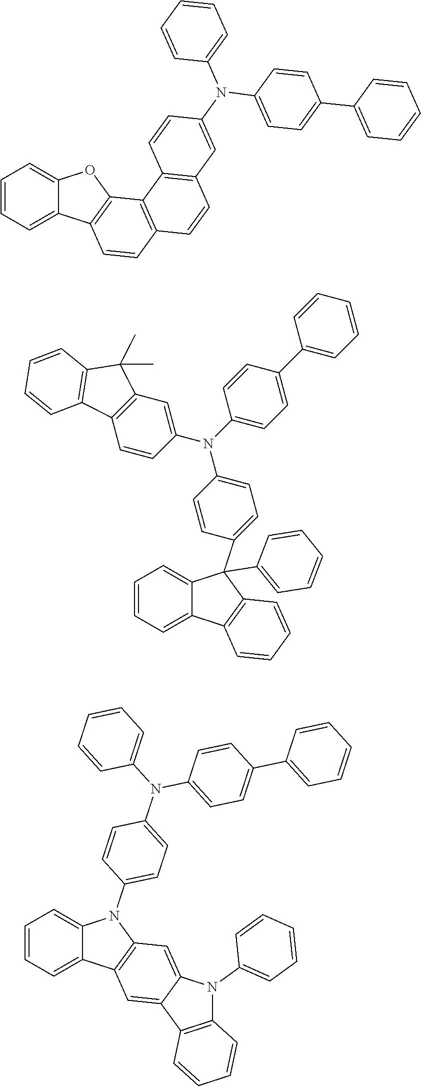 [SMILES: c1ccc(-c2ccc(N(c3ccccc3)c3ccc4c(ccc5ccc6c7ccccc7oc6c54)c3)cc2)cc1 ; CC1(C)c2ccccc2-c2ccc(N(c3ccc(-c4ccccc4)cc3)c3ccc(C4(c5ccccc5)c5ccccc5-c5ccccc54)cc3)cc21 ; c1ccc(-c2ccc(N(c3ccccc3)c3ccc(-n4c5ccccc5c5cc6c7ccccc7n(-c7ccccc7)c6cc54)cc3)cc2)cc1]

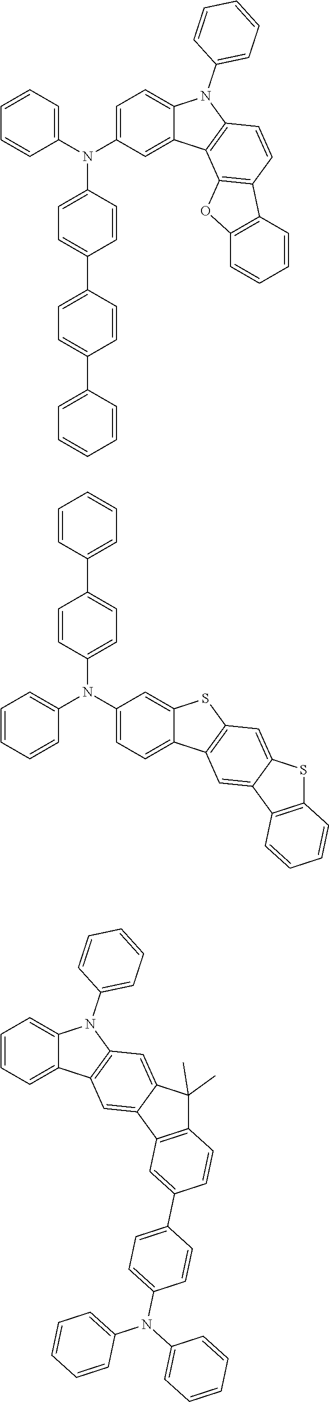

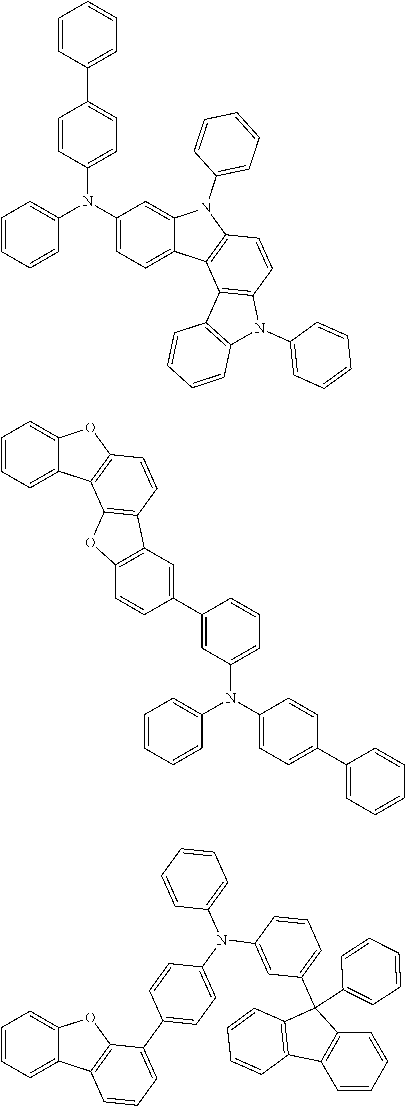 [SMILES: c1ccc(-c2ccc(N(c3ccccc3)c3ccc4c5c6c7ccccc7n(-c7ccccc7)c6ccc5n(-c5ccccc5)c4c3)cc2)cc1 ; c1ccc(-c2ccc(N(c3ccccc3)c3cccc(-c4ccc5oc6c(ccc7oc8ccccc8c76)c5c4)c3)cc2)cc1 ; c1ccc(N(c2ccc(-c3cccc4c3oc3ccccc34)cc2)c2cccc(C3(c4ccccc4)c4ccccc4-c4ccccc43)c2)cc1]

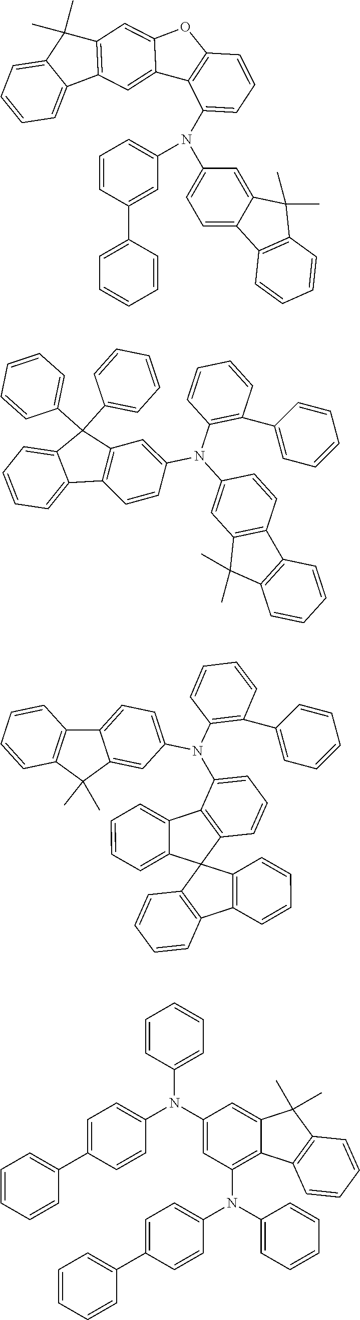

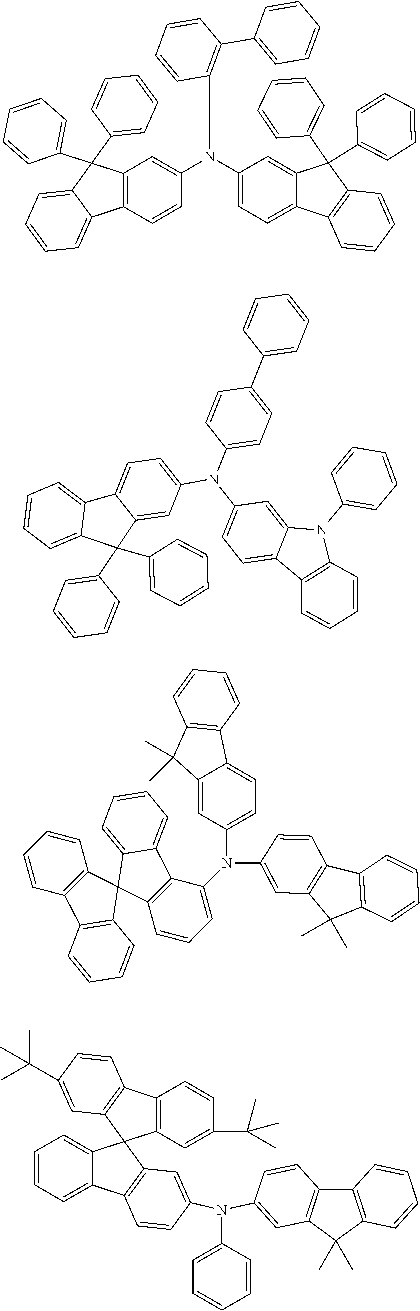 [SMILES: c1ccc(-c2ccccc2N(c2ccc3c(c2)C(c2ccccc2)(c2ccccc2)c2ccccc2-3)c2ccc3c(c2)C(c2ccccc2)(c2ccccc2)c2ccccc2-3)cc1 ; c1ccc(-c2ccc(N(c3ccc4c(c3)C(c3ccccc3)(c3ccccc3)c3ccccc3-4)c3ccc4c5ccccc5n(-c5ccccc5)c4c3)cc2)cc1 ; CC1(C)c2ccccc2-c2ccc(N(c3ccc4c(c3)C(C)(C)c3ccccc3-4)c3cccc4c3-c3ccccc3C43c4ccccc4-c4ccccc43)cc21 ; CC(C)(C)c1ccc2c(c1)C1(c3ccccc3-c3ccc(N(c4ccccc4)c4ccc5c(c4)C(C)(C)c4ccccc4-5)cc31)c1cc(C(C)(C)C)ccc1-2]

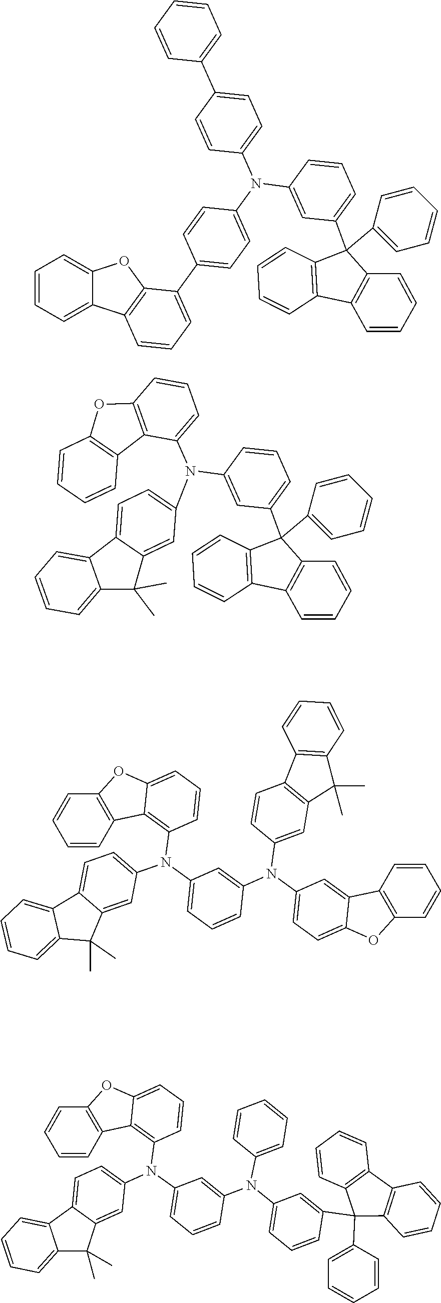

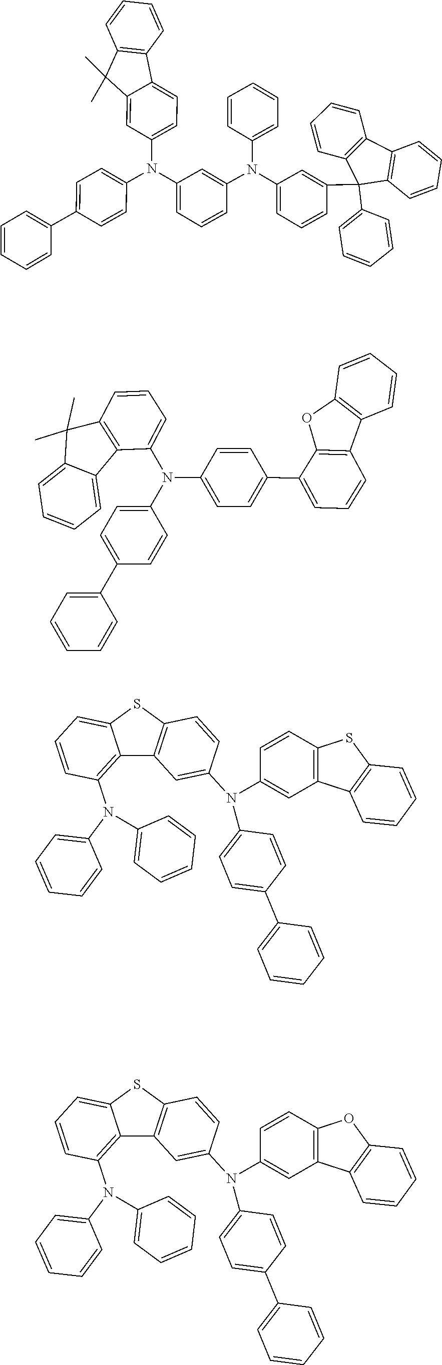 [SMILES: CC1(C)c2ccccc2-c2ccc(N(c3ccc(-c4ccccc4)cc3)c3cccc(N(c4ccccc4)c4cccc(C5(c6ccccc6)c6ccccc6-c6ccccc65)c4)c3)cc21 ; CC1(C)c2ccccc2-c2c(N(c3ccc(-c4ccccc4)cc3)c3ccc(-c4cccc5c4oc4ccccc45)cc3)cccc21 ; c1ccc(-c2ccc(N(c3ccc4sc5ccccc5c4c3)c3ccc4sc5cccc(N(c6ccccc6)c6ccccc6)c5c4c3)cc2)cc1 ; c1ccc(-c2ccc(N(c3ccc4oc5ccccc5c4c3)c3ccc4sc5cccc(N(c6ccccc6)c6ccccc6)c5c4c3)cc2)cc1]

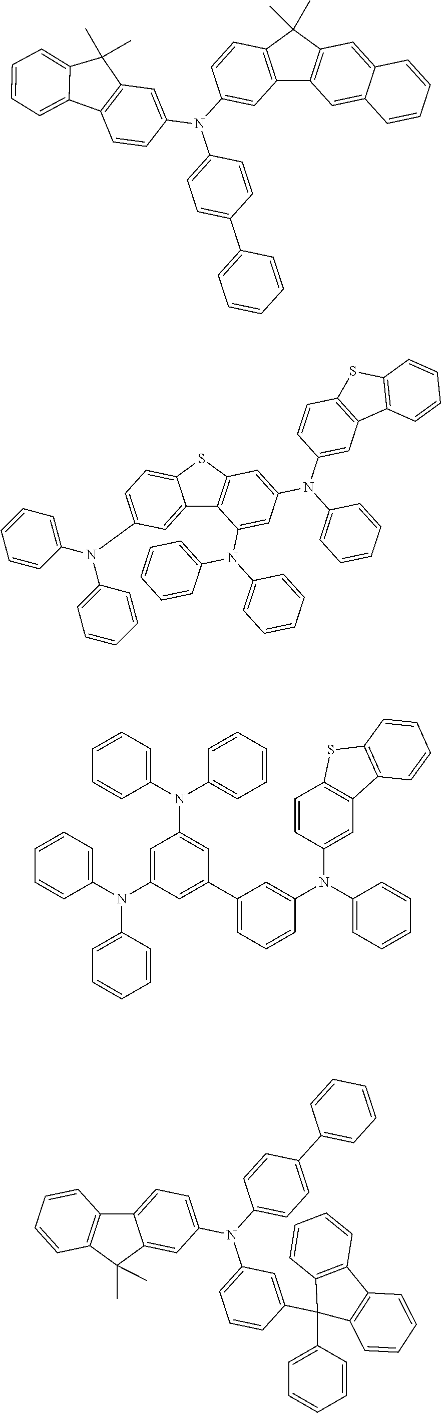 [SMILES: CC1(C)c2ccccc2-c2ccc(N(c3ccc(-c4ccccc4)cc3)c3ccc4c(c3)-c3cc5ccccc5cc3C4(C)C)cc21 ; c1ccc(N(c2cc(N(c3ccccc3)c3ccccc3)c3c(c2)sc2ccc(N(c4ccccc4)c4ccccc4)cc23)c2ccc3sc4ccccc4c3c2)cc1 ; c1ccc(N(c2ccccc2)c2cc(-c3cccc(N(c4ccccc4)c4ccc5sc6ccccc6c5c4)c3)cc(N(c3ccccc3)c3ccccc3)c2)cc1 ; CC1(C)c2ccccc2-c2ccc(N(c3ccc(-c4ccccc4)cc3)c3cccc(C4(c5ccccc5)c5ccccc5-c5ccccc54)c3)cc21]

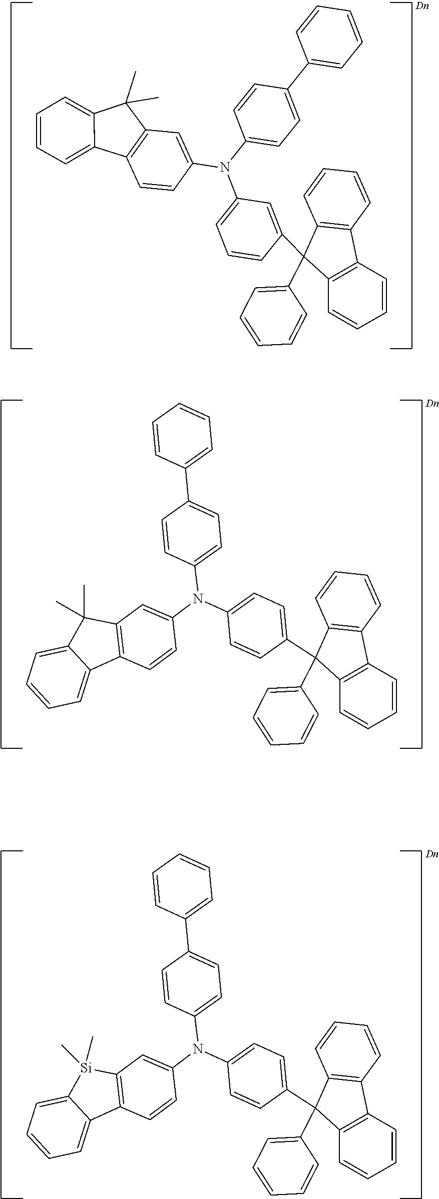

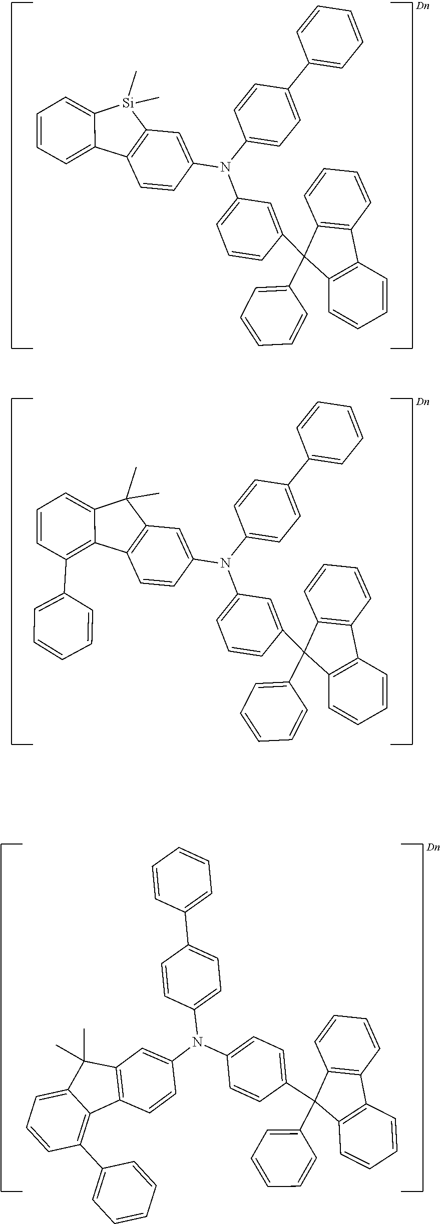

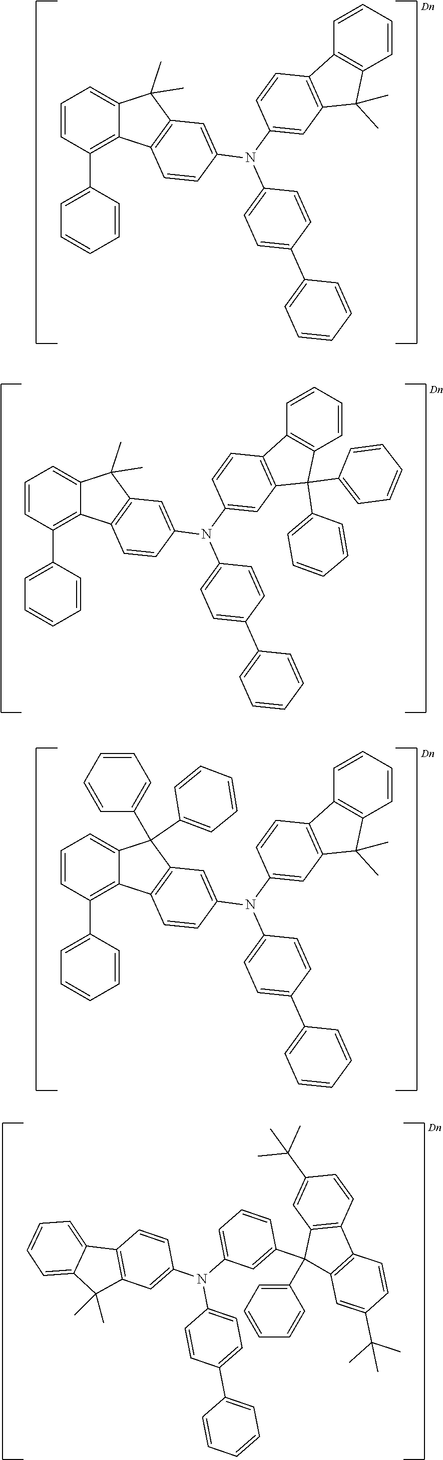

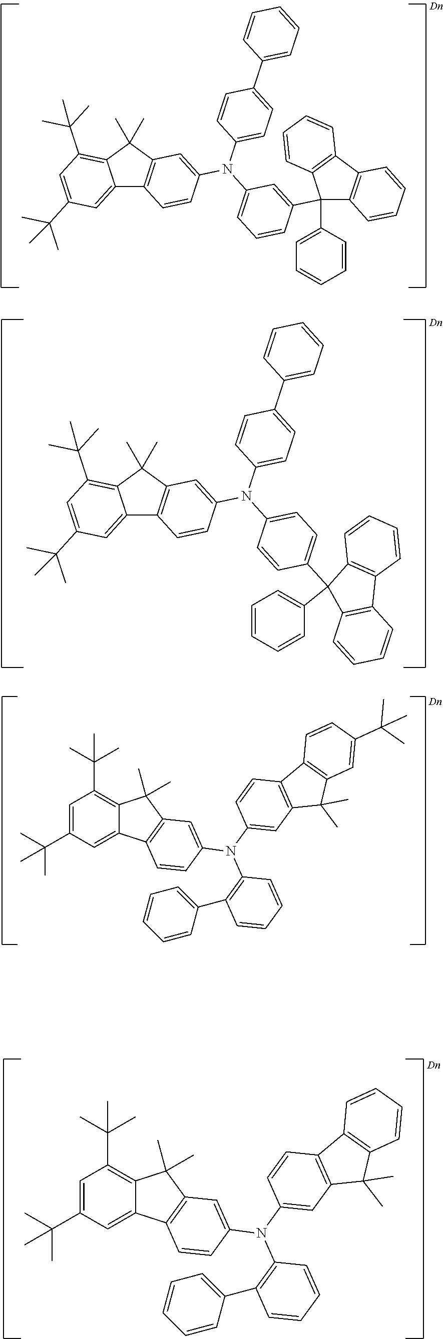

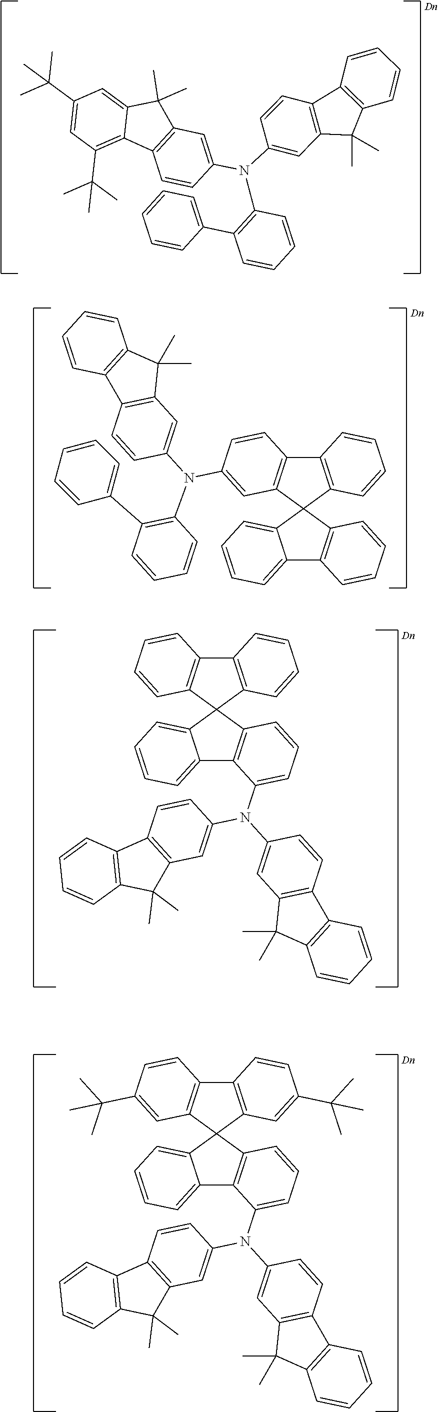

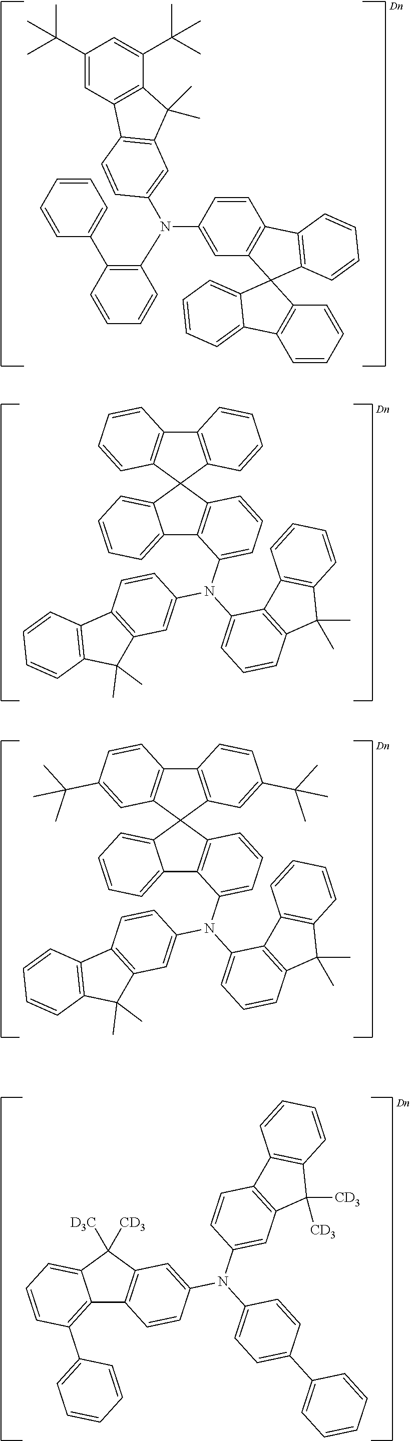

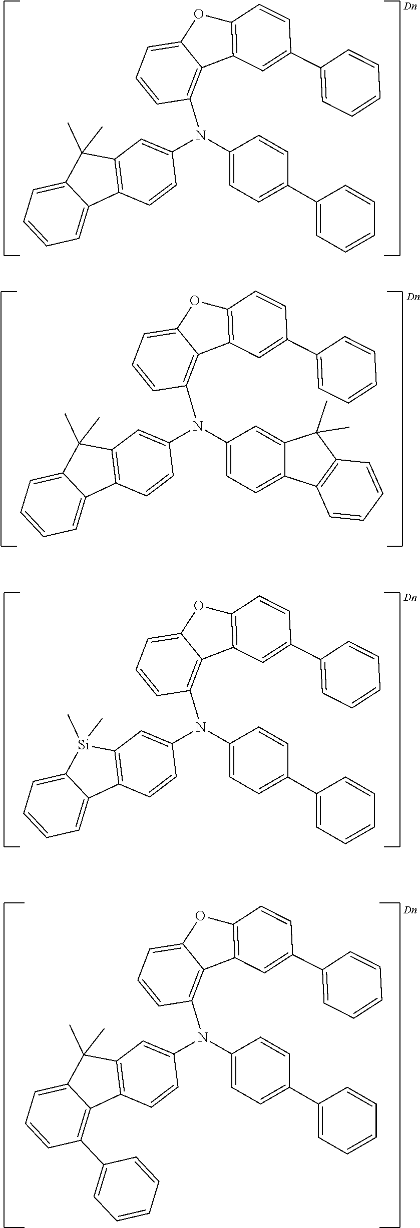

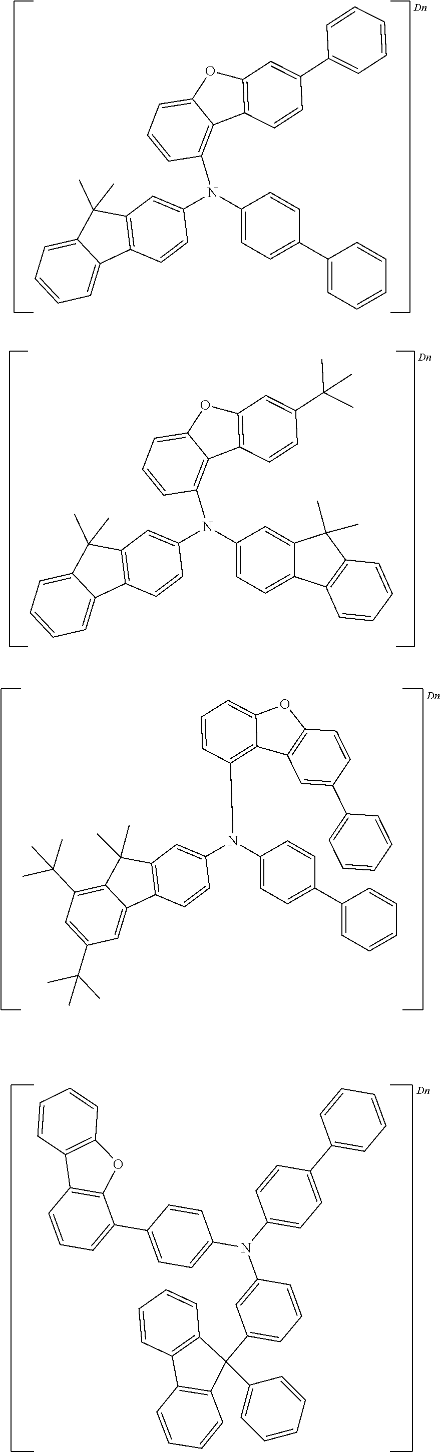

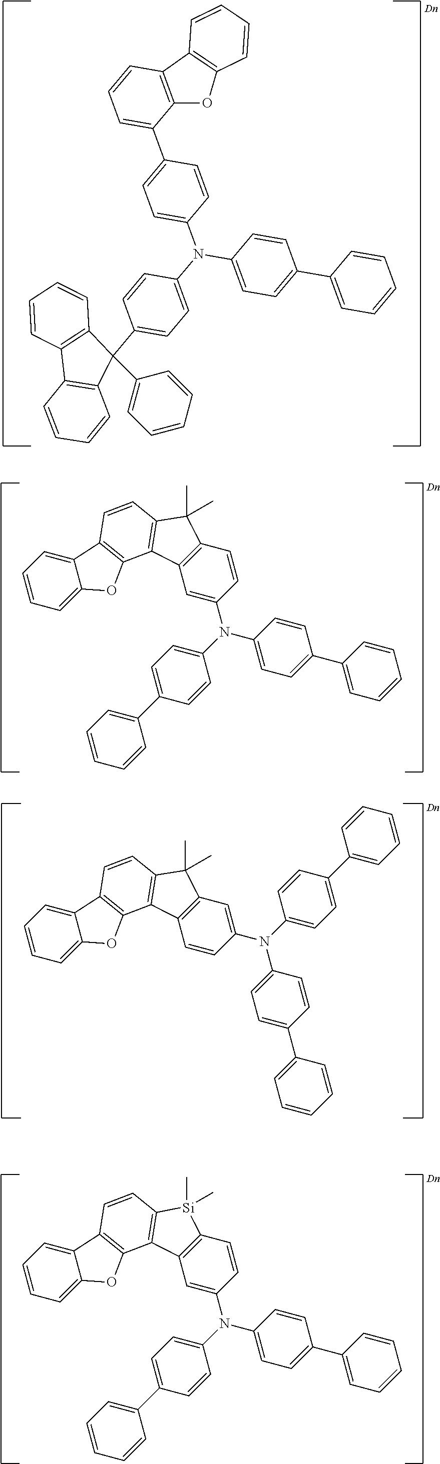

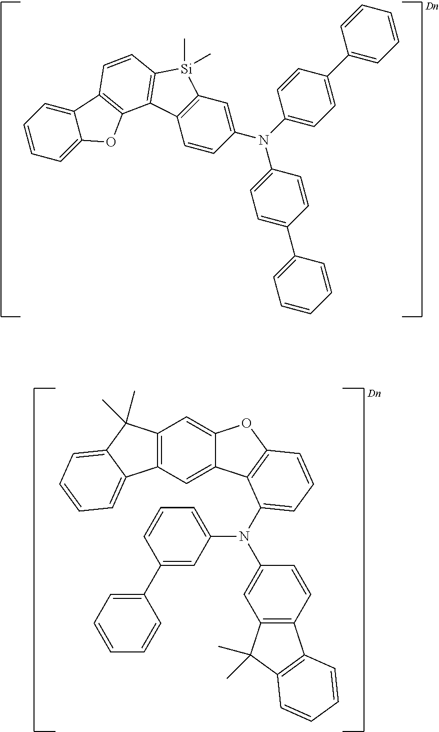

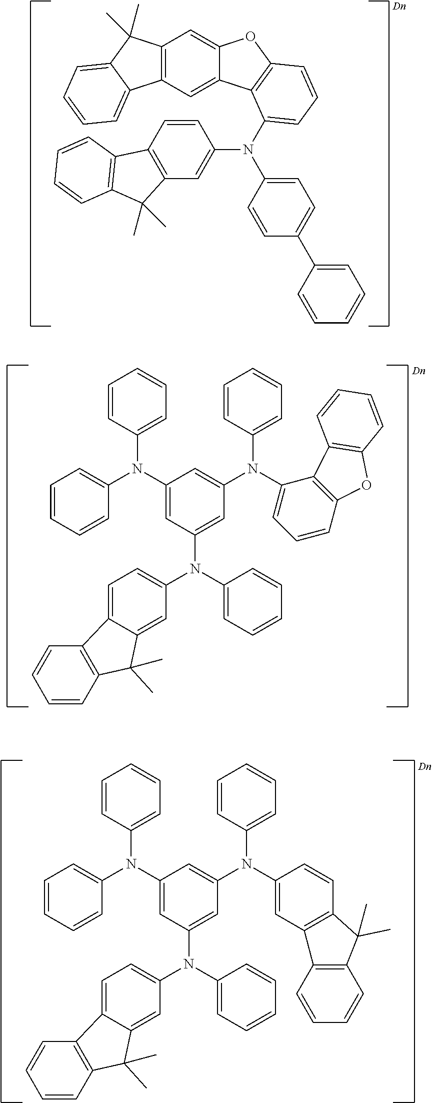

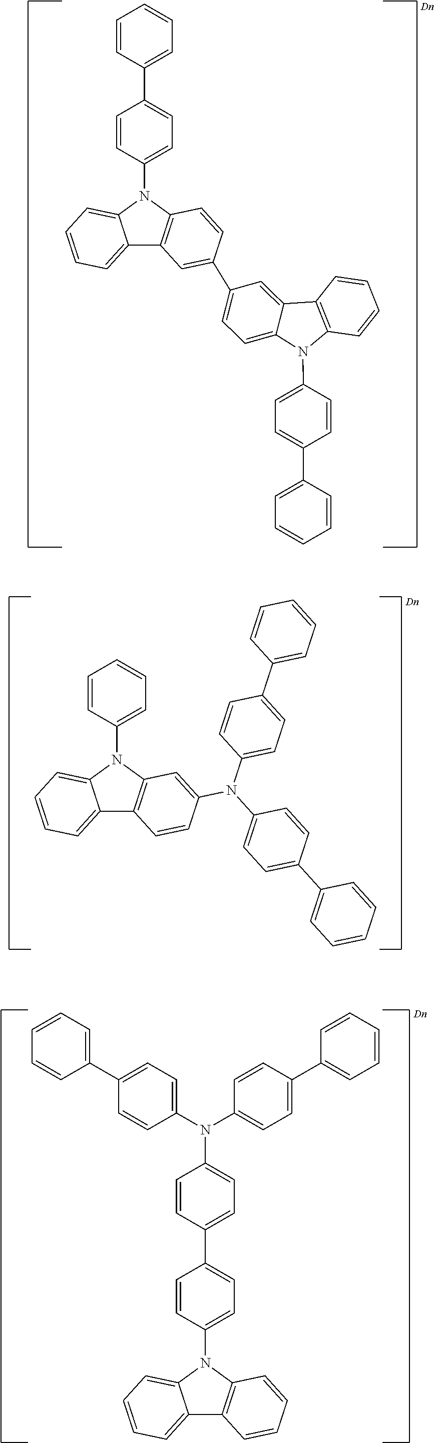

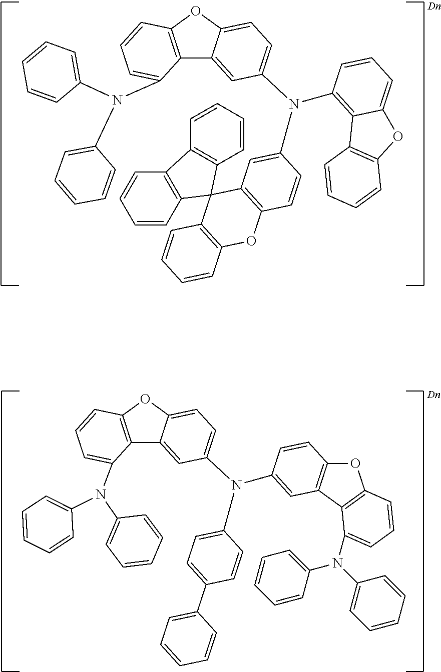

(Dn means the number of deuterium substituted, and represents a structure substituted with one or more deuterium)

In the hole transport region 140, other suitable compounds may be used in addition to the aforementioned compound.

In an implementation, the charge transport region may be, e.g., an electron transport region 150.

The electron transport region 150 may help further increase electron injection and/or electron mobility and block holes between the cathode 110 and the light emitting layer 130.

In an implementation, the electron transport region 150 may include an electron transport layer between the cathode 110 and the light emitting layer 130, and an electron transport auxiliary layer between the light emitting layer 130 and the electron transport layer, and a compound of Group C may be included in at least one of the electron transport layer and the electron transport auxiliary layer.

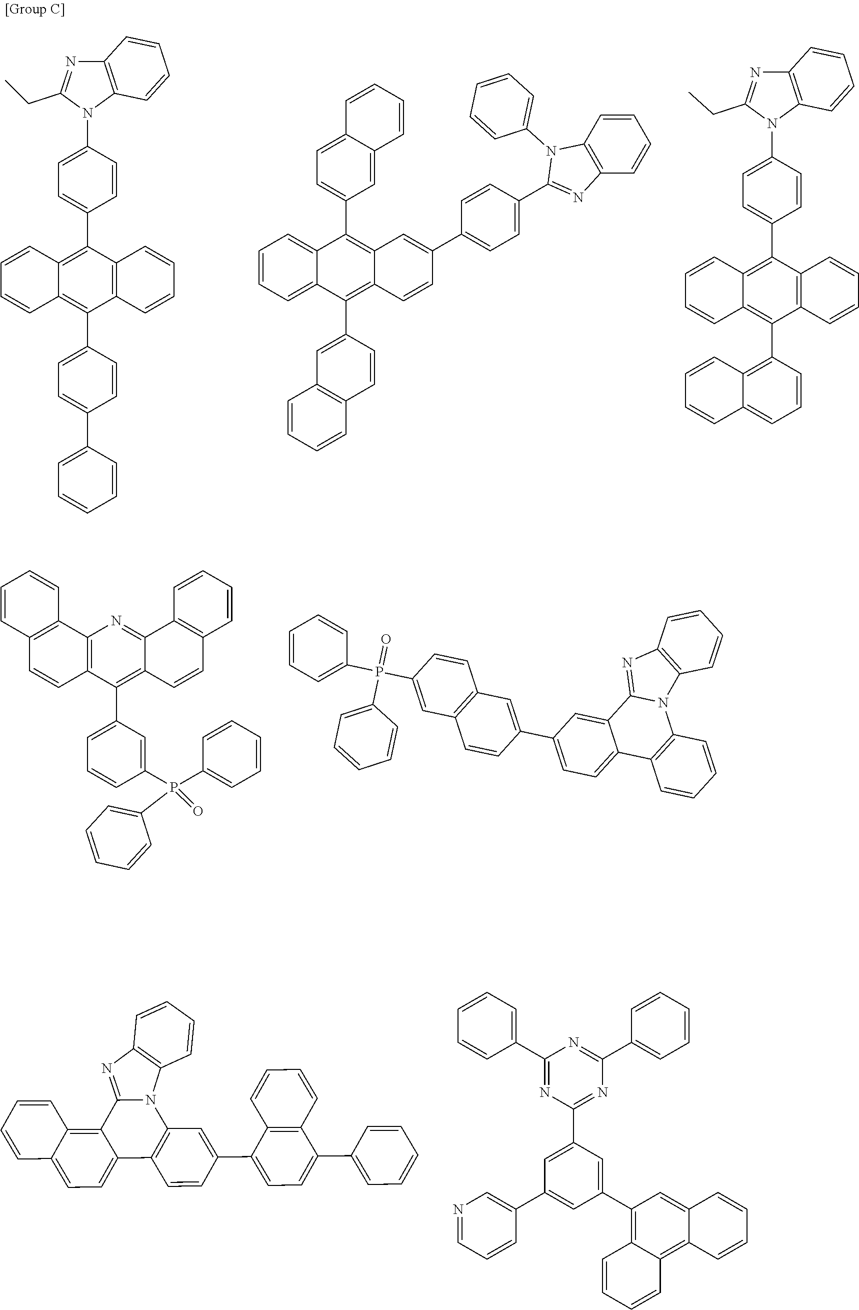

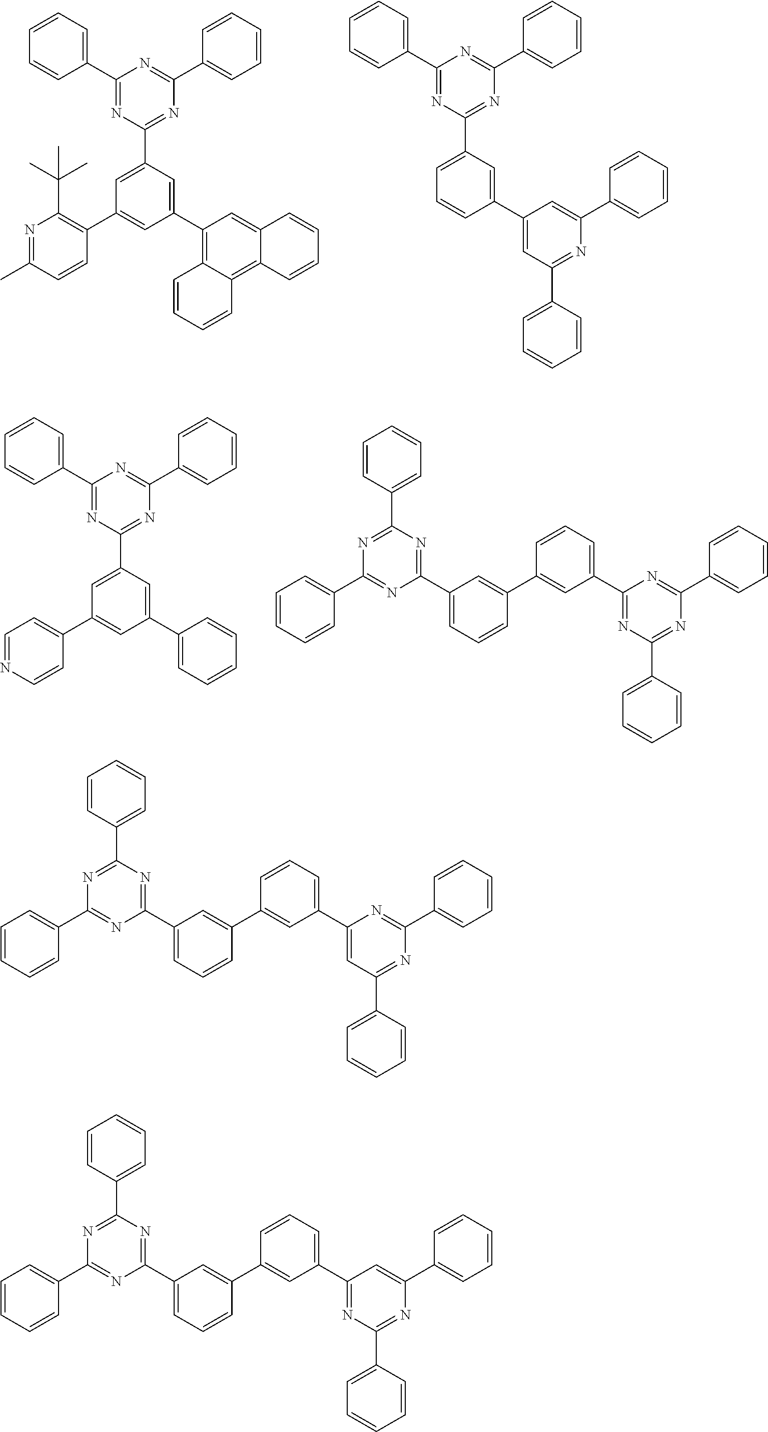

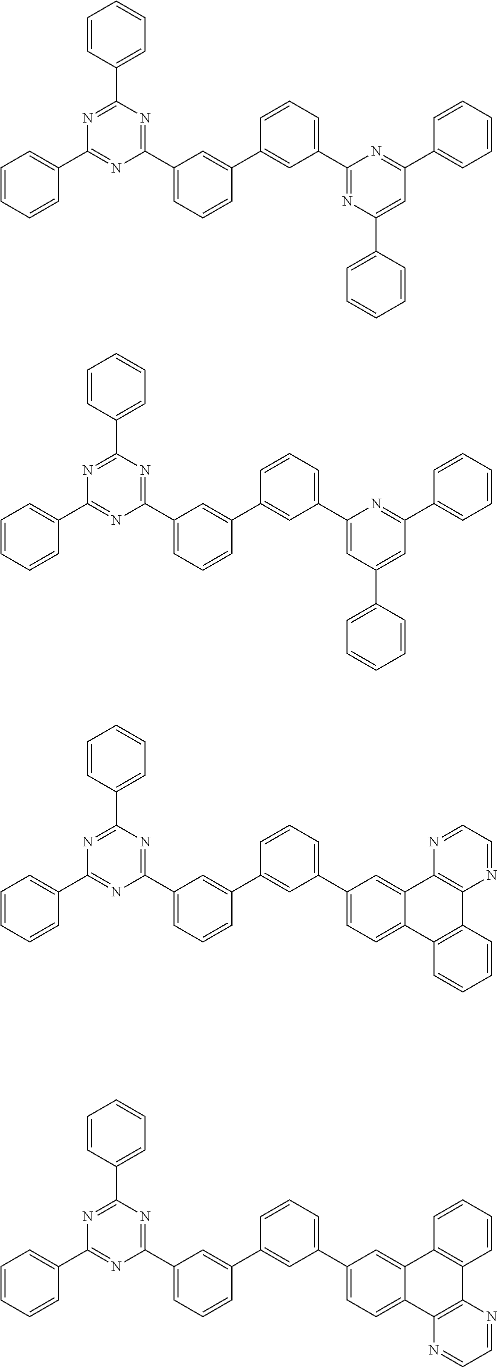 [SMILES: c1ccc(-c2cc(-c3ccccc3)nc(-c3cccc(-c4cccc(-c5nc(-c6ccccc6)nc(-c6ccccc6)n5)c4)c3)n2)cc1 ; c1ccc(-c2cc(-c3ccccc3)nc(-c3cccc(-c4cccc(-c5nc(-c6ccccc6)nc(-c6ccccc6)n5)c4)c3)c2)cc1 ; c1ccc(-c2nc(-c3ccccc3)nc(-c3cccc(-c4cccc(-c5ccc6c7ccccc7c7nccnc7c6c5)c4)c3)n2)cc1 ; c1ccc(-c2nc(-c3ccccc3)nc(-c3cccc(-c4cccc(-c5ccc6c(c5)c5ccccc5c5nccnc65)c4)c3)n2)cc1]

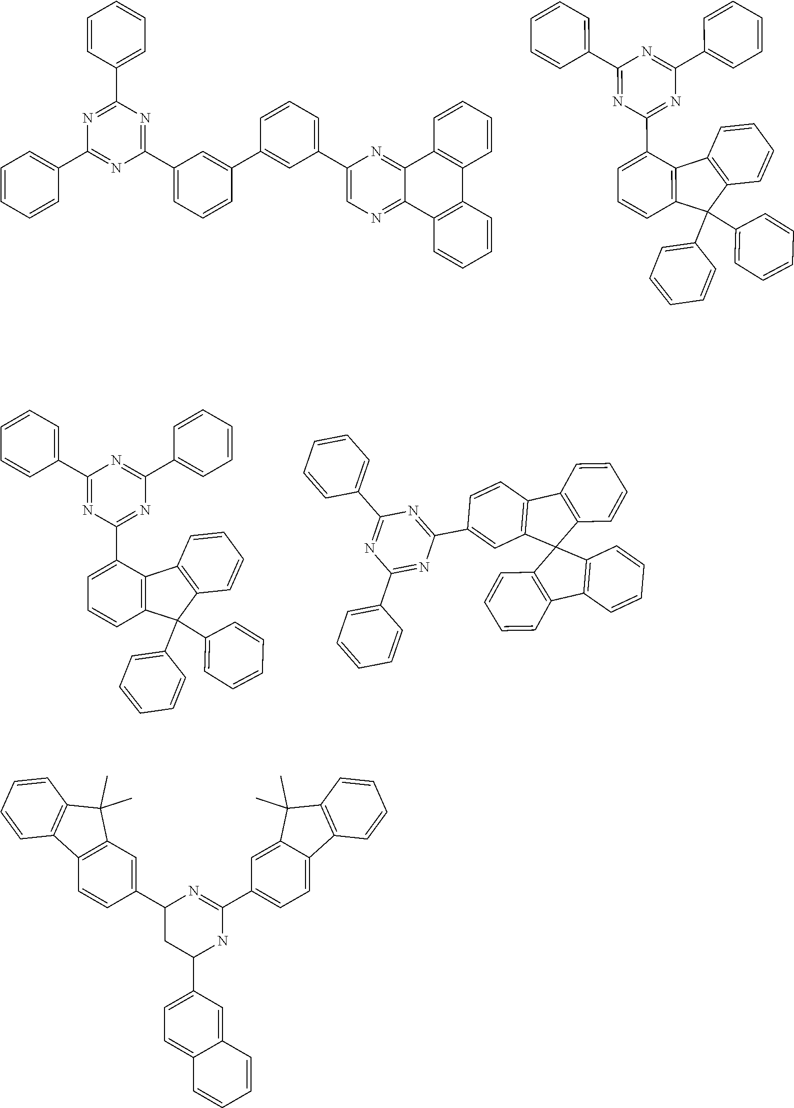

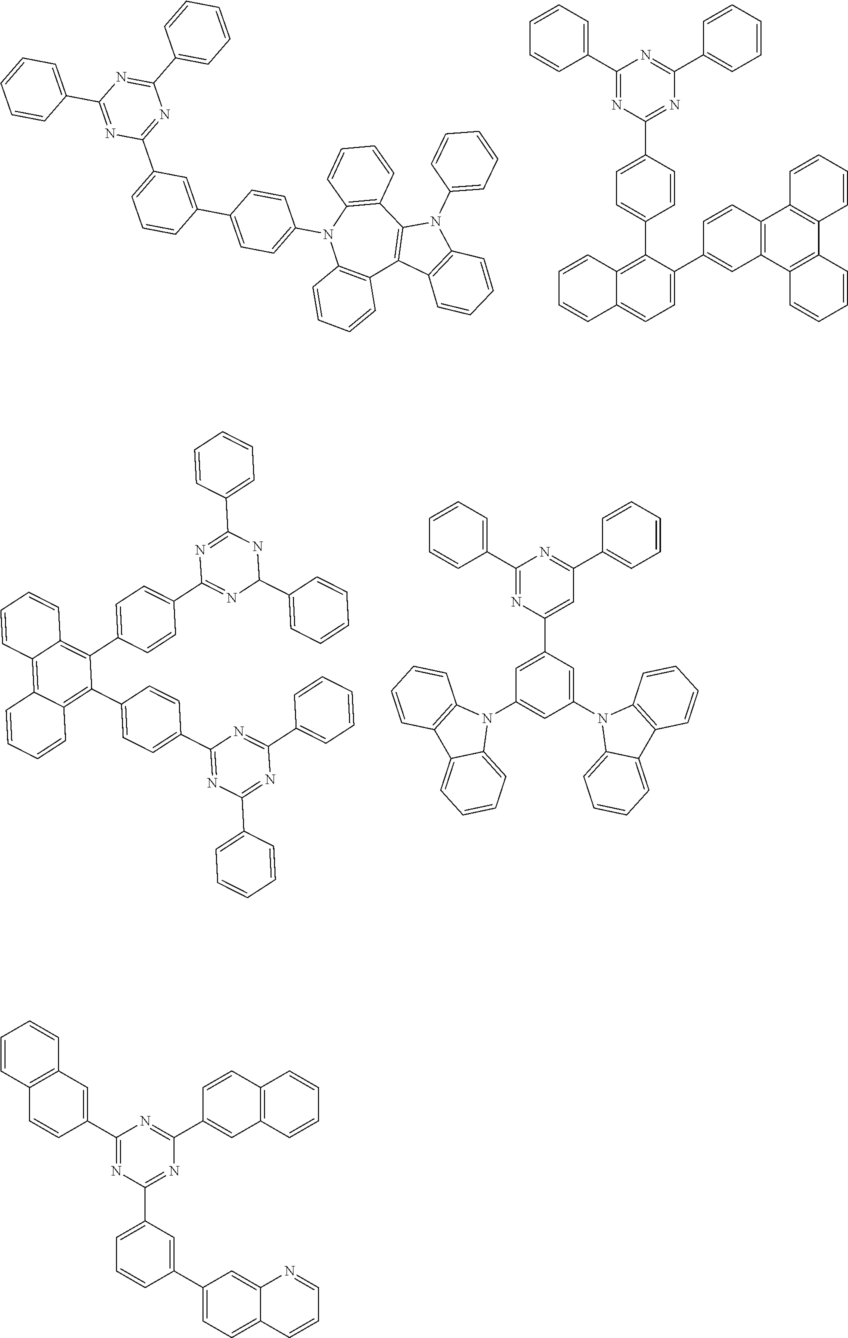

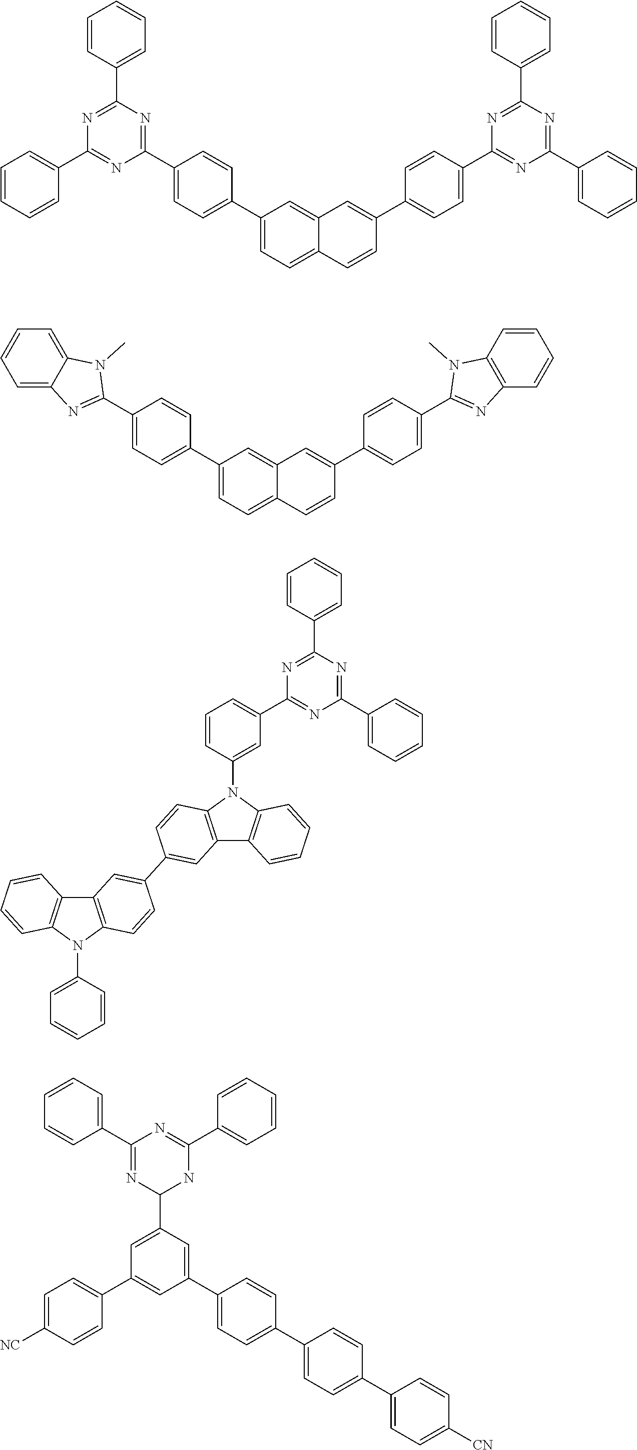

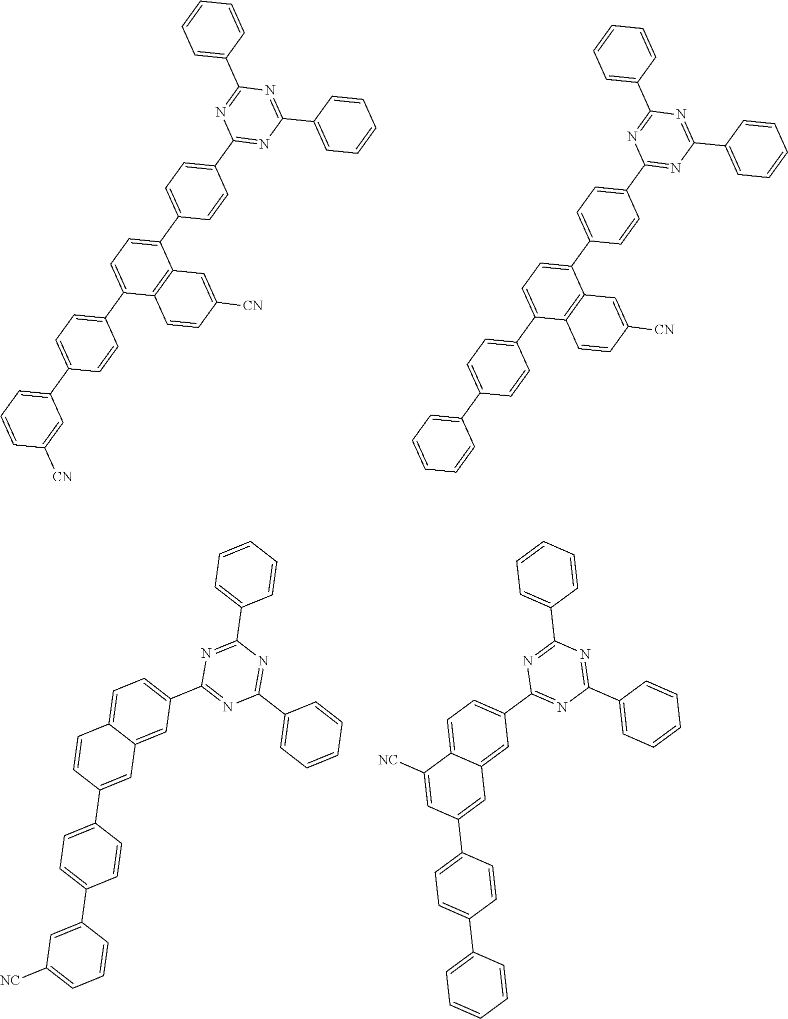 [SMILES: N#Cc1cccc(-c2ccc(-c3ccc(-c4ccc(-c5nc(-c6ccccc6)nc(-c6ccccc6)n5)cc4)c4cc(C#N)ccc34)cc2)c1 ; N#Cc1ccc2c(-c3ccc(-c4ccccc4)cc3)ccc(-c3ccc(-c4nc(-c5ccccc5)nc(-c5ccccc5)n4)cc3)c2c1 ; N#Cc1cccc(-c2ccc(-c3ccc4ccc(-c5nc(-c6ccccc6)nc(-c6ccccc6)n5)cc4c3)cc2)c1 ; N#Cc1cc(-c2ccc(-c3ccccc3)cc2)cc2cc(-c3nc(-c4ccccc4)nc(-c4ccccc4)n3)ccc12]

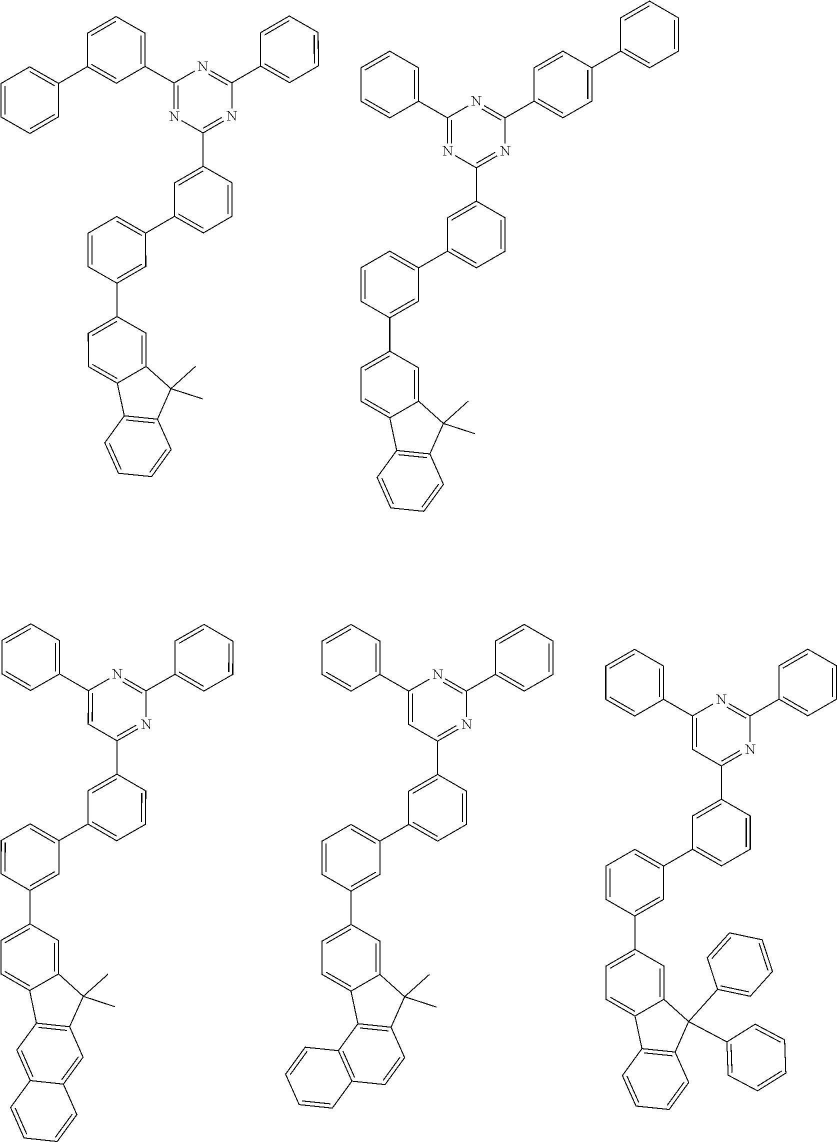

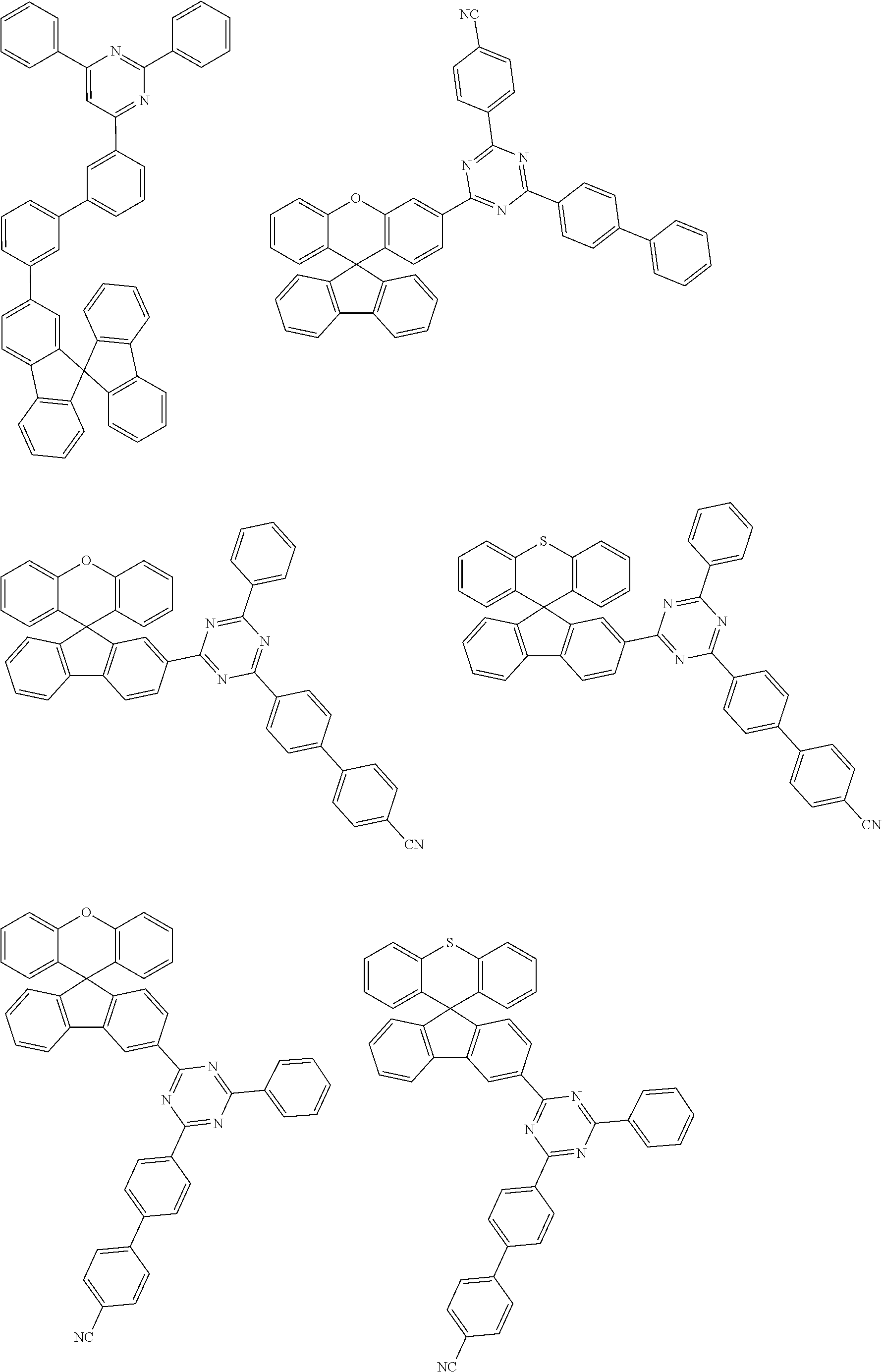

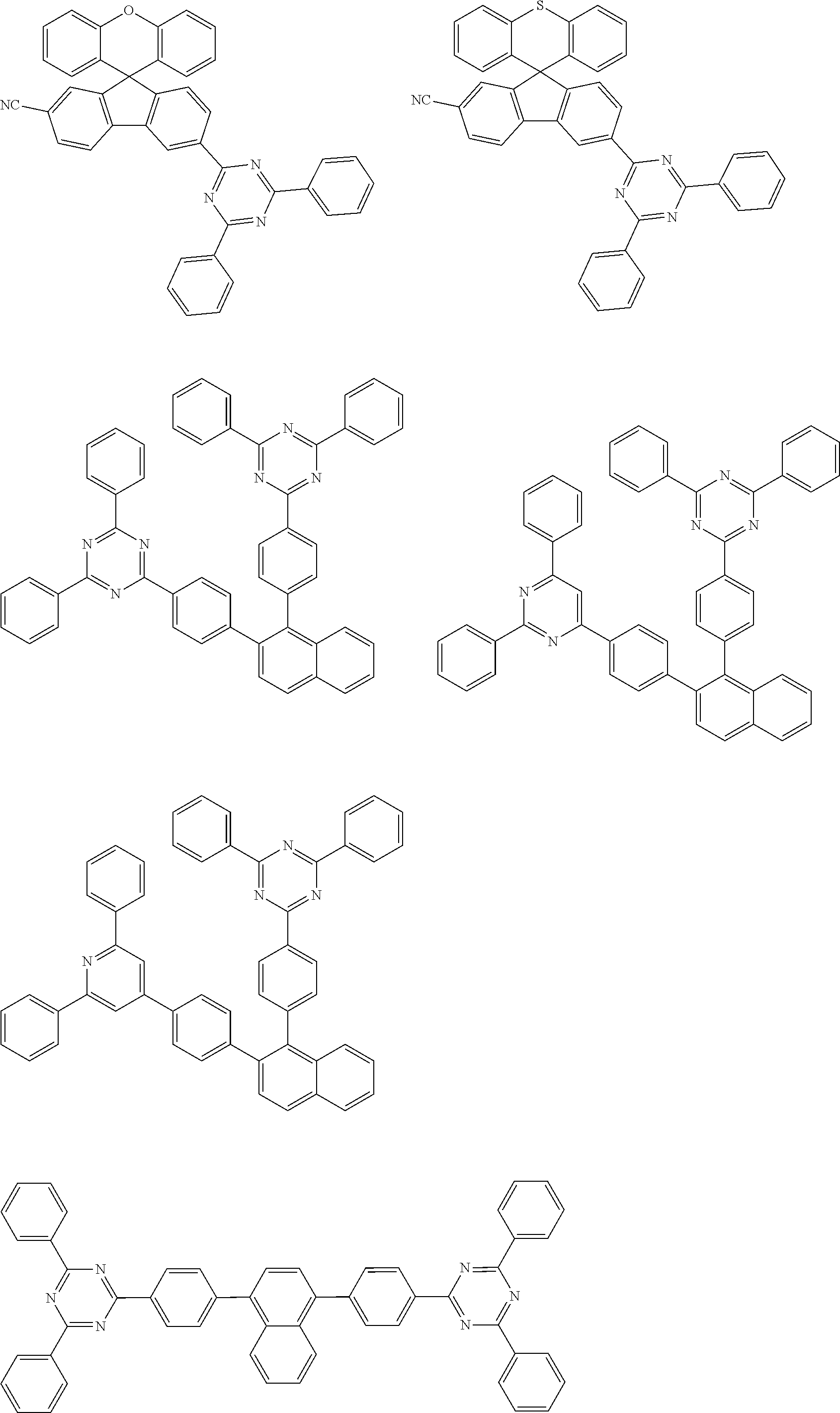

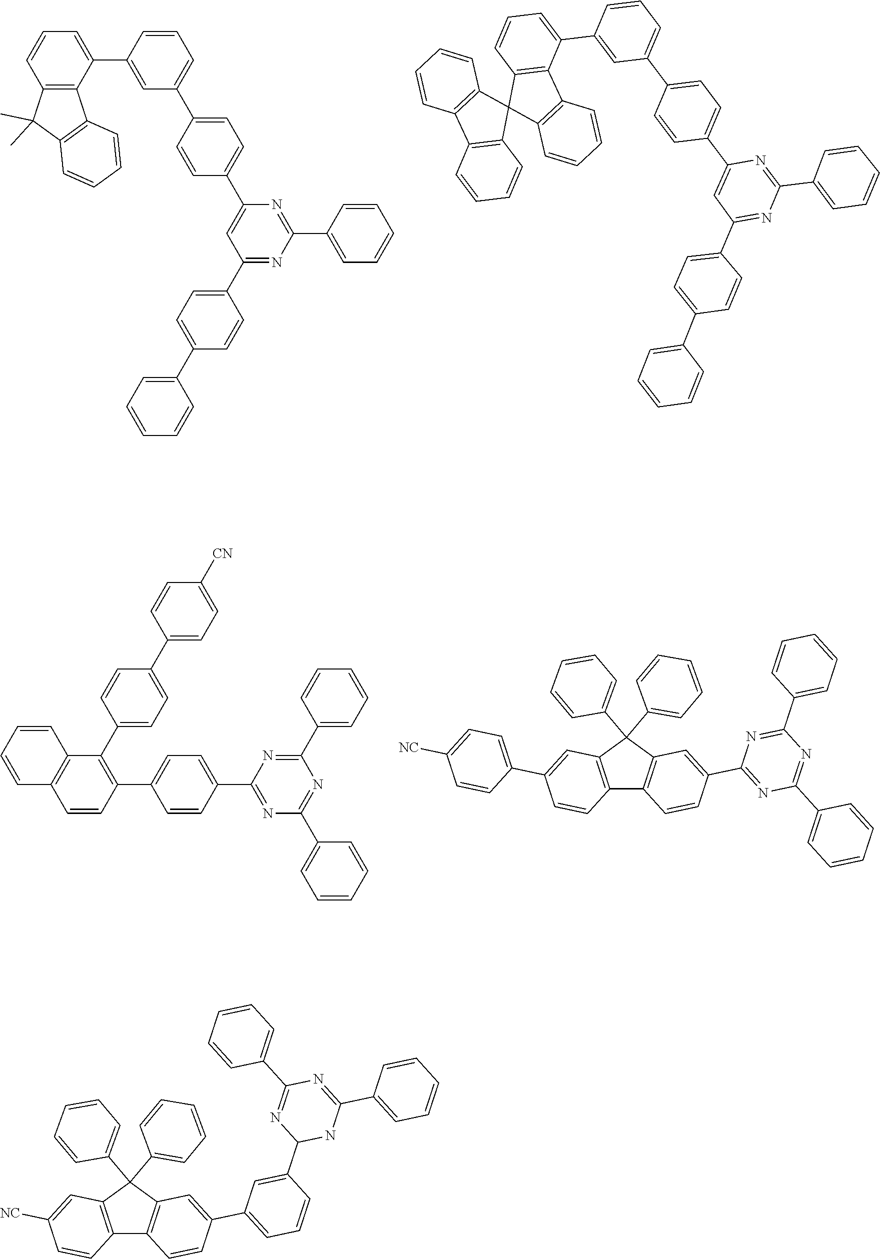

An embodiment may provide an organic light emitting diode including a light emitting layer as an organic layer.

Another embodiment may provide an organic light emitting diode including a light emitting layer and a hole transport region as an organic layer.

Another embodiment may provide an organic light emitting diode including a light emitting layer and an electron transport region as an organic layer.

As shown in the FIGURE, the organic light emitting diode according to an embodiment may include a hole transport region 140 and an electron transport region 150 in addition to the light emitting layer 130 as the organic layer 105.

In an implementation, the organic light emitting diode may further include an electron injection layer, a hole injection layer, or the like, in addition to the light emitting layer as the aforementioned organic layer.

The organic light emitting diode 100 may be produced by forming an anode or a cathode on a substrate, forming an organic layer using a dry film formation method such as a vacuum deposition method (evaporation), sputtering, plasma plating, and ion plating, and forming a cathode or an anode thereon.

The organic light emitting diode may be applied to an organic light emitting display device.

The following Examples and Comparative Examples are provided in order to highlight characteristics of one or more embodiments, but it will be understood that the Examples and Comparative Examples are not to be construed as limiting the scope of the embodiments, nor are the Comparative Examples to be construed as being outside the scope of the embodiments. Further, it will be understood that the embodiments are not limited to the particular details described in the Examples and Comparative Examples.

Hereinafter, starting materials and reactants used in Examples and Synthesis Examples were purchased from Sigma-Aldrich Co. Ltd., TCI Inc., Tokyo chemical industry, or P&H tech as far as there is no particular comment or were synthesized by known methods.

(Preparation of Compound for Organic Optoelectronic Device)

### Synthesis Example 1: Synthesis of Intermediate I-1

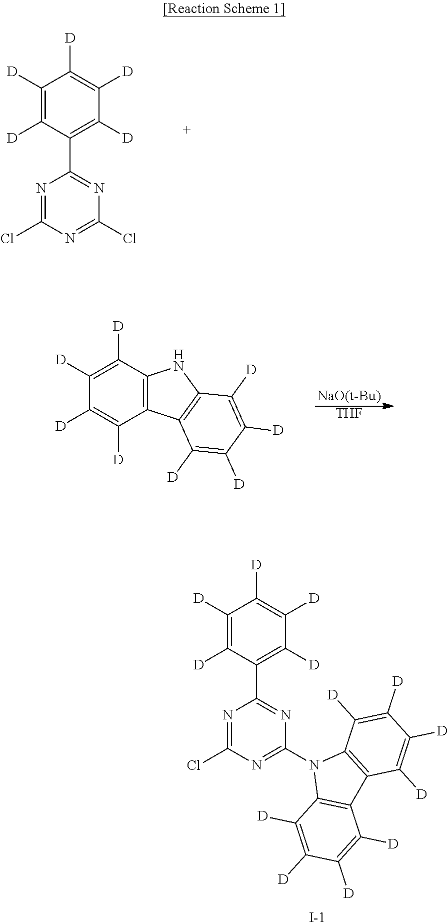 [SMILES: [2H]c1c([2H])c([2H])c(-c2nc(Cl)nc(Cl)n2)c([2H])c1[2H] ; [2H]c1c([2H])c([2H])c2c([nH]c3c([2H])c([2H])c([2H])c([2H])c32)c1[2H] ; [2H]c1c([2H])c([2H])c(-c2nc(Cl)nc(-n3c4c([2H])c([2H])c([2H])c([2H])c4c4c([2H])c([2H])c([2H])c([2H])c43)n2)c([2H])c1[2H] ; "NaO(t-Bu)" ; "THF" ; [I-]]

Under a nitrogen atmosphere, 2,4-dichloro-6-(phenyl-2,3,4,5,6-d5)-1,3,5-triazine (59.3 g, 256 mmol) and 9H-carbazole-1,2,3,4,5,6,7,8-d8 (30 g, 171 mmol) were dissolved in 500 ml of tetrahydrofuran, and sodium tert-butoxide (18.1 g, 188 mmol) was slowly added thereto and then, stirred at 0° C. After 12 hours, water was added to the reaction solution, and the mixture was filtered. The obtained residue was separated and purified through flash column chromatography, obtaining Intermediate I-1 (50 g, 79%).

HRMS (70 eV, EI+): m/z calcd for C21D13ClN4: 369.1645, found: 369.

Elemental Analysis: C, 68%; H, 7%

### Synthesis Example 2: Synthesis of Intermediate I-2

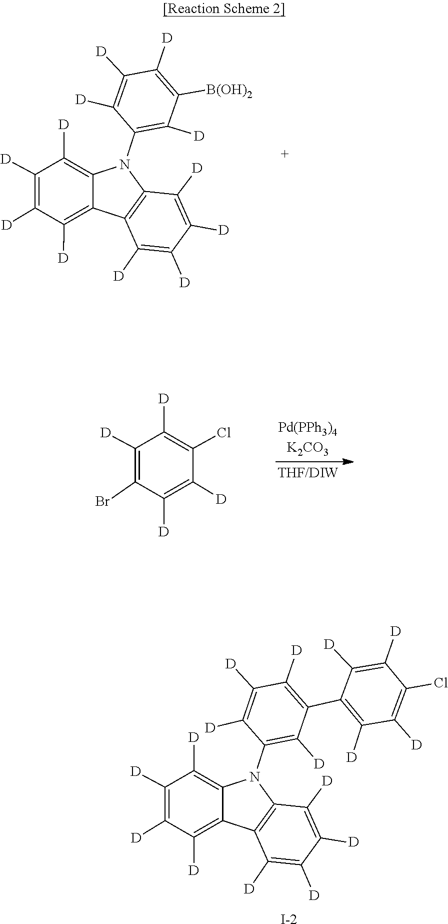

Under a nitrogen atmosphere, (3-(9H-carbazol-9-yl-d8)phenyl-2,4,5,6-d4)boronic acid (30.6 g, 102 mmol) and 1-bromo-4-chlorobenzene-2,3,5,6-d4 (20 g, 102 mmol) were dissolved by adding 300 ml of tetrahydrofuran thereto, and 150 ml of an aqueous solution in which potassium carbonate (28.2 g, 204 mmol) was dissolved was added thereto and then, stirred. Subsequently, tetrakis(triphenylphosphine) palladium (3.54 g, 3.06 mmol) was added thereto and then, heated at 80° C. for 12 hours and stirred under reflux. When a reaction was completed, an organic layer was extracted therefrom, treated with magnesium sulfate anhydrous to remove moisture, filtered, and concentrated under a reduced pressure. The obtained residue was separated and purified through flash column chromatography, obtaining Intermediate I-2 (35 g, 93%).

HRMS (70 eV, EI+): m/z calcd for C24D16ClN: 369.1976, found: 369.

Elemental Analysis: C, 78%; H, 9%

### Synthesis Example 3: Synthesis of Intermediate I-3

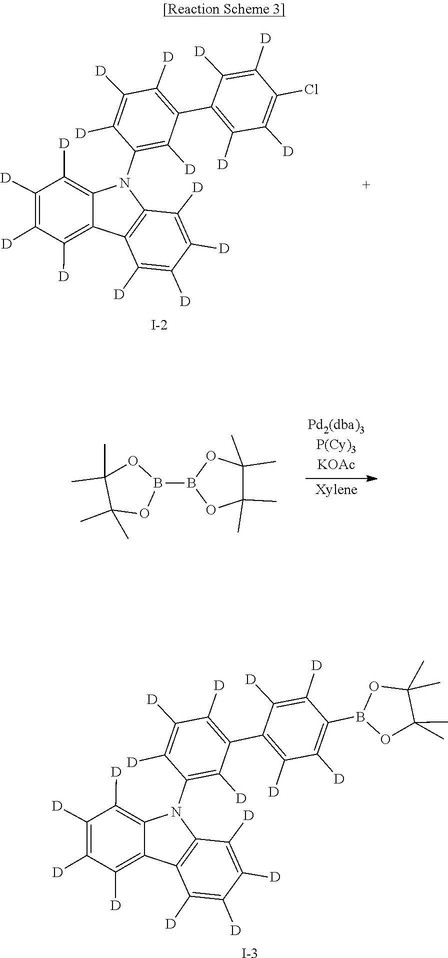

Under a nitrogen atmosphere, Intermediate I-2 (35 g, 94.6 mmol) was dissolved in 500 ml of xylene, and bis(pinacolato) diboron (28.8 g, 113 mmol), tris(dibenzylideneacetone) dipalladium (0) (0.87 g, 0.95 mmol), tricyclohexylphosphine (1.1 g, 3.8 mmol), and potassium acetate (27.8 g, 284 mmol) were added thereto and then, stirred under reflux for 8 hours. When a reaction was completed, after adding water to the reaction solution, the obtained mixture was extracted with dichloromethane, treated with magnesium sulfate anhydrous to remove moisture, filtered, and concentrated under a reduced pressure. The obtained residue was separated and purified through flash column chromatography, obtaining Intermediate I-3 (30 g, 69%).

HRMS (70 eV, EI+): m/z calcd for C30H12D16BNO2: 461.3217, found: 461.

Elemental Analysis: C, 78%; H, 10%

### Synthesis Example 4: Synthesis of Compound A-3

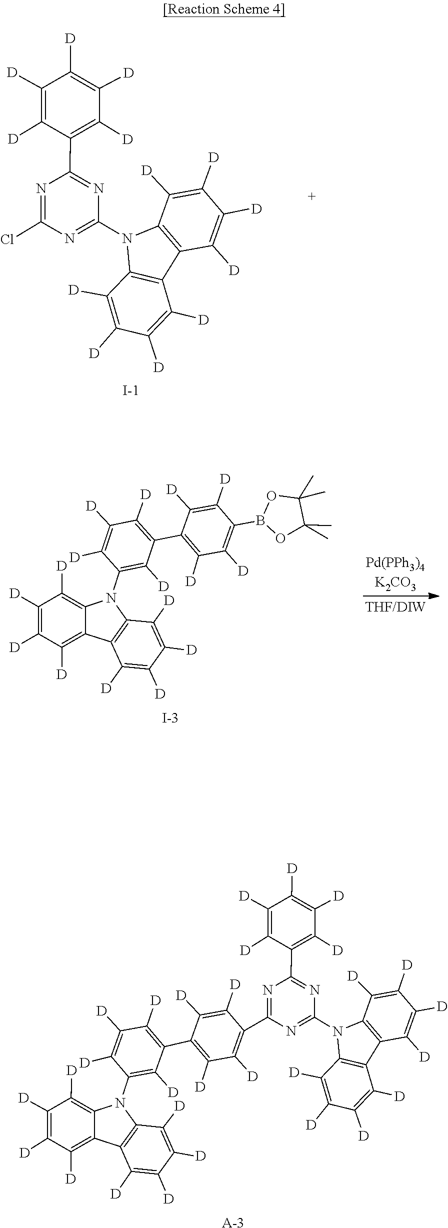

Compound A-3 (15 g, 83%) was obtained in the same manner as in Synthesis Example 2 except that Intermediate I-1 (10 g, 27 mmol) and Intermediate I-3 (12.5 g, 27 mmol) were used.

HRMS (70 eV, EI+): m/z calcd for C45D29N5: 668.4243, found: 668.

Elemental Analysis: C, 81%; H, 9%

### Synthesis Example 5: Synthesis of Compound A-21

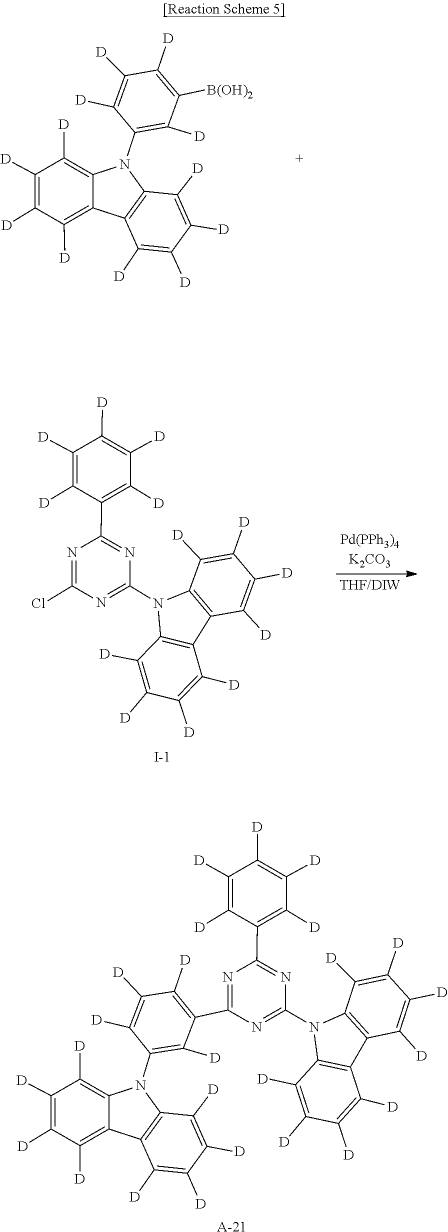

Compound A-21 (16 g, 81%) was obtained in the same manner as in Synthesis Example 2 except that (3-(9H-carbazol-9-yl-d8)phenyl-2,4,5,6-d4)boronic acid (10 g, 33.4 mmol) and Intermediate I-1 (12.4 g, 33.4 mmol) were used.

HRMS (70 eV, EI+): m/z calcd for C39D25N5: 588.3679, found: 588.

Elemental Analysis: C, 80%; H, 9%

### Synthesis Example 6: Synthesis of Intermediate I-4

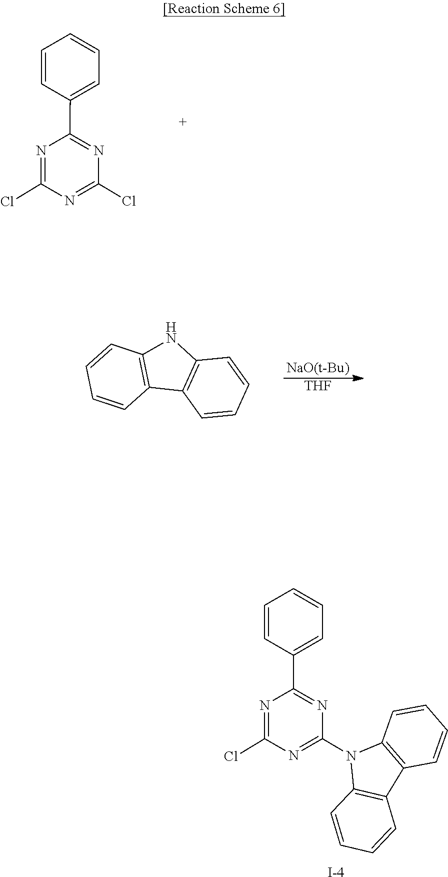

Intermediate I-4 (80 g, 75%) was obtained in the same manner as in Synthesis Example 1 except that 2,4-dichloro-6-phenyl-1,3,5-triazine (101 g, 450 mmol) and 9H-carbazole (50 g, 300 mmol) were used.

HRMS (70 eV, EI+): m/z calcd for C21H13ClN4: 356.0829, found: 356.

Elemental Analysis: C, 71%; H, 4%

### Synthesis Example 7: Synthesis of Intermediate I-5

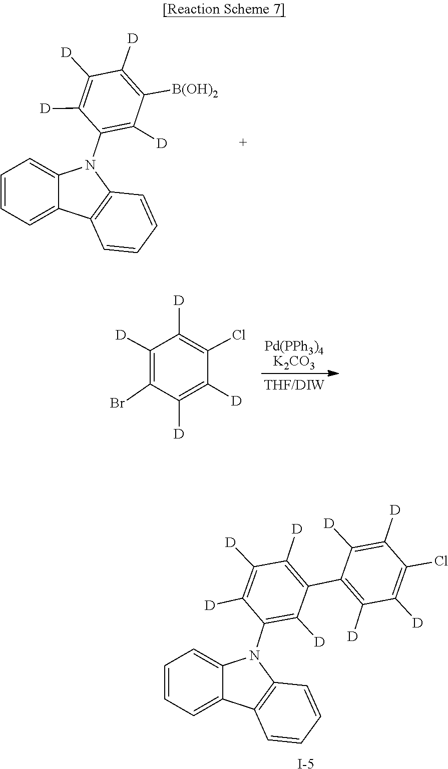

Intermediate I-5 (30 g, 80%) was obtained in the same manner as in Synthesis Example 2 except that B-[5-(9H-carbazol-9-yl)phenyl-2,3,4,6-d4]boronic acid (30 g, 103 mmol) and 1-bromo-4-chlorobenzene-2,3,5,6-d4 (20 g, 103 mmol) were used.

HRMS (70 eV, EI+): m/z calcd for C24H8D8ClN: 361.1473, found: 361.

Elemental Analysis: C, 80%; H, 7%

### Synthesis Example 8: Synthesis of Intermediate I-6

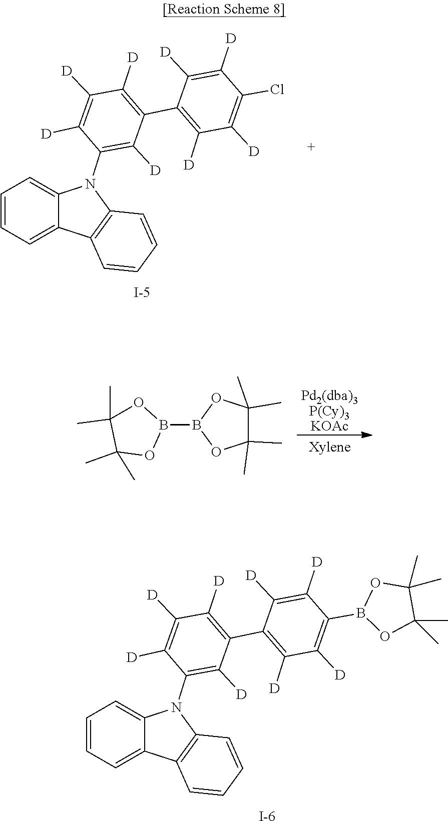

Intermediate I-6 (20 g, 54%) was obtained in the same manner as in Synthesis Example 3 except that Intermediate I-5 (30 g, 82 mmol) instead of Intermediate I-2 was used.

HRMS (70 eV, EI+): m/z calcd for C30H2OD8BNO2: 453.2715, found: 453.

Elemental Analysis: C, 79%; H, 8%

### Synthesis Example 9: Synthesis of Compound R-1

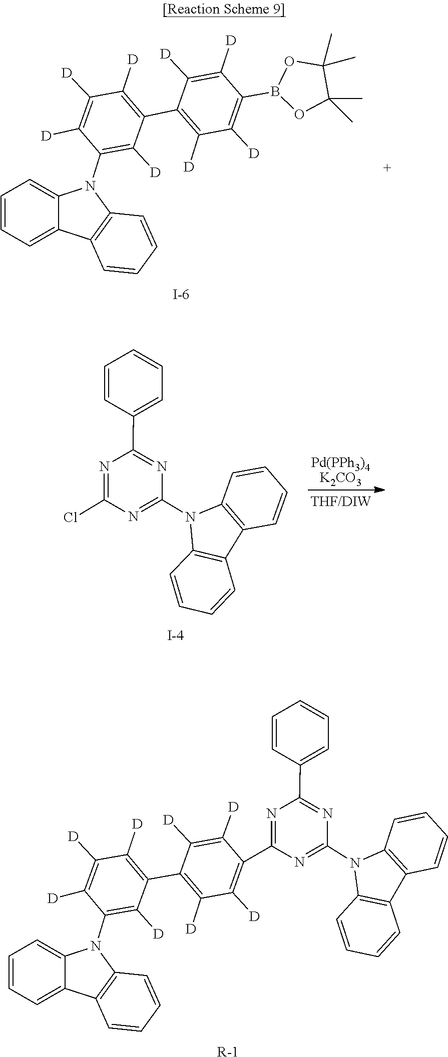

Compound R-1 (10 g, 71%) was obtained in the same manner as in Synthesis Example 2 except that Intermediate I-6 (10 g, 22 mmol) and Intermediate I-4 (7.9 g, 22 mmol) were used.

HRMS (70 eV, EI+): m/z calcd for C45H21D8N5: 647.2925, found: 647.

Elemental Analysis: C, 83%; H, 6%

### Synthesis Example 10: Synthesis of Intermediate I-7

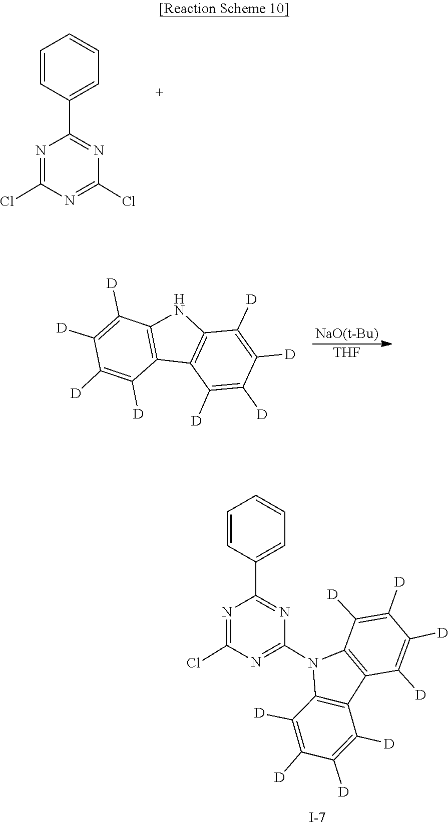

Intermediate I-7 (50 g, 80%) was obtained in the same manner as in Synthesis Example 1 except that 2,4-dichloro-6-phenyl-1,3,5-triazine (58 g, 256 mmol) and 9H-carbazole-1,2,3,4,5,6,7,8-d8 (30 g, 171 mmol) were used.

HRMS (70 eV, EI+): m/z calcd for C21H5D8ClN4: 364.1331, found: 364.

Elemental Analysis: C, 69%; H, 6%

### Synthesis Example 11: Synthesis of Intermediate I-8

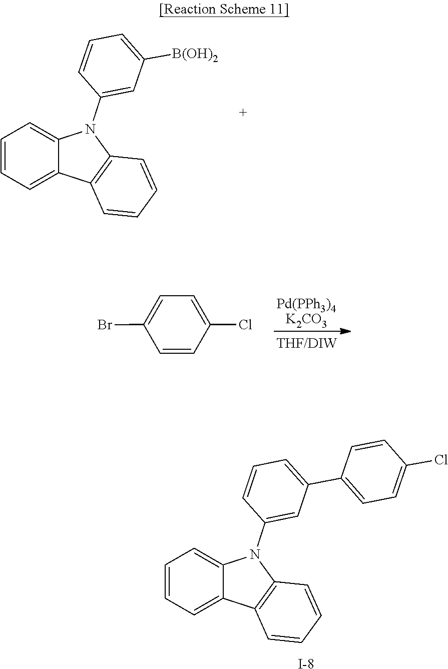

Intermediate I-8 (30 g, 81%) was obtained in the same manner as in Synthesis Example 2 except that (3-(9H-carbazol-9-yl)phenyl)boronic acid (30 g, 104 mmol) and 1-bromo-4-chlorobenzene (20 g, 104 mmol) were used.

HRMS (70 eV, EI+): m/z calcd for C24H16ClN: 353.0971, found: 353.

Elemental Analysis: C, 81%; H, 5%

### Synthesis Example 12: Synthesis of Intermediate I-9

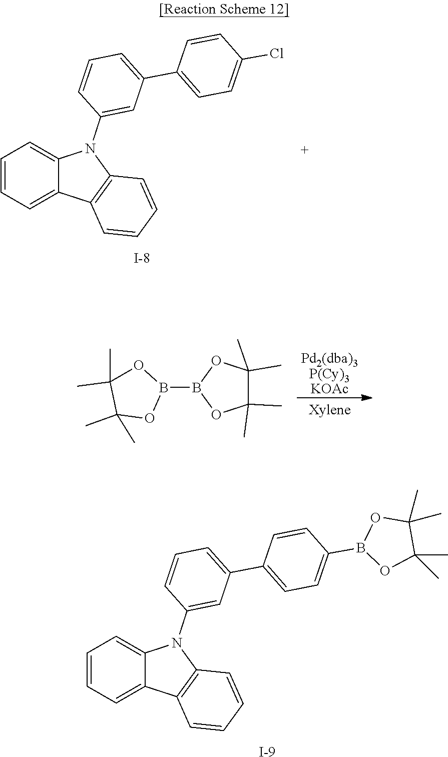

Intermediate I-9 (30 g, 79%) was obtained in the same manner as in Synthesis Example 3 except that Intermediate I-8 (30 g, 85 mmol) instead of Intermediate I-2 was used.

HRMS (70 eV, EI+): m/z calcd for C30H28BNO2: 445.2213, found: 445.

Elemental Analysis: C, 81%; H, 6%

### Synthesis Example 13: Synthesis of Compound R-2

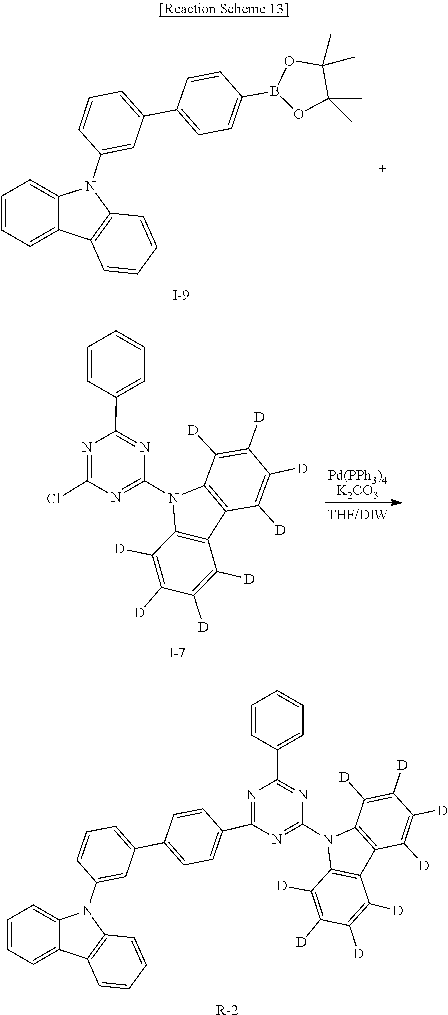

Compound R-2 (10 g, 71%) was obtained in the same manner as in Synthesis Example 2 except that Intermediate I-9 (10 g, 22 mmol) and Intermediate I-7 (8.2 g, 22 mmol) were used.

HRMS (70 eV, EI+): m/z calcd for C45H21D8N5: 647.2925, found: 647.

Elemental Analysis: C, 83%; H, 6%

### Synthesis Example 14: Synthesis of Intermediate I-10

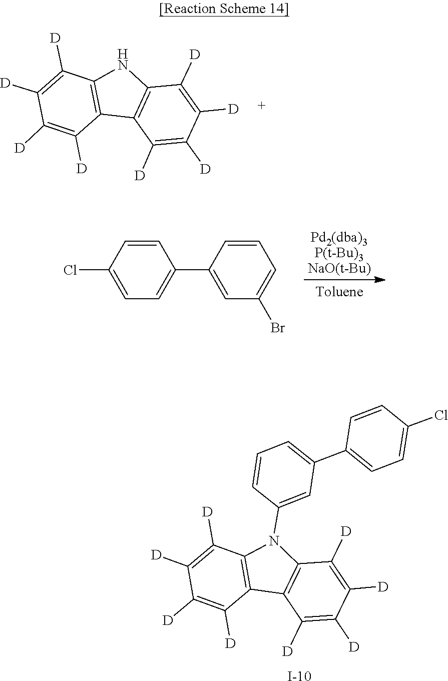

Under a nitrogen atmosphere, 9H-carbazole-1,2,3,4,5,6,7,8-d8 (30 g, 171 mmol) was dissolved in 0.3 L of toluene, and 3-bromo-4′-chloro-1,1′-biphenyl (55 g, 205 mmol), tris(dibenzylideneacetone) dipalladium (0) (1.56 g, 1.71 mmol), tris-tert butylphosphine (1.73 g, 8.55 mmol), and sodium tert-butoxide (19.7 g, 205 mmol) were sequentially added thereto and then, heated under reflux at 110° C. for 12 hours. When a reaction was completed, after adding water to the reaction solution, the obtained mixture was extracted with dichloromethane (DCM), treated with magnesium sulfate anhydrous to remove moisture, filtered, and concentrated under a reduced pressure. The obtained residue was separated and purified through flash column chromatography, obtaining Intermediate I-10 (50 g, 81%).

HRMS (70 eV, EI+): m/z calcd for C24H8D8ClN: 361.1473, found: 361.

Elemental Analysis: C, 80%; H, 7%

### Synthesis Example 15: Synthesis of Intermediate I-11

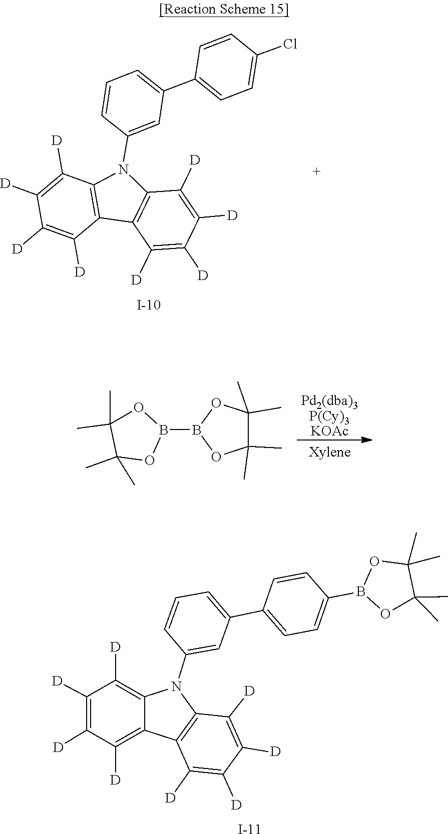

Intermediate I-11 (50 g, 80%) was obtained in the same manner as in Synthesis Example 3 except that Intermediate I-10 (50 g, 138 mmol) instead of Intermediate I-2 was used.

HRMS (70 eV, EI+): m/z calcd for C30H2OD8BNO2: 453.2715, found: 453.

Elemental Analysis: C, 79%; H, 8%

### Synthesis Example 16: Synthesis of Compound R-3

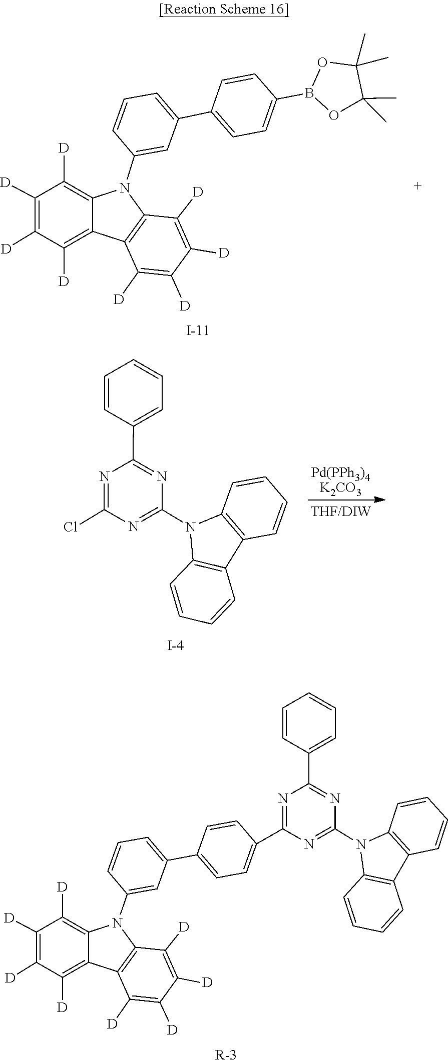

Compound R-3 (20 g, 70%) was obtained in the same manner as in Synthesis Example 2 except that Intermediate I-11 (20 g, 44 mmol) and Intermediate I-4 (16.5 g, 44 mmol) were used.

HRMS (70 eV, EI+): m/z calcd for C45H21D8N5: 647.2925, found: 647.

Elemental Analysis: C, 83%; H, 6%

### Synthesis Example 17: Synthesis of Intermediate I-12

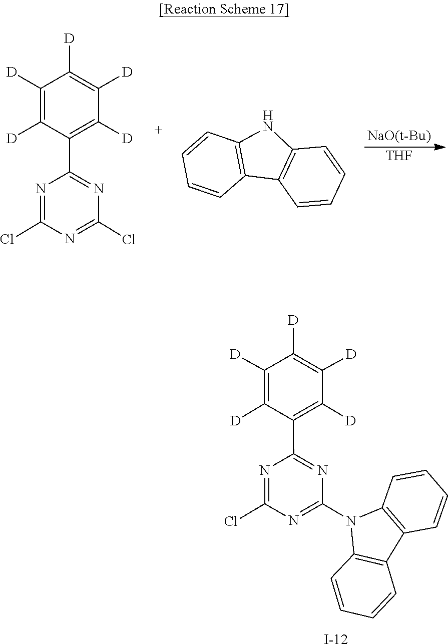

Intermediate I-12 (55 g, 85%) was obtained in the same manner as in Synthesis Example 1 except that 2,4-dichloro-6-(phenyl-d5)-1,3,5-triazine (62.2 g, 269 mmol) and 9H-carbazole (30 g, 179 mmol) were used.

HRMS (70 eV, EI+): m/z calcd for C21H8D5ClN4: 361.1143, found: 361.

Elemental Analysis: C, 70%; H, 5%

### Synthesis Example 18: Synthesis of Compound R-4

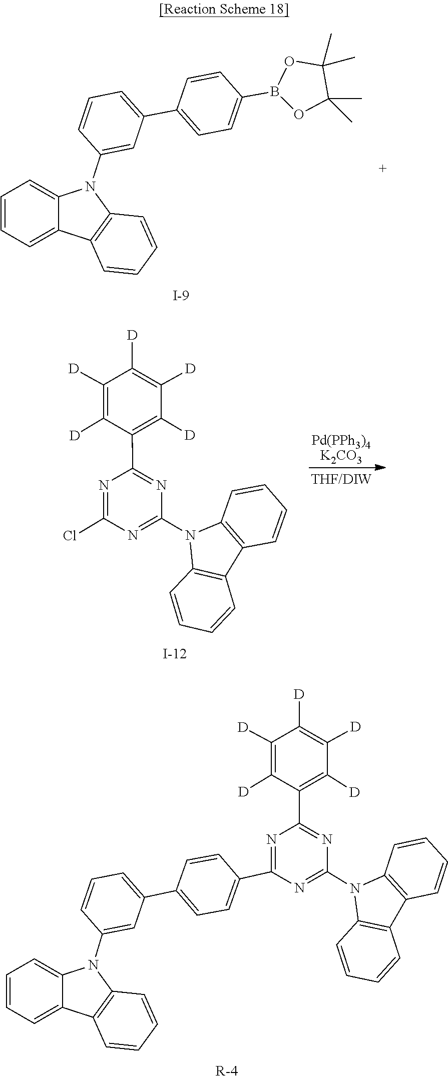

Compound R-4 (10 g, 70%) was obtained in the same manner as in Synthesis Example 2 except that Intermediate I-9 (10 g, 22 mmol) and Intermediate I-12 (7.96 g, 22 mmol) were used.

HRMS (70 eV, EI+): m/z calcd for C45H24D5N5: 644.2737, found: 644.

Elemental Analysis: C, 84%; H, 5%

(Preparation of Second Compound)

### Synthesis Example 19: Synthesis of Compound B-234

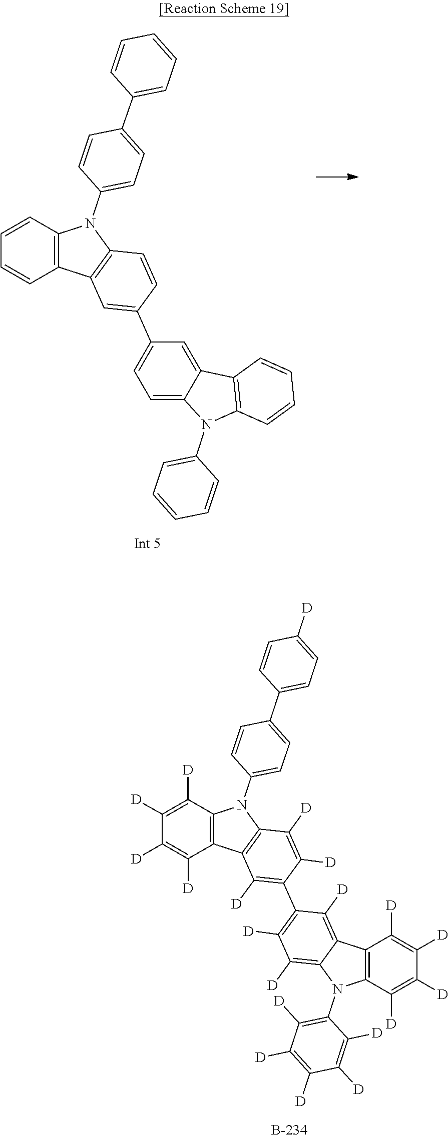 [SMILES: c1ccc(-c2ccc(-n3c4ccccc4c4cc(-c5ccc6c(c5)c5ccccc5n6-c5ccccc5)ccc43)cc2)cc1 ; [2H]c1ccc(-c2ccc(-n3c4c([2H])c([2H])c([2H])c([2H])c4c4c([2H])c(-c5c([2H])c([2H])c6c(c5[2H])c5c([2H])c([2H])c([2H])c([2H])c5n6-c5c([2H])c([2H])c([2H])c([2H])c5[2H])c([2H])c([2H])c43)cc2)cc1]

1st step: Synthesis of Compound Int 5

Compound Int 5 was synthesized by a method disclosed in Korean Laid-Open Publication No. 2016-0049842.

2nd step: Synthesis of Compound B-234

30 g (0.0535 mol) of Compound Int 5, and 40 g (0.267 mol) of trifluoromethanesulfonic acid were added to 282 g (3.35 mol) of D₆-benzene and then, stirred at 10° C. for 24 hours. Subsequently, purified water was added thereto and then, neutralized with a saturated K₃PO₄ solution. An organic layer therefrom was concentrated and column-purified, obtaining 18 g of Compound B-234 (white solid, LC-Mass Mz 578.79, C₄₂H₁₀D₁₈N₂).

(Manufacture of Organic Light Emitting Diode)

### Example 1

A glass substrate coated with ITO (indium tin oxide) was washed with distilled water and ultrasonic waves. After washing with the distilled water, the glass substrate was ultrasonically washed with isopropyl alcohol, acetone, or methanol, and dried and then, moved to a plasma cleaner, cleaned by using oxygen plasma for 10 minutes, and moved to a vacuum depositor. This obtained ITO transparent electrode was used as an anode, Compound A doped with 3% NDP-9 (available from Novaled) was vacuum-deposited on the ITO substrate to form a 100 Å-thick hole injection layer, and Compound A was deposited to on the hole injection layer to form a 1,350 Å-thick hole transport layer. On the hole transport layer, 350 Å-thick hole transport auxiliary layer was formed by depositing Compound B. On the hole transport auxiliary layer, 400 Å-thick light emitting layer was formed by vacuum-depositing Compound A-3 obtained in Synthesis Example 4 as a host, and doping 7 wt % of PhGD as a dopant. Subsequently, Compound C was deposited on the light emitting layer to form a 50 Å-thick electron transport auxiliary layer, and Compound D and LiQ were simultaneously vacuum-deposited at a weight ratio of 1:1 to form a 300 Å-thick electron transport layer. LiQ (15 Å) and Al (1,200 Å) were sequentially vacuum-deposited on the electron transport layer to form a cathode, thereby manufacturing an organic light emitting diode.

ITO/Compound A (3% NDP-9 doping, 100 Å)/Compound A (1,350 Å) /Compound B (350 Å)/EML [93 wt % of host (Compound A-3): 7 wt % of PhGD] (400 Å)/Compound C (50 Å)/Compound D:LiQ (300 Å)/LiQ (15 Å)/Al (1,200 Å).

Compound A: N-(biphenyl-4-yl)-9,9-dimethyl-N-(4-(9-phenyl-9H-carbazol-3-yl)phenyl)-9H-fluoren-2-amine

Compound B: N-[4-(4-Dibenzofuranyl)phenyl]-N-[4-(9-phenyl-9H-fluoren-9-yl)phenyl][1,1′-biphenyl]-4-amine

Compound C: 2,4-Diphenyl-6-(4′,5′,6′-triphenyl[1,1′:2′,1″:3″,1′″:3′″,1″″-quinquephenyl]-3″″-1,3,5-triazine

Compound D: 2-(1,1′-Biphenyl-4-yl)-4-(9,9-diphenylfluoren-4-yl)-6-phenyl-1,3,5-triazine

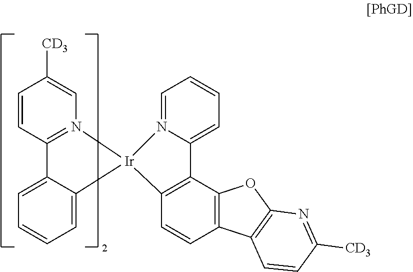

### Example 2

A glass substrate coated with ITO (Indium tin oxide) was washed with distilled water and ultrasonic waves. After washing with the distilled water, the glass substrate was ultrasonically washed with isopropyl alcohol, acetone, or methanol, and dried and then, moved to a plasma cleaner, cleaned by using oxygen plasma for 10 minutes, and moved to a vacuum depositor. This obtained ITO transparent electrode was used as an anode, Compound A doped with 3% NDP-9 (available from Novaled) was vacuum-deposited on the ITO substrate to form a 100 Å-thick hole injection layer, and Compound A was deposited to on the hole injection layer to form a 1,350 Å-thick hole transport layer. On the hole transport layer, 350 Å-thick hole transport auxiliary layer was formed by depositing Compound E. On the hole transport auxiliary layer, 330 Å-thick light emitting layer was formed by simultaneously vacuum-depositing Compound A-3 obtained in Synthesis Example 4 and Compound B-234 obtained in Synthesis Example 19 as a host, and doping 10 wt % of PhGD as a dopant. Subsequently, Compound F was deposited on the light emitting layer to form a 50 Å-thick electron transport auxiliary layer, and Compound G and LiQ were simultaneously vacuum-deposited at a ratio of 1:1 to form a 300 Å-thick electron transport layer. LiQ (15 Å) and Al (1,200 Å) were sequentially vacuum-deposited on the electron transport layer to form a cathode, thereby manufacturing an organic light emitting diode.

ITO/Compound A (3% NDP-9 doping, 100 Å)/Compound A (1,350 Å) /Compound E (350 Å)/EML [90 wt % of host (Compound A-3: Compound B-234=4:6 w/w): 10 wt % of PhGD] (330 Å)/Compound F (50 Å)/Compound G:LiQ (300 Å) /LiQ (15 Å)/Al (1,200 Å).

Compound A: N-(biphenyl-4-yl)-9,9-dimethyl-N-(4-(9-phenyl-9H-carbazol-3-yl)phenyl)-9H-fluoren-2-amine

Compound E: N,N-bis(9,9-dimethyl-9H-fluoren-4-yl)-9,9-spirobi(fluorene)-2-amine

Compound F: 2-[3′-(9,9-Dimethyl-9H-fluoren-2-yl)[1,1′-biphenyl]-3-yl]-4,6-diphenyl-1,3,5-triazine

Compound G: 2-[4-[4-(4′-Cyano-1,1′-biphenyl-4-yl)-1-naphthyl]phenyl]-4,6-diphenyl-1,3,5-triazine

### Example 3, Example 4 and Comparative Example 1 to Comparative Example 9

The diodes according to Examples 3, 4, and Comparative Examples 1 to 9 were manufactured in the same manner as in Example 1, except that the host and composition were changed as shown in Tables 1 and 2.

Evaluation

(1) Measurement of Current Density Change Depending on Voltage Change

The obtained organic light emitting diodes were measured regarding a current value flowing in the unit device, while increasing the voltage from 0 V to 10 V using a current-voltage meter (Keithley 2400), and the measured current value was divided by area to provide the results.

(2) Measurement of Luminance Change Depending on Voltage Change

Luminance was measured by using a luminance meter (Minolta Cs-1000A), while the voltage of the organic light emitting diodes was increased from 0 V to 10 V.

(3) Measurement of Luminous Efficiency

Luminous efficiency (cd/A) at the same current density (10 mA/cm²) were calculated by using the luminance and current density from the items (1) and (2).

Relative values relative to the luminous efficiency of Comparative Example 2 are shown in Table 1.

Relative values relative to the luminous efficiency of Comparative Example 6 are shown in Table 2.

(4) Measurement of Life-Span

T90 life-spans of the organic light emitting diodes were measured as a time when their luminance decreased down to 90% relative to the initial luminance (cd/m²) after emitting light with 24,000 cd/m² as the initial luminance (cd/m²) and measuring their luminance decreases depending on a time with a Polanonix life-span measurement system.

Relative values relative to the T90 life-span of Comparative Example 2 are shown in Table 1.

Relative values relative to the T90 life-span of Comparative Example 6 are shown in Table 2.

(5) Measurement of Driving Voltage

Driving voltages of each diode at 15 mA/cm² were measured by using a current-voltage meter (Keithley 2400).

Relative values relative to the Driving Voltage of Comparative Example 2 are shown in Table 1.

TABLE 1

| | | Driving | Luminous | |
|---|---|---|---|---|
| | | voltage | efficiency | Life-spanT90 |
| | Host | (%) | (%) | (%) |
| Example 1 | A-3 | 99% | 102% | 118% |
| Example 3 | A-21 | 98% | 101% | 115% |
| Comparative | R-1 | 101% | 101% | 99% |
| Example 1 | | | | |
| Comparative | R-2 | 100% | 100% | 100% |
| Example 2 | | | | |
| Comparative | R-3 | 99% | 99% | 99% |
| Example 3 | | | | |
| Comparative | R-4 | 100% | 101% | 102% |
| Example 4 | | | | |

TABLE 2

| | | | Luminous | |
|---|---|---|---|---|
| | First | Second | efficiency | Life-spanT90 |
| | host | host | (%) | (%) |
| Example 2 | A-3 | B-234 | 102% | 115% |
| Example 4 | A-21 | B-234 | 101% | 112% |
| Comparative | R-1 | B-234 | 100% | 100% |
| Example 6 | | | | |
| Comparative | R-2 | B-234 | 98% | 103% |
| Example 7 | | | | |
| Comparative | R-3 | B-234 | 98% | 103% |
| Example 8 | | | | |
| Comparative | R-4 | B-234 | 99% | 104% |
| Example 9 | | | | |

Referring to Table 1, the organic light emitting diodes to which the compounds according to the Examples were applied exhibited significantly improved driving voltage, luminous efficiency, and life-span characteristics, compared to the organic light emitting diodes according to the Comparative Examples.

Referring to Table 2, luminous efficiency and life-span characteristics of the organic light emitting diodes to which the compositions of the Examples were applied were also improved, relative to the Comparative Examples.

In the case of the Examples, by substituting the heterocycle and linker responsible for the HOMO region with deuterium, the stability of the molecule was greatly increased, and intermolecular interaction was reduced, so that packing was well performed during device fabrication, and thus the life-span characteristics were greatly improved.

One or more embodiments may provide a compound for an organic optoelectronic device capable of realizing an organic optoelectronic device having high efficiency and long life-span.

An organic optoelectronic device having high efficiency and a long life-span may be realized.

Example embodiments have been disclosed herein, and although specific terms are employed, they are used and are to be interpreted in a generic and descriptive sense only and not for purpose of limitation. In some instances, as would be apparent to one of ordinary skill in the art as of the filing of the present application, features, characteristics, and/or elements described in connection with a particular embodiment may be used singly or in combination with features, characteristics, and/or elements described in connection with other embodiments unless otherwise specifically indicated. Accordingly, it will be understood by those of skill in the art that various changes in form and details may be made without departing from the spirit and scope of the present invention as set forth in the following claims.

What is claimed is:

1. A compound for an organic optoelectronic device, the compound being represented by Chemical Formula 1: 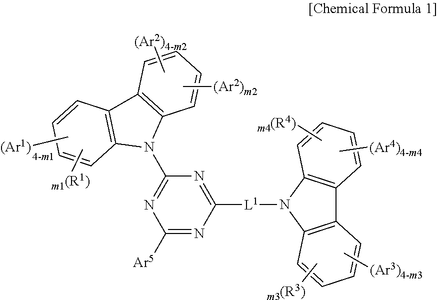
wherein, in Chemical Formula 1,
R¹ to R⁴ and Ar¹ to Ar⁴ are each independently hydrogen, deuterium, a cyano group, a halogen, a substituted or unsubstituted amine group, a substituted or unsubstituted C1 to C30 alkyl group, a substituted or unsubstituted C6 to C30 aryl group, a substituted or unsubstituted C2 to C30 heterocyclic group, or a combination thereof, provided that at least one of R¹ to R⁴ is deuterium, a C1 to C30 alkyl group substituted with one or more deuterium, a C6 to C30 aryl group substituted with one or more deuterium, or a C2 to C30 heterocyclic group substituted with one or more deuterium,
Ar⁵ is a substituted or unsubstituted C6 to C30 aryl group, a substituted or unsubstituted dibenzofuranyl group, or a substituted or unsubstituted dibenzothiophenyl group,
L¹ is a phenylene group substituted with one or more deuterium or a biphenylene group substituted with one or more deuterium, and
m1 to m4 are each independently an integer of 1 to 4.

2. The compound for an organic optoelectronic device as claimed in claim 1, wherein at least one of R¹ and R² is deuterium, a C1 to C30 alkyl group substituted with one or more deuterium, a C6 to C30 aryl group substituted with one or more deuterium, or a C2 to C30 heterocyclic group substituted with one or more deuterium.

3. The compound for an organic optoelectronic device as claimed in claim 1, wherein R¹ and R² are each independently deuterium, a C1 to C30 alkyl group substituted with one or more deuterium, a C6 to C30 aryl group substituted with one or more deuterium, or a C2 to C30 heterocyclic group substituted with one or more deuterium.

4. The compound for an organic optoelectronic device as claimed in claim 1, wherein at least one of R³ and R⁴ is deuterium, a C1 to C30 alkyl group substituted with one or more deuterium, a C6 to C30 aryl group substituted with one or more deuterium, or a C2 to C30 heterocyclic group substituted with one or more deuterium.

5. The compound for an organic optoelectronic device as claimed in claim 1, wherein R³ and R⁴ are each independently deuterium, a C1 to C30 alkyl group substituted with one or more deuterium, a C6 to C30 aryl group substituted with one or more deuterium, or a C2 to C30 heterocyclic group substituted with one or more deuterium.

6. The compound for an organic optoelectronic device as claimed in claim 1, wherein R¹ to R⁴ are each independently deuterium, a C1 to C30 alkyl group substituted with one or more deuterium, a C6 to C30 aryl group substituted with one or more deuterium, or a C2 to C30 heterocyclic group substituted with one or more deuterium.

7. The compound for an organic optoelectronic device as claimed in claim 1, wherein:
L¹ in Chemical Formula 1 is a linking group of Group I: 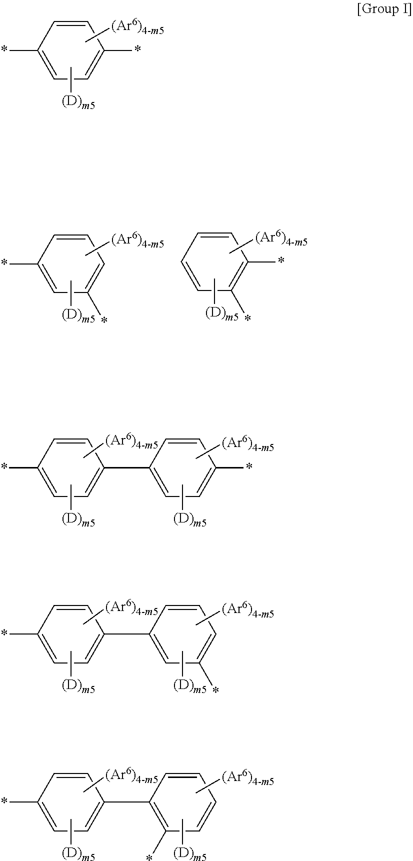
in Group I,
D is deuterium,
each Ar⁶ is independently hydrogen, deuterium, a cyano group, a substituted or unsubstituted C1 to C10 alkyl group, or a substituted or unsubstituted C6 to C12 aryl group, and
m5 is an integer of 1 to 4.

8. The compound for an organic optoelectronic device as claimed in claim 7, wherein m5 is an integer of 2 to 4.

9. The compound for an organic optoelectronic device as claimed in claim 7, wherein m5 is an integer of 3 or 4.

10. The compound for an organic optoelectronic device as claimed in claim 7, wherein m5 is 4.

11. The compound for an organic optoelectronic device as claimed in claim 1, wherein Ar⁵ is a substituted or unsubstituted phenyl group, a substituted or unsubstituted biphenyl group, a substituted or unsubstituted fluorenyl group, a substituted or unsubstituted dibenzofuranyl group, or a substituted or unsubstituted dibenzothiophenyl group.

12. The compound for an organic optoelectronic device as claimed in claim 1, wherein Ar⁵ is a phenyl group substituted with one or more deuterium, a biphenyl group substituted with one or more deuterium, a fluorenyl group substituted with one or more deuterium, a dibenzofuranyl group substituted with one or more deuterium, or a dibenzothiophenyl group substituted with one or more deuterium.

13. A compound for an organic optoelectronic device, the compound being a compound of Group 1:

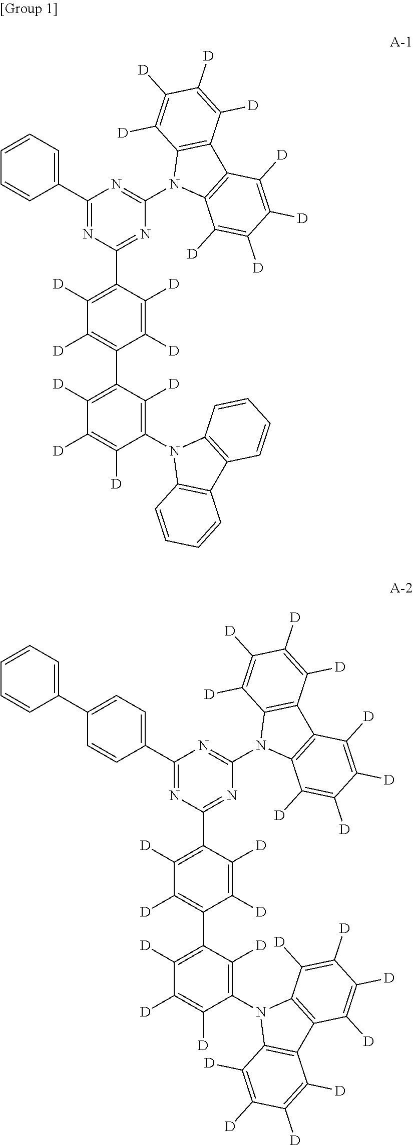 [SMILES: [2H]c1c([2H])c(-c2c([2H])c([2H])c(-c3nc(-c4ccccc4)nc(-n4c5c([2H])c([2H])c([2H])c([2H])c5c5c([2H])c([2H])c([2H])c([2H])c54)n3)c([2H])c2[2H])c([2H])c(-n2c3ccccc3c3ccccc32)c1[2H] ; [2H]c1c([2H])c(-c2c([2H])c([2H])c(-c3nc(-c4ccc(-c5ccccc5)cc4)nc(-n4c5c([2H])c([2H])c([2H])c([2H])c5c5c([2H])c([2H])c([2H])c([2H])c54)n3)c([2H])c2[2H])c([2H])c(-n2c3c([2H])c([2H])c([2H])c([2H])c3c3c([2H])c([2H])c([2H])c([2H])c32)c1[2H]]

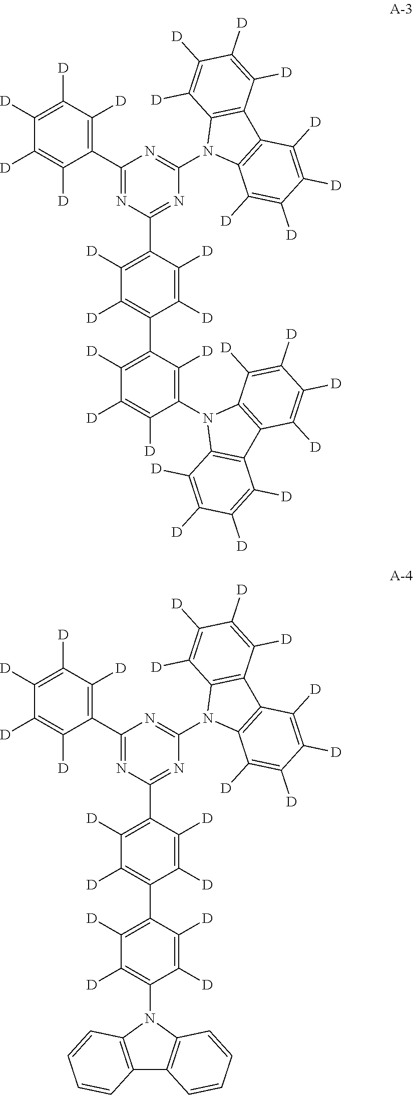

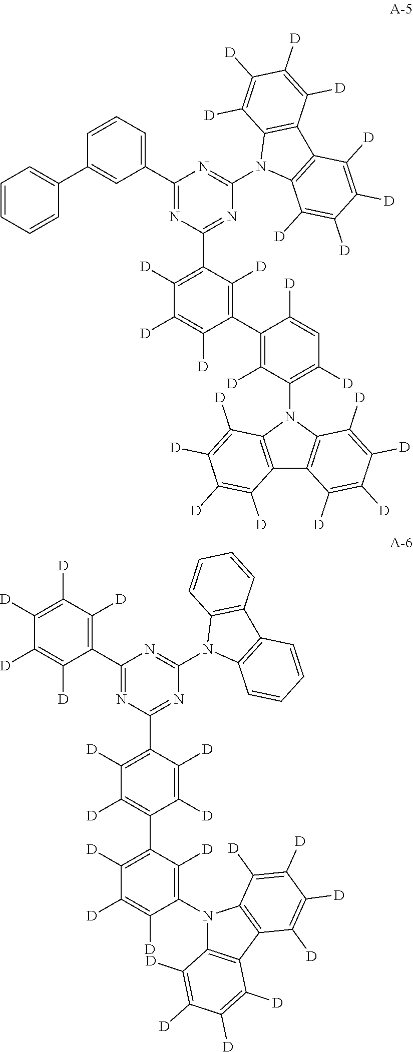

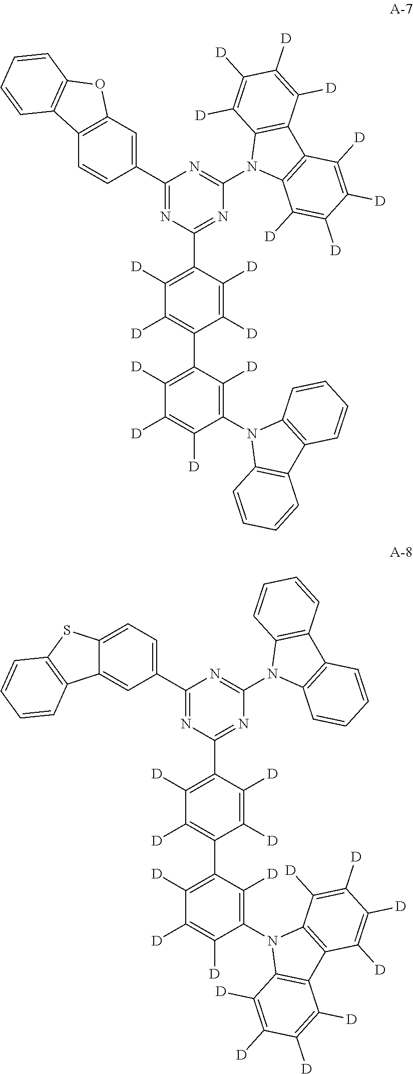 [SMILES: [2H]c1c([2H])c(-c2c([2H])c([2H])c(-c3nc(-c4ccc5c(c4)oc4ccccc45)nc(-n4c5c([2H])c([2H])c([2H])c([2H])c5c5c([2H])c([2H])c([2H])c([2H])c54)n3)c([2H])c2[2H])c([2H])c(-n2c3ccccc3c3ccccc32)c1[2H] ; [2H]c1c([2H])c(-c2c([2H])c([2H])c(-c3nc(-c4ccc5sc6ccccc6c5c4)nc(-n4c5ccccc5c5ccccc54)n3)c([2H])c2[2H])c([2H])c(-n2c3c([2H])c([2H])c([2H])c([2H])c3c3c([2H])c([2H])c([2H])c([2H])c32)c1[2H]]

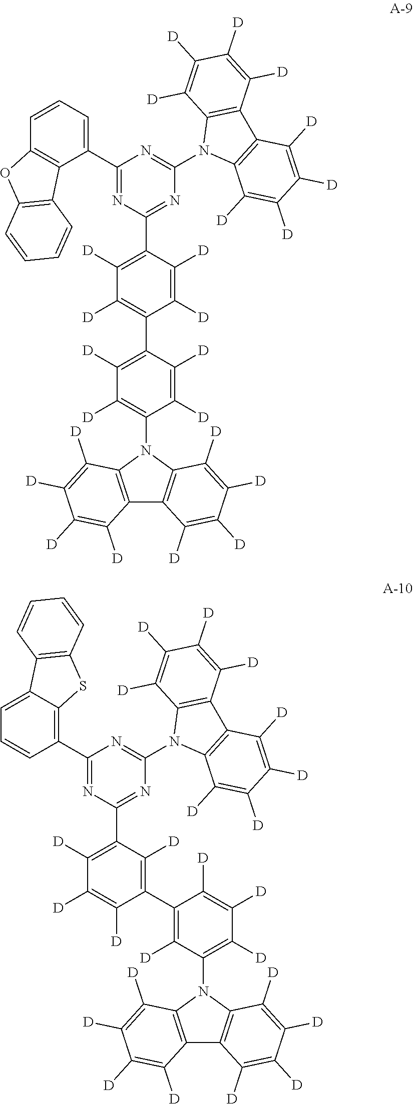 [SMILES: [2H]c1c([2H])c(-c2c([2H])c([2H])c(-n3c4c([2H])c([2H])c([2H])c([2H])c4c4c([2H])c([2H])c([2H])c([2H])c43)c([2H])c2[2H])c([2H])c([2H])c1-c1nc(-c2cccc3oc4ccccc4c23)nc(-n2c3c([2H])c([2H])c([2H])c([2H])c3c3c([2H])c([2H])c([2H])c([2H])c32)n1 ; [2H]c1c([2H])c(-c2nc(-c3cccc4c3sc3ccccc34)nc(-n3c4c([2H])c([2H])c([2H])c([2H])c4c4c([2H])c([2H])c([2H])c([2H])c43)n2)c([2H])c(-c2c([2H])c([2H])c([2H])c(-n3c4c([2H])c([2H])c([2H])c([2H])c4c4c([2H])c([2H])c([2H])c([2H])c43)c2[2H])c1[2H]]

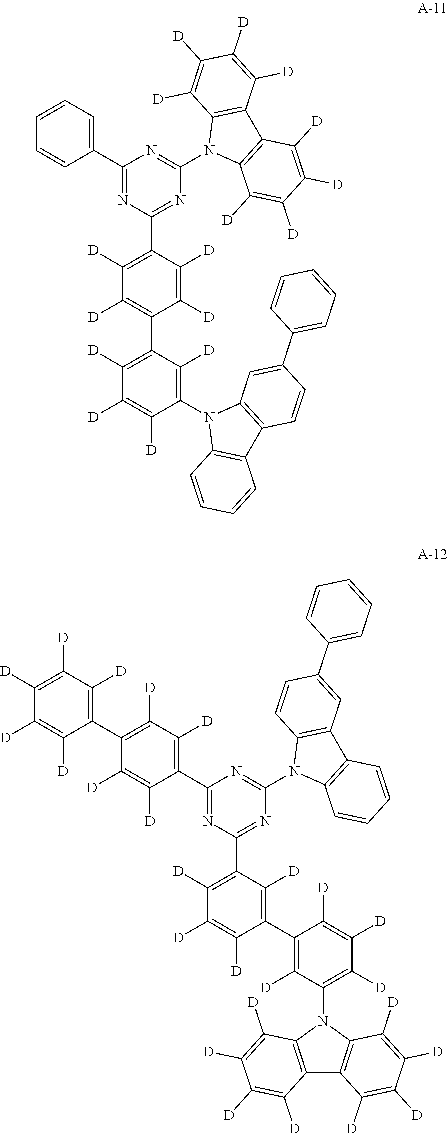 [SMILES: [2H]c1c([2H])c(-c2c([2H])c([2H])c(-c3nc(-c4ccccc4)nc(-n4c5c([2H])c([2H])c([2H])c([2H])c5c5c([2H])c([2H])c([2H])c([2H])c54)n3)c([2H])c2[2H])c([2H])c(-n2c3ccccc3c3ccc(-c4ccccc4)cc32)c1[2H] ; [2H]c1c([2H])c([2H])c(-c2c([2H])c([2H])c(-c3nc(-c4c([2H])c([2H])c([2H])c(-c5c([2H])c([2H])c([2H])c(-n6c7c([2H])c([2H])c([2H])c([2H])c7c7c([2H])c([2H])c([2H])c([2H])c76)c5[2H])c4[2H])nc(-n4c5ccccc5c5cc(-c6ccccc6)ccc54)n3)c([2H])c2[2H])c([2H])c1[2H]]

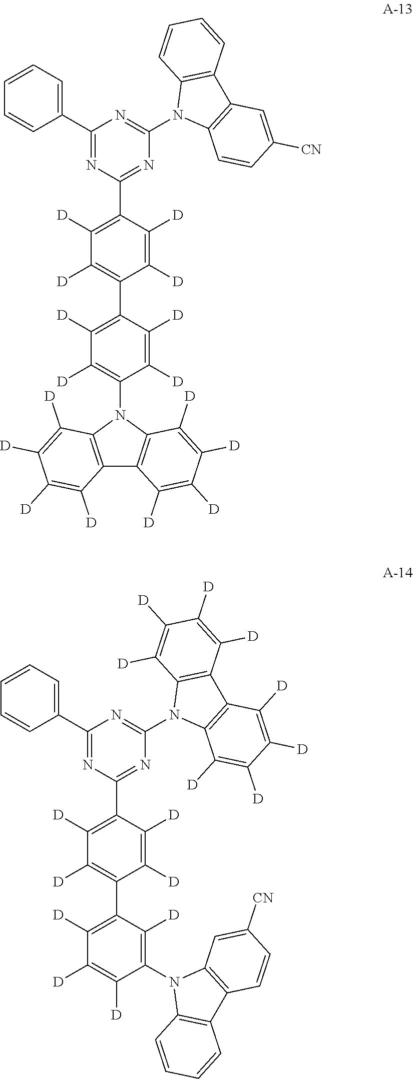

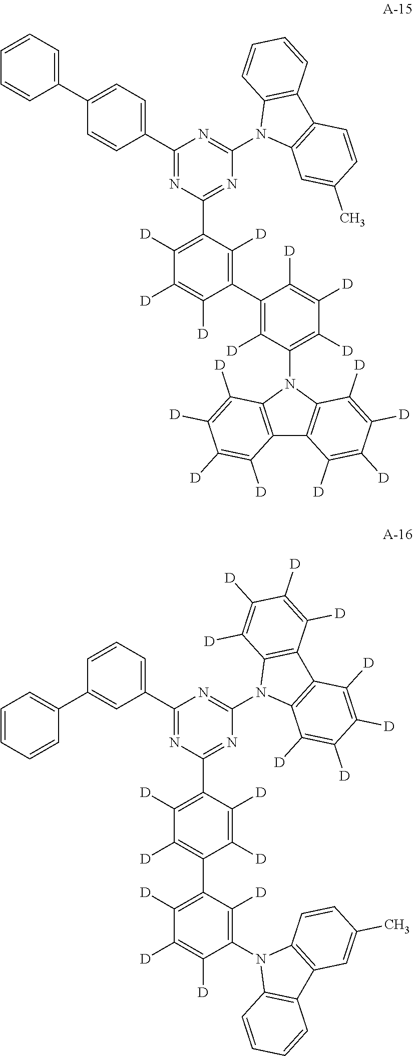 [SMILES: [2H]c1c([2H])c(-c2nc(-c3ccc(-c4ccccc4)cc3)nc(-n3c4ccccc4c4ccc(C)cc43)n2)c([2H])c(-c2c([2H])c([2H])c([2H])c(-n3c4c([2H])c([2H])c([2H])c([2H])c4c4c([2H])c([2H])c([2H])c([2H])c43)c2[2H])c1[2H] ; [2H]c1c([2H])c(-c2c([2H])c([2H])c(-c3nc(-c4cccc(-c5ccccc5)c4)nc(-n4c5c([2H])c([2H])c([2H])c([2H])c5c5c([2H])c([2H])c([2H])c([2H])c54)n3)c([2H])c2[2H])c([2H])c(-n2c3ccccc3c3cc(C)ccc32)c1[2H]]

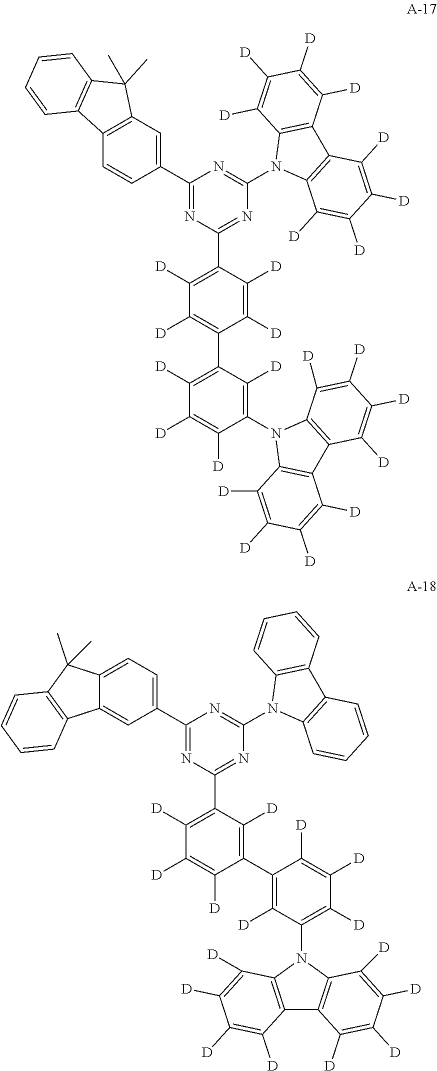 [SMILES: [2H]c1c([2H])c(-c2c([2H])c([2H])c(-c3nc(-c4ccc5c(c4)C(C)(C)c4ccccc4-5)nc(-n4c5c([2H])c([2H])c([2H])c([2H])c5c5c([2H])c([2H])c([2H])c([2H])c54)n3)c([2H])c2[2H])c([2H])c(-n2c3c([2H])c([2H])c([2H])c([2H])c3c3c([2H])c([2H])c([2H])c([2H])c32)c1[2H] ; [2H]c1c([2H])c(-c2nc(-c3ccc4c(c3)-c3ccccc3C4(C)C)nc(-n3c4ccccc4c4ccccc43)n2)c([2H])c(-c2c([2H])c([2H])c([2H])c(-n3c4c([2H])c([2H])c([2H])c([2H])c4c4c([2H])c([2H])c([2H])c([2H])c43)c2[2H])c1[2H]]

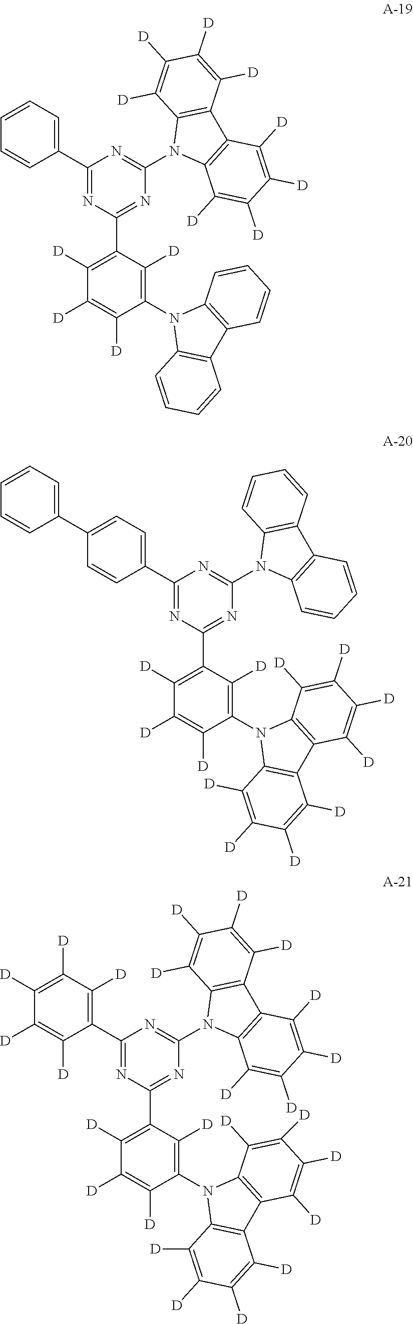

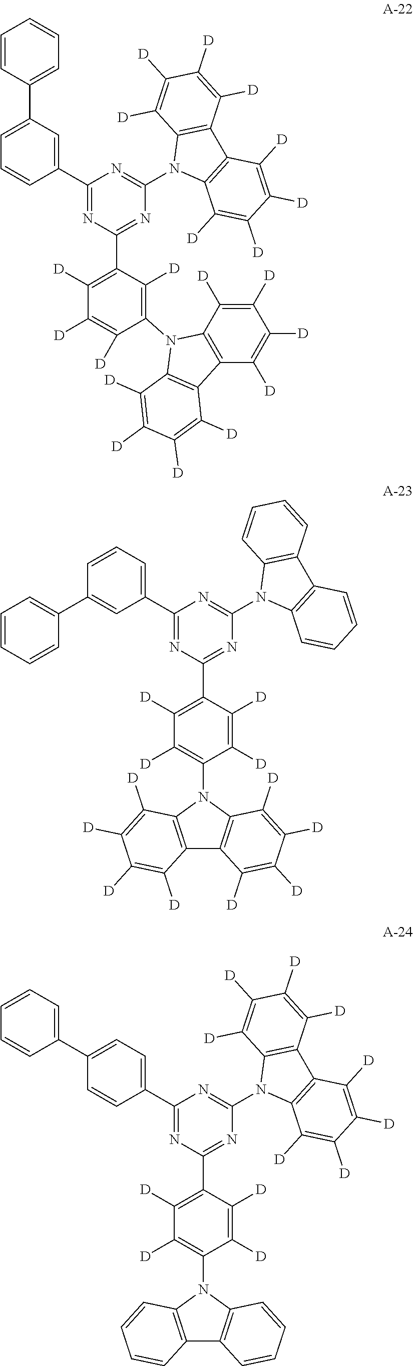

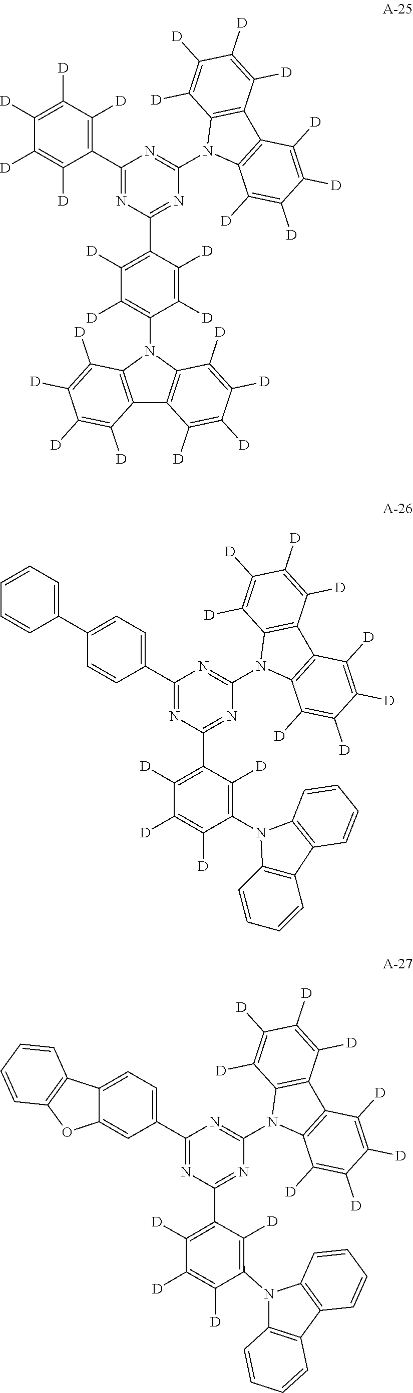

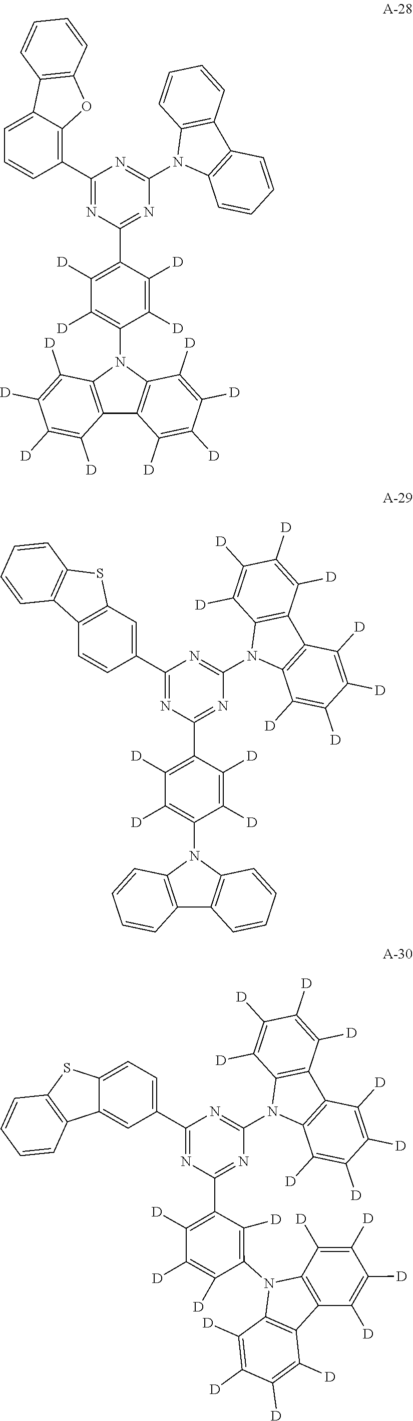

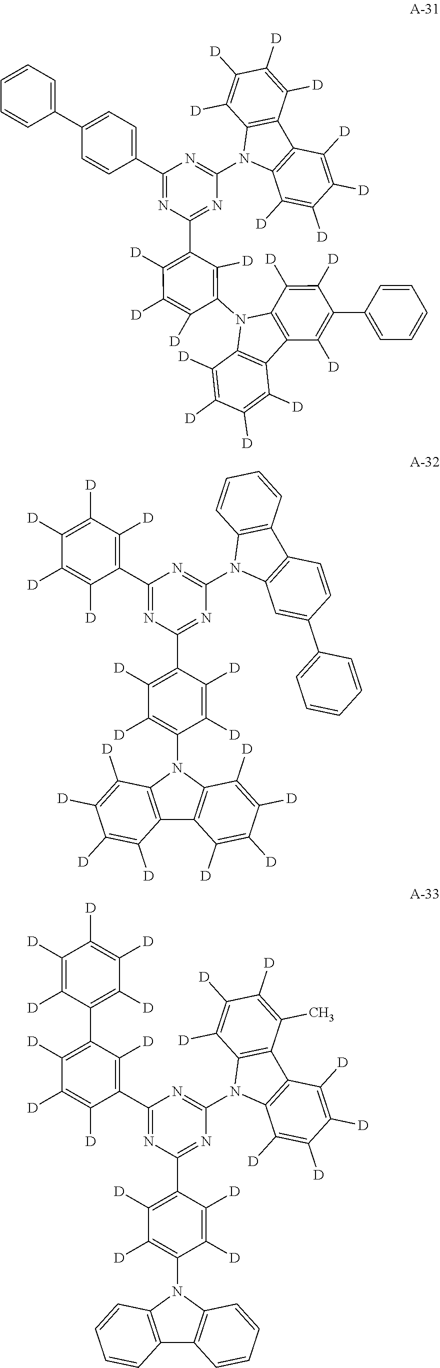

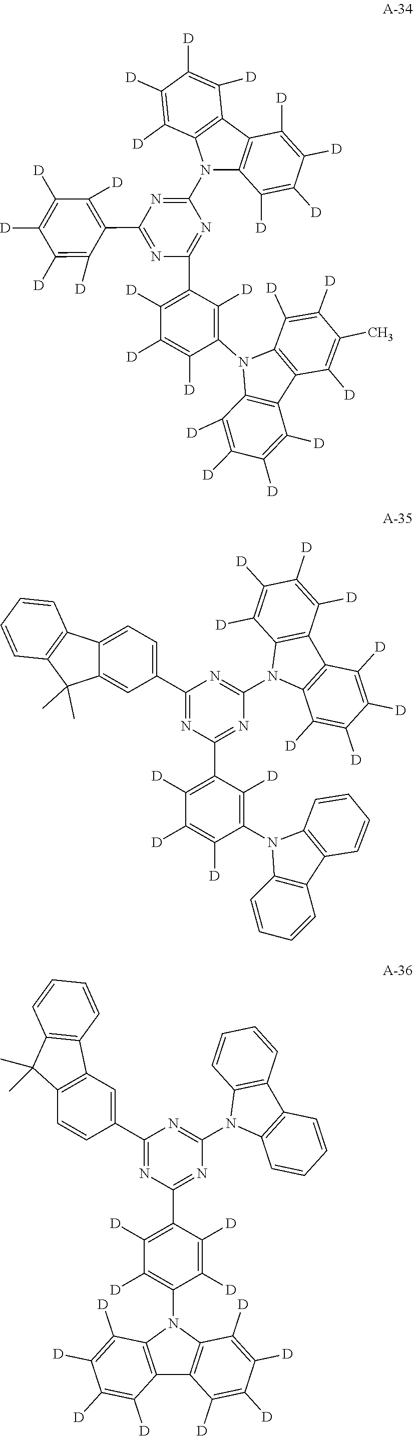

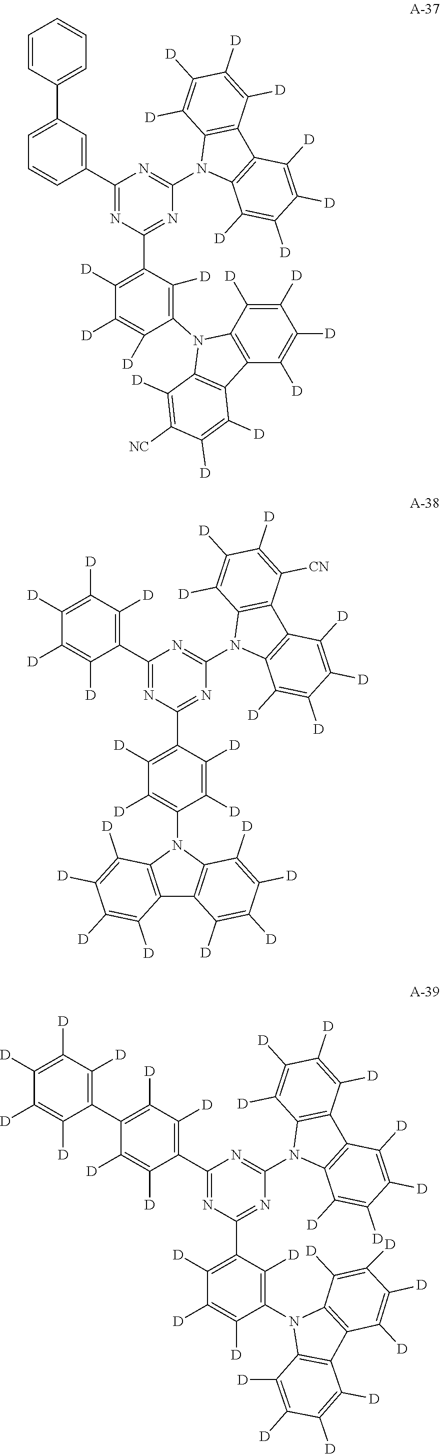

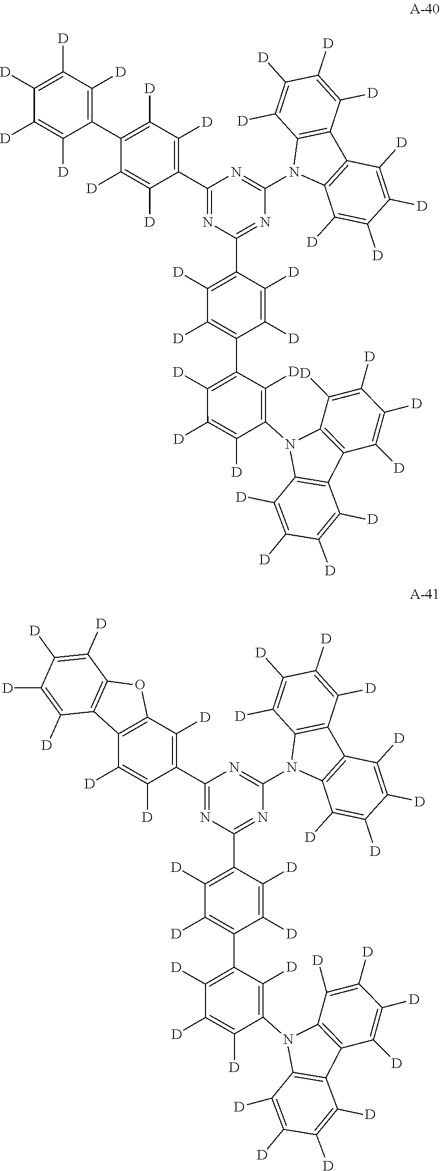

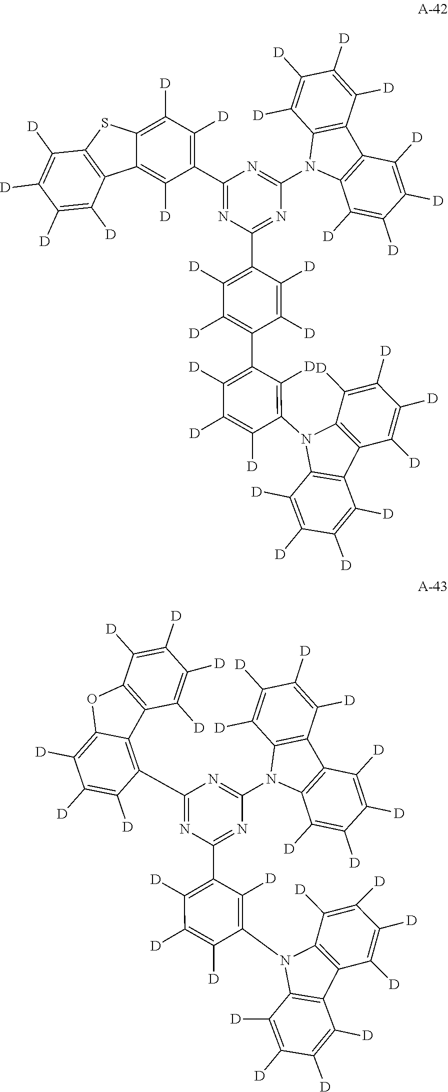

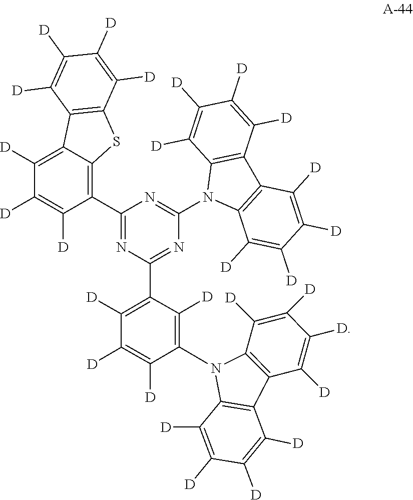

14. A composition for an organic optoelectronic device, the composition comprising:
a first compound; and
a second compound,
wherein:
the first compound is the compound for an organic optoelectronic device as claimed in claim 1, and
the second compound is represented by:
Chemical Formula 2; or
a combination of Chemical Formula 3 and Chemical Formula 4, 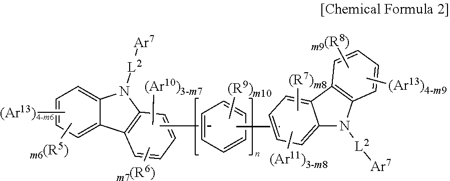
in Chemical Formula 2,
R⁵ to R⁹ and Ar⁹ to Ar¹² are each independently hydrogen, deuterium, a cyano group, a halogen, a substituted or unsubstituted amine group, a substituted or unsubstituted C1 to C30 alkyl group, a substituted or unsubstituted C6 to C30 aryl group or a substituted or unsubstituted C2 to C30 heterocyclic group,
Ar⁷ and Ar⁸ are each independently a substituted or unsubstituted C6 to C20 aryl group or a substituted or unsubstituted C2 to C30 heterocyclic group,
L² and L³ are each independently a single bond or a substituted or unsubstituted C6 to C20 arylene group,
m6, m9 and m10 are each independently an integer of 1 to 4,
m7 and m8 are each independently an integer of 1 to 3, and
n is an integer of 0 to 2, 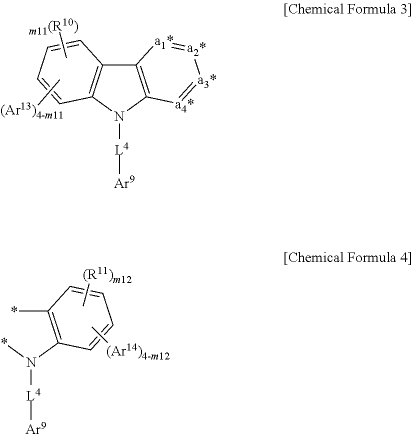
in Chemical Formula 3 and Chemical Formula 4,
two adjacent ones of a1* to a4* of Chemical Formula 3 are linking carbons linked at * of Chemical Formula 4, the remaining two of a1* to a4* of Chemical Formula 3, not linked at * of Chemical Formula 4, are C-L^{a}-R^{a},
L^{a}, L⁴, and L⁵ are each independently a single bond or a substituted or unsubstituted C6 to C20 arylene group,
R^{a}, R¹⁰, R¹¹, Ar¹³, and Ar¹⁴ are each independently hydrogen, deuterium, a cyano group, a halogen, a substituted or unsubstituted amine group, a substituted or unsubstituted C1 to C30 alkyl group, a substituted or unsubstituted C6 to C30 aryl group or a substituted or unsubstituted C2 to C30 heterocyclic group,
Ar⁹ and Ar¹⁰ are each independently a substituted or unsubstituted C6 to C20 aryl group or a substituted or unsubstituted C2 to C30 heterocyclic group, and
m11 and m12 are each independently an integer of 1 to 4.

15. The composition for an organic optoelectronic device as claimed in claim 14, wherein:
the second compound is represented by Chemical Formula 2,
Chemical Formula 2 is represented by Chemical Formula 2-8: 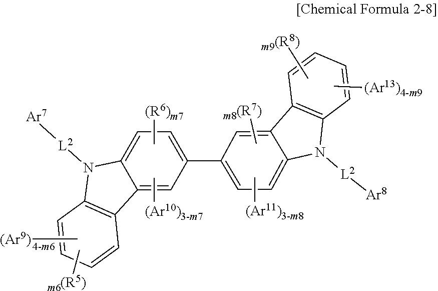
in Chemical Formula 2-8,
Ar⁹ to Ar¹² and R⁵ to R⁸ are each independently hydrogen, deuterium, or a substituted or unsubstituted C6 to C12 aryl group,
m6 and m9 are each independently an integer of 1 to 4,
m7 and m8 are each independently an integer of 1 to 3, and
moieties *-L²-Ar⁷ and *-L³-Ar⁸ are each independently a moiety of Group II, 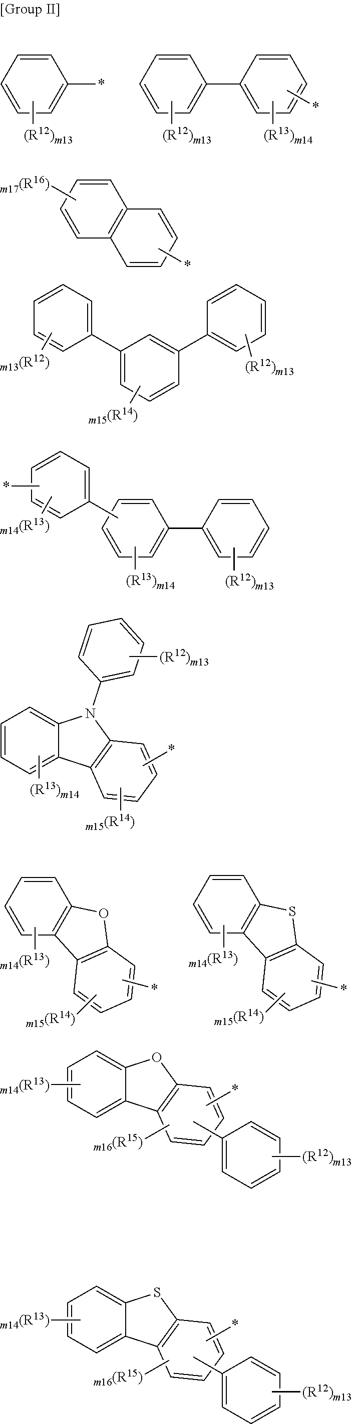 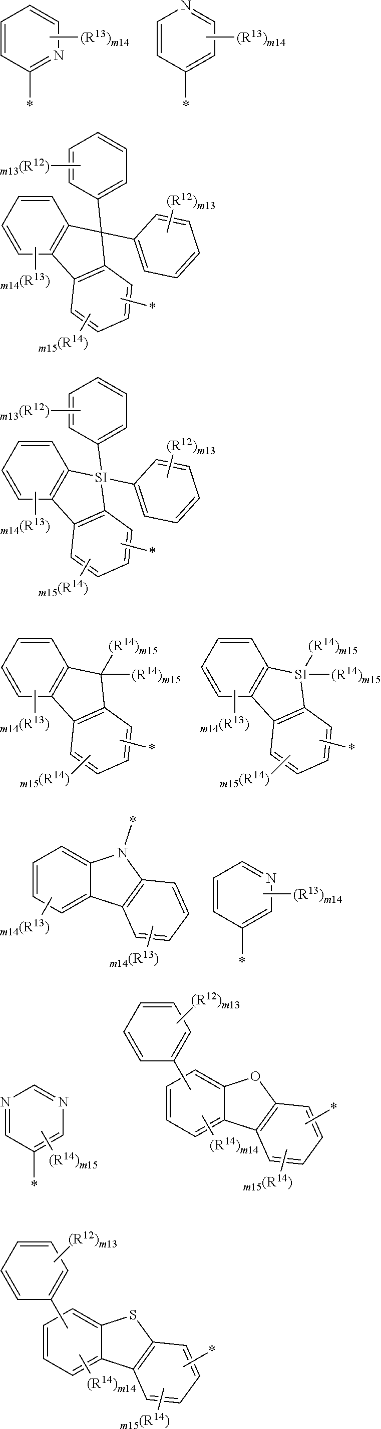 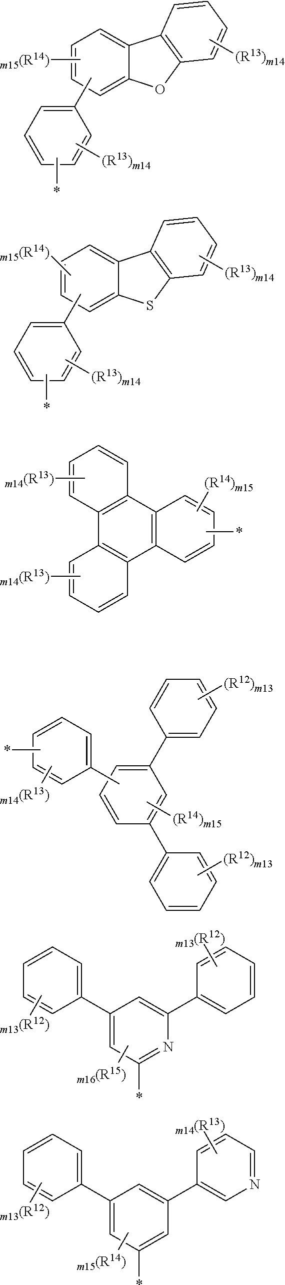 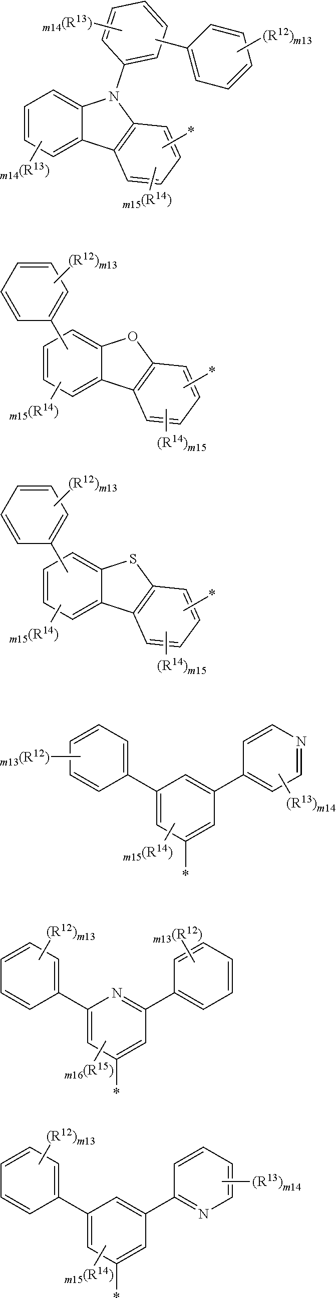
in Group II,
R¹² to R¹⁶ are each independently hydrogen, deuterium, a cyano group, a substituted or unsubstituted C1 to C10 alkyl group, or a substituted or unsubstituted C6 to C12 aryl group,
m13 is an integer of 1 to 5,
m14 is an integer of 1 to 4,
m15 is an integer of 1 to 3,
m16 is 1 or 2,
m17 is an integer of 1 to 7, and
* is a linking point.

16. The composition for an organic optoelectronic device as claimed in claim 14, wherein:
the second compound is represented by a combination of Chemical Formula 3 and Chemical Formula 4,
the combination of Chemical Formula 3 and Chemical Formula 4 is represented by Chemical Formula 3C, 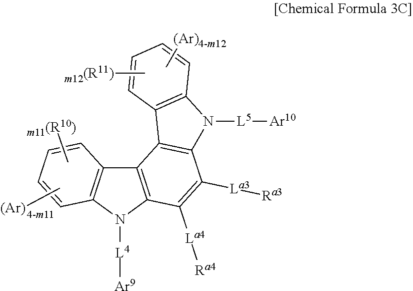
in Chemical Formula 3C,
L^{a3} and L^{a4} are a single bond,
Ar¹³, Ar¹⁴, R¹⁰, R¹¹, R^{a3}, and R^{a4} are each independently hydrogen, deuterium, or a substituted or unsubstituted C6 to C12 aryl group,
m11 and m12 are each independently an integer of 1 to 4, and
moieties *-L⁴-Ar⁹ and *-L⁵-Ar¹⁰ are each independently a moiety of Group II, 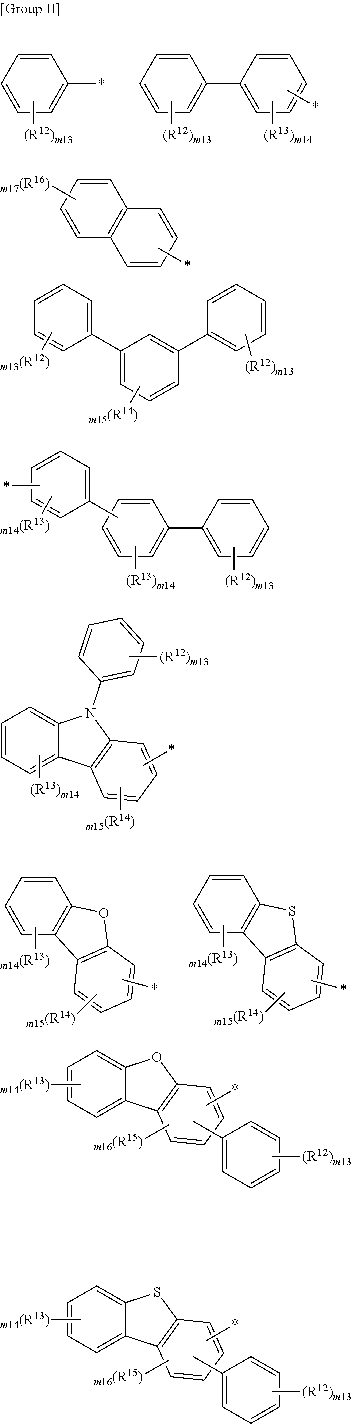 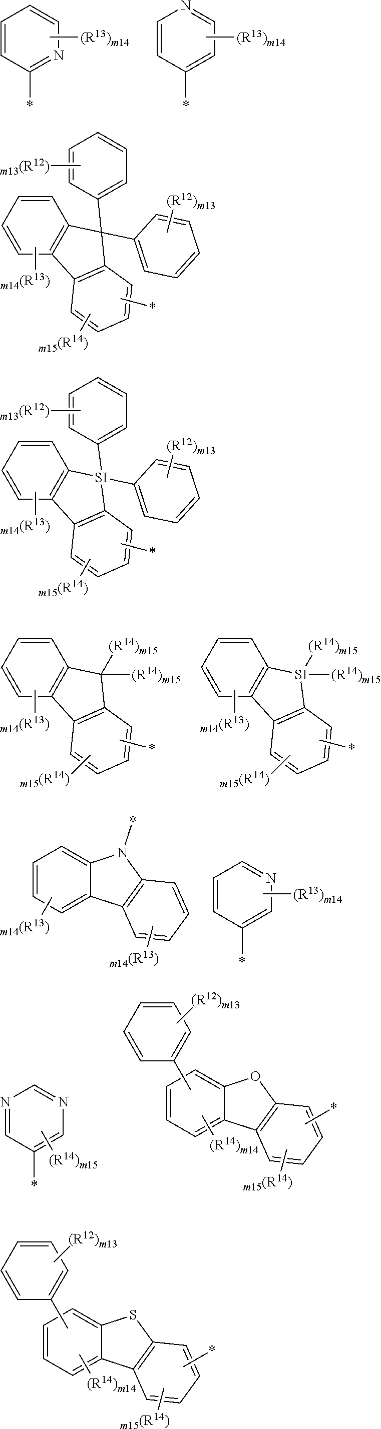 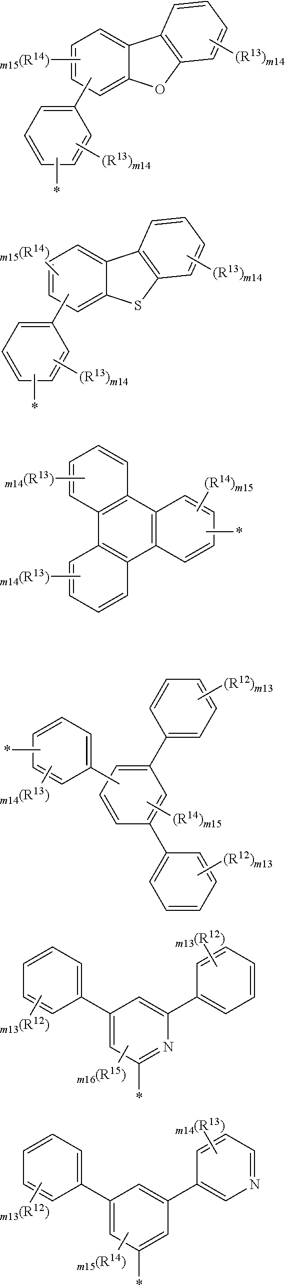 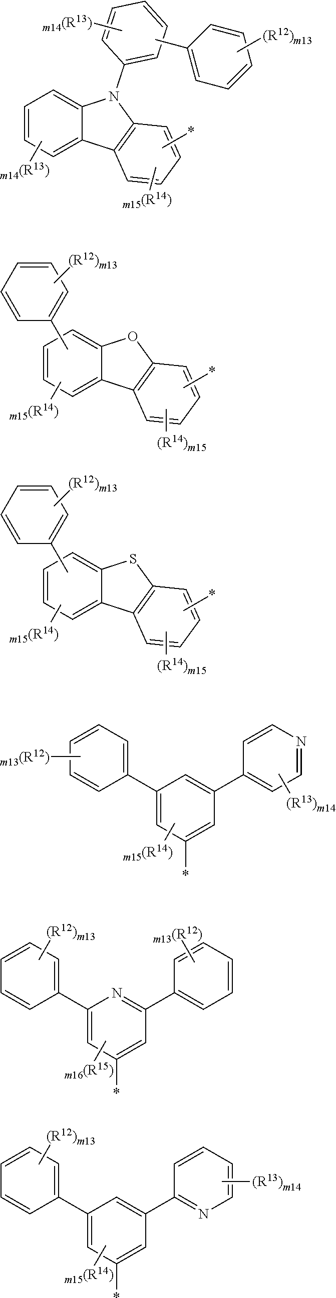
in Group II,
R¹² to R¹⁶ are each independently hydrogen, deuterium, a cyano group, a substituted or unsubstituted C1 to C10 alkyl group, or a substituted or unsubstituted C6 to C12 aryl group,
m13 is an integer of 1 to 5,
m14 is an integer of 1 to 4,
m15 is an integer of 1 to 3,
m16 is 1 or 2,
m17 is an integer of 1 to 7, and
* is a linking point.

17. An organic optoelectronic device, comprising:
an anode and a cathode facing each other, and
at least one organic layer between the anode and the cathode,
wherein the at least one organic layer includes the compound for an organic optoelectronic device as claimed in claim 1.

18. The organic optoelectronic device as claimed in claim 17, wherein
the at least one organic layer includes a light emitting layer, and
the light emitting layer includes the compound.

19. A display device comprising the organic optoelectronic device as claimed in claim 17.

20. An organic optoelectronic device, comprising:
an anode and a cathode facing each other, and
at least one organic layer between the anode and the cathode,
wherein the at least one organic layer includes the composition for an organic optoelectronic device as claimed in claim 14.

21. The organic optoelectronic device as claimed in claim 20, wherein
the at least one organic layer includes a light emitting layer, and
the light emitting layer includes the composition.

22. A display device comprising the organic optoelectronic device as claimed in claim 20.
